SEQUENCE LISTING (112 sequences)

SEQ ID NO: 1 - protein, 9 aa, Hepatitis B virus
ASRELVVSY

SEQ ID NO: 2 - protein, 11 aa, Hepatitis B virus
ENITSGFLGPL

SEQ ID NO: 3 - protein, 9 aa, Hepatitis B virus
FLLTRILTI

SEQ ID NO: 4 - protein, 11 aa, Hepatitis B virus
FSLTKILTIPQ

SEQ ID NO: 5 - protein, 12 aa, Hepatitis B virus
FSLTKILTIPQS

SEQ ID NO: 6 - protein, 9 aa, Hepatitis B virus
FVGLSPTVW

SEQ ID NO: 7 - protein, 10 aa, Hepatitis B virus
FVGLSPTVWL

SEQ ID NO: 8 - protein, 10 aa, Hepatitis B virus
GLSPTVWLSV

SEQ ID NO: 9 - protein, 9 aa, Hepatitis B virus
GMLPVCPLI

SEQ ID NO: 10 - protein, 9 aa, Hepatitis B virus
GMLPVCPLL

SEQ ID NO: 11 - protein, 11 aa, Hepatitis B virus
GSLPQEHIIQK

SEQ ID NO: 12 - protein, 11 aa, Hepatitis B virus
GSLPQEHIVQK

SEQ ID NO: 13 - protein, 11 aa, Hepatitis B virus
GTLPQEHIVHK

SEQ ID NO: 14 - protein, 11 aa, Hepatitis B virus
GTLPQEHIVQK

SEQ ID NO: 15 - protein, 11 aa, Hepatitis B virus
GVWIRTPPAYR

SEQ ID NO: 16 - protein, 9 aa, Hepatitis B virus
HLYSHPIIL

SEQ ID NO: 17 - protein, 9 aa, Hepatitis B virus
IASGLLGPL

SEQ ID NO: 18 - protein, 12 aa, Hepatitis B virus
IASGLLGPLLVL

SEQ ID NO: 19 - protein, 9 aa, Hepatitis B virus
ITSGFLGPL

SEQ ID NO: 20 - protein, 10 aa, Hepatitis B virus
ITSGFLGPLL

SEQ ID NO: 21 - protein, 12 aa, Hepatitis B virus
KILTIPQSLDSW

SEQ ID NO: 22 - protein, 10 aa, Hepatitis B virus
KPRKGMGTNL

SEQ ID NO: 23 - protein, 9 aa, Hepatitis B virus
KYTSFPWLL

SEQ ID NO: 24 - protein, 9 aa, Hepatitis B virus
LPETTVVRR

SEQ ID NO: 25 - protein, 9 aa, Hepatitis B virus
LPFRPTTGR

SEQ ID NO: 26 - protein, 9 aa, Hepatitis B virus
LPQEHIVHK

SEQ ID NO: 27 - protein, 9 aa, Hepatitis B virus
LPSDFFPSI

SEQ ID NO: 28 - protein, 9 aa, Hepatitis B virus
LPYRPTTGR

SEQ ID NO: 29 - protein, 9 aa, Hepatitis B virus
LQDPRVRAL

SEQ ID NO: 30 - protein, 10 aa, Hepatitis B virus
LQDPRVRALY

SEQ ID NO: 31 - protein, 10 aa, Hepatitis B virus
LTIPQSLDSW

SEQ ID NO: 32 - protein, 11 aa, Hepatitis B virus
LTIPQSLDSWW

SEQ ID NO: 33 - protein, 11 aa, Hepatitis B virus
LTRILTIPQSL

SEQ ID NO: 34 - protein, 9 aa, Hepatitis B virus
LYAAVTNFL

SEQ ID NO: 35 - protein, 12 aa, Hepatitis B virus
MENIASGLLGPL

SEQ ID NO: 36 - protein, 12 aa, Hepatitis B virus
MENITSGFLGPL

SEQ ID NO: 37 - protein, 10 aa, Hepatitis B virus
NILSPFMPLL

SEQ ID NO: 38 - protein, 10 aa, Hepatitis B virus
NITSGFLGPL

SEQ ID NO: 39 - protein, 9 aa, Hepatitis B virus
PQSLDSWLT

SEQ ID NO: 40 - protein, 9 aa, Hepatitis B virus
QSPTSNHSL

SEQ ID NO: 41 - protein, 9 aa, Hepatitis B virus
SAISSTFSK

SEQ ID NO: 42 - protein, 9 aa, Hepatitis B virus
STISSTFSK

SEQ ID NO: 43 - protein, 11 aa, Hepatitis B virus
STLPETTVVRR

SEQ ID NO: 44 - protein, 9 aa, Hepatitis B virus
TASPISSIF

SEQ ID NO: 45 - protein, 9 aa, Hepatitis B virus
TCIPIPSSW

SEQ ID NO: 46 - protein, 9 aa, Hepatitis B virus
TIPQSLDSW

SEQ ID NO: 47 - protein, 10 aa, Hepatitis B virus
TRILTIPQSL

SEQ ID NO: 48 - protein, 9 aa, Hepatitis B virus
TVSAISSTF

SEQ ID NO: 49 - protein, 9 aa, Hepatitis B virus
VGLSPTVWL

SEQ ID NO: 50 - protein, 12 aa, Hepatitis B virus
WTSLNFLGGTTV

SEQ ID NO: 51 - protein, 9 aa, Hepatitis B virus
YPALMPLYA

SEQ ID NO: 52 - protein, 9 aa, Hepatitis B virus
IPIPSSWAF

SEQ ID NO: 53 - protein, 9 aa, Hepatitis B virus
TASAISSTF

SEQ ID NO: 54 - protein, 9 aa, Hepatitis B virus
TASPLSSIF

SEQ ID NO: 55 - protein, 4 aa, synthetic construct
GGGS

SEQ ID NO: 56 - protein, 4 aa, synthetic construct
GGSG

SEQ ID NO: 57 - protein, 5 aa, synthetic construct
GSGGS

SEQ ID NO: 58 - protein, 5 aa, synthetic construct
GGSGG

SEQ ID NO: 59 - protein, 5 aa, synthetic construct
GSGSG

SEQ ID NO: 60 - protein, 5 aa, synthetic construct
GSGGG

SEQ ID NO: 61 - protein, 5 aa, synthetic construct
GGGSG

SEQ ID NO: 62 - protein, 5 aa, synthetic construct
GSSSG

SEQ ID NO: 63 - protein, 15 aa, synthetic construct
GCGASGGGGSGGGGS

SEQ ID NO: 64 - protein, 15 aa, synthetic construct
GCGASGGGGSGGGGS

SEQ ID NO: 65 - protein, 10 aa, synthetic construct
GGGGSGGGGS

SEQ ID NO: 66 - protein, 15 aa, synthetic construct
GGGASGGGGSGGGGS

SEQ ID NO: 67 - protein, 15 aa, synthetic construct
GGGGSGGGGSGGGGS

SEQ ID NO: 68 - protein, 10 aa, synthetic construct
GGGASGGGGS

SEQ ID NO: 69 - protein, 15 aa, synthetic construct
GGGGSGGGGSGGGGS

SEQ ID NO: 70 - protein, 20 aa, synthetic construct
GGGGSGGGGSGGGGSGGGGS

SEQ ID NO: 71 - DNA, 12 bp, synthetic construct
  source 1..12: mol_type = other DNA
gggggtggttcc

SEQ ID NO: 72 - DNA, 12 bp, synthetic construct
  source 1..12: mol_type = other DNA
ggtgggtctggg

SEQ ID NO: 73 - DNA, 15 bp, synthetic construct
  source 1..15: mol_type = other DNA
gggtccgggggctcc

SEQ ID NO: 74 - DNA, 15 bp, synthetic construct
  source 1..15: mol_type = other DNA
ggtgggagcggtggt

SEQ ID NO: 75 - DNA, 15 bp, synthetic construct
  source 1..15: mol_type = other DNA
ggcagcggaagcgga

SEQ ID NO: 76 - DNA, 15 bp, synthetic construct
  source 1..15: mol_type = other DNA
gggagtgggggaggt

SEQ ID NO: 77 - DNA, 15 bp, synthetic construct
  source 1..15: mol_type = other DNA
ggtgggggaagtgga

SEQ ID NO: 78 - DNA, 15 bp, synthetic construct
  source 1..15: mol_type = other DNA
ggcagctcatctggt

SEQ ID NO: 79 - DNA, 45 bp, synthetic construct
  source 1..45: mol_type = other DNA
ggatgtggtgcatctggagggggaggctctggggggggtggatct

SEQ ID NO: 80 - DNA, 45 bp, synthetic construct
  source 1..45: mol_type = other DNA
gggtgtggtgctagtgggggtggcggatcaggtggaggcgggagc

SEQ ID NO: 81 - DNA, 30 bp, synthetic construct
  source 1..30: mol_type = other DNA
gggggcggaggatctgggggagggggatca

SEQ ID NO: 82 - DNA, 42 bp, synthetic construct
  source 1..42: mol_type = other DNA
ggggggggcgcttcaggcggaggtggaagtggtggaggaggt

SEQ ID NO: 83 - DNA, 45 bp, synthetic construct
  source 1..45: mol_type = other DNA
ggagggggaggttctggcggcgggggatcaggaggcggtgggagc

SEQ ID NO: 84 - DNA, 30 bp, synthetic construct
  source 1..30: mol_type = other DNA
ggtgggggggcgtcaggtggaggcggaagt

SEQ ID NO: 85 - DNA, 45 bp, synthetic construct
  source 1..45: mol_type = other DNA
ggcggcggaggttctggtgggggtggcagtggaggaggaggcagc

SEQ ID NO: 86 - DNA, 60 bp, synthetic construct
  source 1..60: mol_type = other DNA
ggaggtggaggtagtggcggtggtgggtcagggggaggcgggtccggtggcggtgggagt

SEQ ID NO: 87 - DNA, 12 bp, synthetic construct
  source 1..12: mol_type = other DNA
ggtggcggtagt

SEQ ID NO: 88 - DNA, 12 bp, synthetic construct
  source 1..12: mol_type = other DNA
gggggatctggt

SEQ ID NO: 89 - DNA, 15 bp, synthetic construct
  source 1..15: mol_type = other DNA
ggcagtggcggtagc

SEQ ID NO: 90 - DNA, 15 bp, synthetic construct
  source 1..15: mol_type = other DNA
ggagggagtggaggg

SEQ ID NO: 91 - DNA, 15 bp, synthetic construct
  source 1..15: mol_type = other DNA
gggtctggctcaggc

SEQ ID NO: 92 - DNA, 15 bp, synthetic construct
  source 1..15: mol_type = other DNA
ggttctggcggaggt

SEQ ID NO: 93 - DNA, 15 bp, synthetic construct
  source 1..15: mol_type = other DNA
ggtggtgggagtgga

SEQ ID NO: 94 - DNA, 15 bp, synthetic construct
  source 1..15: mol_type = other DNA
ggctcaagcagtgga

SEQ ID NO: 95 - DNA, 45 bp, synthetic construct
  source 1..45: mol_type = other DNA
ggctgtggggctagtgggggaggtggtagtggtggtggcggttcc

SEQ ID NO: 96 - DNA, 45 bp, synthetic construct
  source 1..45: mol_type = other DNA
ggatgtggggcctcaggtgggggtggcagcggtggtggagggtca

SEQ ID NO: 97 - DNA, 30 bp, synthetic construct
  source 1..30: mol_type = other DNA
ggtggcgggggctctggtggaggaggatct

SEQ ID NO: 98 - DNA, 42 bp, synthetic construct
  source 1..42: mol_type = other DNA
ggaggcggcgcttctgggggcgggggtagtgggggtggaggt

SEQ ID NO: 99 - DNA, 45 bp, synthetic construct
  source 1..45: mol_type = other DNA
ggtggaggtggaagtggaggagggggatcaggcggaggcgggagc

SEQ ID NO: 100 - DNA, 30 bp, synthetic construct
  source 1..30: mol_type = other DNA
ggagggggagcctctggcggtggaggatca

SEQ ID NO: 101 - DNA, 45 bp, synthetic construct
  source 1..45: mol_type = other DNA
gggggaggaggcagtggaggtgggggaagtggtggaggggggtct

SEQ ID NO: 102 - DNA, 60 bp, synthetic construct
  source 1..60: mol_type = other DNA
gggggtggaggatcaggaggcggtggttctgggggaggtggatccggcgggggtggtagt

SEQ ID NO: 103-104 - skipped (2 entries)

SEQ ID NO: 105 - protein, 5 aa, synthetic construct
GSGGS

SEQ ID NO: 106 - protein, 4 aa, synthetic construct
GGGS

SEQ ID NO: 107 - protein, 11 aa, synthetic construct
  VARIANT 2: note = S or T
  VARIANT 6: note = E or D
  VARIANT 9: note = V or I
  VARIANT 10: note = Q, H or L
GXLPQXHIXXK

SEQ ID NO: 108 - protein, 11 aa, synthetic construct
GSLPQEHIVQK

SEQ ID NO: 109 - protein, 6 aa, synthetic construct
HHHHHH

SEQ ID NO: 110 - protein, 11 aa, Hepatitis B virus
GTLPQDHIVQK

SEQ ID NO: 111 - protein, 11 aa, Hepatitis B virus
GSLPQDHIIQK

SEQ ID NO: 112 - protein, 11 aa, Hepatitis B virus
GTLPQEHIVLK

(19) United States
(12) Patent Application Publication
(10) Pub. No.: US 2026/0167675 A1
(43) Pub. Date: Jun. 18, 2026

(54) VIRAL PEPTIDES AND USES THEREOF

(71) Applicant: Regeneron Pharmaceuticals, Inc., Tarrytown, NY (US)

(72) Inventors: Augustine CHOY, Dobbs Ferry, NY (US); Richard COPIN, New York, NY (US); Christos KYRATSOUS, Irvington, NY (US); Robert SALZLER, Ossining, NY (US); Mayank SRIVASTAVA, New Rochelle, NY (US)

(73) Assignee: Regeneron Pharmaceuticals, Inc., Tarrytown, NY (US)

(21) Appl. No.: 18/871,778

(22) PCT Filed: Jun. 6, 2023
(86) PCT No.: PCT/US2023/067999

§ 371 Date: Dec. 4, 2024

Related U.S. Application Data
(60) Provisional application No. 63/349,804, filed on Jun. 7, 2022.
(60) Provisional application No. 63/398,305, filed on Aug. 16, 2022.

(51) Int. Cl. C07K 14/005; A61K 39/29; A61P 35/00; A61P 37/04; C07K 14/74; C12Q 1/70; G16B 30/20

(52) U.S. Cl. CPC C07K 14/005; A61K 39/292; A61P 35/00; A61P 37/04; C07K 14/70539; C12Q 1/706; G16B 30/20; C12N 2730/10122; C12N 2730/10134; C12Q 2600/158

(57) ABSTRACT

The present disclosure provides isolated peptides derived from hepatitis B virus (HBV), peptide-based molecules (e.g., peptide-MHC (pMHC) complexes), polynucleotides and vectors encoding the peptides or peptide-based molecules, pharmaceutical compositions (e.g., vaccine compositions), and their use for treatment or prevention of HBV infection and/or HBV-induced diseases. The present disclosure also provides binding moieties that bind to the peptides or peptide-based molecules disclosed herein, and their use for treatment or prevention of HBV infection and/or HBV-induced diseases. The present disclosure further provides methods and systems for identifying immunogenic virus-derived peptides.

### CROSS-REFERENCE TO RELATED APPLICATIONS

This patent application claims the benefit of U.S. Provisional Application No. 63/398,305, filed Aug. 16, 2022, and U.S. Provisional Application No. 63/349,804, filed Jun. 7, 2022, the disclosure of each of which is incorporated by reference herein in its entirety for all purposes.

### SEQUENCE LISTING

The instant application contains a Sequence Listing which has been submitted electronically in XML file format and is hereby incorporated by reference in its entirety. Said XML copy, created on May 30, 2023, is named 250298_000482_SL.xml and is 98,432 bytes in size.

### TECHNICAL FIELD

The present disclosure relates to methods and compositions that involve isolated peptides derived from hepatitis B virus (HBV), and the use of such methods and compositions for treatment or prevention of HBV infection and/or HBV-induced diseases.

### BACKGROUND

Chronic hepatitis B virus (HBV) infection affects ˜250 million people worldwide and can result in serious complications including acute and chronic hepatitis, cirrhosis, and hepatocellular carcinoma (HCC). Vaccination against HBV is effective at preventing infection, but there is currently no cure for patients chronically infected with HBV.

The persistence or control of HBV infection is determined by the host immune response, which is primarily mediated by cytotoxic T-cells that mediate adaptive immunity by recognizing HLA-peptide complexes presented on the surface of infected cells via interactions via T cell receptors (TCRs). While proteogenomics can be applied to identify HLA-associated mutant peptides in cancer, HBV-HCC tumor cells can contain fragments of HBV DNA that have been inserted into the host genome, ultimately posing a challenge for identifying HBV epitopes. Further complicating this effort is the existence of thousands of HBV strains distributed across 10 different HBV genotypes, which differ from each other by at least 8% at the nucleotide level. Thus, development of methods to identify HBV epitopes could be helpful for patients infected with HBV.

### SUMMARY

As specified in the Background section above, there is a great need in the art for development of methods to identify HBV epitopes that could be helpful for patients infected with HBV. The present application addresses these and other needs.

In one aspect, provided herein is an isolated peptide comprising an amino acid sequence that is at least 90% identical to the amino acid sequence of any one of SEQ ID NOs: 1-54 and 110-112, or a pharmaceutically acceptable salt thereof, or a fragment or derivative thereof, wherein the isolated peptide is 8-12 amino acids in length.

In some embodiments, the isolated peptide comprises an amino acid sequence of any one of SEQ ID NOs: 1-54 and 110-112.

In some embodiments, the isolated peptide consists essentially of an amino acid sequence of any one of SEQ ID NOs: 1-54 and 110-112.

In some embodiments, the isolated peptide consists of an amino acid sequence of any one of SEQ ID NOs: 1-54 and 110-112.

In another aspect, provided herein is an isolated peptide comprising two or more amino acid sequences selected from any one of SEQ ID NO: 1-54 and 110-112, or a pharmaceutically acceptable salt thereof, or a fragment or derivative thereof.

In some embodiments, the isolated peptide consists of an amino acid sequence GX₁LPQX₂HIX₃X₄K (SEQ ID NO: 107), wherein X₁ is S or T, X₂ is E or D, X₃ is V or I, and X₄ is Q, H or L, or a pharmaceutically acceptable salt thereof, or a fragment or derivative thereof.

In some embodiments, the isolated peptide comprises one or more reverse peptide bonds, one or more non-peptide bonds, one or more D-isomers of amino acids, one or more chemical modifications, or any combination thereof.

In some embodiments, the isolated peptide is produced by expression in a heterologous host cell.

In some embodiments, the isolated peptide is produced synthetically.

In some embodiments, the isolated peptide, or pharmaceutically acceptable salt thereof, or fragment or derivative thereof induces a hepatitis B virus (HBV)-specific immune response in a subject when presented in a complex with a major histocompatibility complex (MHC) molecule on the surface of an antigen presenting cell (APC).

In another aspect, provided herein is a fusion protein comprising one or more isolated peptides disclosed herein fused to one or more heterologous molecules.

In some embodiments, the one or more heterologous molecules enhance a peptide-specific immune response in a subject.

In some embodiments, one or more heterologous molecules mediate peptide delivery to a specific site within a subject.

In some embodiments, the one or more heterologous molecules are a MHC molecule, or a fragment or derivative thereof.

In another aspect, provided herein is a conjugate comprising one or more isolated peptides disclosed herein conjugated to one or more heterologous molecules.

In some embodiments, the one or more heterologous molecules enhance a peptide-specific immune response in a subject.

In some embodiments, the one or more heterologous molecules mediate peptide delivery to a specific site within a subject.

In some embodiments, the one or more heterologous molecules are an MHC molecule, or a fragment or derivative thereof.

In some embodiments, the one or more peptides are conjugated to a particle.

In another aspect, provided herein is an oligomeric complex comprising two or more isolated peptides disclosed herein.

In another aspect, provided herein is a non-covalent complex comprising an isolated peptide disclosed herein and an MHC molecule, or a fragment or derivative thereof.

In some embodiments, the MHC molecule, or the fragment thereof, is a class I MHC molecule.

In some embodiments, the class I MHC molecule is a class I human leukocyte antigen (HLA) molecule.

In some embodiments, the MHC molecule, or the fragment thereof, is a class II MHC molecule.

In some embodiments, the class II MHC molecule is a class II HLA molecule.

In another aspect, provided herein is a fusion protein comprising an isolated peptide disclosed herein and an MHC molecule, or a fragment or derivative thereof.

In some embodiments, the MHC molecule, or the fragment thereof, is a class I MHC molecule.

In some embodiments, the class I MHC molecule is a class I human leukocyte antigen (HLA) molecule.

In some embodiments, the MHC molecule, or the fragment thereof, is a class II MHC molecule.

In some embodiments, the class II MHC molecule is a class II HLA molecule.

In another aspect, provided herein is a conjugate comprising an isolated peptide disclosed herein and a MHC molecule, or a fragment or derivative thereof.

In some embodiments, the MHC molecule, or the fragment thereof, is a class I MHC molecule.

In some embodiments, the class I MHC molecule is a class I human leukocyte antigen (HLA) molecule.

In some embodiments, the MHC molecule, or the fragment thereof, is a class II MHC molecule.

In some embodiments, the class II MHC molecule is a class II HLA molecule.

In another aspect, provided herein is a pharmaceutical composition comprising (i) one or more isolated peptides disclosed herein, one or more fusion proteins disclosed herein, one or more conjugates disclosed herein, one or more oligomeric complexes disclosed herein, or one or more non-covalent complexes disclosed herein, or any combination thereof, and (ii) a pharmaceutically acceptable carrier or excipient.

In some embodiments of the pharmaceutical composition disclosed herein further comprises an adjuvant.

In another aspect, provided herein is an isolated molecule that binds an isolated peptide disclosed herein, a fusion protein disclosed herein, a conjugate disclosed herein, an oligomeric complex disclosed herein, or a non-covalent complex disclosed herein.

In some embodiments, the molecule is an antibody or an antigen-binding fragment thereof.

In some embodiments, the antibody is a bispecific antibody.

In some embodiments, the molecule is an alternative scaffold.

In some embodiments, the molecule is a chimeric antigen receptor (CAR).

In some embodiments, the molecule is a T cell receptor (TCR).

In another aspect, provided herein is an isolated cell comprising a CAR disclosed herein.

In some embodiments, the isolated cell is an immune cell.

In some embodiments, the immune cell is a T cell, an NK cell, or a macrophage.

In another aspect, provided herein is an isolated cell comprising a TCR disclosed herein.

In some embodiments, the isolated cell is an immune cell.

In some embodiments, the immune cell is a T cell, an NK cell, or a macrophage.

In another aspect, provided herein is a pharmaceutical composition comprising (i) an isolated molecule disclosed herein, or an isolated cell disclosed herein; and (ii) a pharmaceutically acceptable carrier or excipient.

In another aspect, provided herein is an isolated polynucleotide comprising a nucleotide sequence encoding one or more isolated peptides disclosed herein or a fusion protein disclosed herein.

In some embodiments, the nucleotide sequence is operably linked to a promoter.

In some embodiments, the isolated polynucleotide comprises DNA.

In some embodiments, the isolated polynucleotide comprises RNA.

In some embodiments, the RNA is mRNA.

In some embodiments, the RNA is self-replicating RNA.

In another aspect, provided herein is a vector comprising an isolated polynucleotide disclosed herein.

In some embodiments, the vector is an expression vector.

In some embodiments, the vector is a viral vector.

In another aspect, provided herein is a host cell comprising an isolated polynucleotide disclosed herein or a vector disclosed herein.

In some embodiments, the host cell is a prokaryotic cell.

In some embodiments, the host cell is a eukaryotic cell.

In some embodiments, the host cell is an APC.

In another aspect, provided herein is a pharmaceutical composition comprising (i) an isolated polynucleotide disclosed herein, or a vector disclosed herein; and (ii) a pharmaceutically acceptable carrier or excipient.

In some embodiments, the pharmaceutically acceptable carrier is a lipid nanoparticle carrier.

In another aspect, provided herein is a method of inducing an immune response against a hepatitis B viral (HBV) infection in a subject in need thereof, the method comprising administering to the subject a therapeutically effective amount of:
a) one or more isolated peptides disclosed herein;
b) a fusion protein disclosed herein
c) a conjugate disclosed herein;
d) an oligomeric complex disclosed herein;
e) a non-covalent complex disclosed herein;
f) a pharmaceutical composition disclosed herein;
g) a molecule disclosed herein;
h) an isolated cell disclosed herein;
i) an isolated polynucleotide disclosed herein; or
j) a vector disclosed herein.

In another aspect, provided herein is a method of inducing an immune response against a hepatitis B viral (HBV) infection in a subject in need thereof, the method comprising administering to the subject a therapeutically effective amount of one or more isolated peptides disclosed herein.

In another aspect, provided herein is a method of inducing an immune response against an HBV infection in a subject in need thereof, comprising administering to the subject an activated T cell that is produced by contacting a T cell with an APC that presents an isolated peptide disclosed herein in complex with an MHC molecule.

In another aspect, provided herein is a method of treating an HBV-induced disease or disorder in a subject in need thereof, the method comprising administering to the subject an effective amount of:
a) one or more isolated peptides disclosed herein;
b) a fusion protein disclosed herein;
c) a conjugate disclosed herein;
d) an oligomeric complex disclosed herein;
e) a non-covalent complex disclosed herein;
f) a pharmaceutical composition disclosed herein;
g) a molecule disclosed herein;
h) an isolated cell disclosed herein;
i) an isolated polynucleotide disclosed herein; or
j) a vector disclosed herein.

In another aspect, provided herein is a method of preventing or reducing the likelihood of an HBV-induced disease or disorder in a subject in need thereof, the method comprising administering to the subject an effective amount of:
a) one or more isolated peptides disclosed herein;
b) a fusion protein disclosed herein;
c) a conjugate disclosed herein;
d) an oligomeric complex disclosed herein;
e) a non-covalent complex disclosed herein;
f) a pharmaceutical composition disclosed herein;
g) a molecule disclosed herein;
h) an isolated cell disclosed herein;
i) an isolated polynucleotide disclosed herein; or
j) a vector disclosed herein.

In another aspect, provided herein is a method of treating an HBV-induced disease or disorder in a subject in need thereof, the method comprising administering to the subject an effective amount of one or more isolated peptides disclosed herein.

In another aspect, provided herein is a method of preventing or reducing the likelihood of an HBV-induced disease or disorder in a subject in need thereof, the method comprising administering to the subject an effective amount of one or more isolated peptides disclosed herein.

In some embodiments, the HBV-induced disease or disorder is a liver inflammation, liver fibrosis, liver cirrhosis, or liver cancer.

In some embodiments, the liver cancer is hepatocellular carcinoma (HCC).

In another aspect, provided herein is a kit comprising:
(i) a) one or more isolated peptides disclosed herein;
   b) a fusion protein disclosed herein;
   c) a conjugate disclosed herein;
   d) an oligomeric complex disclosed herein;
   e) a non-covalent complex disclosed herein;
   f) a pharmaceutical composition disclosed herein;
   g) a molecule disclosed herein;
   h) an isolated cell disclosed herein;
   i) an isolated polynucleotide disclosed herein; or
   j) a vector disclosed herein; and
(ii) packaging and/or instructions for use for the same.

In another aspect, provided herein is a method for identifying an immunogenic virus-derived peptide, the method comprising:
a) obtaining a plurality of RNA contig sequences derived from an infected subject infected with a virus, wherein the plurality of RNA contig sequences comprises a plurality of virus-derived RNA contig sequences and a plurality of infected-subject endogenous RNA contig sequences;
b) identifying the plurality of virus-derived RNA contig sequences from within the plurality of RNA contig sequences;
c) assembling a viral RNA sequence based on the plurality of virus-derived RNA contig sequences;
d) identifying a protein sequence based on the viral RNA sequence; and
e) identifying the immunogenic virus-derived peptide based at least in part on the identified protein sequence.

In some embodiments, the plurality of RNA contig sequences are derived from one infected subject.

In some embodiments, the infected subject is a human.

In some embodiments, the plurality of virus-derived RNA contig sequences are derived from the virus infecting the infected subject.

In some embodiments, the plurality of infected-subject endogenous RNA contig sequences are derived from RNA endogenous to the infected subject.

In some embodiments, the identifying the plurality of virus-derived RNA contig sequences from within the plurality of RNA contig sequences comprises:
comparing at least a portion of contig sequences of the plurality of RNA contig sequences to a reference viral sequence; and
identifying the plurality of virus-derived RNA contig sequences such that each contig sequence of the plurality of virus-derived RNA contig sequences comprises at least a portion that corresponds to the reference viral sequence.

In some embodiments, each contig sequence of the plurality of virus-derived RNA contig is distinct from the plurality of infected-subject endogenous RNA contig sequences.

In some embodiments, each contig sequence of the plurality of virus-derived RNA contig sequences lacks infected-subject endogenous RNA contig sequences.

In some embodiments, the reference viral sequence comprises a reference genome.

In some embodiments, the reference genome comprises a hepatitis B virus genome.

In some embodiments, the assembling the viral RNA sequence based on the plurality of virus-derived RNA contig sequences comprises:
overlapping common sequence portions at ends of at least a portion of the plurality of virus-derived RNA contig sequences such that the at least a portion of the plurality of virus-derived RNA contig sequences overlap linearly to assemble the viral RNA sequence.

In some embodiments, the identifying a protein sequence based on the viral RNA sequence such that the identified protein sequence includes a translation of the viral RNA sequence comprises:
identifying the protein sequence without requiring a comparison to a database of viral proteins.

In some embodiments, the identifying the protein sequence based on the viral RNA sequence such that the identified protein sequence includes a translation of the viral RNA sequence further comprises:
identifying a plurality of protein sequences each based on the viral RNA sequence such that each of the plurality of protein sequences respectively include a translation of the viral RNA sequence, and
identifying the protein sequence as a frequently occurring protein sequence within the plurality of protein sequences.

In some embodiments, the protein sequence identified based on the viral RNA sequence is associated with a single infected subject.

In some embodiments, identifying the immunogenic virus-derived peptide based at least in part on the protein sequence comprises:
identifying an MHC molecule associated with the single infected subject;
identifying one or more peptides based at least in part on the protein sequence such that the one or more peptides each form a respective MHC-peptide complex with the MHC molecule; and
identifying the immunogenic virus-derived peptide based on the one or more peptides.

In another aspect, provided herein is a non-transitory computer-readable medium configured to communicate with one or more processor(s) of a computational device, the non-transitory computer-readable medium including instructions thereon, that when executed by the processor(s), cause the computational device to:
a) receive, as an input, a plurality of RNA contig sequences derived from an infected subject infected with a virus such that the plurality of RNA contig sequences comprise a plurality of virus-derived RNA contig sequences and a plurality of infected-subject endogenous RNA contig sequences, and wherein the infected subject is infected with a virus;
b) identify the plurality of virus-derived RNA contig sequences from within the plurality of RNA contig sequences;
c) assemble a viral RNA sequence based on the plurality of virus-derived RNA contig sequences;
d) identify a protein sequence based on the viral RNA sequence;
e) identify an immunogenic virus-derived peptide based at least in part on the protein sequence; and
f) provide, as an output, the immunogenic virus-derived peptide.

In some embodiments, the plurality of RNA contig sequences are derived from only one infected subject.

In some embodiments, the infected subject comprises a human.

In some embodiments, the plurality of virus-derived RNA contig sequences are derived from the virus infecting the infected subject.

In some embodiments, the plurality of infected-subject endogenous RNA contig sequences are derived from RNA endogenous to the infected subject.

In some embodiments, the instructions which cause the computational device to identify the plurality of virus-derived RNA contig sequences from within the plurality of RNA contig sequences further comprise instructions, that when executed by the processor(s), cause the computational device to:
compare at least a portion of contig sequences of the plurality of RNA contig sequences to a reference viral sequence; and
identify the plurality of virus-derived RNA contig sequences such that each contig sequence of the plurality of virus-derived RNA contig sequences comprises at least a portion that corresponds to the reference viral sequence.

In some embodiments, each contig sequence of the plurality of virus-derived RNA contig is distinct from the plurality of infected-subject endogenous RNA contig sequences.

In some embodiments, each contig sequence of the plurality of virus-derived RNA contig sequences lacks portions of infected-subject endogenous RNA contig sequences.

In some embodiments, the reference viral sequences comprises a reference genome.

In some embodiments, the reference genome comprises a hepatitis B virus genome.

In some embodiments, the instructions which cause the computational device to assemble the viral RNA sequence based on the plurality of virus-derived RNA contig sequences further comprise instructions, that when executed by the processor(s), cause the computational device to:
overlap common sequence portions at ends of at least a portion of the plurality of virus-derived RNA contig sequences such that the at least a portion of the plurality of virus-derived RNA contig sequences overlap linearly to assemble the viral RNA sequence.

In some embodiments, the instructions which cause the computational device to identify a protein sequence based on the viral RNA sequence such that the protein sequence includes a translation of the viral RNA sequence further comprise instructions, that when executed by the processor, cause the computational device to:
identify the protein sequence without requiring a comparison to a database of viral proteins.

In some embodiments, the protein sequence identified based on the viral RNA sequence is a novel protein.

In some embodiments, the protein sequence identified based on the viral RNA sequence is associated with a single infected subject.

In some embodiments, the instructions which cause the computational device to identify a protein sequence based on the viral RNA sequence such that the protein sequence includes a translation of the viral RNA sequence further comprise instructions, that when executed by the processor(s), cause the computational device to:
identify a plurality of protein sequences each based on the viral RNA sequence such that each of the plurality of protein sequences respectively include a translation of the viral RNA sequence; and
identify the protein sequence as a frequently occurring protein sequence within the plurality of protein sequences.

In some embodiments, the instructions which cause the computational device to identify the immunogenic virus-derived peptide based at least in part on the protein sequence further comprise instructions, that when executed by the processor(s), cause the computational device to:
identify a major histocompatibility complex (MHC) molecule associated with the single infected subject;
identify one or more peptides based at least in part on the protein sequence such that the one or more peptides are each capable of forming a respective MHC-peptide complex with the MHC molecule; and
identify the immunogenic virus-derived peptide based on the one or more peptides.

In some embodiments, the instructions, when executed by the processor(s), further cause the computational device to: store the protein sequence to a database such that the protein sequence is associated with the infected subject within the database.

In another, provided herein is a method for identifying an integration site of a viral gene within a subject gene, the method comprising:
a) obtaining a plurality of RNA contig sequences derived from an infected subject infected with a virus such that the plurality of RNA contig sequences comprise a plurality of virus-derived RNA contig sequences, a plurality of infected-subject endogenous RNA contig sequences, and a plurality of hybrid RNA contig sequences comprising viral and infected-subject endogenous portions;
b) identifying the plurality of hybrid RNA contig sequences from within the plurality of RNA contig sequences;
c) comparing, for at least a portion of the plurality of hybrid RNA contig sequences, infected-subject endogenous portions to a subject reference genome; and
d) identifying, based at least in part on the comparison of infected-subject endogenous portions to the subject reference genome, an integration site comprising the subject gene.

In some embodiments, the plurality of RNA contig sequences are derived from one infected subject.

In some embodiments, the infected subject is a human.

In some embodiments, the plurality of virus-derived RNA contig sequences are derived from the virus infecting the infected subject.

In some embodiments, the virus is a hepatitis B virus.

In some embodiments, the plurality of infected-subject endogenous RNA contig sequences are derived from RNA endogenous to the infected subject.

In some embodiments, the subject reference genome comprises the human genome.

In another aspect, provided herein is a non-transitory computer-readable medium configured to communicate with one or more processor(s) of a computational device, the non-transitory computer-readable medium including instructions thereon, that when executed by the processor(s), cause the computational device to:
a) receive, as an input, a plurality of RNA contig sequences derived from an infected subject infected with a virus such that the plurality of RNA contig sequences comprise a plurality of virus-derived RNA contig sequences, a plurality of infected-subject endogenous RNA contig sequences, and plurality of hybrid RNA contig sequences comprising viral and infected-subject endogenous portions;
b) identify the plurality of hybrid RNA contig sequences from within the plurality of RNA contig sequences;
c) compare, for at least a portion of the plurality of hybrid RNA contig sequences, infected-subject endogenous portions to a subject reference genome;
d) identify, based at least in part on the comparison of infected-subject endogenous portions to the subject reference genome, an integration site comprising a subject gene; and
e) provide, as an output, the integration site.

In some embodiments, the plurality of RNA contig sequences are derived from only one infected subject.

In some embodiments, the infected subject comprises a human.

In some embodiments, the plurality of virus-derived RNA contig sequences are derived from a virus infecting the infected subject.

In some embodiments, the virus comprises a hepatitis B virus.

In some embodiments, the plurality of infected-subject endogenous RNA contig sequences are derived from RNA endogenous to the infected subject.

In some embodiments, the subject reference genome comprises the human genome.

These and other aspects of the present invention will be apparent to those of ordinary skill in the art in the following description, claims and drawings.

### BRIEF DESCRIPTION OF THE DRAWINGS

FIGS. 1A-1D show an approach to identify HBV-specific signatures using bulk RNA sequencing (RNAseq) of human liver samples. RNA contigs fully matching the HBV reference are used to reconstruct genomes from patient-infected HBV strains. Hybrid contigs matching both human and HBV references are filtered to identify site of integration within the human genomes (FIG. 1A). Coverage of total RNA reads from bulk RNA sequencing of all human liver samples to HBV reference genome (FIG. 1B). Phylogenetic relationship between reconstructed HBV genomes and reference HBV sequences representative of eight HBV lineages (A, B, C, D, E, F, G, H) (FIG. 1C). Location of HBV integrated genome breakpoints across all samples (FIG. 1D).

FIGS. 2A-2B show peptide length distributions and percent binders from identified peptides with 9 amino acid residues (9-mers). Peptide length distributions by mass spectrometry for all HBV-positive patient liver tissues by serology (FIG. 2A). Plot showing the percent (%) binders to HLA-A, HLA-B and HLA-C alleles from 9-mer peptides identified by mass spectrometry (FIG. 2B).

FIGS. 3A-3C show HLA genotypes from HLA-A and HLA-B with the highest number of HBV peptides detected, predicted binders of HLA alleles, and a chart of target peptides with corresponding detection frequency in HBV positive (+) samples. The four most frequent HLA alleles from HLA-A, HLA-B and HLA-C among the patient samples analyzed, along with the number of samples that were HBV positive by Mass Spectrometry (HBV peptides detected). (FIG. 3A). HBV peptide sequences identified from immunopeptidomics of patient liver samples, and their predicted binding to patient HLA alleles predicted by Pan NetMHC 4.0 (FIG. 3B). Target HBV peptides and the frequencies of detection of the target HBV peptides in HBV+ samples (FIG. 3C).

FIGS. 4A-4D demonstrate validation of four HBV peptides, including two polymerase variants (FIG. 4A and FIG. 4C) and two other peptides (FIG. 4B and FIG. 4D).

FIG. 5 shows an example immunoblot demonstrating the enrichment of HLA by HLA-I specific W6/32 antibody conjugated to sepharose beads.

FIG. 6 displays a bar graph showing total peptide count and the peptides with 9-12 residues as identified by mass spectrometry.

FIGS. 7A-7X show motif analysis of all 9-mer peptides identified by a PEAKSX search of mass spectrometry raw files.

FIG. 8 shows that the MS2 fragment ion spectra for the three polymerase peptide variants show extensive fragmentation for confident peptide identifications.

FIGS. 9A-9H depict an MS1 analysis showing co-elution of endogenous peptides and the respective synthetic heavy analogues for the validated HBV peptides in the Mass Spectrometry run. (FIGS. 9A-9G). Retention time and estimated copy number calculated using the peptide intensity for heavy and endogenous peptides for HLA-associated HBV peptides in patient samples (FIG. 9H).

FIGS. 10A-10B show cell binding curves of a pan-HLA-A antibody to K562 cells pulsed with HBV POL₆₀₆₋₆₁₆ peptides at concentrations of 100 g/ml peptide (FIG. 10A) and 33 g/ml peptide (FIG. 10B).

### DETAILED DESCRIPTION

Chronic HBV infection can severely compromise liver function by causing liver inflammation, fibrosis, and cirrhosis, and can eventually lead to Hepatocellular carcinoma (HCC); nearly 25-40% of HBV carriers develop HCC. Vaccination and anti-viral agents (e.g. reverse transcriptase inhibitors and interferon therapy) are the preferred prevention and treatment choices in the management of HBV infection. However, once the infection is established, anti-viral agents can only control viral transcription and are largely ineffective in Hepatitis B surface antigen (HBsAg) seroclearance. Additionally, the integration of HBV DNA fragments into the host genome can introduce genetic damage and chromosomal instability that can further contribute to the development of HCC. Globally, it is estimated that chronic HBV infections account for ˜50% of primary liver cancers.

The persistence or control of HBV infection is determined by the host immune response, which, following HBV evasion of the innate immune system, is primarily mediated by the adaptive immune system. Cytotoxic CD8+ T cells of the adaptive immune system protect a host against intracellular pathogens and tumors. As such, cytotoxic CD8+ T cells play a pivotal role in defending against HBV infection, and are primarily responsible for clearing virus in acute infections, as well as preventing development of HCC. In cases of chronic HBV infection, HBV-specific CD8+ T cells are frequently undetectable, which is thought to result from the chronic exposure of the liver to high antigenic loads and tolerogenic hepatocytes.

Because of the critical role of CD8+ T-cells in defending against viral infections, harnessing CD8+ T-cells have become an important aspect of immunotherapies. Cytotoxic T-cells mediate adaptive immunity by recognizing HLA-peptide complexes presented on the surface of infected cells via interactions with the T cell receptors (TCRs) expressed on their surface. These HLA-peptide complexes can be used as targets for developing different immunotherapeutics, including, for example, bispecific antibodies and CAR-T therapies.

Proteogenomics can be applied to identify HLA-associated mutant peptides in cancer. HBV-HCC tissue can lack the HBV covalently closed circular DNA (cccDNA), the template for viral protein synthesis. Instead, tumor cells can contain fragments of HBV DNA that have been inserted into the host genome that can be transcribed and, ultimately, result in the expression of viral antigens on the cell surface. This poses a challenge for identifying HBV epitopes as the exact regions of the HBV genome that are integrated can differ from patient to patient. Further complicating this effort is the existence of thousands of HBV strains distributed across 10 different HBV genotypes (genotype A to J), which differ from each other by at least 8% at the nucleotide level.

The present disclosure provides, among other things, isolated peptides derived from hepatitis B virus (HBV), and fragments or derivatives thereof. Various peptide-based molecules including complexes (e.g., peptide-MHC (pMHC) complexes), fusion proteins, and conjugates comprising the peptides are also provided. Further provided herein are polynucleotides and vectors encoding the peptides or peptide-based molecules described herein. Binding moieties (e.g., antibodies, alternative scaffolds, T-cell receptors (TCRs) or chimeric antigen receptors (CARs)) that bind to the peptides or peptide-based molecules are also provided. The compositions of the present disclosure can be used to induce an immune response against HBV infection and/or for treatment or prevention of an HBV-induced disease or disorder. In one aspect, the present disclosure also provides methods for identifying an immunogenic virus-derived peptide.

Lymphocytes, such as T cells, play important roles in adaptive anti-infection, antitumor, autoimmune, and transplant rejection responses. Generally, a T cell mediated immune response involves close contact, e.g., an immunological synapse, between a T cell and an antigen presenting cell (APC). The pairing of several molecules is involved in the formation of the immunological synapse, including, but not limited to: (a) a T-cell receptor (TCR) on a T cell, which specifically binds to a peptide presented in the peptide binding groove of a major histocompatibility complex (MHC) molecule on an APC; and (b) CD28 (on the T cell), which pairs with a B7 molecule on the APC. A TCR, together with CD3 molecules, form a TCR complex, and upon pairing of the TCR to the peptide-MHC (pMHC) complex, a signal is sent through CD3. Signaling through both the TCR complex and CD28 on the T cell results in activation of the T cell.

T cell receptors are heterodimeric structures composed of two types of chains (an α (alpha) and β (beta) chain, or a γ (gamma) and δ (delta) chain). The α chain is encoded by the nucleic acid sequence located within the α locus (on human or mouse chromosome 14), which also encompasses the entire δ locus that encodes the δ chain, and the β chain is encoded by the nucleic acid sequence located within the β locus (on mouse chromosome 6 or human chromosome 7). The majority of T cells have an αβ TCR, while a minority of T cells bear a γδ TCR. T cell receptor α and β polypeptides (and similarly γ and δ polypeptides) are linked to each other via a disulfide bond. Each of the two polypeptides that make up the TCR contains an extracellular domain comprising constant and variable regions, a transmembrane domain, and a cytoplasmic tail (the transmembrane domain and the cytoplasmic tail also being a part of the constant region).

The variable region of each TCR comprises a unique and characteristic structure, i.e., an idiotope or idiotype, that determines the specificity of the TCR. Generally, a TCR will bind to a pMHC complex only if the TCR comprises an idiotype that recognizes the peptide being presented in the context of MHC, e.g., the unique conformation of a particular pMHC complex.

Immunotherapeutic approaches to treating disease work to regulate T cell activity in vivo, e.g., to enhance anti-infection and antitumor responses, or, for example, downregulate autoimmune and transplant rejection responses. However, such methods can lack specificity since immunotherapies can target signaling by the TCR complex by binding CD3 and/or the pairing of costimulatory molecules. Such approaches can result in undesirable side effects, e.g., a hyperactive immune response or generalized immune suppression. Accordingly, therapies that take advantage of the uniquely specific interaction between a TCR and pMHC complex may provide the ability to specifically modulate the activity of specific T cells in vivo, and provide treatments based on T cell modulation.

The presentation of virus-derived peptides by MHC molecules on the surface of an infected cell and the recognition of these pMHC complexes by, and subsequent activation of, CD8+ cytotoxic T cells provides an important mechanism for immunity-based protection against viruses. Cells infected with HBV, including those that developed into cancer cells, can express various HBV-associated antigens. Peptides derived from these antigens may be displayed on the cell surface in complex with MHC molecules. Detection of an MHC-presented HBV-derived peptide by a T cell bearing the corresponding TCR, leads to targeted killing of the infected cell. However, because of the selection processes which occur during T cell maturation in the thymus, there is often a scarcity of T cells in the circulating repertoire, which recognize HBV-derived peptides with a sufficiently high level of affinity. As a result, infected cells often escape elimination by the immune system.

The identification of HBV-derived peptides presented on infected cells (e.g., HBV-induced cancer cells) can allow for the development of immunotherapeutic reagents designed to specifically target and destroy HBV-infected cells (e.g., HBV-induced cancer cells). Such reagents may be moieties that bind to the HBV-derived peptide and/or pMHC complexes and the reagents, e.g., moieties that can function by inducing a T cell response. For example, such reagents may be based on antibodies, TCRs, and/or CARs.

The present disclosure, in part, is based on a proteogenomic approach that detects MHC-associated HBV peptides from HBV infected cells. The repertoire of HLA-I-HBV peptides that are expressed in the livers of infected patients characterized herein can provide an accurate representation of HBV epitopes in a population.

HLA-restricted viral peptides as potential targets may be leveraged for delivering immunotherapeutics (such as antibodies (e.g., bispecific antibodies), engineered TCR- or CAR-based cellular therapies) to infected tissues. However, genomic variability between strains of a virus, such as HBV, in combination with differences in patient HLA alleles, can be a challenge in developing therapeutics against these peptide targets. To address this challenge, in one aspect the present disclosure provides a proteogenomics approach for generating patient-specific databases that allows for the comprehensive identification of viral peptides, e.g., HBV-derived peptides, based on the viral transcriptomes sequenced from individual patient samples. The HBV-HLA-associated peptides disclosed herein may be used in the development of immunotherapeutics (such as antibodies (e.g., bispecific antibodies), engineered TCR- or CAR-based cellular therapies) for the treatment of HBV-related diseases or disorders including hepatocellular carcinoma (HCC) and to improve clinical surveillance to monitor patient-specific HBV diversity and inform vaccine development. The proteogenomic discovery platform described herein provides a method for identifying viral-derived peptides as targets for anti-viral related immunotherapy.

### Definitions

Unless defined otherwise, all technical and scientific terms used herein have the same meaning as commonly understood by one of ordinary skill in the art to which this disclosure belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “antigen” encompasses any agent (e.g., protein, peptide, polysaccharide, glycoprotein, glycolipid, nucleotide, portions thereof, or combinations thereof) that, when introduced into an immunocompetent host (directly or upon expression as in, e.g., DNA or RNA vaccines) is recognized by the immune system of the host and is capable of eliciting an immune response by the host. The T-cell receptor (TCR) recognizes a peptide presented in the context of a major histocompatibility complex (MHC) as part of an immunological synapse. The peptide-MHC (pMHC) complex is recognized by TCR, with the peptide (antigenic determinant) and the TCR idiotype providing the specificity of the interaction. Accordingly, the term “antigen” encompasses peptides presented in the context of MHCs, e.g., pMHC complexes. The peptide displayed on MHC may also be referred to as an “epitope” or an “antigenic determinant”. The terms “peptide,” “antigenic determinant,” “epitopes,” etc., encompass not only those presented naturally by antigen-presenting cells (APCs), but may be any desired peptide so long as it is recognized by an immune cell, e.g., when presented appropriately to the cells of an immune system. For example, a peptide having an artificially prepared amino acid sequence may also be used as the epitope.

A single antigen (such as an antigenic polypeptide) may have more than one epitope. Epitopes may be defined as structural or functional. Functional epitopes are generally a subset of structural epitopes and are defined as those residues that directly contribute to the affinity of the interaction between an MHC molecule and the antigen. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The terms “major histocompatibility complex,” and “MHC” encompass the terms “human leukocyte antigen” or “HLA” (the latter two of which are generally reserved for human MHC molecules), naturally occurring MHC molecules (e.g., MHC class I molecule comprising MHC class I α (heavy) chain and β2 microglobulin; MHC class II molecule comprising MHC class II α chain and MHC class II β chain), individual chains of MHC molecules (e.g., MHC class I α (heavy) chain, MHC class II α chain, and MHC class II β chain), individual subunits of such chains of MHC molecules (e.g., α1, α2, and/or α3 subunits of MHC class I α chain, α1-α2 subunits of MHC class II α chain, β1-β2 subunits of MHC class II β chain) as well as portions (e.g., the peptide-binding portions, e.g., the peptide-binding grooves), mutants and various derivatives thereof (including fusions proteins), wherein such portion, mutants and derivatives retain the ability to display an antigenic peptide for recognition by a TCR, e.g., an antigen-specific TCR. An MHC class I molecule comprises a peptide binding groove formed by the α1 and α2 domains of the heavy a chain that can stow a peptide of around 8-10 amino acids. Despite the fact that both classes of MHC bind a core of about 9 amino acids (e.g., 5 to 17 amino acids) within peptides, the open-ended nature of MHC class II peptide binding groove (the α1 domain of a class II MHC α polypeptide in association with the β1 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide binding groove, changing which 9-mer sits directly within the groove at any given time. In some embodiments, the peptide-MHC complex described herein may be a peptide-MHC complex from a non-human animal. In other embodiments, the peptide-MHC complex described herein may include an peptide-HLA complex, i.e., a peptide-MHC complex from a human. Conventional identifications of particular MHC variants are used herein. For example, HLA-A11 refers to a human leucocyte antigen from the A gene group (hence a class I type MHC) gene position (known as a gene locus) number 11; gene HLA-DR11, refers to a human leucocyte antigen coded by a gene from the DR region (hence a class II type MHC) locus number 11.

“MHC-peptide complex,” “peptide-MHC complex,” “pMHC complex,” “peptide-in-groove,” and the like include (i) an MHC molecule, e.g., a human and/or non-human animal MHC molecule, or portion thereof (e.g., the peptide-binding groove thereof, and e.g., the extracellular portion thereof), and (ii) an antigenic peptide (e.g., a HBV-derived peptide), where the MHC molecule and the antigenic peptide are complexed in such a manner that the pMHC complex can specifically bind a T-cell receptor. A pMHC complex encompasses cell surface expressed pMHC complexes and soluble pMHC complexes.

“HLA-peptide complex,” “peptide-HLA complex,” “pHLA complex,” and the like refer to an MHC-peptide complex wherein the MHC molecule is a Human Leukocyte Antigen (HLA) molecule.

The term “T cell” or “T lymphocyte” is used herein in its broadest sense to refer to all types of immune cells expressing CD3, including, but not limited to, T-helper cells (CD4+ cells), cytotoxic T-cells (CD8+ cells), tumor infiltrating cytotoxic T cells (TIL; CD8+ T cell), CD4+CD8+ T cells, T-regulatory cells (Treg), and NK-T cells. T cells can include thymocytes, naïve T cells, memory T cells, immature T cells, mature T cells, resting T cells, or activated T cells. T cells may also include “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain.

The term “antigen presenting cell” or “APC” refers to any cell that presents on the surface of the cell an antigen in association with a major histocompatibility complex molecule, either MHC class I or MHC class II molecule, or both.

Terms “antibody,” “antibodies,” “immunoglobulin”, and the like refer to immunoglobulin molecules and immunologically active portions of immunoglobulin molecules, i.e., molecules that contain an antigen binding site that specifically binds an antigen, whether natural or partly or wholly synthetically produced. The terms include monoclonal antibodies, multispecific antibodies (e.g., bispecific antibodies), human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909, incorporated herein by reference in its entirety, and Ig-DARTS such as those disclosed in U.S. Pat. Appl. Pub. 2009/0060910, incorporated herein by reference in its entirety. Antibodies useful in the present disclosure include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgAQ1 and IgA2) or subclass.

The term “specifically binds,” “binds in a specific manner,” “antigen-specific” or the like, indicates that the molecules involved in the specific binding are able to form a complex with each other that is relatively stable under physiological conditions, and are unable to form stable complexes non-specifically with other molecules outside the specified binding pair. Accordingly, a peptide binding moiety (e.g., an antibody, an alternative scaffold, a CAR, or a TCR) that binds in a specific manner to an HBV-derived peptide, or a peptide-based molecule (such as a complex (e.g., a pMHC complex), fusion protein, or conjugate comprising the described peptide) indicates that the peptide binding moiety forms a stable intermolecular non-covalent bonds with the HBV-derived peptide or peptide-based molecule (such as a complex (e.g., a pMHC complex), fusion protein, or conjugate comprising the described peptide). Specific binding can be characterized by an equilibrium dissociation constant (K_{D}) in the low micromolar to picomolar range (i.e., a smaller K_{D} denotes a tighter binding). High specificity may be in the low nanomolar range, with very high specificity being in the picomolar range. For example, a peptide binding moiety may exhibit binding to an HBV-derived peptide or peptide-based molecule (such as a complex (e.g., a pMHC complex), fusion protein, or conjugate comprising the described peptide) with a K_{D} of about 3000 nM or less, about 2000 nM or less, about 1000 nM or less, about 500 nM or less, about 300 nM or less, about 200 nM or less, about 100 nM or less, about 50 nM or less, about 1 nM or less, or about 0.5 nM or less. Methods for determining whether two molecules specifically bind to one another are well known in the art and include, for example, equilibrium dialysis, surface plasmon resonance, and the like.

The terms “protein” and “polypeptide”, used interchangeably herein, encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, PEGylation, biotinylation, etc.). Small polypeptides of less than 100 amino acids, preferably less than 50 amino acids, may be referred to as “peptides”.

The terms “polynucleotide” and “nucleic acid”, used interchangeably herein, include polymeric forms of nucleotides of any length, including ribonucleotides (RNA), deoxyribonucleotides (DNA), or analogs or modified versions thereof. They include single-, double-, and multi-stranded DNA or RNA, genomic DNA, complementary DNA (cDNA), DNA-RNA hybrids, and polymers comprising purine bases, pyrimidine bases, or other natural, chemically modified, biochemically modified, non-natural, or derivatized nucleotide bases.

The term “operably linked” or the like refers to a juxtaposition wherein the components described are in a relationship permitting them to function in their intended manner. For example, a control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control a gene of interest (or sequence of interest). The term “expression control sequence” includes polynucleotide sequences, which are necessary to affect the expression and processing of coding sequences to which they are ligated. “Expression control sequences” include: appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance polypeptide stability; and when desired, sequences that enhance polypeptide secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing, and can also include additional components whose presence is advantageous, for example, leader sequences and fusion partner sequences.

The term “isolated” refers to a homogenous population of molecules (such as polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The term “derivative” as used herein refers to a peptide, polypeptide, or polynucleotide, or a variant or analog thereof, comprising one or more mutations and/or chemical modifications as compared to a reference peptide, polypeptide or polynucleotide. Mutations and/or chemical modifications are further detailed below and can include, for example, insertions, substitutions, deletions, transversions, and/or inversions at one or more locations in the amino acid or nucleotide sequence.

The terms “treat” or “treatment” of a state, disorder, disease, or condition include: (1) preventing, delaying, or reducing the incidence and/or likelihood of the appearance of at least one clinical or sub-clinical symptom of the state, disorder, disease, or condition developing in a subject that may be afflicted with or predisposed to the state, disorder, disease, or condition, but does not yet experience or display clinical or subclinical symptoms of the state, disorder, disease, or condition; or (2) inhibiting the state, disorder, disease, or condition, i.e., arresting, reducing or delaying the development of the disease or a relapse thereof or at least one clinical or sub-clinical symptom thereof; or (3) relieving the state, disorder, disease, or condition, i.e., causing regression of the state, disorder, disease, or condition or at least one of the clinical or sub-clinical symptoms of the state, disorder, disease, or condition. The benefit to a subject to be treated is either statistically significant or at least perceptible to the patient or to the physician.

An “individual” or “subject” or “animal” refers to humans, veterinary animals (e.g., cats, dogs, cows, horses, sheep, pigs, etc.) and experimental animal models of diseases (e.g., mice, rats). In a preferred embodiment, the subject is a human.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “administration” and the like refers to and includes the administration of a composition to a subject or system (e.g., to a cell, organ, tissue, organism, or relevant component or set of components thereof). The skilled artisan will appreciate that route of administration may vary depending, for example, on the subject or system to which the composition is being administered, the nature of the composition, the purpose of the administration, etc. For example, in certain embodiments, administration to an animal subject (e.g., to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

In accordance with the disclosure herein, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### Peptides Disclosed Herein

In one aspect, the present disclosure provides isolated peptides comprising an amino acid sequence derived from hepatitis B virus (HBV).

The HBV genome demonstrates genetic variability with an estimated rate of 1.4-3.2×10⁻⁵ nucleotide substitutions per year for each site. A substantial number of virus variants occur due to nucleotide misincorporations in the absence of any proofreading capacity via the viral polymerase. This variability has resulted in subtypes of the virus. HBV has been classified into genotypes based on an inter-group divergence of at least 8% in the complete genomic sequence, each having a defined geographical distribution. For instance, genotype A is common in sub-Saharan Africa, Northern Europe, and Western Africa; genotypes B and C are widespread in Asia; genotype C primarily exists in Southeast Asia; genotype D is found in in Africa, Europe, Mediterranean countries, and India; genotype G is observed in France, Germany, and the United States; and genotype H is largely seen in Central and South America. Genotype I has been recently observed in Laos, as well as Vietnam. Genotype J has been reported in the Ryukyu Islands in Japan.

HBV is an enveloped DNA virus and a member of the Hepadnaviridae family. HBV contains a small, partially double-stranded (DS), relaxed-circular DNA (rcDNA) genome that replicates by reverse transcription of pregenomic RNA (pgRNA), an RNA intermediate. The circular DNA genome of HBV is considered atypical since the DNA is not fully double-stranded. One end of the full length strand is linked to the viral DNA polymerase. The genome of the full-length strand is approximately 3020-3320 nucleotides long, while the genome of the short-length strand is 1700-2800 nucleotides long. The negative-sense (i.e., non-coding) strand is complementary to the viral mRNA.

There are four genes encoded by the HBV genome: genes C, X, P, and S. The core protein is coded for by gene C (HBcAg), and the start codon of gene C is preceded by an upstream in-frame AUG start codon from which the pre-core protein is produced. The HBeAg is produced by proteolytic processing of the pre-core protein. The DNA polymerase is encoded by gene P. Gene S codes for the surface antigen (HBsAg (Hepatitis B core antigen)). The HBsAg gene is one long open reading frame containing three in frame start codons (ATG) that sub-divides the gene into three components: pre-S1, pre-S2, and S. As a result of the multiple start codons, proteins of three different sizes called Large (pre-S1/pre-S2/S), Middle (pre-S2/S), and Small (S) are generated. Gene X may be related to the development of liver cancer and may stimulate genes that promote cell growth and inactivate growth regulating molecules.

The viral DNA is localized within the nucleus following infection of the cell. The partially double-stranded DNA is rendered fully double-stranded by completion of the positive sense strand and removal of a protein molecule from the negative sense strand and a short sequence of RNA from the positive sense strand. Non-coding bases are removed from the ends of the negative sense strand and the ends are rejoined.

The HBV life cycle initiates when the virus attaches to the host cell and is then internalized. The sodium-taurocholate co-transporting polypeptide (NTCP) can be a functional receptor in HBV infection. The virion relaxed circular DNA (rcDNA) is transported to the nucleus, where the virion rcDNA is repaired to form a covalently closed-circular DNA (cccDNA). The episomal cccDNA acts as the template for the transcription of the pregenomic RNA (pgRNA), as well as other viral mRNAs by the host RNA polymerase II. The transcripts are then exported to the cytoplasm for subsequent translation of the viral proteins. Reverse transcriptase (RT) binds to the pgRNA and activates assembly of the core proteins into immature RNA-containing nucleocapsids. The immature nucleocapsids mature as the pgRNA is reversed transcribed by RT to make the mature rcDNA. A distinctive feature of hepadnavirus reverse transcription is the RT primed initiation of minus-strand DNA synthesis, thereby leading to the covalent linkage of RT to the 5′ end of the minus-strand DNA.

The mature, rcDNA-containing nucleocapsids are then enveloped by the viral surface proteins and secreted as virions via the secretion pathway or are recycled back to the nucleus for further amplification of the pool of cccDNA via the recycling pathway.

Amino acids sequences derived from HBV disclosed herein may include naturally occurring proteogenic amino acids as well as non-proteogenic amino acids and non-naturally occurring amino acids such as amino acid analogs. In some embodiments, the amino acids that may be used in the practice of the present disclosure may include, for example, without limitation, naturally occurring proteogenic (L)-amino acids, their optical (D)-isomers, chemically modified amino acids, including, e.g., amino acid analogs such as, e.g., selenocysteine (Sec), penicillamine (3-mercapto-D-valine), pyroglutamic acid (5-oxoproline), etc., naturally occurring non-proteogenic amino acids such as norleucine, and chemically synthesized amino acids that have properties known in the art to be characteristic of an amino acid, and amino acid equivalents.

In some embodiments, an isolated peptide of the present disclosure comprises an amino acid sequence that is at least about 60%, at least about 65%, at least about 70%, at least about 75%, at least about 80%, at least about 85%, at least about 90%, at least about 95%, or at least about 99% identical to the amino acid sequence of any one of SEQ ID NOs: 1-54 and 110-112, or a pharmaceutically acceptable salt thereof, or a fragment or derivative thereof. In some embodiments, an isolated peptide of the present disclosure comprises an amino acid sequence of any one of SEQ ID NOs: 1-54 and 110-112. In some embodiments, an isolated peptide of the present disclosure consists essentially of an amino acid sequence of any one of SEQ ID NOs: 1-54 and 110-112. In some embodiments, the isolated peptide consists of an amino acid sequence of any one of SEQ ID NOs: 1-54 and 110-112. In some embodiments, the isolated peptide comprises two or more sequences selected from any one or SEQ ID NOs: 1-54 and 110-112, or a pharmaceutically acceptable salt thereof, or a fragment or derivative thereof.

A list of non-limiting examples of HBV-derived peptides are provided in Table 1 below.

TABLE 1
Examples of HBV-derived Peptides

| SEQ ID NO: | Peptide Sequence |
|---|---|
| 1 | ASRELVVSY |
| 2 | ENITSGFLGPL |
| 3 | FLLTRILTI |
| 4 | FSLTKILTIPQ |
| 5 | FSLTKILTIPQS |
| 6 | FVGLSPTVW |
| 7 | FVGLSPTVWL |
| 8 | GLSPTVWLSV |
| 9 | GMLPVCPLI |
| 10 | GMLPVCPLL |
| 11 | GSLPQEHIIQK |
| 12 | GSLPQEHIVQK |
| 13 | GTLPQEHIVHK |
| 14 | GTLPQEHIVQK |
| 15 | GVWIRTPPAYR |
| 16 | HLYSHPIIL |
| 17 | IASGLLGPL |
| 18 | IASGLLGPLLVL |
| 19 | ITSGFLGPL |
| 20 | ITSGFLGPLL |
| 21 | KILTIPQSLDSW |
| 22 | KPRKGMGTNL |
| 23 | KYTSFPWLL |
| 24 | LPETTVVRR |
| 25 | LPFRPTTGR |
| 26 | LPQEHIVHK |
| 27 | LPSDFFPSI |
| 28 | LPYRPTTGR |
| 29 | LQDPRVRAL |
| 30 | LQDPRVRALY |
| 31 | LTIPQSLDSW |
| 32 | LTIPQSLDSWW |
| 33 | LTRILTIPQSL |
| 34 | LYAAVTNFL |
| 35 | MENIASGLLGPL |
| 36 | MENITSGFLGPL |
| 37 | NILSPFMPLL |
| 38 | NITSGFLGPL |
| 39 | PQSLDSWLT |
| 40 | QSPTSNHSL |
| 41 | SAISSTFSK |
| 42 | STISSTFSK |
| 43 | STLPETTVVRR |
| 44 | TASPISSIF |
| 45 | TCIPIPSSW |
| 46 | TIPQSLDSW |
| 47 | TRILTIPQSL |
| 48 | TVSAISSTF |
| 49 | VGLSPTVWL |
| 50 | WTSLNFLGGTTV |
| 51 | YPALMPLYA |
| 52 | IPIPSSWAF |
| 53 | TASAISSTF |
| 54 | TASPLSSIF |
| 110 | GTLPQDHIVQK |
| 111 | GSLPQDHIIQK |
| 112 | GTLPQEHIVLK |

In some embodiments, an isolated peptide of the present disclosure comprises an amino acid sequence GX₁LPQX₂HIX₃X₄K (SEQ ID NO: 107), wherein X₁ is S or T, X₂ is E or D, X₃ is V or I, and X₄ is Q, H or L, or a pharmaceutically acceptable salt thereof, or a fragment or derivative thereof. In some embodiments, an isolated peptide of the present disclosure consists of an amino acid sequence GX₁LPQX₂HIX₃X₄K (SEQ ID NO: 107), wherein X₁ is S or T, X₂ is E or D, X₃ is V or I, and X₄ is Q, H or L, or a pharmaceutically acceptable salt thereof, or a fragment or derivative thereof.

A peptide of the disclosure may be synthetically produced or produced by hydrolysis. Synthetically produced peptides can include randomly generated peptides, specifically designed peptides, and peptides where at least some of the amino acid positions are conserved among several peptides and the remaining positions are random. Alternatively, a peptide of the present disclosure may be produced by expression in a heterologous host cell.

In nature, peptides that are produced by hydrolysis undergo hydrolysis prior to binding of the antigen to an MHC molecule. Class I MHC typically present peptides derived from proteins actively synthesized in the cytoplasm of the cell. In contrast, class II MHC typically present peptides derived either from exogenous proteins that enter a cell's endocytic pathway or from proteins synthesized in the endoplasmic reticulum (ER). Intracellular trafficking permits a peptide to become associated with an MHC molecule.

The binding of a peptide to an MHC peptide binding groove can control the spatial arrangement of MHC and/or peptide amino acid residues recognized by a TCR. Such spatial control is due in part to hydrogen bonds formed between a peptide and an MHC molecule. Based on the knowledge on how peptides bind to various MHC molecules, the major MHC anchor amino acids and the surface exposed amino acids that are varied among different peptides can be determined.

Preferably, the length of an MHC-binding peptide is from about 5 to about 40 amino acid residues, more preferably from about 6 to about 30 amino acid residues, and even more preferably from about 8 to about 20 amino acid residues, and even more preferably between about 9 and 11 amino acid residues, including any size peptide between 5 and 40 amino acids in length, in whole integer increments (i.e., 5, 6, 7, 8, 9 . . . 40). While naturally MHC class II-bound peptides vary from about 9-40 amino acids, in nearly all cases the peptide can be truncated to an about 9-11 amino acid core without loss of MHC binding activity or T cell recognition.

In some embodiments, the isolated peptides of the disclosure may be about 8-12 amino acids in length. For example, a peptide disclosed herein may be 8 amino acids, 9 amino acids, 10 amino acids, 11 amino acids, or 12 amino acids in length.

The peptides of the disclosure may comprise one or more reverse peptide bonds, one or more non-peptide bonds, one or more chemical modifications, one or more D-isomers of amino acids, or any combination thereof.

In some embodiments, the peptide may be modified to comprise one or more reverse peptide bonds or non-peptide bonds. Such modification may improve stability and/or binding of the peptide to MHC molecules to elicit a stronger immune response. In a reverse peptide bond, amino acid residues are not joined by peptide (—CO—NH—) linkages but the peptide bond is reversed. Such retro-inverso peptidomimetics may be made using methods known in the art, for example such as those described in Meziere et al., 1997 (Meziere C., et al. Immunol 1997). This approach involves making pseudopeptides containing changes involving the backbone, and not the orientation of side chains. Such pseudopeptides may be useful, for example, for desired MHC binding and/or T helper cell responses. Retro-inverse peptides, which contain NH—CO bonds instead of CO—NH peptide bonds, are much more resistant to proteolysis. Additional non-peptide bond that may be used are, for example, —CH₂—NH, —CH₂S—, —CH₂CH₂—, —CH═CH—, —COCH₂—, —CH(OH)CH₂—, and —CH₂SO—.

The amino acid residues comprising the peptides of the disclosure may be chemically modified. Non-limiting examples of chemical modifications include, for example, phosphorylation, acetylation, deamidation acylation, amidination, pyridoxylation of lysine, reductive alkylation, trinitrobenzylation of amino groups with 2,4,6-trinitrobenzene sulphonic acid (TNBS), amide modification of carboxyl groups and sulphydryl modification by performic acid oxidation of cysteine to cysteic acid, formation of mercurial derivatives, formation of mixed disulfides with other thiol compounds, reaction with maleimide, carboxymethylation with iodoacetic acid or iodoacetamide and carbamoylation with cyanate at alkaline pH. Chemical modifications may not correspond to those that may be present in vivo.

For example, modification of, for example, arginyl residues in proteins may be based on the reaction of vicinal dicarbonyl compounds such as phenylglyoxal, 2,3-butanedione, and 1,2-cyclohexanedione to form an adduct. Another example is the reaction of methylglyoxal with arginine residues. Cysteine can be modified without concomitant modification of other nucleophilic sites such as lysine and histidine. Selective reduction of disulfide bonds in proteins can also be performed. Disulfide bonds can be formed and oxidized during the heat treatment of biopharmaceuticals. Woodward's Reagent K may be used to modify specific glutamic acid residues. N-(3-(dimethylamino)propyl)-N′-ethylcarbodiimide can be used to form intra-molecular crosslinks between a lysine residue and a glutamic acid residue. For example, diethylpyrocarbonate and 4-hydroxy-2-nonenal can be used to modify histidyl residues in proteins. The reaction of lysine residues and other α-amino groups is, for example, useful in binding of peptides to surfaces or the cross-linking of proteins/peptides. Lysine is the site of attachment of poly(ethylene)glycol and the major site of modification in the glycosylation of proteins. Methionine residues in proteins can be modified with e.g. iodoacetamide, bromoethylamine, and chloramine T. Tetranitromethane and N-acetylimidazole can be used for the modification of tyrosyl residues. Cross-linking via the formation of dityrosine can be accomplished with hydrogen peroxide/copper ions. N-bromosuccinimide, 2-hydroxy-5-nitrobenzyl bromide or 3-bromo-3-methyl-2-(2-nitrophenylmercapto)-3H-indole (BPNS-skatole) have been used in recent studies for the modification of tryptophan. Successful modification of therapeutic proteins and peptides with PEG can lead to an extension of circulatory half-life while cross-linking of proteins/peptides with glutaraldehyde, polyethylene glycol diacrylate and formaldehyde can be used for the preparation of hydrogels. Chemical modification of allergens for immunotherapy can be achieved by carbamylation with potassium cyanate.

Peptides of the present disclosure may also be synthesized with additional chemical groups present at their N- and/or C-termini, to enhance the stability, bioavailability, and/or affinity of the peptides.

N-terminal modifications can include methylation (e.g., —NHCH₃ or —N(CH₃)₂), acetylation (e.g., with acetic acid or a halogenated derivative thereof such as α-chloroacetic acid, α-bromoacetic acid, or α-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide. Additionally, hydrophobic groups such as carbobenzoxyl, dansyl, or t-butyloxycarbonyl groups may be added to the N-terminus. Likewise, an acetyl group or a 9-fluorenylmethoxy-carbonyl group may be placed at the N-terminus.

C-terminal modifications can include replacing the free acid with a carboxamide group or forming a cyclic lactam at the carboxy terminus to introduce structural constraints. One can also cyclize the peptides of the disclosure, or incorporate a desamino or descarboxy residue at the termini of the peptide, so that there is no terminal amino or carboxyl group, to decrease susceptibility to proteases or to restrict the conformation of the peptide. C-terminal functional groups of the compounds of the present disclosure include amide, amide lower alkyl, amide di(lower alkyl), lower alkoxy, hydroxy, and carboxy, and the lower ester derivatives thereof, and the pharmaceutically acceptable salts thereof. Additionally, the hydrophobic group, t-butyloxycarbonyl, or an amido group may be added to the C-terminus.

Further examples of non-natural modifications include incorporation of non-encoded α-amino acids, photoreactive cross-linking amino acids, N-methylated amino acids, and β-amino acids, backbone reduction, retroinversion by using D-amino acids, and C-terminal amidation and PEGylation.

Peptides described herein may comprise one or more (e.g., 1, 2, 3, or 4) amino acid substitutions and/or insertions and/or deletions. Amino acid substitution means that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent another inserted amino acid residue. One or more (e.g., 1, 2, 3 or 4) amino acids may be substituted and/or inserted and/or deleted from the sequence of any one of SEQ ID NOs: 1-54 and 110-112. Each substitution and/or insertion and/or deletion can take place at any position of any one of SEQ ID NOs: 1-54 and 110-112.

In some embodiments, the peptides of the disclosure may comprise additional amino acids (e.g., 1, 2, 3 or 4) at the C-terminal end and/or at the N-terminal end of the sequence of any one SEQ ID NOs: 1-54 and 110-112. A peptide of the disclosure may comprise the amino acid sequence of any one of SEQ ID NOs: 1-54 and 110-112 except for one or more (e.g., 1, 2, 3, or 4) amino acid substitutions, insertions or deletions.

Inserted amino acids and substituted amino acids may be naturally occurring amino acids or may be non-naturally occurring amino acids and, for example, may contain a non-natural side chain, and/or be linked together via non-native peptide bonds. Such altered peptide ligands are discussed further in Douat-Casassus et al., J. Med. Chem, 2007; 50(7):1598-609 and Hoppes et al., J. Immunol 2014; 193(10):4803-13 and references therein. If more than one amino acid residue is substituted and/or inserted, the replacement/inserted amino acid residues may be the same as each other or different from one another. Each replacement amino acid may have a different side chain to the amino acid being replaced.

D-amino acids may be substituted for the L-amino acids in the antigenic peptides of the disclosure. In addition, non-standard amino acids (i.e., other than the common naturally occurring proteinogenic amino acids such as β-γ-δ-amino acids, as well as many derivatives of L-α-amino acids) may also be used for substitutions or additions to produce peptides of the present disclosure.

Amino acid substitutions may be conservative, by which it is meant the substituted amino acid has similar chemical properties to the original amino acid. For example, the following groups of amino acids share similar chemical properties such as size, charge, and polarity: Group 1—Ala, Ser, Thr, Pro, Gly; Group 2—Asp, Asn, Glu, Gln; Group 3—His, Arg, Lys; Group 4—Met, Leu, Ile, Val, Cys; Group 5—Phe, Thy, Trp.

Substantial changes in function (e.g., affinity for MHC molecules and/or TCRs) can be made by selecting substitutions that are less conservative than those described above, in other words, selecting residues that differ more significantly in their effect on maintaining the structure of the peptide backbone in the area of the substitution (e.g., as a sheet or helical conformation), the bulk of the side chain, or the charge or hydrophobicity of the peptide at the positions involved for MHC or TCR binding. The substitutions which in general are expected to produce the greatest changes in peptide properties will be those in which (a) a hydrophilic residue, e.g. Ser, is substituted for (or by) a hydrophobic residue, e.g. Leu, Ile, Phe, Val or Ala; (b) a residue having an electropositive side chain, e.g., Lys, Arg, or His, is substituted for (or by) an electronegative residue, e.g. Glu or Asp; or (c) a residue having a bulky side chain, e.g. Phe, is substituted for (or by) a residue not having a side chain, e.g., Gly.

One can also replace the naturally occurring side chains of the 20 genetically encoded amino acids (or the stereoisomeric D-amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. For example, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Other examples of amino acid replacements include stereoisomers (e.g., D-amino acids) and unnatural amino acids such as, for example, L-ornithine, L-homocysteine, L-homoserine, L-citrulline, 3-sulfino-L-alanine, N-(L-arginino)succinate, 3,4-dihydroxy-L-phenylalanine, 3-iodo-L-tyrosine, 3,5-diiodo-L-tyrosine, triiodothyronine, L-thyroxine, L-selenocysteine, N-(L-arginino)taurine, 4-aminobutylate, (R,S)-3-amino-2-methylpropanoate, a,a-disubstituted amino acids, N-alkyl amino acids, lactic acid, β-alanine, 3-pyridylalanine, 4-hydroxyproline, O-phosphoserine, N-methylglycine, N-acetylserine, N-formylmethionine, 3-methylhistidine, 5-hydroxylysine, nor-leucine, and other similar amino acids and imino acids.

The amino acid residues that do not substantially contribute to interactions with the T-cell receptor can be modified by replacement with other amino acid whose incorporation does not substantially affect T-cell reactivity and does not eliminate binding to the relevant MHC.

The peptides may also comprise isosteres of two or more residues. An “isostere” as used here refers to a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks.

Combinations of several substitutions/additions/deletions at more than one position can be developed and tested to determine if the combination results in an additive or synergistic effects on the immunogenicity of the peptide. In some embodiments, no more than 4 positions within the peptide are simultaneously altered.

Preferably, peptides of the disclosure bind to an MHC molecule in the peptide binding groove of the MHC molecule. Generally, the amino acid modifications described above will not impair the ability of the peptide to bind to the MHC molecule. In some embodiments, the amino acid modifications improve the ability of the peptide to bind to the MHC molecule. For example, mutations may be made at positions which anchor the peptide to the MHC molecule. Such anchor positions and the preferred residues at these locations for peptides which bind, in particular, HLA-A*02 may comprise, e.g., amino acids residues at position 2, and/or at the C-terminus of the peptide, which may be considered primary anchor positions. Preferred anchor residues may be different for each HLA type. As a non-limiting example, the preferred amino acids in position 2 for HLA-A*02 are Leu, lie, Val, or Met and at the C-terminus are Val or Leu. Multiple positions may be important for stable peptide binding to HLA-A*02, including positions 2, 3, 5-7, and 9. The anchor residues at position 2 and 9 may be of prime importance for peptide binding to HLA-A2. However, other peptide side chains, e.g., at position 3, may contribute to the stability of the interaction. In certain cases, the optimal length for peptide binding can be longer than 9 residues.

The immunologic properties of peptides can be described as a function of binding to MHC molecules (Kₒₙ and K_{off}) and TCR (affinity of interaction between TCR and MHC-peptide complexes). Modifications of primary MHC anchor residues exhibit a significant degree of predictability about overall impact on binding to MHC molecules. Modifications of secondary MHC anchor residues can impact the affinity of interaction of the MHC-peptide complex to TCR as well as with the Kₒₙ and K_{off} relative to peptide-MHC interaction.

When the HBV peptide is a mutant peptide, T cell lines against a natural (non-mutated) epitope are generated, and an immunization strategy potent enough to generate a useful response in transgenic mice carrying human MHC (such as the A2 allele) is used. HBV peptides are interrogated ex vivo in the presence of competent APCs and the functional impact of T cells specific for natural (non-mutated) epitopes is measured. The evaluation is done at various concentrations of HBV peptide, because the expected effect is biphasic in the instance of cross reactive peptides (activating at limited concentrations and inhibiting at higher concentrations, due to antigen-induced cell death [AICD]). Measurement of the following three parameters is used to define basic and useful characteristics of HBV peptides:
1. Minimal required concentration of HBV metrics to induce effects indicative of T cell activation (e.g., cytokine [e.g., IFN-γ] production);
2. Maximal (peak value) effect (e.g., cytokine [e.g., IFN-γ] production) at any HBV peptide concentration; and
3. HBV peptide concentration at peak value of activating effect (e.g., cytokine [e.g., IFN-γ] concentration).

By way of a non-limiting example, HBV peptides that result in reduced values associated with parameters number 1 and number 3, but increased number 2, can be useful. Use of natural epitope and/or unrelated non-cross-reactive peptide as references is valuable for identifying classes of peptides of possible value. Peptides possessing properties quantitatively comparable to or even moderately attenuated from those of natural epitopes are still considered useful since, while they retain cross-reactivity, they may exhibit immunologic properties that are distinct from those of the natural peptide, e.g., reduced capacity to break tolerance or reestablish responsiveness in vivo or lower propensity to induce AICD.

In addition to practicality and rapidity, additional advantages of this screening approach include, but are not limited to, use of more relevant polyclonal T cell lines instead of potentially biased T cell clones as a read out, and the composite value, integrating parameters such as Kₒₙ, K_{off} and TCR affinity that can translate into cross-reactivity and functional avidity of peptide-MHC complexes relative to TCR. These parameters can be predictive of the in vivo immunologic properties and thus can define useful panels of peptides eligible for further evaluation, optimization and practical applications. Peptides that bind to MHC and retain cross-reactivity against TCR specific for the nominal wild-type peptide are predicted to elicit a measurable effect in this assay.

A peptide of the disclosure, or pharmaceutically acceptable salt thereof, or fragment or derivative thereof, may be used to induce an immune response. If this is the case, it is important that the immune response is specific to the intended target (e.g., HBV) to avoid the risk of unwanted side effects that may be associated with an “off target” immune response. Therefore, it is preferred that the amino acid sequence of a peptide of the disclosure does not match the amino acid sequence of a peptide from any other endogenous protein(s), particularly that of another human protein. Also, the amino acid modifications described herein should not impair the ability of the peptide inducing an antigen-specific immune response when presented in a complex with an MHC molecule on the surface of an antigen presenting cell (APC).

The peptides may be also modified to improve half-life and/or bioavailability, for example, by PEGylation, glycosylation, polysialylation, HESylation, recombinant PEG mimetics, Fc fusion, albumin fusion, nanoparticle attachment, nanoparticulate encapsulation, cholesterol fusion, iron fusion, or acylation.

The peptides of the disclosure can also serve as structural models for non-peptidic compounds with similar biological activity. A variety of techniques can be used to construct compounds with the same or similar desired biological activity as the lead peptide compound, but with more favorable activity than the lead with respect to solubility, stability, and susceptibility to hydrolysis and proteolysis. These techniques include replacing the peptide backbone with a backbone composed of amidates, phosphonates, carbamates, sulfonamides, secondary amines, and N-methylamino acids.

Multiple peptides described herein may be operably linked together. Accordingly, in one aspect, the present disclosure provides an isolated peptide or polypeptide comprising two or more amino acid sequences selected from SEQ ID NO: 1-54 and 110-112, or a derivative thereof, or a pharmaceutically acceptable salt thereof. For example, such a multi-epitope peptide or polypeptide may comprise include 2 to 50, 2 to 40, 2 to 30, 5 to 25, 5 to 20, or 10 to 15 single-epitope peptides as described herein (e.g., SEQ ID NO: 1-54 and 110-112). The single-epitope peptides (e.g., SEQ ID NO: 1-54 and 110-112) may be arranged in any order, and may be identical or different.

In some embodiments, the present disclosure provides an isolated peptide or polypeptide comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 amino acid sequences selected from SEQ ID NO: 1-54 and 110-112, or a derivative thereof, or a pharmaceutically acceptable salt thereof.

The single-epitope peptides may be linked via a linker. The linker may comprise of relatively small, neutral molecules, such as amino acids or amino acid mimetics, which are substantially uncharged under physiological conditions. The linker can be selected from, e.g., Table 2, or other neutral spacers of nonpolar amino acids or neutral polar amino acids. It will be understood that the optionally present linker need not be comprised of the same residues and thus may be a hetero- or homo-oligomer. When present, the linker will usually be at least one or two residues, more usually three to six residues.

Peptides of the disclosure can be synthesized by e.g., solid phase synthesis. As such, the peptides may be immobilized, for example to a solid support such as a bead. Peptides of the disclosure may be synthesized by the Fmoc-polyamide mode of solid-phase peptide synthesis. Temporary N-amino group protection is afforded by the 9-fluorenylmethyloxycarbonyl (Fmoc) group. Repetitive cleavage of this highly base-labile protecting group is done using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalizing agent). The peptide-to-resin cleavable linked agent used is the acid-labile 4-hydroxymethyl-phenoxyacetic acid derivative. All amino acid derivatives are added as their preformed symmetrical anhydride derivatives except for asparagine and glutamine, which are added using a reversed N,N-dicyclohexyl-carbodiimide/1-hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used include ethanedithiol, phenol, anisole and water, the exact choice depending on the constituent amino acids of the peptide being synthesized. Also, a combination of solid phase and solution phase methodologies for the synthesis of peptides is possible.

Trifluoroacetic acid is removed by evaporation in vacuo, with subsequent trituration with diethyl ether affording the crude peptide. Any scavengers present are removed by a simple extraction procedure which on lyophilization of the aqueous phase affords the crude peptide free of scavengers.

Purification may be performed by techniques such as re-crystallization, ion-exchange chromatography, size exclusion chromatography, hydrophobic interaction chromatography and reverse-phase high performance liquid chromatography using e.g. acetonitrile/water gradient separation, or a combination thereof.

Peptides may be analyzed using thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

Alternatively, the peptide may be produced by recombinant expression in a heterologous host cell. Such methods typically involve the use of a vector comprising a nucleic acid sequence encoding the peptide to be expressed, to express the polypeptide in vivo; for example, in bacteria, yeast, insect or mammalian cells.

In further embodiments, in vitro cell-free systems may be used. The peptides may be isolated and/or may be provided in substantially pure form. For example, they may be provided in a form which is substantially free of other peptides or proteins.

Peptide-MHC (pMHC) Complexes Disclosed Herein

In another aspect, the disclosure provides a complex of a peptide of the disclosure and an MHC molecule. Preferably, the peptide is bound to the peptide binding groove of the MHC molecule. In some embodiments, the peptide and the MHC molecule form a non-covalent complex. In other embodiments, the peptide and the MHC molecule may be covalently linked, for example, via a linker.

MHC molecules are generally classified into two categories: class I and class II MHC molecules. An MHC class I molecule is an integral membrane protein comprising a glycoprotein heavy chain, also referred to herein as the α chain, which has three extracellular domains (i.e., α1, α2 and α3) and two intracellular domains (i.e., a transmembrane domain (TM) and a cytoplasmic domain (CYT)). The heavy chain is noncovalently associated with a soluble subunit called β2 microglobulin (β2m or β2M). An MHC class II molecule or MHC class II protein is a heterodimeric integral membrane protein comprising one a chain and one R chain in noncovalent association. The α chain has two extracellular domains (α1 and α2), and two intracellular domains (a TM domain and a CYT domain). The β chain contains two extracellular domains (β1 and β2), and two intracellular domains (a TM domain and CYT domain).

The domain organization of class I and class II MHC molecules forms the antigenic determinant binding site, e.g. the peptide-binding portion or peptide binding groove, of the MHC molecule. A peptide binding groove refers to a portion of an MHC molecule that forms a cavity in which a peptide, e.g., antigenic determinant, can bind. The conformation of a peptide binding groove is capable of being altered upon binding of a peptide to enable proper alignment of amino acid residues important for TCR binding to the peptide-MHC (pMHC) complex.

In some embodiments, MHC molecules include fragments of MHC chains that are sufficient to form a peptide binding groove. For example, a peptide binding groove of a class I protein can comprise portions of the α1 and α2 domains of the heavy chain capable of forming two β-pleated sheets and two α helices. Inclusion of a portion of the β2 microglobulin chain stabilizes the MHC class I molecule. While for most versions of MHC class II molecules, interaction of the α and β chains can occur in the absence of a peptide, the two-chain molecule of MHC class II is unstable until the binding groove is filled with a peptide. A peptide binding groove of a class II protein can comprise portions of the α1 and β1 domains capable of forming two β-pleated sheets and two α helices. A first portion of the α1 domain forms a first β-pleated sheet and a second portion of the α1 domain forms a first α helix. A first portion of the β1 domain forms a second β-pleated sheet and a second portion of the β1 domain forms a second α helix. The X-ray crystallographic structure of class II protein with a peptide engaged in the binding groove of the protein shows that one or both ends of the engaged peptide can project beyond the MHC protein. Thus, the ends of the α1 and β1 α helices of class II form an open cavity such that the ends of the peptide bound to the binding groove are not buried in the cavity. Moreover, the X-ray crystallographic structure of class II proteins shows that the N-terminal end of the MHC β chain apparently projects from the side of the MHC protein in an unstructured manner since the first 4 amino acid residues of the β chain could not be assigned by X-ray crystallography.

The peptides of the present disclosure can bind to an MHC molecule in a manner such that the pMHC complex can bind to a TCR, preferably in a specific manner. In certain embodiments, binding of the pMHC complex to the TCR may induce a T cell response.

Whether or not a given peptide will form a complex with an MHC molecule can be determined by assessing whether the MHC can be refolded in the presence of the peptide using the process described in, for example, PCT Application WO2018/083505, which is incorporated herein in its entirety for all purposes. If the peptide does not form a complex with MHC, then MHC will not refold. Refolding can be confirmed using an antibody that recognizes MHC in a folded state only. Alternatively, the ability of a peptide to stabilize MHC on the surface of transporter associated with antigen processing (TAP)-deficient cell lines such as T2 cells, which lack the capacity for TAP-mediated translocation of cytosolic peptides into the endoplasmic reticulum (ER) for peptide loading onto MHC class I molecules, or other biophysical methods to determine interaction parameters can be determined.

The peptides according to the present disclosure may be provided as an MHC groove-binding peptide. In some embodiments, the MHC groove-binding peptide can be designed such that the peptide may vary in some or all the positions involved in MHC binding. For example, the MHCBN is a comprehensive database of MHC binding and non-binding peptides compiled from published literature and existing databases. The latest version of the database has 25,860 entries including 20,717 MHC binders and 4,022 MHC non-binders for more than 450 MHCs. The database has sequence and structure data of (a) source proteins of peptides and (b) MHCs. MHCBN has a number of web tools that include: (i) mapping of peptide on query sequence; (ii) search on any field; (iii) creation of data sets; and (iv) online data submission.

In some cases, a peptide binding tool for prediction of binding to MHC-I or MHC-II can be, for example, Antibody Epitope Prediction, ANTIGENIC, BepiPred, CTLPred, DiscoTope, EPIPREDICT, Epitope Cluster Analysis, Epitope Conservancy Analysis, EUiPro, HLA Peptide Binding Predictions, HLABinding, MAPPP, MHCBench, MIC-I processing predictions, Mosaic Vaccine Tool Suite, NetChop, NetCTL, NetMHC, NetMHCII, NetMHCpan, nHLAPred-I, OptiTope, PAProC, POPI, PREDEP, Prediction of Antigenic Determinants, ProPred, ProPred-1, RankPep, SMM, SVMHC, TAPPred, VaxiJen, or combinations thereof. Additional example programs are used such as BIMAS, SYFPEITHI, or Rankpep.

In one specific embodiment, a library of altered peptides is produced by genetically engineering the library using polymerase chain reaction (PCR) or any other suitable technique to construct a DNA fragment encoding the peptide. With PCR techniques, by using oligonucleotides that are randomly mutated within particular triplet codons, the resultant fragment pool encodes all possible combination of codons at these positions. Preferably, certain of the amino acid positions are maintained constant, which are the conserved amino acids that are required for binding to the MHC peptide binding groove, and which do not contact the T cell receptor (TCR).

In some embodiments, when a library of altered peptides is produced by genetically engineering the library using polymerase chain reaction (PCR) or any other suitable technique to construct a DNA fragment encoding the peptide, the target TCR is a TCR for which it is desired to identify the peptide epitope recognized by the receptor. In some embodiments, the target TCR is from a patient with an HBV infection and/or an HBV-induced disease or disorder (e.g., hepatocellular carcinoma (HCC)). In some embodiments, the TCR includes an α-chain and a β-chain.

MHC molecules used in pMHC complexes described herein include naturally occurring full-length MHC molecules as well as individual chains of MHC molecules (e.g., MHC class I α (heavy) chain, β2-microglobulin, MHC class II α chain, and MHC class II β chain), individual subunits of such chains of MHCs (e.g., α1, α2 and/or α3 subunits of MHC class I α chain, α1 and/or α2 subunits of MHC class II α chain, β1 and/or β2 subunits of MHC class II β chain) as well as fragments, mutants, and various derivatives thereof (including fusion proteins, e.g., fusions with viral envelope proteins or fusogens), wherein such fragments, mutants, and derivatives retain the ability to display an antigenic determinant for recognition by an antigen-specific TCR. In one specific embodiment, the MHC comprises a transmembrane domain embedded in the lipid envelope of a liposome, a recombinant viral particle, or a virus-like particle (VLP).

Naturally-occurring MHC molecules are encoded by a cluster of genes on human chromosome 6 or mouse chromosome 17. MHCs are also referred to as H-2 in mice and Human Leucocyte Antigen (HLA) in humans. MHC class I molecules specifically bind CD8 molecules expressed on cytotoxic T lymphocytes (CD8+ T cells), whereas MHC class II molecules specifically bind CD4 molecules expressed on helper T lymphocytes (CD4+ T cells). MHCs include, but are not limited to, HLA specificities such as A (e.g. A1-A74), B (e.g., B1-B77), C (e.g., C1-C11), D (e.g., D1-D26), E, G, DR (e.g., DR1-DR8), DQ (e.g., DQ1-DQ9) and DP (e.g. DP1-DP6). More preferably, HLA specificities include A1, A2, A3, A11, A23, A24, A28, A30, A33, B7, B8, B35, B44, B53, B60, B62, DR1, DR2, DR3, DR4, DR7, DR8, and DR-11.

In some embodiments, the MHC molecule in a pMHC complex of the present disclosure is a human leukocyte antigen (HLA) molecule. The MHC molecule may be a human HLA molecule selected from the group consisting of HLA-A, HLA-B, HLA-C, HLA-E, HLA-F, and HLA-G. In some embodiments, the MHC class I or MHC II polypeptides may be derived from any functional human HLA-A, B, C, DR, or DQ molecules. Non-limiting examples of HLA-A alleles comprise, without limitation, A*0101, A*0201, A*0202, A*0301, A*1101, A*2301, A*2402, A*2501, A*2601, A*2901, A*2902, A*3101, A*3201, A*3301, A*3401, A*3601, A*4301, A*6601, A*6801, A*6901, A*7401, and A*8001. Non-limiting examples of HLA-B alleles comprise, without limitation, B*0702. B*0801, B*1301, B*1401, B*1402, B*1501, B*1801, B*1802, B*2701, B*2702, B*3501, B*3502, B*3701, B*3801, B*3901, B*4001, B*4101, B*4201, B*4402, B*4501, B*4601, B*4701, B*4801, B*4901, B*5001, B*5101, B*5201, B*5301, B*5401, B*5501, B*5502, B*5601, B*5701, B*5801, B*5901, B*6701, B*7301, B*1517, B*8101, B*8201, and B*8301. Non-limiting examples of HLA-C alleles comprise, without limitation, Cw*0101, Cw*0202, Cw*0303, Cw*0401, Cw*0501, Cw*0602, Cw*0701, Cw*0702, Cw*0802, Cw*1203, Cw*1401, Cw*1502, Cw*1601, Cw*1701, and. Cw*1801. Non-limiting examples of HLA-DR alleles comprise, without limitation, DRB1*0101, DRB1*0103, DRB1*1501, DRB1*1502, DRB1*1601, DRB1*1602, DRB1*0301, DRB1*0401, DRB1*0404, DRB1*1101, DRB1*1201, DRB1*1301, DRB1*1302, DRB1*1401, DRB1*1402, DRB1*0701, DRB1*0801, DRB1*0802, DRB1*0803, DRB1*0901, and DRB1*1001.

In some embodiments, the MHC class I molecule may be selected from HLA-A*02, HLA-A*01, HLA-A*03, HLA-A*11, HLA-A*23, HLA-A*24, HLA-B*07, HLA-B*08, HLA-B*40, HLA-B*44, HLA-B*15, HLA-C*04, HLA*C*03 HLA-C*07. There are also allelic variants of the above HLA types, all of which are encompassed by the present disclosure. In some embodiments, the MHC molecule may be HLA-A*02 or HLA-A*11.

The MHC molecules used herein may also be from any other mammalian or avian species, for example, non-human primates, rodents (e.g., mice), rabbits, equines, bovines, canines, felines, pigs, etc.

Naturally occurring MHC class I molecules bind peptides derived from proteolytically degraded proteins, especially endogenously synthesized proteins, by a cell. Small peptides obtained accordingly are transported into the endoplasmic reticulum where they associate with nascent MHC class I molecules before being routed through the Golgi apparatus and displayed on the cell surface for recognition by cytotoxic T lymphocytes.

Naturally occurring MHC class I molecules consist of an α (heavy) chain associated with β2-microglobulin. The heavy chain consists of subunits α1-α3. The β2-microglobulin protein and α3 subunit of the heavy chain are associated. In certain embodiments, β2-microglobulin and α3 subunit are covalently bound. In certain embodiments, β2-microglobulin and α3 subunit are non-covalently bound. The α1 and α2 subunits of the heavy chain fold to form a groove for a peptide, e.g., antigenic determinant, to be displayed and recognized by TCR.

Class I molecules can bind peptides of about 8-10 amino acids in length. All humans have between three and six different class I molecules, which can each bind many different types of peptides.

In some embodiments, the MHC contained in the pMHC complexes of the disclosure comprises (i) a class I MHC polypeptide or a fragment, mutant or derivative thereof, and, optionally, (ii) a β2 microglobulin polypeptide or a fragment, mutant or derivative thereof. In one specific embodiment, the class I MHC polypeptide is linked to the β2 microglobulin polypeptide by a peptide linker.

In one specific embodiment, the class I MHC polypeptide is a human class I MHC polypeptide selected from the group consisting of HLA-A, HLA-B, HLA-C, HLA-E, HLA-F, and HLA-G. In another specific embodiment, the class I MHC polypeptide is a murine class I MHC polypeptide selected from the group consisting of H-2K, H-2D, H-2L, H2-IA, H2-IB, H2-IJ, H2-IE, and H2-IC.

In some embodiments, the peptide disclosed herein forms a complex with one or more MHC class I α heavy chains. In some embodiments, the MHC class I α heavy chain is fully human. In some embodiments, the MHC class I α heavy chain is humanized. Humanized MHC class I α heavy chains are described, e.g., in U.S. Pat. Pub. Nos. 2013/0111617, 2013/0185819 and 2014/0245467. In some embodiments, the MHC class I α heavy chain comprises a human extracellular domain (human α1, α2, and/or α3 domains) and a cytoplasmic domain of another species. In some embodiments, the class I α heavy chain polypeptide is HLA-A, HLA-B, HLA-C, HLA-E, HLA-F, HLA-G, HLA-K, or HLA-L. In some embodiments, the HLA-A sequence can be an HLA-A*0201 sequence. In various aspects, the peptide-MHC can include all the domains of an MHC class I heavy chain.

In some embodiments, the MHC molecule comprises a β2-microglobulin. In some embodiments, the β2-microglobulin is fully human. In some embodiments, the β2-microglobulin is humanized.

In some embodiments, the MHC class I molecule comprises a mutation in a β2-microglobulin (β2m or B2M) polypeptide and in the Heavy Chain sequence to effect a disulfide bond between the B2M and the Heavy Chain. In some cases, the Heavy Chain is an HLA and wherein the disulfide bond links one of the following pairs of residues: B2M residue 12, HLA residue 236; B2M residue 12, HLA residue 237; B2M residue 8, HLA residue 234; B2M residue 10, HLA residue 235; B2M residue 24, HLA residue 236; B2M residue 28, HLA residue 232; B2M residue 98, HLA residue 192; B2M residue 99, HLA residue 234; B2M residue 3, HLA residue 120; B2M residue 31, HLA residue 96; B2M residue 53, HLA residue 35; B2M residue 60, HLA residue 96; B2M residue 60, HLA residue 122; B2M residue 63, HLA residue 27; B2M residue Arg3, HLA residue Gly120; B2M residue His31, HLA residue Gln96; B2M residue Asp53, HLA residue Arg35; B2M residue Trp60, HLA residue Gln96; B2M residue Trp60, HLA residue Asp122; B2M residue Tyr63, HLA residue Tyr27; B2M residue Lys6, HLA residue Glu232; B2M residue Gln8, HLA residue Arg234; B2M residue Tyr10, HLA residue Pro235; B2M residue Ser11, HLA residue Gln242; B2M residue Asn24, HLA residue Ala236; B2M residue Ser28, HLA residue Glu232; B2M residue Asp98, HLA residue His192; and B2M residue Met99, HLA residue Arg234, first linker position Gly2, Heavy Chain (HLA) position Tyr84; Light Chain (B2M) position Arg12, HLA Ala236; and/or B2M residue Arg12, HLA residue Gly237.

In some embodiments, the antigenic determinant amino acid sequence can be that of a peptide described herein, which can be presented by an MHC class I molecule. In certain embodiments, the sequence can comprise from about 8 to about 15 contiguous amino acids. In certain embodiments, the sequence can comprise from about 8 to about 12 contiguous amino acids.

In some embodiments, at least one chain of the MHC and the peptide are comprised within a fusion protein. In one specific embodiment, the MHC and the peptide are separated by a linker sequence. For example, the single chain molecule can comprise, from amino to carboxy terminal, an antigenic determinant, a β2-microglobulin sequence, and a class I α (heavy) chain sequence. Alternatively, the single chain molecule can comprise, from amino to carboxy terminal, an antigenic determinant, a class I α (heavy) chain sequence, and a β2-microglobulin sequence. The single-chain molecule can further comprise a signal peptide sequence at the amino terminal. In certain embodiments, there can be a linker sequence between the peptide sequence and the β2-microglobulin sequence. In certain embodiments, there can be a linker sequence between the β2-microglobulin sequence and the class I α (heavy) chain sequence. A single-chain molecule can further comprise a signal peptide sequence at the amino terminal, as well as first linker sequence extending between the peptide sequence and the β2-microglobulin sequence, and/or a second linker sequence extending between the β2-microglobulin sequence and the class I heavy chain sequence. In certain embodiments, the β2-microglobulin and the class I α (heavy) chain sequences can be human, murine, or porcine.

In some embodiments, a single-chain molecule can comprise a first flexible linker between the peptide ligand segment and the β2-microglobulin segment. For example, linkers can extend from and connect the carboxy terminal of the peptide ligand segment to the amino terminal of the β2-microglobulin segment. Preferably, the linkers are structured to allow the linked peptide ligand to fold into the binding groove resulting in a functional MHC-antigen peptide. In some embodiments, this linker can comprise at least about 10 amino acids, up to about 15 amino acids. In some embodiments, a single-chain molecule can comprise a second flexible linker inserted between the β2-microglobulin and heavy chain segments. For example, linkers can extend from and connect the carboxy terminal of the β2-microglobulin segment to the amino terminal of the heavy chain segment. In certain embodiments, the β2-microglobulin and the heavy chain can fold into the binding groove resulting in a molecule which can function in promoting T cell expansion.

Suitable linkers used in the MHCs can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Non-limiting examples of linkers include, e.g., glycine polymers (G)n, glycine-serine polymers (including, for example, (GS)n, (GSGGS)n (SEQ ID NO: 105) and (GGGS)n (SEQ ID NO: 106), where n is an integer of at least one), glycine-alanine polymers, alanine-serine polymers, and other flexible linkers. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains). Exemplary linkers can comprise amino acid sequences including, but not limited to, those listed in Table 2. In some embodiments, a linker peptide includes a cysteine residue that can form a disulfide bond with a cysteine residue present in a second polypeptide.

TABLE 2
Examples of Linker Sequences

| | | Linker | | Linker | |
|---|---|---|---|---|---|
| Linker | | codon- | | codon- | |
| amino | | optimized | | optimized | |
| acid | SEQ | nucleotide | SEQ | nucleotide | SEQ |
| se- | ID | sequence | ID | sequence | ID |
| quence | NO: | (human) | NO: | (mouse) | NO: |
| GGGS | 55 | GGGGGTGGTTCC | 71 | GGTGGCGGTAGT | 87 |
| GGSG | 56 | GGTGGGTCTGGG | 72 | GGGGGATCTGGT | 88 |
| GSGGS | 57 | GGGTCCGGGGGC | 73 | GGCAGTGGCGGTA | 89 |
| | | TCC | | GC | |
| GGSGG | 58 | GGTGGGAGCGGT | 74 | GGAGGGAGTGGA | 90 |
| | | GGT | | GGG | |
| GSGSG | 59 | GGCAGCGGAAGC | 75 | GGGTCTGGCTCAG | 91 |
| | | GGA | | GC | |
| GSGGG | 60 | GGGAGTGGGGGA | 76 | GGTTCTGGCGGAG | 92 |
| | | GGT | | GT | |
| GGGSG | 61 | GGTGGGGGAAGT | 77 | GGTGGTGGGAGTG | 93 |
| | | GGA | | GA | |
| GSSSG | 62 | GGCAGCTCATCTG | 78 | GGCTCAAGCAGTG | 94 |
| | | GT | | GA | |
| GCGAS | 63 | GGATGTGGTGCA | 79 | GGCTGTGGGGCTA | 95 |
| GGGGS | | TCTGGAGGGGGA | | GTGGGGGAGGTGG | |
| GGGGS | | GGCTCTGGGGGG | | TAGTGGTGGTGGC | |
| | | GGTGGATCT | | GGTTCC | |
| GCGAS | 64 | GGGTGTGGTGCT | 80 | GGATGTGGGGCCT | 96 |
| GGGGS | | AGTGGGGGTGGC | | CAGGTGGGGGTGG | |
| GGGGS | | GGATCAGGTGGA | | CAGCGGTGGTGGA | |
| | | GGCGGGAGC | | GGGTCA | |
| GGGGS | 65 | GGGGGCGGAGGA | 81 | GGTGGCGGGGGCT | 97 |
| GGGGS | | TCTGGGGGAGGG | | CTGGTGGAGGAGG | |
| | | GGATCA | | ATCT | |
| GGGAS | 66 | GGGGGGGGCGCT | 82 | GGAGGCGGCGCTT | 98 |
| GGGGS | | TCAGGCGGAGGT | | CTGGGGGCGGGGG | |
| GGGGS | | GGAAGTGGTGGA | | TAGTGGGGGTGGA | |
| | | GGAGGT | | GGT | |
| GGGGS | 67 | GGAGGGGGAGGT | 83 | GGTGGAGGTGGAA | 99 |
| GGGGS | | TCTGGCGGCGGG | | GTGGAGGAGGGG | |
| GGGGS | | GGATCAGGAGGC | | GATCAGGCGGAGG | |
| | | GGTGGGAGC | | CGGGAGC | |
| GGGAS | 68 | GGTGGGGGGGCG | 84 | GGAGGGGGAGCCT | 100 |
| GGGGS | | TCAGGTGGAGGC | | CTGGCGGTGGAGG | |
| | | GGAAGT | | ATCA | |
| GGGGS | 69 | GGCGGCGGAGGT | 85 | GGGGGAGGAGGC | 101 |
| GGGGS | | TCTGGTGGGGGT | | AGTGGAGGTGGGG | |
| GGGGS | | GGCAGTGGAGGA | | GAAGTGGTGGAGG | |
| | | GGAGGCAGC | | GGGGTCT | |
| GGGGS | 70 | GGAGGTGGAGGT | 86 | GGGGGTGGAGGAT | 102 |
| GGGGS | | AGTGGCGGTGGT | | CAGGAGGCGGTGG | |
| GGGGS | | GGGTCAGGGGGA | | TTCTGGGGGAGGT | |
| GGGGS | | GGCGGGTCCGGT | | GGATCCGGCGGGG | |
| | | GGCGGTGGGAGT | | GTGGTAGT | |

In certain embodiments, the single-chain molecule can comprise a peptide covalently attached to an MHC class I α (heavy) chain via a disulfide bridge (i.e., a disulfide bond between two cystines). In certain embodiments, the disulfide bond comprises a first cysteine, comprising a linker extending from the carboxy terminal of an antigen peptide, and a second cysteine comprising an MHC class I heavy chain (e.g., an MHC class I α (heavy) chain which has a non-covalent binding site for the antigen peptide). In certain embodiments, the second cysteine can be a mutation (addition or substitution) in the MHC class I α (heavy) chain. In certain embodiments, the single-chain molecule can comprise one contiguous polypeptide chain as well as a disulfide bridge. In certain embodiments, the single-chain molecule can comprise two contiguous polypeptide chains which are attached via the disulfide bridge as the only covalent linkage. In some embodiments, the linking sequences can comprise at least one amino acid in addition to the Cys residues, including one or more Gly residues, one or more Ala residues, and/or one or more Ser residues.

In certain embodiments, the disulfide bridge can link an antigen peptide described herein in the class I groove of the pMHC complex if the pMHC complex comprises a first cysteine in a Gly-Ser linker extending between the C-terminus of the peptide and the 02-microglobulin, and a second cysteine in a proximal heavy chain position.

Attaching the peptide to the MHC class I or MHC class II molecule via a flexible linker has the can help ensure that the peptide will occupy and stay associated with the MHC molecule during biosynthesis, transport, and display. However, there may be situations in which this linker can interfere with peptide binding to the MHC molecule or with TCR recognition of the complex. As an alternate approach, in some embodiments, the MHC molecule and the peptide are expressed separately.

In some embodiments, the β2-microglobulin sequence can comprise a full-length β2-microglobulin sequence. In certain embodiments, the β2-microglobulin sequence lacks the leader peptide sequence. As such, in some configurations, the β2-microglobulin sequence can comprise about 99 amino acids, and can be a mouse β2-microglobulin sequence (e.g., GenBank Accession No. X01838). In some other configurations, the β2-microglobulin sequence can comprise about 99 amino acids, and can be a human β2-microglobulin sequence (e.g., GenBank Accession No. AF072097.1).

In some embodiments, the pMHC complex can contain MHC sequences as disclosed in U.S. Pat. Nos. 4,478,823; 6,011,146; 8,518,697; 8,895,020; 8,992,937; WO 96/04314; Mottez et al. J. Exp. Med. 181: 493-502, 1995; Madden et al. Cell 70: 1035-1048, 1992; Matsumura et al., Science 257: 927-934, 1992; Mage et al., Proc. Natl. Acad. Sci. USA 89: 10658-10662, 1992; Toshitani et al, Proc. Nat'l Acad. Sci. 93: 236-240, 1996; Chung et al, J. Immunol. 163:3699-3708, 1999; Uger and Barber, J. Immunol. 160: 1598-1605, 1998; Uger et al., J. Immunol. 162, pp. 6024-6028, 1999; White et al., J. Immunol. 162: 2671-2676, 1999; Yu et al., J. Immunol. 168:3145-3149, 2002; Truscott et al., J. Immunol. 178: 6280-6289, 2007, all of which are incorporated by reference in their entireties.

In some embodiments, the MHC comprises a class II MHC polypeptide or a fragment, mutant or derivative thereof. In one specific embodiment, the MHC comprises a and R polypeptides of a class II MHC complex or a fragment, mutant or derivative thereof. In one specific embodiment, the α and β polypeptides are linked by a peptide linker. In one specific embodiment, the MHC comprises a and R polypeptides of a human class II MHC complex selected from the group consisting of HLA-DP, HLA-DR, HLA-DQ, HLA-DM and HLA-DO. In another specific embodiment, the MHC comprises a and R polypeptides of a murine H-2A or H-2E class II MHC complex.

Naturally occurring MHC class II molecules can contain two polypeptide chains, α and β. The chains may come from the DP, DQ, or DR gene groups. There are about 40 known different human MHC class II molecules. All have the same basic structure but can vary subtly in their molecular structure. MHC class II molecules can bind peptides of 13-18 amino acids in length.

In some embodiments, the MHC class II α chain is fully human. In some embodiments, the MHC class II α chain is humanized. Humanized MHC class II α chains are described, e.g., in U.S. Pat. Nos. 8,847,005, 9,043,996, and 10,154,658, which are incorporated herein by reference in their entireties. In some embodiments, the humanized MHC class II α chain polypeptide comprises a human extracellular domain and a cytoplasmic domain of another species. In some embodiments, the class II α chain is HLA-DMA, HLA-DOA, HLA-DPA, HLA-DQA or HLA-DRA. In some embodiments, the class II α chain polypeptide is humanized HLA-DMA, HLA-DOA, HLA-DPA, HLA-DQA and/or HLA-DRA.

In some embodiments, the peptide of the present disclosure forms a complex with one or more MHC class II β chains. In some embodiments, the MHC class II β chain is fully human. In some embodiments, the MHC class II β chain polypeptide is humanized. Humanized MHC class II β chain polypeptides are described, e.g., in U.S. Pat. Nos. 8,847,005, 9,043,996, and 10,154,658, which are incorporated herein by reference in their entireties. In some embodiments, the humanized MHC class II β chain comprises a human extracellular domain and a cytoplasmic domain of another species. In some embodiments, the class II β chain is HLA-DMB, HLA-DOB, HLA-DPB, HLA-DQB or HLA-DRB. In some embodiments, the class II β chain is humanized HLA-DMB, HLA-DOB, HLA-DPB, HLA-DQB and/or HLA-DRB.

The pMHC complexes of the disclosure may be isolated and/or in a substantially pure form. For example, the complex may be provided in a form which is substantially free of other peptides or proteins. MHC molecules as disclosed herein can include recombinant MHC molecules, non-naturally occurring MHC molecules, and functionally equivalent fragments of MHC, including derivatives or variants thereof, provided that peptide binding is retained. For example, MHC molecules may be fused to a therapeutic moiety, attached to a solid support, in soluble form, attached to a tag, biotinylated and/or in multimeric form. A peptide disclosed herein may be covalently attached to the MHC.

Methods to produce soluble recombinant MHC molecules with which peptides disclosed herein can form a complex include, but are not limited to, expression and purification from E. coli cells or insect cells. Alternatively, MHC molecules may be produced synthetically, or using cell free systems.

The peptides disclosed herein may be presented on the surface of a cell in complex with MHC. Thus, the present disclosure also provides a cell presenting on its surface a pMHC complex disclosed herein. Such a cell may be a mammalian cell, preferably a cell of the immune system, and a specialized antigen-presenting cell (APC) such as a dendritic cell or a B cell. Other preferred cells include T2 cells. Cells presenting the peptide or pMHC complex of the disclosure may be isolated, preferably in the form of a homogenous population, or provided in a substantially pure form. Such cells may not naturally present the complex of the disclosure, or alternatively said cells may present the complex at a level higher than they would in nature. Such cells may be obtained by pulsing said cells with one or more peptides (e.g., 2 to 50, 2 to 40, 2 to 30, 5 to 25, 5 to 20, or 10 to 15 peptides) of the disclosure, or genetically modifying the cells (via DNA or RNA transfer) to express one or more peptides (e.g., 2 to 50, 2 to 40, 2 to 30, 5 to 25, 5 to 20, or 10 to 15 peptides) of the disclosure. Pulsing involves incubating the cells with the peptide for several hours using peptide concentrations typically ranging from 10⁻⁵ to 10⁻¹² M. Such cells may additionally be transduced with HLA molecules, such as HLA-A*02 to further induce presentation of the peptide(s). Cells may be produced recombinantly. Cells presenting peptides of the disclosure may be used to isolate T cells and TCRs which are activated by, or bind to, the cells.

### Fusion Proteins, Conjugates and Oligomeric Complexes Disclosed Herein

Peptides or pMHC complexes disclosed herein may be fused or conjugated to one or more heterologous molecules. Peptides or pMHC complexes of the disclosed herein may also be in multimeric form. Accordingly, the present disclosure also provides fusion proteins, conjugates, and oligomeric complexes comprising a peptide or a pMHC complex of the disclosure.

In some embodiments, peptides are fused or conjugated to one or more heterologous molecules which includes an MHC molecule (or fragments thereof).

Heterologous molecules suitable for genetical fusion and/or chemical conjugation with the peptides or the pMHC complexes of the disclosure include, but are not limited to, peptides, polypeptides, small molecules, polymers, nucleic acids, lipids, sugars, etc. The heterologous molecule(s) may be fused at the N- and/or C-terminus of the peptide and/or another polypeptide chain in the pMHC complex.

Heterologous peptides and polypeptides include, but are not limited to, an epitope (e.g., FLAG) or a tag sequence (e.g., His₆ (SEQ ID NO: 109), and the like) to allow for the detection and/or isolation of a fusion protein; a transmembrane receptor protein or a portion thereof, such as an extracellular domain or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region (e.g., an Fc domain); a half-life-extending sequence comprising a combination of two or more (e.g., 2, 5, 10, 15, 20, 25, etc.) naturally occurring or non-naturally occurring charged and/or uncharged amino acids (e.g., Ser, Gly, Glu or Asp) designed to form a predominantly hydrophilic or predominantly hydrophobic fusion partner for a fusion protein; a functional or non-functional antibody (e.g., an antibody that is specific for dendritic cells), or a heavy or light chain thereof, and a polypeptide which has an activity, such as a therapeutic activity, different from fusion proteins of the present disclosure. In some embodiments, the one or more heterologous molecules enhances a peptide-specific immune response in a subject. In some embodiments, the one or more heterologous molecules mediates peptide delivery to a specific site within a subject.

In some embodiments, fusion proteins of the disclosure may comprise one or more affinity tags, e.g., to allow for affinity purification or coupling to another molecule. Examples of affinity tags include, but are not limited to, a His₆ tag (SEQ ID NO: 109), an Avi-tag, a biotin, a hemagglutinin (HA) tag, a FLAG tag, a Myc tag, a GST tag, a MBP tag, a chitin binding protein tag, a calmodulin tag, a V5 tag, a streptavidin binding tag, a green fluorescent protein (GFP), YFP, RFP, CFP, mCherry, tdTomato, SUMO tag, and Ubiquitin tag.

In some embodiments, fusion proteins of the disclosure may comprise one or more epitopes that is not present in the antigen. One such example is the use of fusion peptides where a promiscuous T helper epitope is covalently linked (e.g., via a polypeptide linker or spacer) to the peptide sequence. Non-limiting examples of promiscuous T helper epitopes include the PADRE peptide, tetanus toxoid peptide (830-843), or influenza haemagglutinin, HA(307-319).

Peptides or pMHC complexes of the disclosure may be conjugated to additional moieties such as carrier molecules or adjuvants for use as vaccines. Examples of adjuvants used in vaccines include microbes, such as the bacterium Bacillus Calmette-Guerin (BCG), and/or substances produced by bacteria, such as Detox B (an oil droplet emulsion of monophosphoryl lipid A and mycobacterial cell wall skeleton). KLH (keyhole limpet hemocyanin), bovine serum albumin (BSA), the E2 core protein of the pyruvate dehydrogenase complex are examples of suitable carrier proteins used in vaccine compositions. Additional examples of carrier proteins suitable for use in the compositions of the present disclosure include, but are not limited to, ovalbumin (OVA), blue carrier protein (BCP), thyroglobulin (THY), a soybean trypsin inhibitor (STI), and multiple attachment peptide (MAP), albumin, serum albumin, c-reactive protein, conalbumin, lactalbumin, ion carrier protein, acyl carrier protein, signal transduction adapter protein, androgen binding protein, calcium binding protein, calmodulin binding protein, ceruloplasmin, cholesterol Ester transfer protein, f box protein, fatty acid binding protein, follistatin, follistatin related protein, GTP binding protein, insulin-like growth factor binding protein, iron binding protein, latent TGF beta binding protein, light-harvesting protein complex, lymph Sphere antigen, membrane transport protein, neurophysin, periplasmic binding protein, phosphate binding protein, phosphatidylethanolamine binding protein, phospholipid transport protein, retinol binding protein, RNA binding protein, s-phase kinase related protein, sex hormone binding globulin, Thyroxine binding protein, transcobalamin, transcortin, transferrin binding protein, and/or vitamin D binding protein.

As a further example, a peptide or pMHC complex of the present disclosure may be fused to, for example, the 80 N-terminal amino acids of the HLA-DR antigen-associated invariant chain (p33 or Ii) as derived from the NCBI, GenBank Accession-number X00497). The Ii fragment may facilitate an efficient introduction of the peptide or pMHC complex into the cells.

Peptides or pMHC complexes of the present disclosure may also be attached, covalently (e.g., via a linker) or non-covalently, to a moiety capable of eliciting a therapeutic effect, such as antibodies, or cytokines, such as interleukin 2, interferon-α, and granulocyte-macrophage colony-stimulating factor. Alternatively or additionally, the peptides or pMHC complexes may be encapsulated into liposomes.

Other suitable heterologous molecules include, but are not limited to, fluorescent, or luminescent labels, radiolabels, nucleic acid probes, and contrast reagents, antibodies, or enzymes that produce a detectable product. Methods for detecting heterologous molecules may include flow cytometry, microscopy, electrophoresis, or scintillation counting.

In some embodiments, peptides or pMHC complexes of the disclosure may be conjugated with fluorocarbon to increase cellular immunogenicity. Where the peptide or another polypeptide chain of the pMHC complex is linked to a fluorocarbon, the terminus of the peptide or polypeptide chain, such as the terminus that is not conjugated to the fluorocarbon, or other attachment, can be altered, for example to promote solubility of the fluorocarbon-peptide/polypeptide construct via the formation of micelles. To facilitate large-scale synthesis of the construct, the N- or C-terminal amino acid residues of the peptide or another polypeptide chain of the pMHC complex can be modified. When the desired peptide or another polypeptide chain of the pMHC complex is particularly sensitive to cleavage by peptidases, the normal peptide bond can be replaced by a non-cleavable peptide mimetic. Such bonds and methods of synthesis are well known in the art.

Peptides or pMHC complexes of the disclosure may be provided in soluble form, or may be immobilized by attachment to a suitable solid support. Examples of solid supports include, but are not limited to, a bead, a membrane, sepharose, a magnetic bead, a plate, a tube, a column. pMHC complexes may be attached to an ELISA plate, a magnetic bead, or a surface plasmon resonance biosensor chip. Methods of attaching peptides or pMHC complexes to a solid support are known to the skilled person, and include, for example, using an affinity binding pair, e.g. biotin and streptavidin, or antibodies and antigens. In some embodiments, peptides or pMHC complexes are labeled with biotin and attached to streptavidin-coated surfaces.

Peptides or pMHC complexes of the disclosure may be in multimeric form, for example, dimeric, or tetrameric, or pentameric, or octameric, or greater. Accordingly, in some aspects, the present disclosure provides oligomeric complexes comprising the peptides or pMHC complexes of the present disclosure. As used herein, the terms “oligomer”, “oligomeric”, “oligomerize” and “oligomerization” or the like encompass a dimer, trimer, tetramer, pentamer, hexamer, heptamer, octamer, or higher species of polymerized monomers that comprise the peptide or pMHC complex. Having multiple copies of the peptides or pMHC complexes in a large complex may enhance their biological activity, e.g., immunogenic activity.

For example, the peptides of the disclosure may be oligomerized using the biotin/streptavidin system. Biotinylated analogs of peptide monomers may be synthesized by standard techniques. For example, the peptide may be C-terminally biotinylated. These biotinylated peptide monomers are then oligomerized by incubation with streptavidin [e.g., at a 4:1 molar ratio at room temperature in phosphate buffered saline (PBS) or HEPES-buffered RPMI medium for 1 hour]. In a variation of this embodiment, biotinylated peptide monomers may be oligomerized by incubation with anti-biotin antibodies [e.g., goat anti-biotin IgG].

In general, oligomeric pMHC complexes may be produced using pMHC tagged with a biotin residue and complexed through fluorescently labeled streptavidin. A biotinylation site may be introduced to the pMHC complex to which biotin can be added, for example, using the BirA enzyme. Alternatively, oligomeric pMHC complexes may be formed by using immunoglobulin as a molecular scaffold. In this system, the extracellular domains of MHC molecules are fused with the constant region of an immunoglobulin heavy chain separated by a short amino acid linker. Oligomeric pMHC complexes have also been produced using carrier molecules such as dextran. Oligomeric pMHC complexes can be useful for improving the detection of binding moieties, such as T cell receptors, which bind said complex, because of avidity effects.

In other embodiments, the peptides or pMHC complexes of the disclosure can be oligomerized by covalent attachment to at least one linker. The linker moiety can be a peptide linker, such as those described herein (e.g., in Table 2). In some embodiments, polyethylene glycol (PEG) may serve as the linker that oligomerizes the peptide monomers. For example, a single PEG moiety may be simultaneously attached to the N-termini of both peptide chains of a peptide dimer.

Alternatively, oligomeric peptide or pMHC complexes may also contain one or more intramolecular disulfide bonds between cysteine residues of the peptide or pMHC monomers. Preferably, the two monomers contain at least one intramolecular disulfide bond. Most preferably, both monomers contain an intramolecular disulfide bond, such that each monomer contains a cyclic group. Such disulfide bonds may be formed by oxidation of the cysteine residues of the peptide core sequence. In one embodiment the control of cysteine bond formation is exercised by choosing an oxidizing agent of the type and concentration effective to optimize formation of the desired isomer. For example, oxidation of a peptide dimer to form two intramolecular disulfide bonds (one on each peptide chain) is preferentially achieved (over formation of intermolecular disulfide bonds) when the oxidizing agent is DMSO. The formation of cysteine bonds can be controlled by the selective use of thiol-protecting groups during peptide synthesis.

In some embodiments, peptides or pMHC complexes described herein may be fused or conjugated to a dimerization moiety. The dimerization moiety may contain, for example, an immunoglobulin domain, such as from an IgG antibody (e.g., human IgG), which connects two monomers generating a homodimer or heterodimer molecule. As a non-limiting example, the dimerization motif in the proteins according to the present disclosure may be constructed to include a hinge region and an immunoglobulin domain (e.g. Cy3 domain), e.g., carboxyterminal C domain (CH3 domain), or a sequence that is substantially identical to the C domain. The hinge region may be Ig derived and contributes to the dimerization through the formation of an interchain covalent bond(s), e.g. disulfide bridge(s). In addition, such homodimer or heterodimer molecules may further comprise one or more targeting moieties that bind to target molecules present on, for example, antigen-presenting cells (APCs) such as dendritic cells or B cells. In such instances, the hinge region may function as a flexible spacer between the domains allowing the two targeting units to bind simultaneously to two target molecules on the APC expressed with variable distances. The immunoglobulin domains contribute to dimerization through non-covalent interactions, e.g. hydrophobic interactions. In a preferred embodiment the CH3 domain is derived from IgG. These dimerization moieties may be exchanged with other multimerization moieties from e.g., other Ig isotypes/subclasses. Preferably the dimerization motif is derived from native human proteins, such as human IgG. Examples of such homodimer protein construct are described in U.S. Pat. No. 10,590,195, which is incorporated herein by reference in its entirety.

### Nucleic Acids and Vectors

In another aspect, the disclosure provides an isolated polynucleotide comprising a nucleic acid sequence encoding one or more peptide(s) and/or peptide-based molecules (such as complexes (e.g., pMHC complexes), fusion proteins, or conjugates comprising the described peptides) of the disclosure. The polynucleotide may be, for example, DNA, cDNA, PNA, RNA or combinations thereof, either single- and/or double-stranded, or native or stabilized forms of polynucleotides, such as, for example, polynucleotides with a phosphorothioate backbone and it may or may not contain introns so long as it codes for the peptide.

In some embodiments, the polynucleotide described herein encodes a peptide comprising an amino acid sequence that is at least about 60%, at least about 65%, at least about 70%, at least about 75%, at least about 80%, at least about 85%, at least about 90%, at least about 95%, or at least about 99% identical to the amino acid sequence of any one of SEQ ID NOs: 1-54 and 110-112, or a fragment or derivative thereof. In some embodiments, the polynucleotide described herein encodes a peptide comprising an amino acid sequence of any one of SEQ ID NOs: 1-54 and 110-112, or a fragment or derivative thereof.

In some embodiments, the polynucleotide described herein encodes more than one peptide selected from any one of SEQ ID NOs: 1-54 and 110-112, or fragments thereof. For example, the polynucleotide described herein may encodes 2 to 50, 2 to 40, 2 to 30, 5 to 25, 5 to 20, or 10 to 15 peptides as described herein (e.g., SEQ ID NO: 1-54 and 110-112), or fragments thereof. The peptides may be arranged in any order, and may be identical or different.

In some embodiment, the polynucleotide described herein encodes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 amino acid sequences selected from SEQ ID NO: 1-54 and 110-112, or fragments thereof.

In some embodiment, the polynucleotide described herein is a DNA molecule.

Methods to deliver DNA to a subject include, for example, direct delivery, as naked DNA. Delivery may also be achieved by nanoparticles; gene gun, microneedle array and in situ electroporation. The nucleic acids can also be administered using ballistic delivery. Particles comprised solely of DNA can be administered. Alternatively, DNA can be adhered to particles, such as gold particles.

In some embodiments, the polynucleotide described herein is an RNA molecule. For example, the RNA molecule may be mRNA or a self-replicating RNA.

A polynucleotide encoding RNA disclosed herein can be used to make a vaccine. RNA cannot integrate into the genome and has therefore little oncogenic potential; thus, RNA can be useful for making a vaccine. Also, RNA only needs to enter the cytoplasm, contrary to DNA which needs to enter the nucleus. An RNA molecule disclosed herein may be chemically modified and/or incorporate modified nucleosides to overcome susceptibility to degradation. RNA vaccines may comprise mRNA and/or self-replicating RNA (also known as RNA replicons). Delivery techniques for RNA vaccines may also encompass, for example, condensation with protamine and encapsulation into liposomes or nanoparticles.

The nucleic acids (either DNA or RNA) can also be delivered complexed to cationic compounds, such as cationic lipids. Lipid-mediated gene delivery methods are described, for example, in WO 91/06309; WO 93/24640; WO 96/18372; U.S. Pat. No. 5,279,833, which are incorporated herein by reference in their entireties.

A nucleic acid molecule described herein may be generated synthetically. One method is the phosphoramidite method. Without wishing to be bound by theory, in this chemistry, a phosphoramidite (a nucleoside with side protecting groups that preserve the integrity of the sugar, the phosphodiester linkage, and the base during chain extension steps) is coupled through its reactive 3′ phosphorous group to the 5′ hydroxyl group of a nucleoside immobilized on a solid support column. The steps of oligonucleotide synthesis can include the following: (1) Detritylation, in which the dimethoxytrityl (DMT or trityl) group on the 5′ hydroxyl of the support nucleoside is removed by treatment with trichloroacetic acid (TCA). (2) In the coupling step, a phosphoramidite, made reactive by tetrazole (a weak acid), is chemically coupled to the last base added to the column support material. (3) In the capping step, any free 5′ hydroxyl groups of unreacted column nucleotides are acetylated by treatment with acetic anhydride and N-methylimidazole. (4) In the oxidation step, the unstable internucleotide phosphate linkage between the previously coupled base and the most recently added base is oxidized by treatment with iodine and water to a more stable phosphotriester linkage. Following coupling of all bases in the oligonucleotide's sequence, the completed nucleic acid chain may be cleaved from the column by treatment with ammonium hydroxide, and the base protecting groups are removed by heating in the ammonium hydroxide solution.

By way of a non-limiting example, a synthesis cycle may comprise growth of the nucleotide chain from an initial protected nucleoside derivatized via its terminal 3′ hydroxyl to a solid support. Reagents and solvents can be pumped through the support to induce the consecutive removal and addition of sugar protecting groups in order to isolate the reactivity of a specific chemical moiety on the monomer and effect its stepwise addition to the growing oligonucleotide chain. Assembly of the protected oligonucleotide chain can be carried out in chemical steps, for example, without limitation, deblocking, activation/coupling, oxidation, and capping. Cleavage and deprotection then reveal the single-stranded nucleic acid.

Nucleic acid synthesis methods disclosed herein can comprise, for example, oligonucleotide synthesis, column-based oligonucleotide synthesis, microarray-based oligonucleotide synthesis, gene synthesis from oligonucleotides, gene synthesis from array-derived oligonucleotide pools, and any of various error correction and sequence validation steps, or any combination thereof.

RNA chemical synthesis may be similar to that used for DNA. In some embodiments, RNA chemical synthesis methods may comprise an additional protecting group at the 2′ hydroxyl of ribose. The 2′ hydroxyl of ribose position may be protected with tert-butyldimethyl silyl groups, which can be stable throughout the synthesis, and can be removed at the final deprotection step by addition of a basic fluoride ion such as tetrabutylammonium fluoride (TBAF). The remaining positions on both the sugar and the bases can be protected in the same fashion as for DNA. By adjusting several parameters in the DNA synthesis protocol such as, but not limited to the coupling times, monomer delivery rate, frequency of washing steps, and types of capping reagents, stepwise coupling efficiencies of up to 99% can be obtained.

Viral nucleic acid synthesis may be catalyzed by both viral and host enzymes, the relative contribution of which can be determined by the type of virus and the specific molecule. Viruses with RNA genomes, except for the retroviruses, synthesize mRNA and replicate their genomes using virus-encoded RNA-dependent RNA polymerases. In contrast, retroviruses synthesize a double-stranded complementary DNA (cDNA) copy of their single-stranded RNA genome using a virion-encoded RNA-dependent DNA polymerase (reverse transcriptase). In subsequent steps, the retroviral cDNA may be integrated into the host chromosome and transcribed by host-encoded DNA-dependent RNA polymerase II (pol II) to yield viral messages and genomic RNA. DNA viruses, except for poxviruses, also use host-encoded pol II to transcribe their messages. Poxviruses, because they replicate in the cytoplasm and do not have access to pol II, assemble a novel transcriptase composed of multiple poxvirus-specific (and possibly one or more host-derived) subunits. Most DNA virus families (e.g. Poxviridae, Iridoviridae, Herpesviridae, Adenoviridae) synthesize a virus-encoded DNA-dependent, DNA polymerase. However, two families (i.e., Parvoviridae and Papovaviridae) utilize host DNA polymerase, and the Hepadnaviridae replicate viral DNA through an RNA intermediate using a virus-encoded reverse transcriptase.

Due to the degeneracy of the genetic code, nucleic acid molecules of different nucleotide sequence can encode the same amino acid sequence. For expression in various hosts, the polynucleotides may be codon-optimized.

In a further aspect, the disclosure provides a vector comprising a nucleic acid sequence according to the third aspect of the disclosure. The vector may include, in addition to a nucleic acid sequence encoding only a peptide of the disclosure, one or more additional nucleic acid sequences encoding one or more additional peptides. Such additional peptides may, once expressed, be fused to the N-terminus or the C-terminus of the peptide of the disclosure. Examples of such additional peptides are detailed in the sections above. In one embodiment, the vector includes a nucleic acid sequence encoding a peptide or protein tag such as, for example, a biotinylation site, a FLAG-tag, a MYC-tag, an HA-tag, a GST-tag, a Strep-tag or a poly-histidine tag.

The vector utilized in the context of the present disclosure desirably comprises sequences appropriate for introduction into cells. For instance, the vector may be an expression vector, a vector in which the coding sequence of the polypeptide is under the control of its own cis-acting regulatory elements, a vector designed to facilitate gene integration or gene replacement in host cells, and the like.

In the context of the present disclosure, the term “vector” encompasses a DNA molecule, such as a plasmid, bacteriophage, phagemid, virus or other vehicle, which contains one or more heterologous or recombinant nucleotide sequences (e.g., an above-described nucleic acid molecule of the disclosure, under the control of a functional promoter and, possibly, also an enhancer) and is capable of functioning as a vector in the sense understood by those of ordinary skill in the art.

The following vectors are provided by way of example: bacteriophages such as lambda (λ) bacteriophage, EMBL bacteriophage; bacterial vectors such as pBs, phagescript, PsiX174, pBluescript SK, pBs KS, pNH8a, pNH16a, pNH18a, pNH46a; pTrc99A, pKK223-3, pKK233-3, pDR540, and pRIT5; eukaryotic vectors such as pWLneo, pSV2cat, pOG44, PXR1, pSG, pSVK3, pBPV, pMSG and pSVL; and transposons such as Sleeping Beauty transposon and PiggyBac transposon.

In some embodiments, the vector is a viral vector. Viral vectors can be derived from naturally occurring virus genomes, which typically are modified to be replication incompetent, e.g. non-replicating. Non-replicating viruses require the provision of proteins in trans for replication. Typically, those proteins are stably or transiently expressed in a viral producer cell line, thereby allowing replication of the virus. The viral vectors are, thus, typically infectious and non-replicating. Viral vectors may be adenovirus vectors, adeno-associated virus (AAV) vectors (e.g., AAV type 5 and type 2), alphavirus vectors (e.g., Venezuelan equine encephalitis virus (VEE), Sindbis virus (SIN), Semliki forest virus (SFV), and VEE-SIN chimeras), herpes virus vectors (e.g., vectors derived from cytomegaloviruses, like rhesus cytomegalovirus (RhCMV)), arena virus vectors (e.g. lymphocytic choriomeningitis virus (LCMV) vectors), measles virus vectors, pox virus vectors (e.g., vaccinia virus, modified vaccinia virus Ankara (MVA), NYVAC (derived from the Copenhagen strain of vaccinia), and avipox vectors (canarypox (ALVAC) and fowlpox (FPV) vectors), vesicular stomatitis virus (VSV) vectors, retrovirus vectors, lentivirus vectors, simian virus 40 (SV40), bovine papilloma viruses, Epstein-Barr viruses, Moloney murine leukemia viruses, Harvey murine sarcoma viruses, murine mammary tumor viruses, Rous sarcoma viruses, poxvirus viral like particles, baculoviral vectors and bacterial spores.

As further examples, adenovirus vectors may be derived from human adenovirus (Ad) but also from adenoviruses that infect other species, such as bovine adenovirus (e.g. bovine adenovirus 3, BAdV3), a canine adenovirus (e.g. CAdV2), a porcine adenovirus (e.g. PAdV3 or 5), or great apes, such as Chimpanzee (Pan), Gorilla (Gorilla), Orangutan (Pongo), Bonobo (Pan paniscus) and common chimpanzee (Pan troglodytes). Poxvirus (Poxviridae) vectors may be derived from smallpox virus (variola), vaccinia virus, cowpox virus or monkeypox virus. Exemplary vaccinia viruses are the Copenhagen vaccinia virus (W), New York Attenuated Vaccinia Virus (NYVAC), ALVAC, TROVAC and Modified Vaccinia Ankara (MVA).

Many expression systems are known in the art, including bacteria (for example E. coli and Bacillus subtilis), yeasts (for example Saccharomyces cerevisiae), filamentous fungi (for example Aspergillus spec.), plant cells, animal cells (e.g., mammalian cells), and insect cells.

In yet another aspect, the disclosure provides a host cell comprising the vector of the disclosure. The host cell can be either prokaryotic or eukaryotic. Bacterial cells may be preferred prokaryotic host cells in some circumstances and typically are a strain of E. coli such as, for example, the E. coli strains DH5 and RR1. Non-limiting examples of eukaryotic host cells include yeast, insect, and mammalian cells (e.g., from a mouse, rat, monkey, or human cell lines). Non-limiting examples of yeast host cells include, e.g., YPH499, YPH500, and YPH501. Non-limiting examples of mammalian host cells include Chinese hamster ovary (CHO) cells, NIH Swiss mouse embryo cells NIH/3T3, monkey kidney-derived COS-1 cells, and 293 cells which are human embryonic kidney cells. Examples of insect cells include Sf9 cells, which can be transfected with baculovirus expression vectors.

Transformation of appropriate cell hosts with a DNA construct of the present disclosure is accomplished by well-known methods that typically depend on the type of vector used. Successfully transformed cells, i.e. cells that contain a DNA construct of the present disclosure, can be identified by, for example, PCR. Alternatively, the presence of the protein in the supernatant can be detected using antibodies.

It will be appreciated that certain host cells of the disclosure are useful in the preparation of the peptides or peptide-based molecules of the disclosure, for example bacterial, yeast, and insect cells. However, other host cells may be useful in certain therapeutic methods. For example, antigen-presenting cells (APCs), such as dendritic cells or B cells, may be used to express the peptides of the disclosure such that the peptides may be loaded into appropriate MHC molecules.

A further aspect of the disclosure provides a method of producing peptides or peptide-based molecules of the disclosure, the method comprising culturing a host cell and isolating the peptide or peptide-based molecule from the host cell or its culture medium.

Peptide and pMHC Binding Moieties

Peptides, pMHC complex, or other peptide-based molecules (such as a complex, fusion protein, or conjugate comprising a peptide disclosed herein) of the present disclosure can be used to identify and/or isolate binding moieties that bind specifically to a peptide, pMHC complex, or other peptide-based molecule of the disclosure. Such binding moieties may be used as immunotherapeutic reagents and may include, e.g., antibodies (or antigen-binding fragments thereof), alternative scaffolds, TCRs, and CARs.

In one aspect, the disclosure provides a peptide binding moiety that binds a peptide of the disclosure. Preferably the peptide binding moiety binds a peptide when the peptide is in complex with MHC. In the latter instance, the peptide binding moiety may bind partially to the MHC, provided that the peptide binding moiety also binds to the peptide. The peptide binding moiety may bind only the peptide, and that binding may be specific. The peptide binding moiety may bind only the pMHC complex and that binding may be specific.

The disclosure also provides a method of identifying a peptide binding moiety that binds a pMHC complex of the disclosure, the method comprising contacting a candidate peptide binding moiety with the pMHC complex and determining whether the candidate peptide binding moiety binds the complex. Methods to determine binding to pMHC complexes include, for example, surface plasmon resonance, or any other biosensor technique, ELISA, flow cytometry, chromatography, microscopy. Alternatively, or in addition, binding may be determined by functional assays in which a biological response is detected upon binding, for example, cytokine release or cell apoptosis.

The candidate peptide binding moiety may be a peptide binding moiety of the type already described, such as an antibody or a TCR.

For example, antibodies and TCRs may be obtained from display libraries in which the pMHC complex of the disclosure is used to pan the library. TCRs can be displayed on the surface of phage particles and yeast particles, for example, and such libraries have been used for the isolation of high affinity variants of TCR derived from T cell clones. TCR phage libraries can be used to isolate TCRs with novel antigen specificity. Such libraries can be constructed with α- and β-chain sequences corresponding to those found in a natural repertoire. However, the random combination of these α- and β-chain sequences, which occurs during library creation, can produce a repertoire of TCRs that may not be naturally occurring.

In some embodiments, the pMHC complex of the disclosure may be used to screen a library of diverse TCRs displayed on the surface of phage particles. The TCRs displayed by said library may not correspond to those contained in a natural repertoire, for example, they may contain α- and β-chain pairing that would not be present in vivo, and/or the TCRs may contain non-natural mutations and/or the TCRs may be in soluble form. Screening may involve panning the phage library with pMHC complexes of the disclosure and subsequently isolating bound phage particles. For this purpose, pMHC complexes may be attached to a solid support, such as a magnet bead, or column matrix and phage bound pMHC complexes isolated, with a magnet, or by chromatography, respectively. The panning steps may be repeated several times. Isolated phage may be further expanded in E. coli cells. Isolated phage particles may be tested for specific binding to pMHC complexes of the disclosure. Binding can be detected using techniques including, but not limited to, ELISA, or SPR for example using a BiaCore instrument. The DNA sequence of the T cell receptor displayed by pMHC binding phage can be further identified by PCR methods.

Alternatively, antigen binding T cells and TCRs can be isolated from fresh blood obtained from patients or healthy donors. Such a method involves stimulating T cells using autologous dendritic cells (DCs), followed by autologous B cells, and then pulsed with a peptide disclosed herein. Several rounds of stimulation may be carried out, for example three or four rounds. Activated T cells may then be tested for specificity by measuring cytokine release in the presence of T2 cells pulsed with the peptide of the disclosure (for example using an IFNγ ELISpot assay). Activated cells may then be sorted by fluorescence-activated cell sorting (FACS) using labelled antibodies to detect intracellular cytokine production (e.g. IFNγ), or expression of a cell surface marker (such as CD137). Sorted cells may be expanded and further validated, for example, by ELISpot assay and/or cytotoxicity against target cells and/or staining by peptide-MHC tetramer. The TCR chains from validated clones may then be amplified by rapid amplification of cDNA ends (RACE) and sequenced.

A peptide binding moiety disclosed herein can include, for example, without limitation, an antibody, a TCR, or a CAR.

In some embodiments, the peptide binding moiety of the disclosure may be an antibody or antigen-binding fragment thereof. Antibodies or antigen-binding fragments thereof encompass derivatives, functional equivalents, and homologues of antibodies, humanized antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. A humanized antibody may be a modified antibody having the variable regions of a non-human, e.g. murine, antibody, and the constant region of a human antibody. Examples of antibodies are the immunoglobulin isotypes (e.g., IgG, IgE, IgM, IgD and IgA) and their isotypic subclasses; or fragments that comprise an antigen binding domain such as Fab, scFv, Fv, dAb, Fd; and diabodies. Antibodies may be polyclonal or monoclonal. A monoclonal antibody may be referred to herein as “mAb”.

In some embodiments, the antibody is a multispecific antibody. In some embodiments, the antibody is a bispecific antibody. The bispecific antibody may comprise a second targeting moiety that targets to the desired cell or tissue, e.g., liver, or to another desired antigen associated with the same or similar disease or disorder (e.g., liver cancer antigen).

It is possible to take an antibody, for example a monoclonal antibody, and use recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. A hybridoma (or other cell that produces antibodies) may be subject to genetic mutation or other changes, which may or may not alter the binding specificity of antibodies produced.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers, (ix) “diabodies”, multivalent or multispecific fragments constructed by gene fusion, and (x) VHH or VNAR antibodies, also known as single-domain antibodies or nanobodies (Nb), which may be derived from heavy-chain antibodies from e.g., dromedaries, camels, llamas, alpacas, or sharks.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). Where bispecific antibodies are to be used, these may be conventional bispecific antibodies, which can be manufactured in a variety of ways, e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins”. Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display from libraries. If one arm of the diabody is to be kept constant, for instance, with a specificity directed against an antigen of interest, then a library can be made where the other arm is varied, and an antibody of appropriate specificity selected. An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

In some embodiments, the peptide binding moiety may be an antibody-like molecule that has been designed to specifically bind a peptide or peptide-MHC complex of the disclosure. In some embodiments the peptide binding moiety may comprise a TCR-mimic antibody. In some embodiments, such TCR-mimic antibodies can comprise high-affinity soluble antibody molecules endowed with a TCR-like specificity towards tumor or viral epitopes that can target tumor and/or virus-infected cells and mediate their specific killing.

Also encompassed within the present disclosure are binding moieties based on engineered protein scaffolds or “alternative scaffolds”. Alternative scaffolds are derived from stable, soluble, natural protein structures which have been modified to provide a binding site for a target molecule of interest. Examples of alternative scaffolds include, but are not limited to, affibodies, which are based on the Z-domain of staphylococcal protein A that provides a binding interface on two of its α-helices; anticalins, derived from lipocalins, that incorporate binding sites for small ligands at the open end of a β-barrel fold; monobodies, designed to incorporate the fibronectin type III domain (Fn3) of fibronectin or tenascin as a protein scaffold or synthetic FN3 domains (e.g., tencon); nanobodies, and DARPins. Additional alternative scaffolds include Adnectin™, iMab, EETI-II/AGRP, Kunitz domain, thioredoxin peptide aptamer, Affilin, Tetranectin, Fynomer, and Avimer. Alternative scaffolds are typically targeted to bind the same antigenic proteins as antibodies, and are potential therapeutic agents. They may act as inhibitors or antagonists, or as delivery vehicles to target molecules, such as toxins, to a specific tissue in vivo. Short peptides may also be used to bind a target protein. Phylomers are natural structured peptides derived from bacterial genomes. Such peptides represent a diverse array of protein structural folds and can be used to inhibit/disrupt protein-protein interactions in vivo.

Alternative scaffolds are typically single chain polypeptidic frameworks that contain a highly structured core associated with variable domains of high conformational tolerance allowing insertions, deletions, or other substitutions within the variable domains. Libraries introducing diversity to one or more variable domains, and in some cases to the structured core, may be generated using known protocols and the resulting libraries may be screened for binding to the peptide and/or the pMHC complex of the disclosure, and the identified binders may be further characterized for their specificity using known methods. Alternative scaffolds may be derived from Protein A, in particular, the Z-domain thereof (affibodies), ImmE7 (immunity proteins), BPTI/APPI (Kunitz domains), CTLA-4, charybdotoxin (Scorpion toxin), Min-23 (knottins), lipocalins (anticalins), Ras-binding protein AF-6 (PDZ-domains), neokarzinostatin, a fibronectin domain, an ankyrin consensus repeat domain, or thioredoxin.

In some embodiments, the antibodies or alternative scaffolds described herein can be immobilized on viral vectors. Such modified recombinant viral vectors can be useful for the targeted introduction of genetic materials encoded by the viral vectors into cells and/or tissues (e.g., liver cells and/or liver tissues). Various means can be used to mobilize the antibodies or alternative scaffolds to the viral vectors, for example, by using an affinity binding pair, such as c-Myc/anti-Myc antibody, streptavidin/biotin, or via spy-tag/spy-catcher system. Exemplary vectors that may be modified with the antibodies or alternative scaffolds described herein include, but are not limited to, adeno-associated virus (AAV) vectors (e.g., AAV1, AAV2, AAV6, AAV9, or AAV9.PHP), retroviral vectors, lentiviral vectors, and targeted oncolytic viruses (e.g., herpes simplex virus (HSV)).

In some embodiments, the peptide binding moiety may be a TCR. TCRs are described using the International Immunogenetics (IMGT) TCR nomenclature, and the IMGT public database of TCR sequences.

The TCRs of the present disclosure may be in any format. For example, the TCRs may be αβ heterodimers, or aa or ββ homodimers.

α/β heterodimeric TCRs have an α-chain and a β-chain. Broadly, each chain comprises variable, joining and constant region, and the β-chain also usually contains a short diversity region between the variable and joining regions, but this diversity region is often considered as part of the joining region. Each variable region comprises three hypervariable CDRs (Complementarity Determining Regions) embedded in a framework sequence; CDR3 is believed to be the main mediator of antigen recognition. There are several types of α-chain variable (Vα) regions and several types of β-chain variable (Vβ) regions distinguished by their framework, CDR1 and CDR2 sequences, and by a partly defined CDR3 sequence.

The TCRs of the disclosure may not correspond to TCRs as they exist in nature. For example, they may comprise α- and β-chain combinations that are not present in a natural repertoire. Alternatively or additionally, a TCR described herein may be soluble, and/or the α- and/or β-chain constant domain may be truncated relative to the native/naturally occurring TRAC/TRBC sequences such that, for example, the C-terminal transmembrane domain and intracellular regions are not present. Such truncation may result in removal of the cysteine residues from TRAC/TRBC that form the native interchain disulfide bond.

In addition, the TRAC/TRBC domains may contain modifications. For example, the α-chain extracellular sequence may include a modification relative to the native/naturally occurring TRAC whereby amino acid T48 of TRAC, with reference to IMGT numbering, is replaced with C48. Likewise, the β-chain extracellular sequence may include a modification relative to the native/naturally occurring TRBC1 or TRBC2 whereby S57 of TRBC1 or TRBC2, with reference to IMGT numbering, is replaced with C57. These cysteine substitutions relative to the native α- and β-chain extracellular sequences enable the formation of a non-native interchain disulfide bond which stabilizes the refolded soluble TCR, i.e. the TCR formed by refolding extracellular α- and β-chains. This non-native disulfide bond facilitates the display of correctly folded TCRs on phage. In addition, the use of the stable disulfide linked soluble TCR enables more convenient assessment of binding affinity and binding half-life. Alternative positions for the formation of a non-native disulfide include, for example, Thr 45 of exon 1 of TRAC*01 and Ser 77 of exon 1 of TRBC1*01 or TRBC2*01; Tyr 10 of exon 1 of TRAC*01 and Ser 17 of exon 1 of TRBC1*01 or TRBC2*01; Thr 45 of exon 1 of TRAC*01 and Asp 59 of exon 1 of TRBC1*01 or TRBC2*01; and Ser 15 of exon 1 of TRAC*01 and Glu 15 of exon 1 of TRBC1*01 or TRBC2*01. TCRs with a non-native disulfide bond may be full length or may be truncated.

TCRs of the disclosure may be in single chain format. Single chain TCRs include αβ TCR polypeptides of the type: Vα-L-Vβ, Vβ-L-Vα, Vα-Cα-L-Vβ, Vα-L-Vβ-Cβ or Vα-Cα-L-Vβ-Cβ, optionally in the reverse orientation, wherein Vα and Vβ are TCR α and β variable regions respectively, Cα and Cβ are TCR α and β constant regions respectively, and L is a linker sequence. Single chain TCRs may contain a non-native disulfide bond. The TCR may be in a soluble form (i.e. having no transmembrane or cytoplasmic domains) or may contain full length α- and β-chains. The TCR may be provided on the surface of a cell, such as a T cell.

TCRs of the disclosure may be engineered to include mutations. Methods for producing mutated high affinity TCR variants such as phage display and site directed mutagenesis. Preferably, mutations to improve affinity are made within the variable regions of α- and/or β-chains. More preferably mutations to improve affinity are made within the CDRs. There may be between 1 and 15 mutations in the α- and or β-chain variable regions.

TCRs of the disclosure may also be labeled with an imaging compound, for example a label that is suitable for diagnostic purposes. Such labelled high affinity TCRs are useful in a method for detecting a TCR ligand selected from CD1-antigen complexes, bacterial superantigens, and MHC-peptide/superantigen complexes, which method comprises contacting the TCR ligand with a high affinity TCR (or a multimeric high affinity TCR complex) which is specific for the TCR ligand; and detecting binding to the TCR ligand. In multimeric high affinity TCR complexes (formed, for example, using biotinylated heterodimers) fluorescent streptavidin can be used to provide a detectable label. A fluorescently-labelled multimer is suitable for use in FACS analysis, for example to detect antigen presenting cells carrying the peptide for which the high affinity TCR is specific.

A TCR of the present disclosure (or multivalent complex thereof) may alternatively or additionally be associated with (e.g. covalently or otherwise linked to) a therapeutic agent which may be, for example, a toxic moiety for use in cell killing, or an immunostimulating agent such as an interleukin or a cytokine. A multivalent high affinity TCR complex of the present disclosure may have enhanced binding capability for a TCR ligand compared to a non-multimeric wild-type or high affinity T cell receptor heterodimer. Thus, the multivalent high affinity TCR complexes according to the disclosure are particularly useful for tracking or targeting cells presenting particular antigens in vitro or in vivo, and are also useful as intermediates for the production of further multivalent high affinity TCR complexes having such uses. The high affinity TCR or multivalent high affinity TCR complex may therefore be provided in a pharmaceutically acceptable formulation for use in vivo.

High affinity TCRs of the disclosure may be used in the production of soluble bi-specific reagents. A preferred embodiment is a reagent which comprises a soluble TCR, fused via a linker to an anti-CD3 specific antibody fragment.

In a further aspect, the disclosure provides nucleic acid encoding the TCR of the disclosure, a TCR expression vector comprising nucleic acid encoding a TCR of the disclosure, as well as a cell harboring such a vector. The TCR may be encoded either in a single open reading frame or two distinct open reading frames. Also included in the scope of the disclosure is a cell harboring a first expression vector which comprises nucleic acid encoding an α-chain of a TCR of the disclosure, and a second expression vector which comprises nucleic acid encoding a β-chain of a TCR of the disclosure. Alternatively, one vector may encode both an α- and a β-chain of a TCR of the disclosure.

A further aspect of the present disclosure provides a cell displaying on its surface a TCR of the disclosure. The cell may be a T cell, or other immune cell. The T cell may be modified such that it does not correspond to a T cell as it exists in nature. For example, the cell may be transfected with a vector encoding a TCR of the disclosure such that the T cell expresses a further TCR in addition to the native TCR. Additionally or alternatively, the T cell may be modified such that it is not able to present the native TCR. There are a number of methods suitable for the transfection of T cells with DNA or RNA encoding the TCRs of the disclosure. As a non-limiting example, the transfection method may comprise a rapid RNA-based transfection system. T cells expressing the TCRs of the disclosure are suitable for use in adoptive therapy-based treatment of diseases such as cancers. There are a number of suitable methods by which adoptive therapy can be carried out. For example, adoptive cell therapy (ACT) may comprise use of autologous tumor-infiltrating lymphocytes, and may include a lymphodepletion preparative regimen prior to ACT. In some embodiments, viruses, e.g., retroviruses, that encode TCRs may be used for genetic modification of lymphocytes to convert normal lymphocytes into lymphocytes with anti-cancer activity. The adoptive transfer of lymphocytes with anti-cancer activity into patients requiring treatment of, e.g., metastatic melanoma, can mediate tumor regression. In some embodiments, ACT may comprise treatment of patients with cancers expressing viral or alloantigens, treatment of patients with cancers expressing viral antigens, and/or ACT using gene-modified lymphocytes. In some embodiments, ACT methods may include, for example, genetic modification of lymphocytes to introduce new recognition specificities using, e.g., αβTCR(s) and/or chimeric TCR(s); genetic modification of lymphocytes to alter function of T cells using, e.g., co-stimulatory molecules (e.g., CD28, 41BB), cytokines (e.g., IL2, IL15), homing molecules (e.g., CD62L, CCR7), and/or molecules capable of preventing apoptosis (BCL2); modification of host lymphodepletion using, e.g., selective depletion of CD4+ cells or T regulatory cells; blocking of inhibitory signals on reactive lymphocytes using, e.g., antibodies to CTLA4 and/or PD-1; administration of vaccines to stimulate transferred cells using, e.g., recombinant virus encoding antigen(s); administration of alternative cytokines to support cell growth using, e.g., IL15 and/or IL21; stimulation of APCs using, e.g., toll-like receptor agonists; generation of less differentiated lymphocytes using, e.g., alternate culture conditions and growth promoting cytokines in vitro; and, overcoming antigen escape variants using, e.g., natural killer cells.

The TCRs of the disclosure intended for use in adoptive therapy are generally glycosylated when expressed by the transfected T cells. The glycosylation pattern of transfected TCRs may be modified by mutations of the transfected gene.

In some embodiments, the peptide binding moiety may be a chimeric antigen receptor (CAR). CARs are genetically engineered receptors. CARs may be generated that bind the peptides or pMHC complexes of the present disclosure by incorporating an antigen binding domain that specifically binds the peptide or pMHC complex to the extracellular domain of the CAR. CARs may be introduced into and expressed by immune cells, such as T cells, NK cells, or macrophages. CARs can be programmed to both recognize a specific antigen and, when bound to that antigen, activate the immune cell to attack and destroy the cell presenting that antigen. When these antigens exist on tumor cells, an immune cell that expresses the CAR can target and kill the tumor cell.

The general structure of a CAR typically comprises an extracellular domain that binds the antigen (e.g. the peptides or pMHC complexes of the present disclosure), a hinge, a transmembrane domain, and an intercellular domain comprising a signaling domain and optionally one or more co-stimulatory domains.

Extracellular domains of the CAR may contain any polypeptide that specifically binds the desired antigen (e.g. the peptides or pMHC complexes of the present disclosure). For example, the extracellular domain may comprise an antibody fragment such as scFv or VHH. The CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

A hinge domain may be present between the extracellular domain and the transmembrane domain of the CAR, e.g., to provide flexibility to allow effective binding of the extracellular domain to its intended target. The hinge domain may be a polypeptide of about 2 to 100 amino acids in length. The hinge may include or be composed of flexible residues such as Gly and Ser so that the adjacent protein domains are free to move relative to one another. Longer hinges may be used when it is desirable to ensure that two adjacent domains do not sterically interfere with one another. The hinge may be derived from a hinge region or portion of the hinge region of any immunoglobulin. Non-limiting examples of linkers include a part of human CD8α chain, extracellular domain of CD28, an Ig hinge from IgG, IgM, IgA, IgD, or IgE, FcγRIIIa receptor, or a functional fragment thereof.

Transmembrane domains of the CAR may be derived transmembrane proteins, such as an alpha, beta or zeta chain of a T-cell receptor, CD28, CD3 epsilon, CD2, CD4, CD5, CD8, CD9, CD16, CD18, CD19, CD22, CD27, CD29, CD33, CD37, CD40, CD45, CD49a, CD64, CD80, CD84, CD86, CD96 (Tactile), CD100 (SEMA4D), CD103, CD134, CD154, CD160 (BY55), KIRDS2, OX40, LFA-1 (CD11a, CD18), CD11b, CD11c, CD11d, ICOS (CD278), 4-1 BB (CD137), 4-1 BBL, GITR, BAFFR, HVEM (LIGHTR), SLAMF7, NKp80 (KLRFI), IL2R beta, IL2R gamma, IL7Ra, ITGA1, VLA1, ITGA4, IA4, CD49D, ITGA6, VLA-6, CD49f, ITGAD, ITGAE, ITGAL, LFA-1, ITGAM, ITGAX, ITGB1, ITGB2, LFA-1, ITGB7, TNFR2, DNAM1 (CD226), SLAMF4 (CD244, 2B4), CEACAMI, CRT AM, Ly9 (CD229), PSGL1, SLAMF6 (NTB-A, Ly108), SLAM (SLAMF1, CD150, IPO-3), BLAME (SLAMF8), SELPLG (CD162), LTBR, PAG/Cbp, NKp30, NKp44, NKp46, NKG2D, and NKG2C, or functional fragment thereof.

The intracellular signaling domain of a CAR participates in transducing the signal of effective CAR binding to a target antigen into the interior of the immune effector cell to elicit an effector cell function, e.g., activation, cytokine production, proliferation, and cytotoxic activity, including the release of cytotoxic factors to the CAR-bound target cell, or other cellular responses elicited following antigen binding to the extracellular CAR domain. Non-limiting examples of intracellular signaling domains of the CAR include those derived from CD3ζ, CD3ε, CD3δ, CD3γ, CD5, CD22, CD39, CD79A, CD79B, CD66d, CD226, DAP10, DAP12, Fc epsilon receptor I gamma chain (FCER1G), or FcR β.

Intracellular co-stimulatory domains of the CAR can provide a second signal required for efficient activation and function of T lymphocytes upon binding to antigen. Such co-stimulatory domains may be derived from one or more co-stimulatory molecules, such as, but not limited to, 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, BTLA, GITR, CD226, HVEM, and ZAP70.

The CARs can be generated by standard molecular biology techniques. The extracellular domain that binds the desired antigen may be derived from the antibodies or their antigen binding fragments described herein.

In another aspect, the disclosure further provides cells that comprise peptide a binding moiety (e.g., TCRs and CARs) of the present disclosure. In some embodiments, the host cell is an immune cell. In some embodiments, the immune cell is T cell, NK cell, or a macrophage. The host cell may be autologous or allogeneic with respective to the subject receiving the cell (as treatment).

In some embodiments, TCRs of the present disclosure are provided as TCR-T cells. In some embodiments, CARs of the present disclosure are provided as CAR-T cells. Any methods known in the art for modifying T cells to express a TCR or CAR can be employed to generate the TCR-T or CAR-T cells of the present disclosure.

The cells expressing a peptide binding moiety (e.g., TCRs and CARs) of the present disclosure may also contain one or more additional genes. The additional genes can be used to increase the effector function of the cells expressing the peptide binding moiety (e.g., TCRs and CARs). Non-limiting examples of classes of additional genes include (a) a second targeting moiety, such as antibodies, including fragments thereof and bispecific antibodies (e.g., bispecific T cell engagers (BiTEs)), (b) secretable cytokines (e.g., GM-CSF, IL-7, IL-12, IL-15, IL-18), (c) membrane bound cytokines (e.g., IL-15), (d) chimeric cytokine receptors (e.g., IL-2/IL-7, IL-4/IL-7), (e) constitutive active cytokine receptors (e.g., C7R), (f) dominant negative receptors (DNR; e.g., TGFRII DNR), (g) ligands of co-stimulatory molecules (e.g., CD80, 4-1BBL), (h) nuclear factor of activated T cells (NFATs) (e.g., NFATc1, NFATc2, NFATc3, NFATc4, and NFAT5), or (j) suicide genes (e.g., CD20, truncated EGFR or HER2, inducible caspase 9 molecules). In some embodiments, the cells expressing a peptide binding moiety (e.g., TCRs and CARs) of the present disclosure may express a second targeting moiety that targets to the liver or to another known liver cancer antigen.

### Pharmaceutical Compositions, Dosage Forms, and Administration

In a further aspect, the disclosure provides a pharmaceutical composition comprising a peptide, a peptide-based molecule (such as a complex (e.g., peptide-MHC (pMHC) complex), fusion protein, or conjugate comprising the peptide), a nucleic acid molecule, a vector, a cell, or a peptide binding moiety of the disclosure together with a pharmaceutically acceptable carrier and/or excipient. The pharmaceutical compositions of the disclosure may be in any suitable form (depending upon the desired method of administering to a patient). Suitable compositions and methods of administration are known to those skilled in the art, for example see, Johnson et al., Blood. 2009; 114(3):535-46.

The pharmaceutical compositions may comprise the peptides or peptide-based molecules of the disclosure either in the free form or in the form of a pharmaceutically acceptable salt. The term “pharmaceutically acceptable salt” as used herein refers to a derivative of the disclosed peptides wherein the peptide is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids, e.g., acetic acid, benzoic acid, citric acid, propionic acid, glycolic acid, trifluoroacetic acid, pyruvic acid, oxalic acid, malic acid, malonic acid, maleic acid, succinic acid, fumaric acid, tartaric acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

Compositions of the disclosure may comprise multiple peptides, e.g., 2 to 50, 2 to 40, 2 to 30, 5 to 25, 5 to 20, or 10 to 15 peptides as described herein (e.g., SEQ ID NO: 1-54 and 110-112). In some embodiment, the compositions of the disclosure may comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 amino acid sequences selected from SEQ ID NO: 1-54 and 110-112, or a derivative thereof, or a pharmaceutically acceptable salt thereof.

In some embodiments, the peptides or peptide-based molecules may be present in a solution at a concentration of about 1 g/mL to 50 mg/mL, for example, about 0.1 mg/mL to 10 mg/mL, about 0.2 mg/mL to 5 mg/mL, about 0.5 mg/mL to 8 mg/mL, about 0.8 mg/mL to 12 mg/mL, about 1 mg/mL to 15 mg/mL, about 2 mg/mL to 20 mg/mL, or about 5 mg/mL to 25 mg/mL, or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.25 mg/mL, 1.5 mg/mL, 1.75 mg/mL, 2 mg/mL, 2.25 mg/mL, 2.5 mg/mL, 2.75 mg/mL, 3 mg/mL, 3.25 mg/mL, 3.5 mg/mL, 3.75 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL or 20 mg/mL.

The pharmaceutical composition may be adapted for administration by any appropriate route such as, e.g., parenteral (including subcutaneous, intramuscular, or intravenous), enteral (including oral or rectal), inhalation, or intranasal routes.

Such compositions may be prepared by any method known in the art of pharmacy, for example, by mixing the active ingredient with the carrier(s) or excipient(s) under sterile conditions.

In addition, disclosed herein are pharmaceutical dosage forms comprising the peptides, peptide-based molecules (such as complexes (e.g., peptide-MHC (pMHC) complexes), fusion proteins, or conjugates comprising the peptide(s)), nucleic acid molecules, vectors, cells, or binding moieties of the disclosure.

Pharmaceutical compositions based on the peptides, peptide-based molecules (such as complexes (e.g., peptide-MHC (pMHC) complexes), fusion proteins, or conjugates comprising the peptide(s)), nucleic acid molecules, vectors, cells, or binding moieties disclosed herein can be formulated in any conventional manner using one or more physiologically acceptable carriers and/or excipients. The peptides, peptide-based molecules (such as complexes (e.g., peptide-MHC (pMHC) complexes), fusion proteins, or conjugates comprising the peptide(s)), nucleic acid molecules, vectors, cells, or binding moieties may be formulated for administration by, for example, injection, inhalation, or insulation (either through the mouth or the nose) or by oral, buccal, parenteral or rectal administration, or by administration directly to an organ or tissue.

The pharmaceutical compositions can be formulated for a variety of modes of administration, including systemic, topical, or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

In some embodiments, the pharmaceutical compositions of the present disclosure may be lyophilized. As a non-limiting example, the obtained lyophilizate can be reconstituted into a hydrous composition by adding a hydrous solvent. In some embodiments, the hydrous composition may be able to be directly administered parenterally to a patient. Therefore, a further embodiment of the present disclosure is a hydrous pharmaceutical composition, obtainable via reconstitution of the lyophilizate with a hydrous solvent.

In some embodiments, the pharmaceutical composition disclosed herein may comprise a lyophilized formulation. As a non-limiting example, the lyophilization formulation may comprise peptides of the disclosure, mannitol, and/or TWEEN 80®. As another non-limiting example, the lyophilization formulation may comprise the peptides disclosed herein, mannitol and poloxamer 188. In some embodiments, the pharmaceutical composition may comprise a lyophilization formulation comprising a reconstituted-liquid composition.

In some embodiments, pharmaceutical compositions of the present disclosure may provide a formulation with an enhanced solubility and/or moistening of the lyophilizate over previously known compositions. As a non-limiting example, enhanced solubility and/or moistening of the lyophilizate may be achieved using an appropriate composition of excipients. In this way, pharmaceutical compositions of the present disclosure comprising peptides of SEQ ID NO: 1 to 54 and variants thereof may be developed to show a desired shelf stability at (e.g., at −20° C., +5° C., or +25° C.) and can be easily resolubilized such that the lyophilizate can be completely dissolved through the use of a buffer or other excipients from seconds up to two or more minutes, with or without the use of an of ultrasonic homogenizer. Furthermore, the composition can be easily provided to a patient in need of treatment via any appropriate delivery route disclosed herein, e.g., parenteral (including subcutaneous, intramuscular, or intravenous), enteral (including oral or rectal), inhalation, or intranasal routes. As a non-limiting example, the pH-value of the resulting solution may be between pH 2.7 and pH 9.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. ationd oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is men slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the vector particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered peptides, peptide-based molecules (such as complexes (e.g., peptide-MHC (pMHC) complexes), fusion proteins, or conjugates comprising the peptide(s)), nucleic acid molecules, vectors, cells, or binding moieties described herein on a daily or weekly basis for a time period or on a monthly, bi-yearly or yearly basis depending on need or exposure to a pathogenic organism (e.g., HBV) or to a condition in the subject (e.g. cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compounds sucrose as a sweetening agent methyl and propylparabens as preservatives, a dye and flavoring, such as cherry or orange flavor.

Further embodiments disclosed herein can concern kits for use with methods and compositions. Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the peptides, peptide-based molecules (such as complexes (e.g., peptide-MHC (pMHC) complexes), fusion proteins, or conjugates comprising the peptide(s)), nucleic acid molecules, vectors, cells, or binding moieties and any other reagent containers in close confinement for commercial sale. Such containers may include injection or blow-molded plastic containers into which the desired vials are retained. Optionally, one or more additional active agents may be needed for compositions described.

Dose ranges and frequency of administration can vary depending on the nature of the composition and the medical condition as well as parameters of a specific patient and the route of administration used. A dose can also depend on the subject in which it is being administered. For example, a lower dose may be required if the subject is juvenile, and a higher dose may be required if the subject is an adult human subject. In certain embodiments, a more accurate dose can depend on the weight of the subject. A suitable, non-limiting example of a dosage of a pharmaceutical composition containing the same disclosed herein may vary depending upon the age and the size of a subject to be administered, target disease, the purpose of the treatment, conditions, route of administration, and the like. Non-limiting examples of suitable dosages include, e.g., 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. In certain embodiments, the initial dose may be followed by administration of a second or a plurality of subsequent doses in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Certain additional agents used in the combination therapies can be formulated and administered by any means known in the art.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. For example, adjuvants can stimulate Th1 or Th2 immunity. Compositions and methods as disclosed herein can also include adjuvant therapy.

The peptides or peptides-based molecules of the disclosure may be provided in the form of a vaccine composition. The vaccine composition may be useful for the treatment or prevention of an HBV infection and/or HBV-induced diseases or disorders. As will be appreciated, vaccines may take several forms (see, e.g., Schlom, J Natl Cancer Inst. 2012; 104(8):599-613; Salgaller, Cancer Res. 1996; 56(20):4749-57 and Marchand, Int J Cancer. 1999; 80(2):219-30). The vaccine composition may include additional peptides or peptides-based molecules such that the peptide or peptides-based molecule of the disclosure is one of a mixture of peptides or peptides-based molecules. Adjuvants may be added to the vaccine composition to augment the immune response. In particular for peptide-containing vaccines compositions of the disclosure, pharmaceutically acceptable adjuvants include, but are not limited to, aluminum salts, Amplivax, AS 15, Aquila's QS21 stimulon, AsA404 (DMXAA), beta-glucan, BCG, CP-870,893, CpG7909, CyaA, dSLIM, GM-CSF, IC30, IC31, Imiquimod, ImuFact EV1P321, IS Patch, ISS, 1018 ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, poly-ICLC, PepTel®, Pam3Cys, PLGA microparticles, resiquimod, SRL172, Virosomes and other Virus-like particles, YF-17D, VEGF trap, R848, and/or vadimezan.

Alternatively, the vaccine composition may take the form of an APC displaying the peptide of the disclosure in complex with MHC. Preferably the APC is an immune cell, more preferably a dendritic cell or a B cell. The peptide may be pulsed onto the surface of the cell (Thurner, J Exp Med. 1999; 190(11):1669-78), or nucleic acid encoding for the peptide of the disclosure may be introduced into dendritic cells or B cells (e.g., by electroporation. Van Tendeloo, Blood. 2001; 98(1):49-56).

The pharmaceutical compositions of the disclosure may be administered directly into the patient, into the affected organ or systemically i.d., i.m., s.c., i.p. and i.v., or applied ex vivo to cells derived from the patient or a human cell line which are subsequently administered to the patient, or used in vitro to select a subpopulation of immune cells derived from the patient, which are then re-administered to the patient. If the nucleic acid is administered to cells in vitro, it may be useful for the cells to be transfected so as to co-express immune-stimulating cytokines, such as interleukin-2. The peptide or peptide-based molecule may be substantially pure, or combined with an immune-stimulating adjuvant or used in combination with immune-stimulatory cytokines, or be administered with a suitable delivery system, e.g., liposomes, viral particles, VLPs. The peptide or peptide-based molecule may also be conjugated to a suitable carrier such as keyhole limpet haemocyanin (KLH) or mannan (see, e.g., WO 95/18145 and Longenecker et al., 1993).

In some embodiments, the peptide-containing compositions described herein further comprise an accessory molecule which can modulate a survival or an activity of TCR-expressing cells.

Non-limiting examples of useful accessory molecules include, e.g., an anti-CD28 antibody, an anti-CD80 (B7.1) antibody, an anti-CD86 (B7.2) antibody, an anti-anti-CD3 antibody, an anti-CD2 antibody, an anti-CD4 antibody, an anti-CD8 antibody, an anti-CD47 antibody, and functional derivatives, mutants and fragments thereof.

Accessory molecules used in the peptide-containing compositions described herein include molecules that provide a signal which, in addition to the primary signal provided by, for instance, binding of a TCR/CD3 complex with a pMHC complex, mediates a T cell response, including, but not limited to, proliferation, activation, differentiation, and the like.

The accessory molecule can be, for example, an inhibitory or stimulatory antibody, a peptide ligand, a costimulatory peptide, a cytokine, etc. Non-limiting examples of accessory molecules that can be used in the peptide-containing compositions described herein include, e.g., CD7, B7.1 (CD80), B7.2 (CD86), PD-L1, PD-L2, 4-1BBL, OX40L, Fas ligand (FasL), inducible co stimulatory ligand (ICOS-L), intercellular adhesion molecule (ICAM), CD30L, CD40, CD70, CD83, HLA-G, MICA, MICB, FIVEM, lymphotoxin β receptor, 3/TR6, ILT3, ILT4, HVEM, an agonist or antibody that binds Toll ligand receptor and a ligand that specifically binds to B7-H3 as well as antibodies that specifically bind to CD27, CD28, B7.1 (CD80), B7.2 (CD86), 4-1BB, OX40, CD30, CD40, PD-1, ICOS, lymphocyte function-associated antigen-1 (LFA-1), CD2, CD3, CD7, LIGHT, NKG2C, B7-H3, and a ligand that specifically binds to CD83.

Additional non-limiting examples of accessory molecules include, e.g., TNF/TNF family members (e.g., OX40L, ICOSL, FASL, LTA, LTB TRAIL, CD153, TNFSF9, RANKL, TWEAK, TNFSF13, TNFSF13b, TNFSF14, TNFSF15, TNFSF18, CD40LG, CD70); members of the Immunoglobulin superfamily (e.g., VISTA, PD1, PD-L1, PD-L2, B71, B72, CTLA4, CD28, TIM3, CD4, CD8, CD19, T cell receptor chains, ICOS, ICOS ligand, HHLA2, butyrophilms, BTLA, B7-H3, B7-H4, CD3, CD79a, CD79b, IgSF CAMS (including CD2, CD58, CD48, CD150, CD229, CD244, ICAM-1), Leukocyte immunoglobulin like receptors (LILR), killer cell immunoglobulin like receptors (KIR)), lectin superfamily members, selectins, cytokines/chemokine and cytokine/chemokine receptors, growth factors and growth factor receptors), adhesion molecules (integrins, fibronectins, cadherins), or ecto-domains of multi-span integral membrane proteins, or antibodies directed to any of these molecules.

In some embodiments, the peptide-containing compositions described herein further comprise a cytotoxic agent. In one specific embodiment, the cytotoxic agent is a toxin or a radioactive isotope (e.g., a radioconjugate) or a suicide gene. Non-limiting examples of toxins which can be used in the peptide-containing compositions described herein include, e.g., enzymatically active toxins of bacterial, fungal, plant, or animal origin, or fragments, mutants or derivatives thereof. Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. Non-limiting examples of suicide genes include, e.g., thymidine kinase, cytosine deaminase, purine nucleoside phosphorylase, nitroreductase, β-galactosidase, hepatic cytochrome P450-2B1, linamarase, horseradish peroxidase, and carboxypeptidase.

Methods for introducing polypeptide or polynucleotides of the present disclosure into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. In some embodiments, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule.

The use of nanoparticles to deliver the polypeptide or polynucleotides compositions of the disclosure is contemplated herein. Exemplary nanoparticles include, but are not limited to, polymeric nanoparticles, inorganic nanoparticles, liposomes, lipid nanoparticles (LNP), an immune stimulating complex (ISCOM), a virus-like particle (VLP), or a self-assembling protein. The nanoparticles may be calcium phosphate nanoparticles, silicon nanoparticles or gold nanoparticles. For examples, the polymeric nanoparticles may comprise one or more synthetic polymers, such as poly(d,l-lactide-co-glycolide) (PLG), poly(d,l-lactic-coglycolic acid) (PLGA), poly(g-glutamic acid) (g-PGA), poly(ethylene glycol) (PEG), or polystyrene or one or more natural polymers such as a polysaccharide, for example pullulan, alginate, inulin, and chitosan. The use of a polymeric nanoparticles may be advantageous due to the properties of the polymers that may be include in the nanoparticle. For instance, the natural and synthetic polymers recited above may have good biocompatibility and biodegradability, a non-toxic nature and/or the ability to be manipulated into desired shapes and sizes. The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles. Inorganic nanoparticles typically have a rigid structure and comprise a shell in which an antigen is encapsulated or a core to which the antigen may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

Other molecules suitable for complexing with the polypeptide or polynucleotides of the disclosure include cationic molecules, such as, polyamidoamine, dendritic polylysine, polyethylene irinine or polypropylene imine, polylysine, chitosan, DNA-gelatin coarcervates, DEAE dextran, dendrimers, or polyethylenimine (PEI).

In some embodiments, antibodies of the present disclosure can be conjugated to nanoparticles. Nanoparticles that may be used for conjugation with antibodies of the present disclosure include but not are limited to PEGylated liposomes, poly(d,l-lactide-co-glycolide)/montmorillonite nanoparticles (PLGA/MMT NPs), poly(lactide-co-glycolide) (PLGA) nanoparticles, poly-(malic acid)-based nanoparticles, chitosan-shelled nanoparticles, carbon nanotubes, and other inorganic nanoparticles (such as nanoparticles made of magnesium-aluminium layered double hydroxides with disuccinimidyl carbonate (DSC), and TiO₂ nanoparticles). Nanoparticles can be developed and conjugated to an antibody contained in a pharmaceutical composition for targeting virus-infected cells.

### Treatment Methods

Compositions of the present disclosure, including the peptides, peptide-based molecules (such as complexes (e.g., peptide-MHC (pMHC) complexes), fusion proteins, or conjugates comprising the peptide(s)), nucleic acid molecules, vectors, cells, or binding moieties of the disclosure, may be used in the prophylaxis and/or treatment of a viral infection (e.g., HBV infection) and/or diseases or disorders caused by the viral infection (e.g., HBV infection).

In one aspect, disclosed herein is a method for modulating an activity, proliferation or survival of a cell comprising a TCR, comprising contacting the cell with a composition (e.g., peptide, complex (e.g., pMHC complex), fusion protein, or conjugate) of the disclosure.

In some embodiments, the cell is a lymphocyte such as, e.g., a T-cell (e.g., a CD4+ T-cell or a CD8+ T-cell). In some embodiments, the target T cell is a CD4+ T cell such as, e.g., a helper T cell (e.g., a Th1, Th2, or Th17 cell) or a CD4+/CD25+/FOXP3+ regulatory T (Treg) cell. In some cases, the target T cell is a CD8+ T cell such as, e.g., a cytotoxic T cell. In some cases, the target T cell is a memory T cell, which can be a CD4+ T cell or a CD8+ T cell, where memory T cells are generally CD45RO+. In some cases, the target T cell is an NK-T cell.

In some embodiments, the contacting is ex vivo. In some embodiments, the contacting is in vivo in a subject (e.g., human).

In some embodiments, the cell is a mammalian cell (e.g., a human cell).

In some embodiments, e.g., where the target T cell is a CD8+ T cell, the peptide is presented by a class I MHC polypeptide. In some embodiments, e.g., where the target T cell is a CD4+ T cell, the peptide is presented by class II MHC polypeptides.

The interaction of a T cell with the peptides described herein can result in, e.g., activation, induction of anergy, or death of a T cell that occurs when the TCR of the T cell is bound by a TCR-binding molecule (e.g., pMHC complex). “Activation of a T cell” refers to induction of signal transduction pathways in the T cell resulting in production of cellular products (e.g., interleukin-2) by that T cell. “Anergy” refers to the diminished reactivity by a T cell to an antigen. Activation and anergy can be measured by, for example, measuring the amount of IL-2 produced by a T cell after a pMHC complex has bound to the TCR. Anergic cells will have decreased IL-2 production when compared with stimulated T cells. Another method for measuring the diminished activity of anergic T cells includes measuring intracellular and/or extracellular calcium mobilization by a T cell upon engagement of its TCR's. “T cell death” refers to the permanent cessation of substantially all functions of the T cell.

In another aspect, provided herein is a method of inducing an immune response against a HBV infection in a subject in need thereof, the method comprising administering to the subject a therapeutically effective amount of a composition (e.g., one or more peptides, complexes (e.g., pMHC complex), fusion proteins, conjugates, nucleic acid molecules, vectors, cells, or binding moieties) of the present disclosure.

In certain embodiments, generating an immune response comprises an increase in target antigen-specific cytotoxic T lymphocytes (CTL) activity of about 1.5-fold to 20-fold, or more fold in a subject administered a composition of the disclosure as compared to a control. In certain embodiments, generating an immune response comprises an increase in target-specific CTL activity of about 1.5-fold to 20-fold, or more fold in a subject administered the composition of the disclosure as compared to a control. In a further embodiment, generating an immune response that comprises an increase in target antigen-specific cell-mediated immunity activity as measured by ELISpot assays measuring cytokine secretion, such as interferon-gamma (IFN-γ), interleukin-2 (IL-2), tumor necrosis factor-alpha (TNF-α), or other cytokines, of about 1.5-fold to 20-fold, or more fold as compared to a control.

In a further embodiment, generating an immune response comprises an increase in target-specific antibody production of between 1.5-fold and 5-fold in a subject administered the composition of the disclosure as compared to an appropriate control. In another embodiment, generating an immune response comprises an increase in target-specific antibody production of about 1.5-fold to 20-fold, or more fold in a subject administered the composition of the disclosure as compared to a control.

T cell activation may be determined, e.g., by measuring changes in the level of expression of cytokines and/or T cell activation markers, and/or the induction of antigen-specific proliferating cells. Techniques known to those of skill in the art, including, but not limited to, immunoprecipitation followed by western blot analysis, ELISAs, flow cytometry, northern blot analysis, and RT-PCR can be used to measure the expression cytokines and T cell activation markers. Cytokine release may be measured by measuring secretion of cytokines including but not limited to Interleukin-2 (IL-2), Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, IFN-7, TFN-7, IGF-I, and IGF-II.

T cell modulation may also be evaluated by measuring, e.g., proliferation by, e.g., ³H-thymidine incorporation, trypan blue cell counts, and fluorescence activated cell sorting (FACS).

The anti-tumor responses of T cells may be determined in xenograft tumor models. Tumors may be established using any human cancer cell line expressing the relevant tumor associated antigen. To establish xenograft tumor models, about 5×10⁶ viable cells, may be injected, e.g., subcutaneously into nude athymic mice using for example Matrigel (Becton Dickinson). The endpoint of the xenograft tumor models can be determined based on the size of the tumors, weight of animals, survival time and histochemical and histopathological examination of the cancer, using methods known to one skilled in the art.

In a related aspect, disclosed herein is a method of treating an HBV infection in a subject in need thereof, the method comprising administering to the subject an effective amount of a composition (e.g., one or more peptides, complexes (e.g., pMHC complex), fusion proteins, conjugates, nucleic acid molecules, vectors, cells, binding moieties) of the present disclosure.

In a related aspect, disclosed herein is a method of preventing or reducing the likelihood of an HBV-induced disease or disorder in a subject in need thereof, the method comprising administering to the subject an effective amount of a composition (e.g., one or more peptides, complexes (e.g., pMHC complex), fusion proteins, conjugates, nucleic acid molecules, vectors, a cells, binding moieties) of the present disclosure.

The HBV-induced disease or disorder can be, e.g., liver inflammation (either chronic or acute), liver fibrosis, liver cirrhosis, liver failure, or liver cancer. The liver cancer can be hepatocellular carcinoma (HCC), or intrahepatic cholangiocarcinoma and hepatoblastoma. In one embodiment, the liver cancer is hepatocellular carcinoma (HCC).

Hepatitis B virus infection may either be acute (self-limiting) or chronic (long-standing). Persons with self-limiting infection clear the infection spontaneously within weeks to months. In some cases, “chronic hepatitis” may result. Chronic hepatitis occurs when the body is unable to completely clear the virus even though the symptoms may not persist.

Acute infection with hepatitis B virus is associated with acute viral hepatitis—an illness that begins with general ill-health, loss of appetite, nausea, vomiting, body aches, mild fever, dark urine, and then progresses to development of jaundice. It has been noted that itchy skin has been an indication as a possible symptom of all hepatitis virus types. The illness lasts for a few weeks and then gradually improves in most affected people. A few patients may have more severe liver disease (e.g., fulminant hepatic failure), and may die as a result of it. The infection may be entirely asymptomatic and may go unrecognized. Continued presence of the virus over a number of years can lead to cirrhosis. This type of infection dramatically increases the incidence of liver cancer (e.g., hepatocellular carcinoma).

Carriers of the HBV can be identified by detecting in serum or blood the presence of either HBV viral antigens or antibodies produced by the host. The hepatitis B surface antigen (HBsAg) is most frequently used to screen for the presence of this infection. PCR tests may also be used to detect and measure the amount of viral nucleic acid in clinical specimens.

When the disease being treated is a cancer, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

It is contemplated that when used to treat various diseases, the compositions and methods can be combined with other therapeutic agents suitable for the same or similar diseases. Also, two or more embodiments described herein may be also co-administered to generate additive or synergistic effects. When co-administered with a second therapeutic agent, the embodiment described herein and the second therapeutic agent may be simultaneously or sequentially (in any order). Suitable therapeutically effective dosages for each agent may be lowered due to the additive action or synergy.

In some embodiments, the compositions and methods disclosed herein are useful to enhance the efficacy of vaccines directed to HBV infection or HBV-induced diseases (e.g., liver cancer such as HCC). Thus, the compositions and methods described herein can be administered to a subject either simultaneously with or before (e.g., 1-30 days before) a reagent (including but not limited to small molecules, antibodies, or cellular reagents) that acts to elicit an immune response (e.g., to treat HBV infection or liver cancer) is administered to the subject.

The compositions and methods described herein can be also administered in combination with an anti-tumor antibody or an antibody directed at a pathogenic antigen (e.g., HBV) or allergen.

The compositions and methods described herein can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments described herein can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents.

Therapeutic methods described herein can be combined with additional immunotherapies and therapies. For example, when used for treating cancer, NKT cells described herein can be used in combination with cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor 3, interferon-α, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

For treatment of viral infections, combined therapy described herein can encompass co-administering compositions and methods described herein with an anti-viral drug. Non-limiting examples of useful anti-viral drugs include, adefovir and entecavir, telbivudine, immune system modullators such as interferon-α, -β or -γ, didanosine, lamivudine, zanamavir, lopanivir, nelfinavir, efavirenz, indinavir, valacyclovir, zidovudine, amantadine, rimantidine, ribavirin, ganciclovir, foscarnet, and acyclovir or any salts or variants thereof. See also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### Kits

The present disclosure further comprises a kit which may comprise any of various compositions of the present disclosure, including the peptides, peptide-based molecules (such as complexes (e.g., peptide-MHC (pMHC) complexes), fusion proteins, or conjugates comprising the peptide(s)), nucleic acid molecules, vectors, cells, or binding moieties of the disclosure.

In one aspect, the present disclosure may include a kit comprising, for example: (a) a container that contains a pharmaceutical composition disclosed herein, for example, a pharmaceutical composition in solution or in lyophilized form; (b) optionally, a second container containing a diluent or reconstituting solution for the lyophilized formulation; and/or (c) optionally, instructions for (i) use of the solution or (ii) reconstitution and/or use of the lyophilized formulation.

In some embodiments, the kit may further comprise, for example, without limitation, one or more of (i) a buffer, (ii) a diluent, (iii) a filter, (iv) a needle, and/or (v) a syringe. As a non-limiting example, the container may be a bottle, a vial, a syringe or test tube. In some embodiments, the container may be a multi-use container. In some the pharmaceutical composition may be lyophilized.

Kits of the present disclosure may comprise a lyophilized formulation of the present disclosure in a suitable container and instructions for its reconstitution and/or use. Suitable containers include, for example, bottles, vials (e.g. dual chamber vials), syringes (such as dual chamber syringes) and test tubes. The container may be formed from a variety of materials such as glass or plastic. The kit and/or container may contain instructions on or associated with the container that indicate directions for reconstitution of the lyophilized formulation and/or use of the kit. For example, the label may indicate that the lyophilized formulation is to be reconstituted to an appropriate peptide concentration. The label may indicate that the formulation is useful or intended for any route of administration disclosed herein, e.g., parenteral administration routes disclosed herein.

The container holding the formulation may be a multi-use vial, which may allow for repeat administrations (e.g., from 2-6 administrations) of the reconstituted formulation. The kit may further comprise a second container comprising a suitable diluent (e.g., sodium bicarbonate solution).

Upon mixing of the diluent and the lyophilized formulation, the final peptide concentration in the reconstituted formulation is reached. The kit may further include other materials desirable from a commercial and/or user standpoint, including, for example, without limitation, other buffers, diluents, filters, needles, syringes, and/or package inserts which may comprise, e.g., instructions for use.

Kits of the present disclosure may have a single container that contains the formulation of the pharmaceutical compositions according to the present disclosure with or without other components (e.g., other compounds or pharmaceutical compositions of these other compounds) or may have a distinct container for each component.

In some embodiments, kits of the disclosure may include a formulation of the disclosure packaged for use in combination with the coadministration of a second compound (such as adjuvants (e.g., GM-CSF, a chemotherapeutic agent, a natural product, a hormone or antagonist, an anti-angiogenesis agent or inhibitor, an apoptosis-inducing agent or a chelator) or a pharmaceutical composition thereof. The components of the kit may be pre-complexed or each component may be in a separate distinct container prior to administration to a patient. The components of the kit may be provided in one or more liquid solutions. A liquid solutions described herein may be an aqueous solution, for example, a sterile aqueous solution. The components of the kit may also be provided as solids, which may be converted into liquids such as by addition of suitable solvents, which may be provided in another distinct container.

The container of a therapeutic kit may be a vial, test tube, flask, bottle, syringe, or any other means of enclosing a solid or liquid. When there is more than one component, the kit may contain a second vial or other container, which may allow for separate dosing. The kit may also contain another container for a pharmaceutically acceptable liquid. In some embodiment, a kit may contain an apparatus (e.g., one or more needles, syringes, eye droppers, pipettes, etc.), which may allow for administration of the agents of the disclosure that are components of the present kit.

### Method and System for Identifying an Immunogenic Virus-Derived Peptide

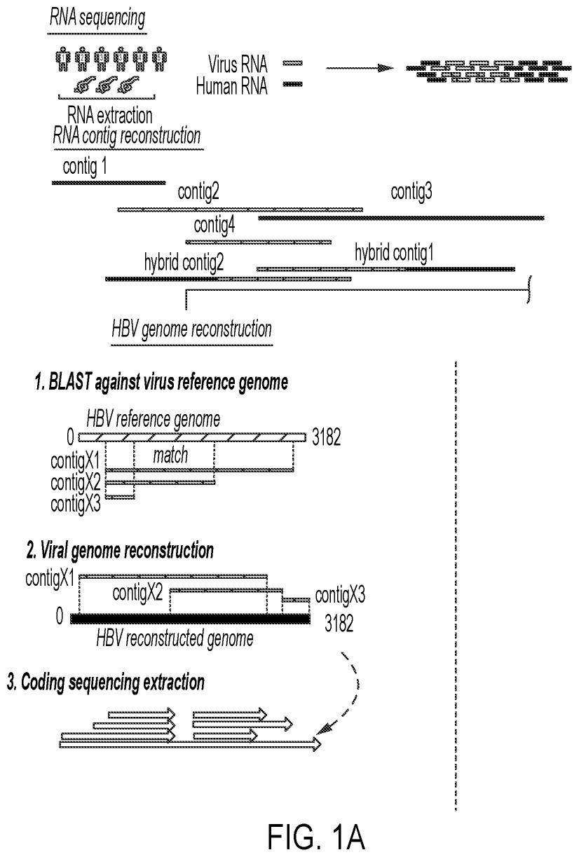

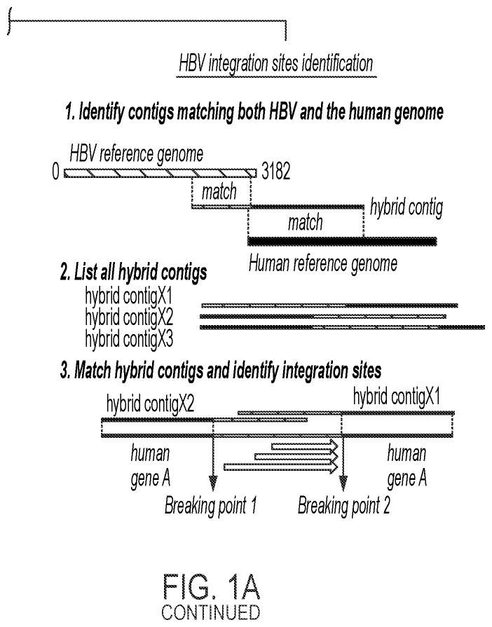

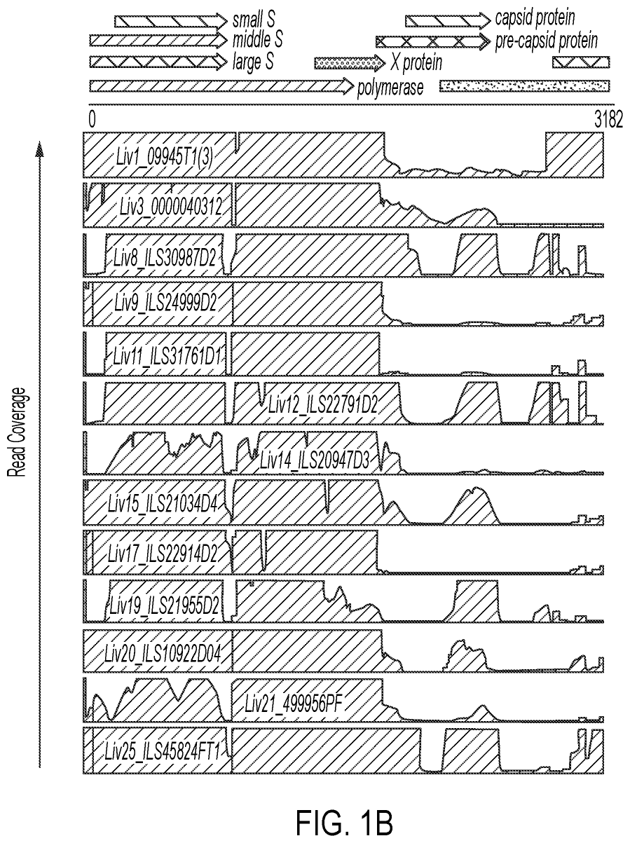

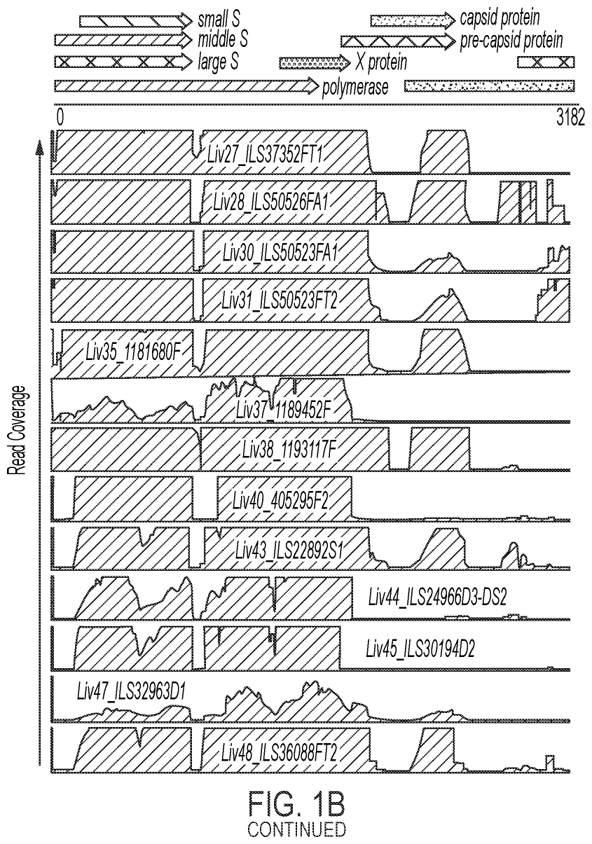

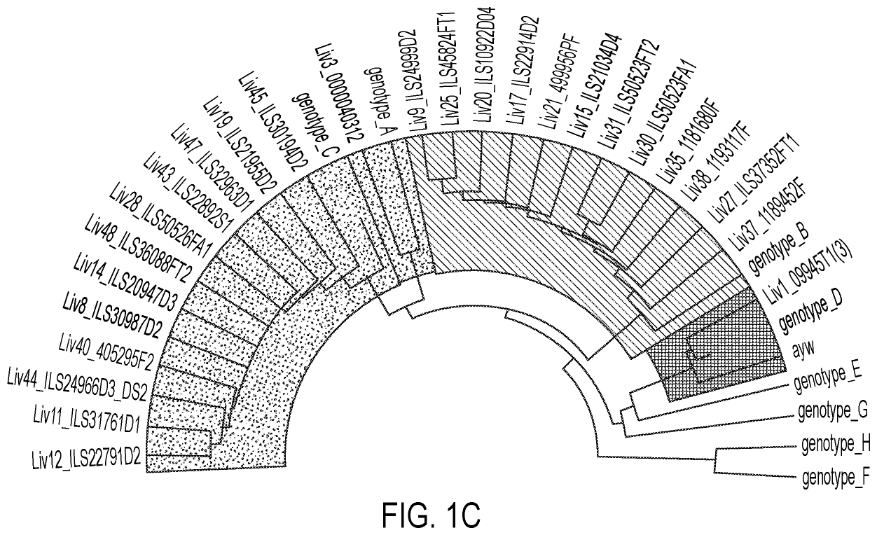

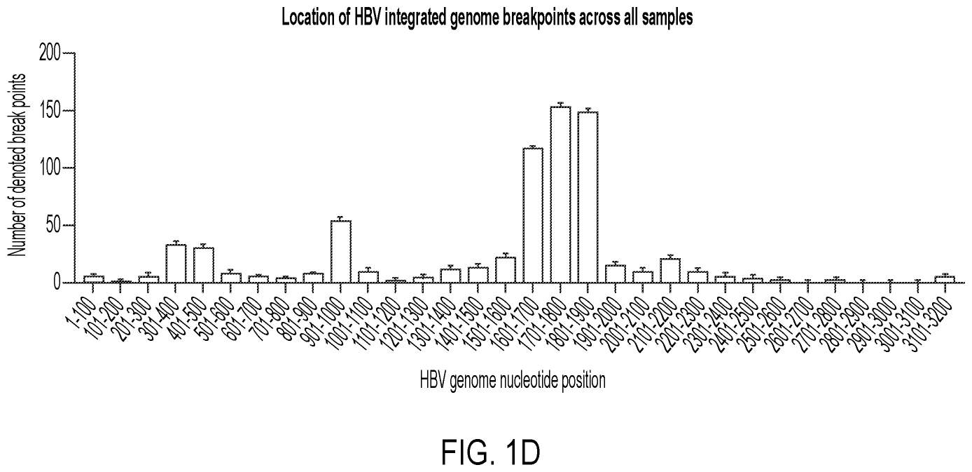

The present disclosure further comprises a method and system for identifying an immunogenic virus-derived peptide. Some or all the steps of the method can be executed by a computational device. In some aspects, some or all of the steps of the method can be stored as computer-readable instructions within a memory such that the instructions can be executed by one or more processors to perform functions associated with the method. As a non-limiting example, the method and system for identifying an immunogenic virus-derived peptide can include aspects of the HBV genome reconstruction flow illustrated in FIG. 1A.

In one aspect, the present disclosure may include a method for identifying an immunogenic virus-derived peptide that includes the following steps: (a) obtaining a plurality of RNA contig sequences derived from an infected subject infected with a virus, wherein the plurality of RNA contig sequences comprise a plurality of virus-derived RNA contig sequences and a plurality of infected-subject endogenous RNA contig sequences; (b) identifying the plurality of virus-derived RNA contig sequences from within the plurality of RNA contig sequences; (c) assembling a viral RNA sequence based on the plurality of virus-derived RNA contig sequences; (d) identifying a protein sequence based on the viral RNA sequence; and (e) identifying the immunogenic virus-derived peptide based at least in part on the identified protein sequence.

In one aspect, a system configured to perform functions associated with the foregoing method can include a non-transitory computer-readable medium configured to communicate with one or more processor(s) of a computational device. The non-transitory computer-readable medium may include instructions thereon, that when executed by the processor(s), cause the computational device to: (a) receive, as an input, a plurality of RNA contig sequences derived from an infected subject infected with a virus such that the plurality of RNA contig sequences comprise a plurality of virus-derived RNA contig sequences and a plurality of infected-subject endogenous RNA contig sequences, and wherein the infected subject is infected with a virus; (b) identify the plurality of virus-derived RNA contig sequences from within the plurality of RNA contig sequences; (c) assemble a viral RNA sequence based on the plurality of virus-derived RNA contig sequences; (d) identify a protein sequence based on the viral RNA sequence; (e) identify an immunogenic virus-derived peptide based at least in part on the protein sequence; and (f) provide, as an output, the immunogenic virus-derived peptide.

In some embodiments, the plurality of RNA contig sequences may be derived from one infected subject.

In some embodiments, the infected subject may be a human.

In some embodiments, the plurality of virus-derived RNA contig sequences are derived from the virus infecting the infected subject.

In some embodiments, the plurality of infected-subject endogenous RNA contig sequences are derived from RNA endogenous to the infected subject.

In some embodiments, identifying the plurality of virus-derived RNA contig sequences from within the plurality of RNA contig sequences (step b) can further include: comparing at least a portion of contig sequences of the plurality of RNA contig sequences to a reference viral sequence; and identifying the plurality of virus-derived RNA contig sequences such that each contig sequence of the plurality of virus-derived RNA contig sequences comprises at least a portion that corresponds to the reference viral sequence.

In some embodiments, each contig sequence of the plurality of virus-derived RNA contig may be distinct from the plurality of infected-subject endogenous RNA contig sequences.

In some embodiments, each contig sequence of the plurality of virus-derived RNA contig sequences may lack infected-subject endogenous RNA contig sequences.

In some embodiments, the reference viral sequence may include a reference genome.

In some embodiments, the reference genome may include a hepatitis B virus genome.

In some embodiments, assembling the viral RNA sequence based on the plurality of virus-derived RNA contig sequences (step c) may include: overlapping common sequence portions at ends of at least a portion of the plurality of virus-derived RNA contig sequences such that the at least a portion of the plurality of virus-derived RNA contig sequences overlap linearly to assemble the viral RNA sequence.

In some embodiments, identifying a protein sequence based on the viral RNA sequence such that the protein sequence includes a translation of the viral RNA sequence (step d) may include: identifying the protein sequence without requiring a comparison to a database of viral proteins.

In some embodiments, identifying the protein sequence based on the viral RNA sequence such that the protein sequence includes a translation of the viral RNA sequence (step d) may include: identifying a plurality of protein sequences each based on the viral RNA sequence such that each of the plurality of protein sequences respectively include a translation of the viral RNA sequence, and identifying the protein sequence as a frequently occurring protein sequence within the plurality of protein sequences.

In some embodiments, the protein sequence may be identified based on the viral RNA sequence associated with a single infected subject.

In some embodiments, identifying the immunogenic virus-derived peptide based at least in part on the protein sequence (step 3) may include: identifying a MHC molecule associated with the single infected subject; identifying one or more peptides based at least in part on the protein sequence such that the one or more peptides each form a respective MHC-peptide complex with the MHC molecule; and identifying the immunogenic virus-derived peptide based on the one or more peptides.

Method and System for Identifying an Integration Site of a Viral Gene within a Subject Gene

The present disclosure further comprises a method and system for identifying an integration site of a viral gene within a subject gene. Some or all the steps of the method can be executed by a computational device. In some aspects, some or all of the steps of the method can be stored as computer-readable instructions within a memory such that the instructions can be executed by one or more processors to perform functions associated with the method. As a non-limiting example, the method and system for identifying an integration site of a viral gene within a subject gene can include aspects of the HBV integration sites integration flow illustrated in FIG. 1A.

In one aspect, the present disclosure may include a method for identifying an integration site of a viral gene within a subject gene that includes the following steps: (a) obtaining a plurality of RNA contig sequences derived from an infected subject infected with a virus such that the plurality of RNA contig sequences comprise a plurality of virus-derived RNA contig sequences, a plurality of infected-subject endogenous RNA contig sequences, and a plurality of hybrid RNA contig sequences comprising viral and infected-subject endogenous portions; (b) identifying the plurality of hybrid RNA contig sequences from within the plurality of RNA contig sequences; (c) comparing, for at least a portion of the plurality of hybrid RNA contig sequences, infected-subject endogenous portions to a subject reference genome; and (d) identifying, based at least in part on the comparison of infected-subject endogenous portions to the subject reference genome, an integration site comprising the subject gene.

In one aspect, a system configured to perform functions associated with the foregoing method can include a non-transitory computer-readable medium configured to communicate with one or more processor(s) of a computational device. The non-transitory computer-readable medium may include instructions thereon, that when executed by the processor(s), cause the computational device to: (a) receive, as an input, a plurality of RNA contig sequences derived from an infected subject infected with a virus such that the plurality of RNA contig sequences comprise a plurality of virus-derived RNA contig sequences, a plurality of infected-subject endogenous RNA contig sequences, and plurality of hybrid RNA contig sequences comprising viral and infected-subject endogenous portions; (b) identify the plurality of hybrid RNA contig sequences from within the plurality of RNA contig sequences; (c) compare, for at least a portion of the plurality of hybrid RNA contig sequences, infected-subject endogenous portions to a subject reference genome; (d) identify, based at least in part on the comparison of infected-subject endogenous portions to the subject reference genome, an integration site comprising a subject gene; and (e) provide, as an output, the integration site.

In some embodiments, the plurality of RNA contig sequences may be derived from one infected subject.

In some embodiments, the infected subject may be a human.

In some embodiments, the plurality of virus-derived RNA contig sequences may be derived from the virus infecting the infected subject.

In some embodiments, the virus may include a hepatitis B virus.

In some embodiments, the plurality of infected-subject endogenous RNA contig sequences may be derived from RNA endogenous to the infected subject.

In some embodiments, the subject reference genome can include the human genome.

Certain embodiments and implementations of the disclosed technology are described above with reference to systems and methods and/or computer program products according to example embodiments or implementations of the disclosed technology. It will be understood that method steps can be implemented by computer-executable program instructions. Likewise, some method steps need not be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions associated with method steps. These computer program instructions may also be stored in a computer-readable memory that can direct a computer or other programmable data processing apparatus to function in a particular manner, such that the instructions stored in the computer-readable memory produce an article of manufacture including instruction means that implement one or more functions associated with method steps.

Non-transitory computer-readable media may include, but is not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable information.

Embodiments or implementations of the disclosed technology may provide for a computer program product, including a computer-usable medium having a computer-readable program code or program instructions embodied therein, said computer-readable program code adapted to be executed to implement one or more functions related to example methods presented herein. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions related to example methods presented herein.

Accordingly, illustrations and descriptions of methods support combinations of means for performing the specified functions, combinations of elements or steps for performing the specified functions, and program instruction means for performing the specified functions. It will also be understood that at least some method steps, and combinations of method steps, can be implemented by special-purpose, hardware-based computer systems that perform the specified functions, elements or steps, or combinations of special-purpose hardware and computer instructions.

Certain implementations of the disclosed technology can be utilized with customer devices that may include mobile computing devices. Those skilled in the art will recognize that there are several categories of mobile devices, generally known as portable computing devices that can run on batteries but are not usually classified as laptops. For example, mobile devices can include, but are not limited to portable computers, tablet PCs, internet tablets, PDAs, ultra-mobile PCs (UMPCs), wearable devices, and smart phones.

Certain implementations of the disclosed technology can be utilized with medical equipment, medical devices, and/or associated peripherals.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1. Generation of RNAseq Databases from HBV-Infected Patient Samples

Comparative genomic analyses are often guided by a handful of “reference” genomes. As of January 2022, the National Center for Biotechnology and Information (NCBI) database contains a repository of more than 13,000 HBV genome sequences classified into at least ten distinct phylogenetic lineages (A to J). While each lineage is represented by a single HBV genome, these reference sequences do not capture the full breadth of genomic diversity present in nature. This lack of coverage can negatively impact identification of new viral peptides as the sequence of patient isolates can greatly diverge from the reference genome sequence used for the comparative analysis.

To circumvent this problem, an RNAseq-based approach was developed to generate a database of patient-specific HBV genome sequences directly reconstituted from the RNA reads amplified from patient liver samples from Asian populations (FIG. 1A). A de-novo sequencing strategy was used to reconstruct patient specific HBV genomes and identify HBV-integration sites within human chromosomes. Total human RNA reads were converted into large contigs, and contigs with loose homology to HBV reference genomes are marked as HBV-specific sequences. HBV contigs were then grouped into two categories based on the percentage of the contig sequence covering the HBV reference. Fully-mapped contigs to HBV reference were re-arranged in a reference free fashion to assemble patient-specific HBV genomic sequences. Specific coding sequences from each reconstructed genome were saved into a large patient-specific HBV database. Contigs with hybrid matches to the HBV reference, were mapped against the human genome and the hybrid contigs matching both HBV and human sequences were selected. Specifically, hybrid contigs matching both human and HBV references were filtered to identify site of integration within the human genomes using a homology sequence alignment approach. The patient-specific viral sequence was then used to extract and translate all HBV coding sequences. Coverage of total RNA reads from bulk RNA sequencing of all human liver samples to HBV reference genome is shown in FIG. 1B. Using this approach, a total of 80 unique coding sequences (large S=14, middle S=14, small S=16, pre-capsid=6, capsid=12, X=16 and polymerase=2) retrieved from virus isolates that are representative of four phylogenetic lineages (A, B, C, D) were identified (FIG. 1C). The phylogenetic relationship between reconstructed HBV genomes and reference HBV sequences representative of eight HBV lineages (A, B, C, D, E, F, G, H) is depicted in FIG. 1C, where the tree was generated using a Neighbor-joining phylogenetic approach rooted on the mid-point. Read mapping analysis showed that the depth of coverage of virus reads was high and consistent in the genomic region encoding the S and X proteins, while coverage for the rest of the genome contained significant gaps (FIG. 1B). These gaps in the genome suggested that the reads could be coming from partially integrated HBV DNA sequences. The N-terminal region of the genome-spanning polymerase gene was poorly covered, whereas the C-terminal portion of the polymerase gene was well detected. For the majority of HBV+ samples, hybrid contigs combining both HBV and human sequences were also identified. These sequences likely result from integration of HBV DNA into the coding sequence of a human gene. The genomic location of the HBV sequence in these junctions was referred to as “breakpoints”. The HBV breakpoints found in RNAseq reads with HBV/human junctions were mapped and the majority of HBV breakpoints was found to occur between nucleotide positions 1601-1900 in the HBV genome as shown in FIG. 1D, where the frequency of the genomic locations of the breakpoints across all studied samples is displayed in 100 nucleotide intervals; no clear pattern was observed for host genome (Table 3). These data suggest that at least some of the detected HBV sequences are coming from integrated DNA and not cccDNA. The reconstitution of HBV genome and the HBV sequences form patient liver samples were utilized to discover HBV peptides presented on the surface of HCC tumors.

TABLE 3
Information on Sites of Chromosomal HBV Integration in Each Patient

| | Integration | Number of | | |
|---|---|---|---|---|
| Sample ID | Signal | Insertion Sites | Gene with Breakpoints* | Coordinates in Chromosome |
| Liv1_09945T1(3) | Yes | 4 | ZBTB5, SCARB1, ASPH, IL1RAP | chr9: 37459289, chr12: 124861446, |
| | | | | chr8: 61655359, chr3: 190576913 |
| Liv2_0000032532 | No | — | — | — |
| Liv3_0000040312 | Yes | 2 | LINC02315, ADH4 | chr14: 41085340, chr4: 99139827 |
| Liv4_0000031295 | Yes | 1 | ARHGAP10 | chr4: 148057202 |
| Liv5_0000049186 | No | — | — | — |
| Liv6_0000049219 | No | — | — | — |
| Liv7_ILS37316FT2 | No | — | — | — |
| Liv8_ILS30987D2 | Yes | 1 | SENP7 | chr3: 101353476 |
| Liv9_ILS24999D2 | Yes | 9 | SUCO, CRLS1, MIR924HG, | chr1: 172540958, chr20: 6034464, |
| | | | TENT4B, ASIC2, ZNF532, | chr18: 39347751, chr16: 50197960, |
| | | | DMGDH, LINC01572, CTSK | chr17: 33055297, chr18: 58965789, |
| | | | | chr5: 79045221, chr16: 72464716, |
| | | | | chr1: 150795258 |
| Liv10_ILS39940FT1 | No | — | — | — |
| Liv11_ILS31761D1 | Yes | 3 | ADARB2, ADARB2, CEP72 | chr10: 1678796, chr10: 1685665, chr5: 651689 |
| Liv12_ILS22791D2 | Yes | 4 | FN1, KCNMB2, TNRC6C, | chr2: 215433744, chr3: 178641757, |
| | | | CCDC178 | chr17: 78099692, chr18: 33018813 |
| Liv13_ILS23304D2 | No | — | — | — |
| Liv14_ILS20947D3 | No | — | — | — |
| Liv15_ILS21034D4 | Yes | 4 | CCDC178, SLC5A10, DLEU2, TES | chr18: 33070497, chr17: 19020322, |
| | | | | chr13: 50023953, chr7: 116223975 |
| Liv16_ILS42208FT1 | No | — | — | — |
| Liv17_ILS22914D2 | Yes | 2 | MIR548AI, EDIL3 | chr16: 51407105, chr5: 84167194 |
| Liv18_ILS20954D2 | No | — | — | — |
| Liv19_ILS21955D2 | Yes | 1 | GLIS3 | chr9: 4254987 |
| Liv20_ILS10922D04 | Yes | 1 | CBR4 | chr4: 168994461 |
| Liv21_499956PF | No | — | — | — |
| Liv22_524614VF | No | — | — | — |
| Liv23_ILS35989FT2 | No | — | — | — |
| Liv24_ILS42195FT1 | No | — | — | — |
| Liv25_ILS45824FT1 | Yes | 1 | TERT | chr5: 1294669 |
| Liv26_ILS36725FT2 | No | — | — | — |
| Liv27_ILS37352FT1 | Yes | 5 | TERT, OR2W5, STK38, STK38, | chr5: 1295319, chr1: 247491008, |
| | | | SMURF1 | chr6: 36515494, chr6: 36535136, |
| | | | | chr7: 99045160 |
| Liv28_ILS50526FA1 | Yes | 5 | PCCA, NFX1, C5, TYW1, AGBL4 | chr13: 100175575, chr9: 33337882, |
| | | | | chr9: 120969895, chr7: 67038335, |
| | | | | chr1: 48942206 |
| Liv29_ILS50526FT2 | No | — | — | — |
| Liv30_ILS50523FA1 | Yes | 29 | SLC35D2, METTL15, TMEM123, | chr9: 96314404, chr11: 28309909, |
| | | | AFG1L, IGF1R, PID1, NAA25, | chr11: 102406055, chr6: 108374456, |
| | | | ITPR2, SERBP1, SNAP25-AS1, | chr15: 98752500, chr2: 229228844, |
| | | | MASP1, POLR2D, LOC285074, | chr12: 112054336, chr12: 26456046, |
| | | | HEXD, ANKRD30BL, PLEKHH2, | chr1: 67425391, chr20: 10127800, |
| | | | LINC01483, CALN1, GPATCH8, | chr3: 187249181, chr2: 127845635, |
| | | | RANBP17, ADIPOR1, KIF1A, | chr2: 87048545, chr17: 82432780, |
| | | | BPY2, PPIP5K2, C13orf42, GRB10, | chr2: 132185698, chr2: 43643006, |
| | | | OPRM1, CCT7, OR10J1 | chr17: 69700125, chr7: 72133928, |
| | | | | chr17: 44496391, chr5: 170957700, |
| | | | | chr1: 202945951, chr2: 240739315, |
| | | | | chrY: 23002941, chr5: 103157280, |
| | | | | chr13: 51125862, chr7: 50761442, |
| | | | | chr6: 154152272, chr2: 73249661, |
| | | | | chr1: 159419156 |
| Liv31_ILS50523FT2 | Yes | 6 | GTF2IP20, LOC100506990, FUT8, | chr1: 223962074, chr8: 12551594, |
| | | | CERS6, EHBP1, OSBPL5 | chr14: 65549640, chr2: 168503060, |
| | | | | chr2: 63043084, chr11: 3114444 |
| Liv32_1176935F | No | — | — | — |
| Liv33_1176559F | No | — | — | — |
| Liv34_1182970F | No | — | — | — |
| Liv35_1181680F | No | — | — | — |
| Liv36_1182812F | No | — | — | — |
| Liv37_1189452F | Yes | 1 | KLHL14 | chr18: 32730774 |
| Liv38_1193117F | Yes | 8 | SLC25A51, ASAP1, GPC6, CRYL1, | chr9: 37886881, chr8: 130125106, |
| | | | CNTNAP2, CUX1, CDH4, | chr13: 93769564, chr13: 20506360, |
| | | | STARD13 | chr7: 148204370, chr7: 102032820, |
| | | | | chr20: 61450282, chr13: 33190765 |
| Liv39_1192879F | Yes | 1 | NCOA2 | chr8: 70195515 |
| Liv40_405295F2 | No | — | — | — |
| Liv41_ILS32961D2 | No | — | — | — |
| Liv42_ILS33989D2 | No | — | — | — |
| Liv43_ILS22892S1 | Yes | 7 | TK2, CROCC, PDZK1, IKZF2, | chr16: 66522016, chr1: 16926565, |
| | | | MAN2A1, DNAJC22, NME8 | chr1: 145706208, chr2: 213096395, |
| | | | | chr5: 109840521, chr12: 49350794, |
| | | | | chr7: 37871706 |
| Liv44_ILS24966D3- | No | — | — | — |
| DS2 | | | | |
| Liv45_ILS30194D2 | No | — | — | — |
| Liv46_ILS32365D1 | No | — | — | — |
| Liv47_ILS32963D1 | No | — | — | — |
| Liv48_ILS36088FT2 | Yes | 1 | COLQ | chr3: 15491811 |

*Description of gene symbols and gene full names for genes with breakpoints: ADARB2, Adenosine Deaminase RNA Specific B2 (Inactive); ADH4, Alcohol Dehydrogenase 4 (Class II), Pi Polypeptide; ADIPOR1, Adiponectin Receptor 1; AFG1L, AFG1 Like ATPase; AGBL4, AGBL Carboxypeptidase 4; ANKRD30BL, Ankyrin Repeat Domain 30B Like; ARHGAP10, Rho GTPase Activating Protein 10; ASAP1, ArfGAP With SH3 Domain, Ankyrin Repeat And PH Domain 1; ASIC2, Acid Sensing Ion Channel Subunit 2; ASPH, Aspartate Beta-Hydroxylase; BPY2, Basic Charge Y-Linked 2; C13orf42, Chromosome 13 Open Reading Frame 42; C5, Complement C5; CALN1, Calneuron 1; CBR4, Carbonyl Reductase 4; CCDC178, Coiled-Coil Domain Containing 178; CCT7, Chaperonin Containing TCP1 Subunit 7; CDH4, Cadherin 4; CEP72, Centrosomal Protein 72; CERS6, Ceramide Synthase 6; CNTNAP2, Contactin Associated Protein 2; COLQ, Collagen Like Tail Subunit Of Asymmetric Acetylcholinesterase; CRLS1, Cardiolipin Synthase 1; CROCC, Ciliary Rootlet Coiled-Coil, Rootletin; CRYL1, Crystallin Lambda 1; CTSK, Cathepsin K; CUX1, Cut Like Homeobox 1; DLEU2, Deleted In Lymphocytic Leukemia 2; DMGDH, Dimethylglycine Dehydrogenase; DNAJC22, DnaJ Heat Shock Protein Family (Hsp40) Member C22; EDIL3, EGF Like Repeats And Discoidin Domains 3; EHBP1, EH Domain Binding Protein 1; FN1, Fibronectin 1; FUT8, Fucosyltransferase 8; GLIS3, GLIS Family Zinc Finger 3; GPATCH8, G-Patch Domain Containing 8; GPC6, Glypican 6; GRB10, Growth Factor Receptor Bound Protein 10; GTF2IP20, General Transcription Factor IIi Pseudogene 20; HEXD, Hexosaminidase D; IGF1R, Insulin Like Growth Factor 1 Receptor; IKZF2, IKAROS Family Zinc Finger 2; IL1RAP, Interleukin 1 Receptor Accessory Protein; ITPR2, Inositol 1,4,5-Trisphosphate Receptor Type 2; KCNMB2, Potassium Calcium-Activated Channel Subfamily M Regulatory Beta Subunit 2; KIF1A, Kinesin Family Member 1A; KLHL14, Kelch Like Family Member 14; LINC01483, Long Intergenic Non-Protein Coding RNA 1483; LINC01572, Long Intergenic Non-Protein Coding RNA 1572; LINC02315, Long Intergenic Non-Protein Coding RNA 2315; MAN2A1, Mannosidase Alpha Class 2A Member 1; MASP1, MBL Associated Serine Protease 1; METTL15, Methyltransferase Like 15; MIR548AI, MicroRNA 548ai; MIR924HG, MIR924 Host Gene; NAA25, N-Alpha-Acetyltransferase 25, NatB Auxiliary Subunit; NCOA2, Nuclear Receptor Coactivator 2; NFX1, Nuclear Transcription Factor, X-Box Binding 1; NME8, NME/NM23 Family Member 8; OPRM1, Opioid Receptor Mu 1; OR10J1, Olfactory Receptor Family 10 Subfamily J Member 1; OR2W5, Olfactory Receptor Family 2 Subfamily W Member 5 Pseudogene; OSBPL5, Oxysterol Binding Protein Like 5; PCCA, Propionyl-CoA Carboxylase Subunit Alpha; PDZK1, PDZ Domain Containing 1; PID1, Phosphotyrosine Interaction Domain Containing 1; PLEKHH2, Pleckstrin Homology, MyTH4 And FERM Domain Containing H2; POLR2D, RNA Polymerase II Subunit D; PPIP5K2, Diphosphoinositol Pentakisphosphate Kinase 2; RANBP17, RAN Binding Protein 17; SCARB1, Scavenger Receptor Class B Member 1; SENP7, SUMO Specific Peptidase 7; SERBP1, SERPINE1 MRNA Binding Protein 1; SLC25A51, Solute Carrier Family 25 Member 51; SLC35D2, Solute Carrier Family 35 Member D2; SLC5A10, Solute Carrier Family 5 Member 10; SMURF1, SMAD Specific E3 Ubiquitin Protein Ligase 1; SNAP25-AS1, SNAP25 Antisense RNA 1; STARD13, StAR Related Lipid Transfer Domain Containing 13; STK38, Serine/Threonine Kinase 38; SUCO, SUN Domain Containing Ossification Factor; TENT4B, Terminal Nucleotidyltransferase 4B; TERT, Telomerase Reverse Transcriptase; TES, Testin LIM Domain Protein; TK2, Thymidine Kinase 2; TMEM123, Transmembrane Protein 123; TNRC6C, Trinucleotide Repeat Containing Adaptor 6C; TYW1, TRNA-YW Synthesizing Protein 1 Homolog; ZBTB5, Zinc Finger And BTB Domain Containing 5; ZNF532, Zinc Finger Protein 532.

### Example 2. Characterization of the Immunopeptidome of HBV-Infected Patient Samples

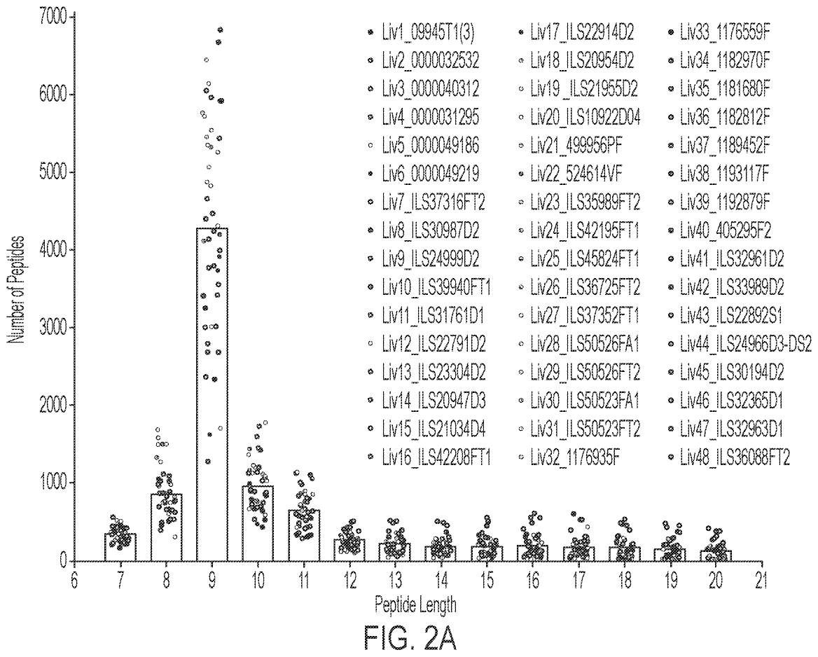

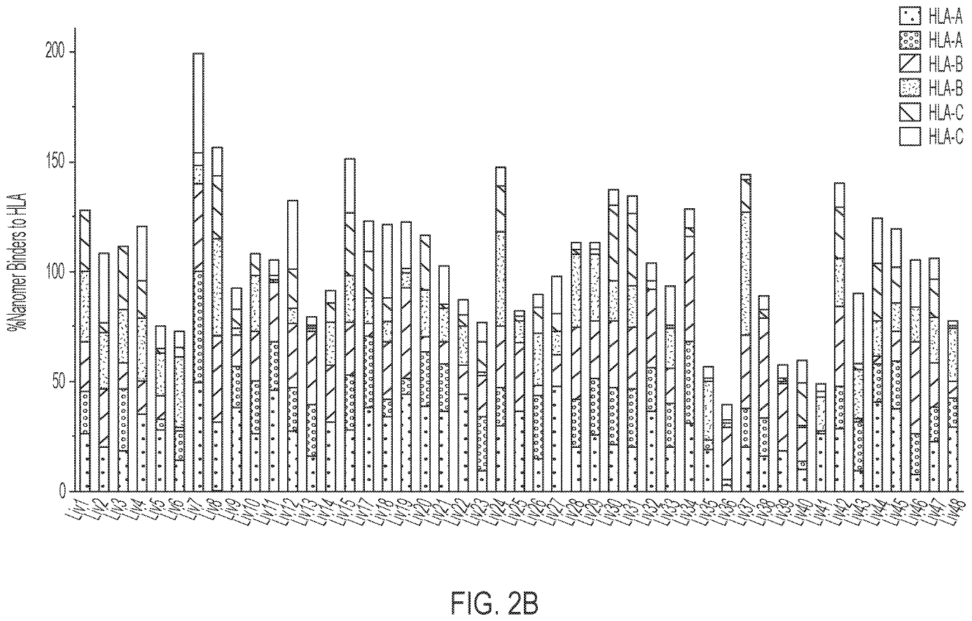

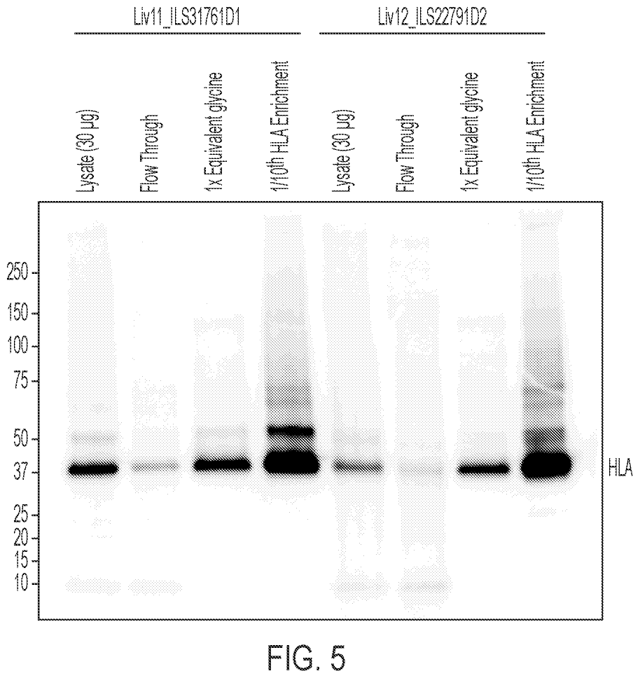

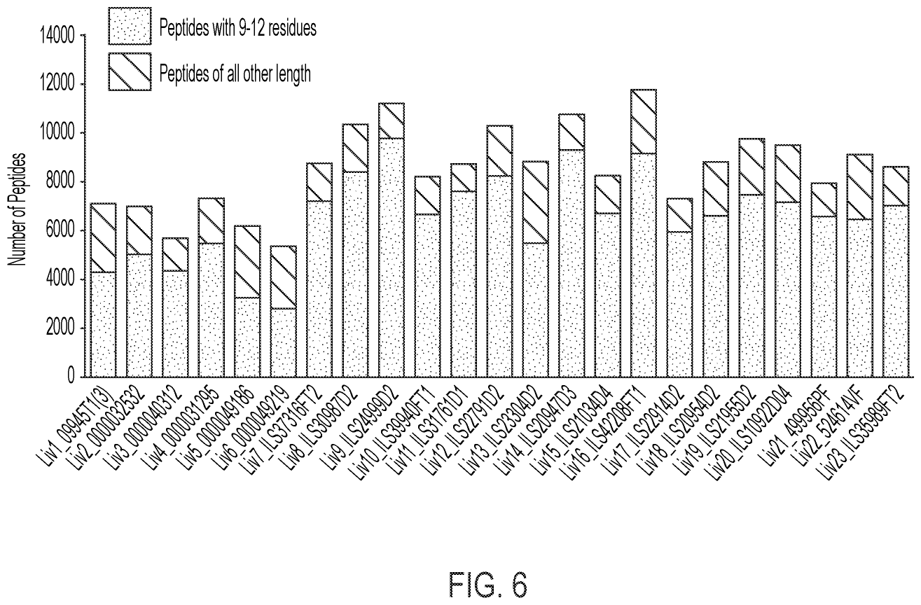

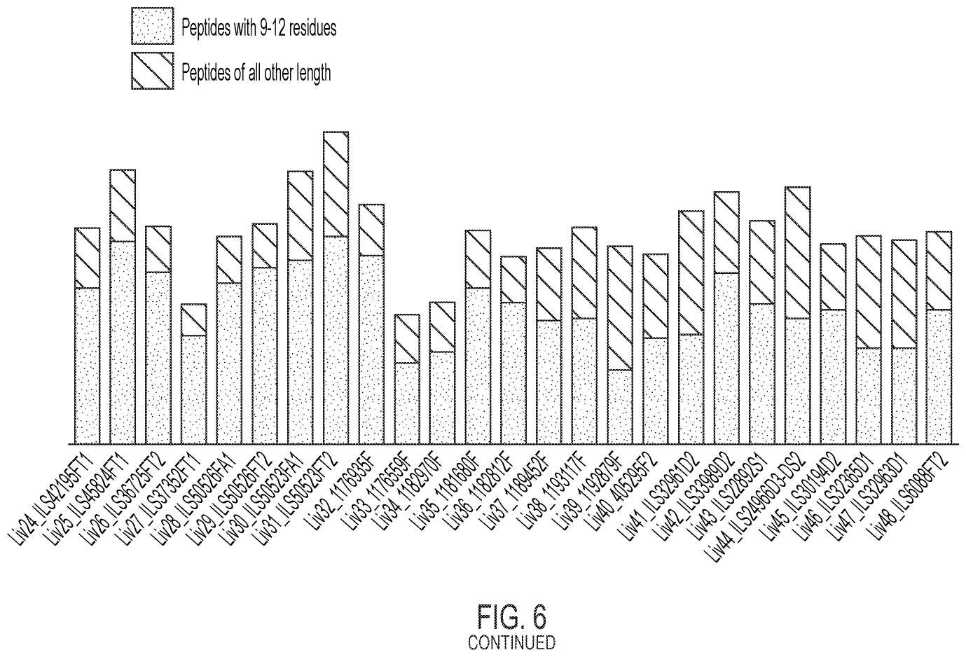

HLA Class-I complexes were immunopurified from homogenized hepatocellular carcinoma (HCC) liver tissue samples from 48 HBV seropositive patients (Proteogenex and BioIVT, Tables 4-5) using an anti-HLA Class-I antibody (W6/32). Specifically, the tissue lysate in NP-40 was incubated with W6/32-conjugated sepharose beads in column format, followed by beads washing and HLA elution in glycine under acidic condition. The peptides and the HLA molecules were further concentrated on a C18 sep-pak, and sequentially eluted by 30% Acetonitrile, 0.1% TFA and 70% Acetonitrile, and 0.1% TFA respectively. The proteins were separated under non-reduced conditions. Only 10% of the 70% acetonitrile elution ( 1/10th of HLA enrichment) was loaded on to the gel. The immunoblot was probed with anti-HLA-I W6/32 and goat-anti-mouse-HRP. Western blotting of liver lysates, flow throughs, and enrichment elution demonstrated a near complete capture of HLA-I by W6/32 linked sepharose beads (FIG. 5). Following elution of the complexes, HLA-I bound peptides were identified using an Orbitrap Fusion Lumos mass spectrometer coupled to a nanoLC system. The raw mass spectrometry (MS) data files were searched against a combined database of human UniProt and HBV sequences derived from the RNA HBV reads from patient samples. On average, approximately 8,500 total peptides were detected from liver samples from variably aged patients (FIG. 6). FIG. 2A depicts peptide length distributions by mass spectrometry for all HBV-positive patient liver tissues by serology. For generation of these data, the raw files were searched with PEAKSX with 5% false discovery rate (FDR) at the peptide level. The distribution of peptide lengths isolated from the majority of liver tissue samples was consistent with those for Class I HLA enrichment, with peptides nine residues in length (9-mers) being the most abundant, followed by peptides ten (10-mers) and eleven (11-mers) residues in length. Thus, a vast majority of 9-mers were detected from the HLA class-I enrichment from a high percent of patient liver samples. Peptides with nine to twelve residues constituted over 50% of the total peptides isolated (FIG. 6).

Table 4 shows the patient sample ID, diagnosis (from vendor), and HBV serological test results (i.e., HBV status, from vendor) for all patient samples analyzed, together with number of HBV unique peptides detected by mass spectrometry (MS), HBV POL₆₀₆₋₆₁₆ variants detected, detection status for peptides STLPETTVVRR (SEQ ID NO: 43), FLLTRTLTI (SEQ ID NO: 3) and SAISSTFSK (SEQ ID NO: 41), and HBV genotype identified from RNAseq results. HCC, Hepatocellular Carcinoma; CCA, Cholangiocarcinoma; QC, Quality Control.

TABLE 4
Patient Sample Data and Number of HBV Unique Peptides

| | | | | # of | | | | | | | |
|---|---|---|---|---|---|---|---|---|---|---|---|
| | | | | HBV | | | | | | | |
| | | | | pep- | | | | | | | |
| | | | HBV | tides | | | | | | | |
| | | | Pep- | de- | | | STLPE | | | | |
| | | | tides | tected | | | TTVV | FLLTRI | SAISST | | |
| | | | by MS | by MS | | | RR | LTI | FSK | | |
| | | | (5% | (5% | | | (SEQ ID | (SEQ ID | (SEQ ID | HBV | |
| | | | FDR, | FDR, | HBV POL₆₀₆₋₆₁₆ | | NO: 43) | NO: 3) | NO: 41) | Geno- | |
| | | HBV | 9-12 | 9-12 | Variants | | (De- | (De- | (De- | type | |
| Sample | | sta- | resi- | resi- | Detected | Se- | tected, | tected, | tected, | from | Virus |
| ID | Diagnosis | tus | dues) | dues) | (+/−) | quence | +/−) | +/−) | +/−) | RNAseq | reads |
| Liv1_0994 | Liver | HBV | Yes | 4 | − | NA | − | − | − | D | 11865 |
| 5T1(3) | cancer | | | | | | | | | | |
| Liv2_0000 | Cirrhosis | HBV | No | 0 | − | NA | − | − | − | low | 13 |
| 032532 | | | | | | | | | | coverage | |
| Liv3_0000 | Cirrhosis | HBV | Yes | 3 | − | NA | − | − | − | A | 5205 |
| 040312 | | | | | | | | | | | |
| Liv4_0000 | Cirrhosis | HBV | No | 0 | − | NA | − | − | − | low | 68 |
| 031295 | | | | | | | | | | coverage | |
| Liv5_0000 | Chronic | HBV | Yes | 12 | − | NA | − | − | − | poor QC | 0 |
| 049186 | inflam- | | | | | | | | | | |
| | mation | | | | | | | | | | |
| Liv6_0000 | Hepatitis | HBV | Yes | 1 | − | NA | − | − | − | poor QC | 0 |
| 049219 | | | | | | | | | | | |
| Liv7_ILS3 | HCC | HBV | No | 0 | − | NA | − | − | − | low | 26 |
| 7316FT2 | | | | | | | | | | coverage | |
| Liv8_ILS3 | HCC | HBV | Yes | 6 | + | GTLPQE | + | − | − | C | 14633 |
| 0987D2 | | | | | | HIVHK | | | | | |
| | | | | | | (SEQ ID | | | | | |
| | | | | | | NO: 13) | | | | | |
| Liv9_ILS2 | HCC | HBV | Yes | 5 | − | NA | − | − | − | B | 9423 |
| 4999D2 | | | | | | | | | | | |
| Liv10_ILS | HCC | HBV | Yes | 4 | + | GSLPQE | + | − | + | poor QC | 0 |
| 39940FT1 | | | | | | HIVQK | | | | | |
| | | | | | | (SEQ ID | | | | | |
| | | | | | | NO: 12) | | | | | |
| Liv11_ILS | HCC | HBV | Yes | 3 | + | GTLPQE | − | − | − | C | 9935 |
| 31761D1 | | | | | | HIVQK | | | | | |
| | | | | | | (SEQ ID | | | | | |
| | | | | | | NO: 14) | | | | | |
| Liv12_ILS | HCC | HBV | Yes | 7 | + | GTLPQE | + | + | − | C | 26154 |
| 22791D2 | | | | | | HIVQK | | | | | |
| | | | | | | (SEQ ID | | | | | |
| | | | | | | NO: 14) | | | | | |
| Liv13_ILS | HCC | HBV | No | 0 | − | NA | − | − | − | low | 31 |
| 23304D2 | | | | | | | | | | coverage | |
| Liv14_ILS | HCC | HBV | Yes | 2 | + | GTLPQE | + | − | − | C | 2070 |
| 20947D3 | | | | | | HIVHK | | | | | |
| | | | | | | (SEQ ID | | | | | |
| | | | | | | NO: 13) | | | | | |
| Liv15_ILS | HCC | HBV | Yes | 4 | + | GSLPQE | − | − | − | B | 5369 |
| 21034D4 | | | | | | HIVQK | | | | | |
| | | | | | | (SEQ ID | | | | | |
| | | | | | | NO: 12) | | | | | |
| Liv16_ILS | HCC | HBV | Yes | 4 | − | NA | − | − | − | poor QC | 0 |
| 42208FT1 | | | | | | | | | | | |
| Liv17_ILS | HCC | HBV | Yes | 1 | + | GSLPQE | − | − | − | B | 12853 |
| 22914D2 | | | | | | HIVQK | | | | | |
| | | | | | | (SEQ ID | | | | | |
| | | | | | | NO: 12) | | | | | |
| Liv18_ILS | HCC | HBV | No | 0 | − | NA | − | − | − | low | 23 |
| 20954D2 | | | | | | | | | | coverage | |
| Liv19_ILS | HCC | HBV | Yes | 1 | − | NA | − | − | − | C | 3909 |
| 21955D2 | | | | | | | | | | | |
| Liv20_ILS | HCC | HBV | Yes | 11 | − | NA | − | − | − | B | 26200 |
| 10922D04 | | | | | | | | | | | |
| Liv21_499 | HCC | HBV | Yes | 2 | + | GSLPQE | − | − | + | B | 9010 |
| 956PF | | | | | | HIVQK | | | | | |
| | | | | | | (SEQ ID | | | | | |
| | | | | | | NO: 12) | | | | | |
| Liv22_524 | HCC | HBV | No | 0 | − | NA | − | − | − | low | 30 |
| 614VF | | | | | | | | | | coverage | |
| Liv23_ILS | HCC | HBV | No | 0 | − | NA | − | − | − | low | 4 |
| 35989FT2 | | | | | | | | | | coverage | |
| Liv24_ILS | HCC | HBV | No | 0 | − | NA | − | − | − | low | 6 |
| 42195FT1 | | | | | | | | | | coverage | |
| Liv25_ILS | HCC | HBV | Yes | 4 | − | NA | − | − | − | B | 43486 |
| 45824FT1 | | | | | | | | | | | |
| Liv26_ILS | HCC | HBV | No | 0 | − | NA | − | − | − | low | 6 |
| 36725FT2 | | | | | | | | | | coverage | |
| Liv27_ILS | HCC | HBV | Yes | 2 | + | GSLPQE | − | − | + | B | 15594 |
| 37352FT1 | | | | | | HIVQK | | | | | |
| | | | | | | (SEQ ID | | | | | |
| | | | | | | NO: 12) | | | | | |
| Liv28_ILS | Hepatitis, | HBV | Yes | 4 | + | GTLPQE | + | − | − | C | 22294 |
| 50526FA1 | chronic | | | | | HIVHK | | | | | |
| | (Di- | | | | | (SEQ ID | | | | | |
| | seased) | | | | | NO: 13) | | | | | |
| Liv29_ILS | HCC | HBV | No | 0 | − | NA | − | − | − | low | 30 |
| 50526FT2 | | | | | | | | | | coverage | |
| Liv30_ILS | Cirrhosis | HBV | Yes | 6 | − | NA | − | − | + | B | 18692 |
| 50523FA1 | (Di- | | | | | | | | | | |
| | seased) | | | | | | | | | | |
| Liv31_ILS | HCC | HBV | Yes | 5 | − | NA | − | − | + | B | 14716 |
| 50523FT2 | | | | | | | | | | | |
| Liv32_117 | HCC | HBV | No | 0 | − | NA | − | − | − | low | 1 |
| 6935F | | | | | | | | | | coverage | |
| Liv33_117 | HCC | HBV | No | 0 | − | NA | − | − | − | low | 18 |
| 6559F | | | | | | | | | | coverage | |
| Liv34_118 | HCC | HBV | No | 0 | − | NA | − | − | − | low | 88 |
| 2970F | | | | | | | | | | coverage | |
| Liv35_118 | HCC | HBV | Yes | 4 | + | GSLPQE | − | − | − | B | 9563 |
| 1680F | | | | | | HIVQK | | | | | |
| | | | | | | (SEQ ID | | | | | |
| | | | | | | NO: 12) | | | | | |
| Liv36_118 | HCC | HBV | No | 0 | − | NA | − | − | − | low | 990 |
| 2812F | | | | | | | | | | coverage | |
| Liv37_118 | HCC | HBV | Yes | 2 | − | NA | − | − | − | B | 1468 |
| 9452F | | | | | | | | | | | |
| Liv38_119 | HCC | HBV | Yes | 9 | + | GSLPQE | + | − | − | B | 57394 |
| 3117F | | | | | | HIIQK | | | | | |
| | | | | | | (SEQ ID | | | | | |
| | | | | | | NO: 11) | | | | | |
| Liv39_119 | HCC | HBV | No | 0 | − | NA | − | − | − | low | 25 |
| 2879F | | | | | | | | | | coverage | |
| Liv40_405 | HCC | HBV | Yes | 1 | − | NA | − | − | − | C | 52627 |
| 295F2 | | | | | | | | | | | |
| Liv41_ILS | HCC | HBV | No | 0 | − | NA | − | − | − | low | 1 |
| 32961D2 | | | | | | | | | | coverage | |
| Liv42_ILS | HCC and | HBV | No | 0 | − | NA | − | − | − | low | 17 |
| 33989D2 | CCA | | | | | | | | | coverage | |
| Liv43_ILS | HCC | HBV | Yes | 2 | − | NA | − | − | − | C | 9174 |
| 22892S1 | | | | | | | | | | | |
| Liv44_ILS | HCC and | HBV | Yes | 3 | − | NA | − | − | − | C | 2980 |
| 24966D3- | CCA | | | | | | | | | | |
| DS2 | | | | | | | | | | | |
| Liv45_ILS | HCC | HBV | Yes | 3 | − | NA | − | + | − | C | 4486 |
| 30194D2 | | | | | | | | | | | |
| Liv46_ILS | HCC | HBV | No | 0 | − | NA | − | − | − | low | 39 |
| 32365D1 | | | | | | | | | | coverage | |
| Liv47_ILS | HCC | HBV | Yes | 1 | + | GTLPQE | − | − | − | C | 675 |
| 32963D1 | | | | | | HIVHK | | | | | |
| | | | | | | (SEQ ID | | | | | |
| | | | | | | NO: 13) | | | | | |
| Liv48_ILS | HCC | HBV | Yes | 1 | + | GTLPQE | − | − | − | C | 16030 |
| 36088FT2 | | | | | | HIVHK | | | | | |
| | | | | | | (SEQ ID | | | | | |
| | | | | | | NO: 13) | | | | | |

Table 5A and Table 5B display patient and sample information provided from the vendor. All samples were flash-frozen (FF) and the specimen type for all samples was liver. The HBV diagnosis corresponding to all samples was HBV positive. A single sample (Sample ID: ILS24966D3-DS2), was obtained from a patient of menopausal status. The vendor used for all samples was BioIVT, except for the first 6 samples (Sample IDs: 09945T1(3), 0000032532, 0000040312, 0000031295, 0000049186, and 0000049219), where Proteogenex was used as the vendor. The country (Ctry) column indicates the country of sample collection site. Smoking years and alcohol years refers to the duration of smoking and alcohol use, respectively. M, Male; F, Female; API, Asian Pacific Islander; Dx, diagnosis; Histo, Histological; Grp, Group; Wt, weight; Ht, Height; Cigs, Cigarettes; Ctry, Country; VN, Vietnam; n/a, not applicable.

TABLE 5A
Sample and Patient Information

| | | | Ethnic | | Histo | Tumor | TNM | Stage | Wt |
|---|---|---|---|---|---|---|---|---|---|
| Sample ID | Sex | Age | Grp | Dx | Dx | Grade | Stage | Grp | (g) |
| 09945T1(3) | M | 74 | Caucasian | | Liver cancer | | | | 0.70 |
| 0000032532 | M | 66 | Hispanic | | Cirrhosis | | | | 0.91 |
| | | | or Latino | | | | | | |
| 0000040312 | M | 37 | Not | | Cirrhosis | | | | 0.52 |
| | | | Hispanic | | | | | | |
| | | | or Latino | | | | | | |
| 0000031295 | M | 64 | Not | | Cirrhosis | | | | 0.57 |
| | | | Hispanic | | | | | | |
| | | | or Latino | | | | | | |
| 0000049186 | F | 65 | Not | | Chronic | | | | 1.10 |
| | | | Hispanic | | inflammation | | | | |
| | | | or Latino | | | | | | |
| 0000049219 | M | 32 | Not | | Hepatitis | | | | 0.97 |
| | | | Hispanic | | | | | | |
| | | | or Latino | | | | | | |
| ILS37316FT2 | M | 42 | API | Cancer | HCC | | T2N0M0 | II | 3.50 |
| ILS30987D2 | F | | API | Cancer | HCC | | T3N0M0 | IIIA | 3.10 |
| ILS24999D2 | M | | API | Cancer | HCC | | | N/A | 3.00 |
| ILS39940FT1 | M | 47 | API | Cancer | HCC | | T2N0M0 | II | 2.70 |
| ILS31761D1 | M | 57 | API | Cancer | HCC | | T2N0M0 | II | 2.60 |
| ILS22791D2 | M | | API | Cancer | HCC | | | N/A | 2.60 |
| ILS23304D2 | F | | API | Cancer | HCC | | | N/A | 2.60 |
| ILS20947D3 | M | | API | Cancer | HCC | | | | 2.60 |
| ILS21034D4 | M | | API | Cancer | HCC | | | | 2.50 |
| ILS42208FT1 | M | 52 | API | Cancer | HCC | | T2N0M0 | II | 2.50 |
| ILS22914D2 | M | | API | Cancer | HCC | | | N/A | 2.40 |
| ILS20954D2 | F | | API | Cancer | HCC | | | N/A | 2.30 |
| ILS21955D2 | M | | API | Cancer | HCC | | | N/A | 2.30 |
| ILS10922D04 | M | | API | Cancer | HCC | | T3N0Mx | IIIA | 2.20 |
| 499956PF | M | 45 | Asian | Cancer | HCC | Poorly | T3bNXM0 | IIIB | 1.32 |
| | | | | | | differ- | | | |
| | | | | | | entiated | | | |
| 524614VF | M | 54 | Asian | Cancer | HCC | Moderately | T1NXM0 | I | 2.83 |
| | | | | | | differ- | | | |
| | | | | | | entiated | | | |
| ILS35989FT2 | M | 54 | Asian | Cancer | HCC | Moderately | T1bNXM0 | IB | 1.97 |
| | | | | | | differ- | | | |
| | | | | | | entiated | | | |
| ILS42195FT1 | M | 35 | Asian | Cancer | HCC | Moderately | T3NXM0 | IIIA | 2.09 |
| | | | | | | differ- | | | |
| | | | | | | entiated | | | |
| ILS45824FT1 | M | 55 | Asian | Cancer | HCC | Moderately | T1bNXM0 | IB | 2.02 |
| | | | | | | differ- | | | |
| | | | | | | entiated | | | |
| ILS36725FT2 | M | 54 | Asian | Cancer | HCC | | T1bNXM0 | IB | 1.61 |
| ILS37352FT1 | M | 43 | Asian | Cancer | HCC | Moderately | T2NXM0 | II | 0.34 |
| | | | | | | differ- | | | |
| | | | | | | entiated | | | |
| ILS50526FA1 | M | 55 | Asian | Diseased | Hepatitis, | | T1bNXM0 | IB | 0.41 |
| | | | | | chronic | | | | |
| ILS50526FT2 | M | 55 | Asian | Cancer | HCC | Poorly | T1bNXM0 | IB | 1.86 |
| | | | | | | differ- | | | |
| | | | | | | entiated | | | |
| ILS50523FA1 | M | 29 | Asian | Diseased | Cirrhosis | | T2NXM0 | II | 1.07 |
| ILS50523FT2 | M | 29 | Asian | Cancer | HCC | Moderately | T2NXM0 | II | 1.29 |
| | | | | | | differ- | | | |
| | | | | | | entiated | | | |
| 1176935F | M | 57 | Asian | Tumor | HCC | Moderately | T1NXM0 | I | 1.39 |
| | | | | | | differ- | | | |
| | | | | | | entiated | | | |
| 1176559F | M | 51 | Asian | Tumor | HCC | Moderately | T2NXM0 | II | 2.39 |
| | | | | | | differ- | | | |
| | | | | | | entiated | | | |
| 1182970F | M | 52 | Asian | Tumor | HCC | Moderately | T1NXM0 | I | 1.25 |
| | | | | | | differ- | | | |
| | | | | | | entiated | | | |
| 1181680F | M | 62 | Asian | Tumor | HCC | Moderately | T1NXM0 | I | 1.39 |
| | | | | | | differ- | | | |
| | | | | | | entiated | | | |
| 1182812F | M | 44 | Asian | Tumor | HCC | High grade | T3aN0M0 | IIIA | 0.98 |
| 1189452F | M | 46 | Asian | Tumor | HCC | Moderately | T2N0M0 | II | 1.41 |
| | | | | | | differ- | | | |
| | | | | | | entiated | | | |
| 1193117F | M | 45 | Asian | Tumor | HCC | Poorly | T2N0M0 | II | 0.96 |
| | | | | | | differ- | | | |
| | | | | | | entiated | | | |
| 1192879F | M | 49 | Asian | Tumor | HCC | Moderately | T3aN0M0 | IIIA | 1.58 |
| | | | | | | differ- | | | |
| | | | | | | entiated | | | |
| 405295F2 | M | 48 | Asian | Tumor | HCC | Moderately | T3aNXM0 | IIIA | 3.33 |
| | | | | | | differ- | | | |
| | | | | | | entiated | | | |
| ILS32961D2 | M | 46 | Asian | Tumor | HCC | Moderately | T1bNXM0 | IB | 1.23 |
| | | | | | | differ- | | | |
| | | | | | | entiated | | | |
| ILS33989D2 | M | 57 | Asian | Tumor | HCC | Moderately | T1bNXM0 | IB | 0.85 |
| | | | | | | differ- | | | |
| | | | | | | entiated | | | |
| ILS22892S1 | M | 50 | Asian | Diseased | Inflammation, | | TXNXM0 | UNK | 1.35 |
| | | | | | chronic | | | | |
| ILS24966D3-DS2 | F | 75 | Asian | Tumor | HCC | Well to | T3NXM0 | IIIA | 0.61 |
| | | | | | | moderately | | | |
| | | | | | | differ- | | | |
| | | | | | | entiated | | | |
| ILS30194D2 | M | 50 | Asian | Unknown | | | T3NXM0 | IIIA | 1.08 |
| ILS32365D1 | M | 54 | Asian | Unknown | | | T2NXM0 | II | 0.46 |
| ILS32963D1 | M | 31 | Asian | Tumor | HCC | Moderately | T2NXM0 | II | 1.60 |
| | | | | | | differ- | | | |
| | | | | | | entiated | | | |
| ILS36088FT2 | M | 50 | Asian | Tumor | HCC | Well to | T2NXM0 | II | 0.76 |
| | | | | | | moderately | | | |
| | | | | | | differ- | | | |
| | | | | | | entiated | | | |

TABLE 5B
Additional Sample and Patient Information

| | | | | | | Cigs | | | Drinks | | Clinical | Re- | |
|---|---|---|---|---|---|---|---|---|---|---|---|---|---|
| | | Ht | Wt | | Smoking | per | Smoking | Alcohol | per | Alcohol | Dx | covery | Cate- |
| Sample ID | Ctry | (cm) | (kg) | BMI | Status | Day | Years | Status | Day | Years | (Liver) | Type | gory |
| 499956PF | VN | | | | | | | | | | | | |
| 524614VF | VN | | | | | | | | | | | | |
| ILS35989FT2 | Not | | | | | | | | | | | | |
| | dis- | | | | | | | | | | | | |
| | closed | | | | | | | | | | | | |
| ILS42195FT1 | Not | | | | | | | | | | | | |
| | dis- | | | | | | | | | | | | |
| | closed | | | | | | | | | | | | |
| ILS45824FT1 | Not | | | | | | | | | | | | |
| | dis- | | | | | | | | | | | | |
| | closed | | | | | | | | | | | | |
| ILS36725FT2 | Not | | | | | | | | | | | | |
| | dis- | | | | | | | | | | | | |
| | closed | | | | | | | | | | | | |
| ILS37352FT1 | Not | | | | | | | | | | | | |
| | dis- | | | | | | | | | | | | |
| | closed | | | | | | | | | | | | |
| ILS50526FA1 | VN | | | | | | | | | | | | |
| ILS50526FT2 | VN | | | | | | | | | | | | |
| ILS50523FA1 | VN | | | | | | | | | | | | |
| ILS50523FT2 | VN | | | | | | | | | | | | |
| 1176935F | VN | 169 | 53 | 18.56 | Current | 10 | 20 | Occa- | 1 | 20 | HCC | Surgical | C |
| | | | | | | | | sional | | | | | |
| 1176559F | VN | 161 | 51 | 19.68 | Never | | | Previous | 1 | 20 | HCC | Surgical | C |
| 1182970F | VN | 160 | 52 | 20.31 | Never | | | Previous | 1 | 20 | HCC | Surgical | C |
| 1181680F | VN | 163 | 50 | 18.82 | Never | | | Previous | 1 | 15 | HCC | Surgical | C |
| 1182812F | VN | 164 | 63 | 23.42 | Current | 10 | 20 | Current | 1 | 20 | HCC | Surgical | C |
| 1189452F | VN | 167 | 53 | 19 | Never | | | Occa- | 1 | 15 | HCC | Surgical | C |
| | | | | | | | | sional | | | | | |
| 1193117F | VN | 165 | 55 | 20.2 | Never | | | Current | 1 | 10 | HCC | Surgical | C |
| 1192879F | VN | 168 | 60 | 21.26 | Never | | | Occa- | 1 | 20 | HCC | Surgical | C |
| | | | | | | | | sional | | | | | |
| 405295F2 | VN | 165 | 56 | 20.57 | Occa- | 5 | 20 | Occa- | 1 | 20 | HCC | Surgical | C |
| | | | | | sional | | | sional | | | | | |
| ILS32961D2 | Not | 160 | 45 | 17.578125 | Current | 4 | | Current | 0 | | HCC | Surgical | C |
| | dis- | | | | | | | | | | | Archive | |
| | closed | | | | | | | | | | | | |
| ILS33989D2 | Not | 160 | 50 | 19.53 | Current | 10 | 20 | Current | | 10 | HCC and | Surgical | C |
| | dis- | | | | | | | | | | CCA, | Archive | |
| | closed | | | | | | | | | | combined | | |
| ILS22892S1 | Not | | | | Never | | | Never | | | HCC | Surgical | C |
| | dis- | | | | | | | | | | | Archive | |
| | closed | | | | | | | | | | | | |
| ILS24966D3-DS2 | Not | 155 | 48 | 19.98 | Never | | | Never | | | HCC and | Surgical | C |
| | dis- | | | | | | | | | | CCA, | Archive | |
| | closed | | | | | | | | | | combined | | |
| ILS30194D2 | Not | 168 | 68 | 24.09 | Current | | n/a | Current | | | HCC | Surgical | E |
| | dis- | | | | | | | | | | | Archive | |
| | closed | | | | | | | | | | | | |
| ILS32365D1 | Not | 165 | 52 | 19.1 | Never | | | Current | | 20 | HCC | Surgical | E |
| | dis- | | | | | | | | | | | Archive | |
| | closed | | | | | | | | | | | | |
| ILS32963D1 | Not | 169 | 55 | 19.26 | Current | | 10 | Never | | | HCC | Surgical | C |
| | dis- | | | | | | | | | | | Archive | |
| | closed | | | | | | | | | | | | |
| ILS36088FT2 | Not | 168 | 59 | 20.9 | Current | 2 | 25 | Current | | 25 | HCC | Surgical | C |
| | dis- | | | | | | | | | | | Archive | |
| | closed | | | | | | | | | | | | |

### Example 3. High Percentage of Identified Peptides Predicted to Bind HLA Using NetMHC Pan 4.0 Analysis

The polymorphic nature of HLA-I has a profound effect on the epitopes presented on the tissue surface. Hence, DNA sequencing of liver tissue samples was performed to determine the HLA genotype (Table 6), which was used to predict the binding of detected peptides by NetMHC pan 4.0.

TABLE 6
HLA class I alleles identified for all HBV-infected patient samples from DNA sequencing.

| | HLA-A | | | | HLA-B | | | | HLA-C | | | |
|---|---|---|---|---|---|---|---|---|---|---|---|---|
| Sample ID | Allele 1 | | Allele 2 | | Allele 1 | | Allele 2 | | Allele 1 | | Allele 2 | |
| Liv1_09945T1(3) | *02 | 01 | *24 | 02 | *38 | 01 | *39 | 01 | *12 | 03 | *12 | 03 |
| Liv2_0000032532 | *03 | 01 | *03 | 01 | *15 | 16 | *53 | 01 | *04 | 01 | *14 | 02 |
| Liv3_0000040312 | *11 | 01 | *24 | 02 | *27 | 06 | *38 | 02 | *07 | 02 | *07 | 02 |
| Liv4_0000031295 | *24 | 02 | *24 | 02 | *13 | 01 | *40 | 01 | *03 | 04 | *07 | 02 |
| Liv5_0000049186 | *02 | 01 | *03 | 01 | *07 | 02 | *38 | 01 | *04 | 01 | *07 | 02 |
| Liv6_0000049219 | *11 | 01 | *30 | 01 | *13 | 02 | *40 | 01 | *06 | 02 | *07 | 02 |
| Liv7_ILS37316FT2 | *02 | 03 | *02 | 06 | *15 | 25 | *55 | 02 | *07 | 02 | *12 | 03 |
| Liv8_ILS30987D2 | *11 | 01 | *11 | 01 | *15 | 02 | *35 | 01 | *03 | 03 | *08 | 01 |
| Liv9_ILS24999D2 | *02 | 03 | *33 | 03 | *38 | 02 | *48 | 03 | *07 | 02 | *08 | 01 |
| Liv10_ILS39940FT1 | *11 | 01 | *30 | 01 | *07 | 02 | *38 | 02 | *07 | 02 | *07 | 02 |
| Liv11_ILS31761D1 | *02 | 03 | *11 | 01 | *15 | 02 | *46 | 01 | *01 | 02 | *08 | 01 |
| Liv12_ILS22791D2 | *02 | 01 | *11 | 01 | *39 | 01 | *51 | 01 | *07 | 02 | *14 | 02 |
| Liv13_ILS23304D2 | *11 | 01 | *24 | 02 | *35 | 05 | *46 | 01 | *01 | 02 | *04 | 01 |
| Liv14_ILS20947D3 | *11 | 01 | *11 | 01 | *15 | 02 | *38 | 02 | *07 | 02 | *08 | 01 |
| Liv15_ILS21034D4 | *11 | 01 | *33 | 03 | *15 | 02 | *58 | 01 | *03 | 02 | *14 | 02 |
| Liv16_ILS42208FT1 | HLA genotype could not be identified | | | | | | | | | | | |
| Liv17_ILS22914D2 | *11 | 01 | *31 | 01 | *13 | 01 | *51 | 02 | *03 | 04 | *15 | 02 |
| Liv18_ILS20954D2 | *02 | 01 | *74 | 02 | *15 | 02 | *51 | 01 | *08 | 01 | *14 | 02 |
| Liv19_ILS21955D2 | *02 | 03 | *31 | 01 | *15 | 25 | *51 | 01 | *04 | 03 | *16 | 02 |
| Liv20_ILS10922D04 | *02 | 264 | *33 | 03 | *18 | 02 | *58 | 01 | *03 | 02 | *07 | 04 |
| Liv21_499956PF | *02 | 1 | *11 | 1 | *13 | 1 | *55 | 2 | *01 | 2 | *03 | 4 |
| Liv22_524614VF | *11 | 1 | *11 | 1 | *18 | 2 | *38 | 2 | *04 | 3 | *07 | 2 |
| Liv23_ILS35989FT2 | *02 | 7 | *11 | 1 | *38 | 2 | *46 | 1 | *01 | 12 | *07 | 2 |
| Liv24_ILS42195FT1 | *11 | 1 | *29 | 1 | *07 | 5 | *35 | 1 | *03 | 3 | *15 | 5 |
| Liv25_ILS45824FT1 | *24 | 2 | *24 | 2 | *40 | 1 | *54 | 1 | *01 | 2 | *04 | 3 |
| Liv26_ILS36725FT2 | *01 | 1 | *33 | 3 | *13 | 1 | *57 | 1 | *03 | 4 | *06 | 2 |
| Liv27_ILS37352FT1 | *11 | 1 | *11 | 1 | *38 | 2 | *51 | 1 | *07 | 2 | *14 | 2 |
| Liv28_ILS50526FA1 | *11 | 1 | *33 | 3 | *15 | 25 | *35 | 5 | *04 | 1 | *04 | 3 |
| Liv29_ILS50526FT2 | *11 | 1 | *33 | 3 | *15 | 25 | *35 | 5 | *04 | 1 | *04 | 3 |
| Liv30_ILS50523FA1 | *11 | | *33 | 3 | *15 | 2 | *58 | 1 | *03 | 2 | *08 | 1 |
| Liv31_ILS50523FT2 | *11 | | *33 | 3 | *15 | 2 | *58 | 1 | *03 | 2 | *08 | 1 |
| Liv32_1176935F | *02 | 6 | *11 | 1 | *15 | 2 | *15 | 2 | *07 | 2 | *08 | 1 |
| Liv33_1176559F | *24 | 2 | *26 | 1 | *38 | 2 | *54 | 1 | *01 | 2 | *07 | 2 |
| Liv34_1182970F | *11 | 1 | *29 | 1 | *15 | 2 | *15 | 2 | *04 | 3 | *08 | 1 |
| Liv35_1181680F | *11 | 1 | *33 | 3 | *44 | 3 | *56 | 4 | *07 | 6 | *08 | 1 |
| Liv36_1182812F | *11 | 1 | *26 | 1 | *44 | 3 | *56 | 4 | *07 | 6 | *08 | 1 |
| Liv37_1189452F | *11 | 1 | *24 | 2 | *15 | 12 | *15 | 25 | *03 | 3 | *04 | 3 |
| Liv38_1193117F | *11 | 1 | *24 | 3 | *15 | 2 | *15 | 2 | *04 | 3 | *08 | 1 |
| Liv39_1192879F | *11 | 1 | *11 | 1 | *15 | 2 | *46 | 1 | *01 | 2 | *08 | 1 |
| Liv40_405295F2 | *02 | 7 | *02 | 65 | *38 | 2 | *46 | 1 | *01 | 12 | *07 | 2 |
| Liv41_ILS32961D2 | *01 | 1 | *02 | 7 | *46 | 1 | *57 | 1 | *01 | 2 | *06 | 2 |
| Liv42_ILS33989D2 | *02 | 1 | *24 | 2 | *15 | 2 | *15 | 12 | *03 | 3 | *08 | 1 |
| Liv43_ILS22892S1 | *02 | 7 | *33 | 3 | *46 | 1 | *58 | 1 | *01 | 2 | *03 | 2 |
| Liv44_ILS24966D3-DS2 | *02 | 3 | *33 | 3 | *13 | 1 | *58 | 1 | *03 | 2 | *03 | 4 |
| Liv45_ILS30194D2 | *02 | 3 | *24 | 2 | *15 | 12 | *40 | 1 | *03 | 3 | *07 | 2 |
| Liv46_ILS32365D1 | *02 | 7 | *24 | 3 | *15 | 1 | *15 | 12 | *01 | 3 | *03 | 3 |
| Liv47_ILS32963D1 | *11 | 1 | *31 | 1 | *51 | 1 | *51 | 2 | *14 | 2 | *15 | 2 |
| Liv48_ILS36088FT2 | *02 | 3 | *11 | 1 | *15 | 21 | *56 | 4 | *01 | 2 | *04 | 3 |

A high percentage (50-100% for most of the samples) of the detected 9-mer peptides were predicted binders of the identified HLA-I in the corresponding patient samples (FIG. 2B and Table 7). In particular, FIG. 2B depicts a plot showing the percent (0) binders to HLA-A, HLA-B and HLA-C alleles from 9-mer peptides identified by mass spectrometry. The HLA alleles were identified from DNA sequencing of each patient sample. A vast majority of detected nanomer peptides by mass spectrometry were predicted binders to the patient HLA alleles predicted by NetMHC pan 4.0. Table 7 shows the number of nanomers (peptides with 9 residue length, 9-mers) and their predicted percent binding to the respective Patient alleles by NetMHC Pan4.0. Strong and weak binders are defined by their rank by NetMHC prediction, with rank below 0.5 considered as strong binders and between 0.5-2 considered as weak binders. The combined percent binders to the HLA-A, LA-B and HLA-C alleles was over 100% for some of the samples.

TABLE 7
Number of nanomers and their predicted precent (%) binding
to the respective patient alleles by NetMHC Pan4.0.

| | | | Total | | | |
|---|---|---|---|---|---|---|
| | | 9- | Predicted | Strong | Weak | Percent |
| Sample ID | HLA Allele | mers | Binders | Binders | Binders | Binders |
| Liv1_09945T1 | HLA-A02:01 | 3238 | 841 | 675 | 166 | 26 |
| (3) | HLA-A24:02 | 3238 | 624 | 322 | 302 | 19 |
| | HLA-B38:01 | 3238 | 751 | 57 | 694 | 23 |
| | HLA-B39:01 | 3238 | 1045 | 414 | 631 | 32 |
| | HLA-C12:03 | 3238 | 907 | 319 | 588 | 28 |
| Liv2_0000032532 | HLA-A03:01 | 3396 | 665 | 379 | 286 | 20 |
| | HLA-B15:16 | 3396 | 869 | 267 | 602 | 26 |
| | HLA-B53:01 | 3396 | 869 | 396 | 473 | 26 |
| | HLA-C04:01 | 3396 | 144 | 12 | 132 | 4 |
| | HLA-C14:02 | 3396 | 1092 | 464 | 628 | 32 |
| Liv3_0000040312 | HLA-A11:01 | 2986 | 526 | 448 | 78 | 18 |
| | HLA-A24:02 | 2986 | 830 | 494 | 336 | 28 |
| | HLA-B27:06 | 2986 | 358 | 199 | 159 | 12 |
| | HLA-B38:02 | 2986 | 714 | 68 | 646 | 24 |
| | HLA-C07:02 | 2986 | 875 | 115 | 760 | 29 |
| Liv4_0000031295 | HLA-A24:02 | 3716 | 1314 | 688 | 626 | 35 |
| | HLA-B13:01 | 3716 | 541 | 12 | 529 | 15 |
| | HLA-B40:01 | 3716 | 1081 | 745 | 336 | 29 |
| | HLA-C03:04 | 3716 | 579 | 381 | 198 | 16 |
| | HLA-C07:02 | 3716 | 914 | 112 | 802 | 25 |
| Liv5_0000049186 | HLA-A02:01 | 1700 | 473 | 385 | 88 | 28 |
| | HLA-A03:01 | 1700 | 60 | 39 | 21 | 4 |
| | HLA-B07:02 | 1700 | 192 | 159 | 33 | 11 |
| | HLA-B38:01 | 1700 | 322 | 46 | 276 | 19 |
| | HLA-C04:01 | 1700 | 46 | 4 | 42 | 3 |
| | HLA-C07:02 | 1700 | 174 | 15 | 159 | 10 |
| Liv6_0000049219 | HLA-A11:01 | 1622 | 234 | 197 | 37 | 14 |
| | HLA-A30:01 | 1622 | 214 | 56 | 158 | 13 |
| | HLA-B13:02 | 1622 | 37 | 0 | 37 | 2 |
| | HLA-B40:01 | 1622 | 526 | 383 | 143 | 32 |
| | HLA-C06:02 | 1622 | 65 | 21 | 44 | 4 |
| | HLA-C07:02 | 1622 | 106 | 21 | 85 | 7 |
| Liv7_ILS37316FT2 | HLA-A02:03 | 5922 | 2894 | 2008 | 886 | 49 |
| | HLA-A02:06 | 5922 | 3007 | 1711 | 1296 | 51 |
| | HLA-B15:25 | 5922 | 2395 | 1315 | 1080 | 40 |
| | HLA-B55:02 | 5922 | 498 | 191 | 307 | 8 |
| | HLA-C07:02 | 5922 | 341 | 24 | 317 | 6 |
| | HLA-C12:03 | 5922 | 2643 | 700 | 1943 | 45 |
| Liv8_ILS30987D2 | HLA-A11:01 | 5391 | 1649 | 1225 | 424 | 31 |
| | HLA-B15:02 | 5391 | 2114 | 681 | 1433 | 39 |
| | HLA-B35:01 | 5391 | 2425 | 1288 | 1137 | 45 |
| | HLA-C03:03 | 5391 | 1491 | 790 | 701 | 28 |
| | HLA-C08:01 | 5391 | 679 | 133 | 546 | 13 |
| Liv9_ILS24999D2 | HLA-A02:03 | 6765 | 2556 | 2126 | 430 | 38 |
| | HLA-A33:03 | 6765 | 1219 | 737 | 482 | 18 |
| | HLA-B38:02 | 6765 | 1039 | 97 | 942 | 15 |
| | HLA-B48:03 | 6765 | 255 | 27 | 228 | 4 |
| | HLA-C07:02 | 6765 | 537 | 43 | 494 | 8 |
| | HLA-C08:01 | 6765 | 582 | 126 | 456 | 9 |
| Liv10_ILS39940FT1 | HLA-A11:01 | 4375 | 1133 | 926 | 207 | 26 |
| | HLA-A30:01 | 4375 | 1066 | 388 | 678 | 24 |
| | HLA-B07:02 | 4375 | 999 | 858 | 141 | 23 |
| | HLA-B38:02 | 4375 | 1105 | 105 | 1000 | 25 |
| | HLA-C07:02 | 4375 | 424 | 72 | 352 | 10 |
| Liv11_ILS31761D1 | HLA-A02:03 | 5871 | 2681 | 2255 | 426 | 46 |
| | HLA-A11:01 | 5871 | 1318 | 1019 | 299 | 22 |
| | HLA-B15:02 | 5871 | 1608 | 623 | 985 | 27 |
| | HLA-B46:01 | 5871 | 82 | 0 | 82 | 1 |
| | HLA-C01:02 | 5871 | 98 | 1 | 97 | 2 |
| | HLA-C08:01 | 5871 | 436 | 82 | 354 | 7 |
| Liv12_ILS22791D2 | HLA-A02:01 | 6082 | 1671 | 1434 | 237 | 27 |
| | HLA-A11:01 | 6082 | 1201 | 999 | 202 | 20 |
| | HLA-B39:01 | 6082 | 1776 | 865 | 911 | 29 |
| | HLA-B51:01 | 6082 | 440 | 61 | 379 | 7 |
| | HLA-C07:02 | 6082 | 1122 | 143 | 979 | 18 |
| | HLA-C14:02 | 6082 | 1905 | 537 | 1368 | 31 |
| Liv13_ILS23304D2 | HLA-A11:01 | 3528 | 534 | 414 | 120 | 15 |
| | HLA-A24:02 | 3528 | 864 | 469 | 395 | 24 |
| | HLA-B35:05 | 3528 | 1196 | 615 | 581 | 34 |
| | HLA-B46:01 | 3528 | 25 | 0 | 25 | 1 |
| | HLA-C01:02 | 3528 | 30 | 0 | 30 | 1 |
| | HLA-C04:01 | 3528 | 126 | 8 | 118 | 4 |
| Liv14_ILS20947D3 | HLA-A11:01 | 6613 | 2031 | 1483 | 548 | 31 |
| | HLA-B15:02 | 6613 | 1716 | 636 | 1080 | 26 |
| | HLA-B38:02 | 6613 | 1349 | 121 | 1228 | 20 |
| | HLA-C07:02 | 6613 | 554 | 63 | 491 | 8 |
| | HLA-C08:01 | 6613 | 406 | 82 | 324 | 6 |
| Liv15_ILS21034D4 | HLA-A11:01 | 4627 | 1120 | 738 | 382 | 24 |
| | HLA-A33:03 | 4627 | 1273 | 741 | 532 | 28 |
| | HLA-B15:02 | 4627 | 1211 | 459 | 752 | 26 |
| | HLA-B58:01 | 4627 | 935 | 723 | 212 | 20 |
| | HLA-C03:02 | 4627 | 1342 | 475 | 867 | 29 |
| | HLA-C14:02 | 4627 | 1102 | 347 | 755 | 24 |
| Liv17_ILS22914D2 | HLA-A11:01 | 3884 | 1469 | 1040 | 429 | 38 |
| | HLA-A31:01 | 3884 | 1235 | 685 | 550 | 32 |
| | HLA-B13:01 | 3884 | 231 | 17 | 214 | 6 |
| | HLA-B51:02 | 3884 | 455 | 72 | 383 | 12 |
| | HLA-C03:04 | 3884 | 802 | 418 | 384 | 21 |
| | HLA-C15:02 | 3884 | 541 | 83 | 458 | 14 |
| Liv18_ILS20954D2 | HLA-A02:01 | 5207 | 1765 | 1446 | 319 | 34 |
| | HLA-A74:02 | 5207 | 356 | 64 | 292 | 7 |
| | HLA-B15:02 | 5207 | 1393 | 522 | 871 | 27 |
| | HLA-B51:01 | 5207 | 457 | 51 | 406 | 9 |
| | HLA-C08:01 | 5207 | 561 | 105 | 456 | 11 |
| | HLA-C14:02 | 5207 | 1696 | 438 | 1258 | 33 |
| Liv19_ILS21955D2 | HLA-A02:03 | 6392 | 2791 | 2243 | 548 | 44 |
| | HLA-A31:01 | 6392 | 458 | 373 | 85 | 7 |
| | HLA-B15:25 | 6392 | 2625 | 1518 | 1107 | 41 |
| | HLA-B51:01 | 6392 | 442 | 59 | 383 | 7 |
| | HLA-C04:03 | 6392 | 156 | 16 | 140 | 2 |
| | HLA-C16:02 | 6392 | 1311 | 64 | 1247 | 21 |
| Liv20_ILS10922D04 | HLA-A02:264 | 5498 | 2119 | 1863 | 256 | 39 |
| | HLA-A33:03 | 5498 | 1333 | 853 | 480 | 24 |
| | HLA-B18:02 | 5498 | 366 | 125 | 241 | 7 |
| | HLA-B58:01 | 5498 | 1147 | 892 | 255 | 21 |
| | HLA-C03:02 | 5498 | 1371 | 464 | 907 | 25 |
| | HLA-C07:04 | 5498 | 2 | 0 | 2 | 0 |
| Liv21_499956PF | HLA-A02:01 | 4839 | 1755 | 1312 | 443 | 36 |
| | HLA-A11:01 | 4839 | 1057 | 895 | 162 | 22 |
| | HLA-B13:01 | 4839 | 461 | 32 | 429 | 10 |
| | HLA-B55:02 | 4839 | 702 | 300 | 402 | 15 |
| | HLA-C01:02 | 4839 | 88 | 1 | 87 | 2 |
| | HLA-C03:04 | 4839 | 842 | 310 | 532 | 17 |
| Liv22_524614VF | HLA-A11:01 | 4161 | 1820 | 1470 | 350 | 44 |
| | HLA-B18:02 | 4161 | 535 | 169 | 366 | 13 |
| | HLA-B38:02 | 4161 | 737 | 74 | 663 | 18 |
| | HLA-C04:03 | 4161 | 190 | 28 | 162 | 5 |
| | HLA-C07:02 | 4161 | 308 | 54 | 254 | 7 |
| Liv23_ILS35989FT2 | HLA-A02:07 | 5409 | 466 | 75 | 391 | 9 |
| | HLA-A11:01 | 5409 | 1344 | 1104 | 240 | 25 |
| | HLA-B38:02 | 5409 | 1005 | 90 | 915 | 19 |
| | HLA-B46:01 | 5409 | 44 | 0 | 44 | 1 |
| | HLA-C01:12 | 5409 | 784 | 32 | 752 | 14 |
| | HLA-C07:02 | 5409 | 457 | 54 | 403 | 8 |
| Liv24_ILS42195FT1 | HLA-A11:01 | 4097 | 1210 | 876 | 334 | 30 |
| | HLA-A29:01 | 4097 | 697 | 498 | 199 | 17 |
| | HLA-B07:05 | 4097 | 1157 | 686 | 471 | 28 |
| | HLA-B35:01 | 4097 | 1758 | 1046 | 712 | 43 |
| | HLA-C03:03 | 4097 | 861 | 307 | 554 | 21 |
| | HLA-C15:05 | 4097 | 329 | 44 | 285 | 8 |
| Liv25_ILS45824FT1 | HLA-A24:02 | 5307 | 1935 | 967 | 968 | 36 |
| | HLA-B40:01 | 5307 | 1633 | 1153 | 480 | 31 |
| | HLA-B54:01 | 5307 | 607 | 334 | 273 | 11 |
| | HLA-C01:02 | 5307 | 47 | 0 | 47 | 1 |
| | HLA-C04:03 | 5307 | 159 | 18 | 141 | 3 |
| Liv26_ILS36725FT2 | HLA-A01:01 | 4283 | 583 | 425 | 158 | 14 |
| | HLA-A33:03 | 4283 | 1224 | 742 | 482 | 29 |
| | HLA-B13:01 | 4283 | 236 | 17 | 219 | 6 |
| | HLA-B57:01 | 4283 | 983 | 561 | 422 | 23 |
| | HLA-C03:04 | 4283 | 493 | 267 | 226 | 12 |
| | HLA-C06:02 | 4283 | 226 | 71 | 155 | 5 |
| Liv27_ILS37352FT1 | HLA-A11:01 | 3003 | 1402 | 1138 | 264 | 47 |
| | HLA-B38:02 | 3003 | 446 | 38 | 408 | 15 |
| | HLA-B51:01 | 3003 | 288 | 30 | 258 | 10 |
| | HLA-C07:02 | 3003 | 282 | 41 | 241 | 9 |
| | HLA-C14:02 | 3003 | 518 | 208 | 310 | 17 |
| Liv28_ILS50526FA1 | HLA-A11:01 | 4792 | 965 | 616 | 349 | 20 |
| | HLA-A33:03 | 4792 | 1030 | 597 | 433 | 21 |
| | HLA-B15:25 | 4792 | 1567 | 1014 | 553 | 33 |
| | HLA-B35:05 | 4792 | 1620 | 755 | 865 | 34 |
| | HLA-C04:01 | 4792 | 103 | 10 | 93 | 2 |
| | HLA-C04:03 | 4792 | 130 | 18 | 112 | 3 |
| Liv29_ILS50526FT2 | HLA-A11:01 | 5022 | 1271 | 887 | 384 | 25 |
| | HLA-A33:03 | 5022 | 1302 | 746 | 556 | 26 |
| | HLA-B15:25 | 5022 | 1370 | 935 | 435 | 27 |
| | HLA-B35:05 | 5022 | 1491 | 756 | 735 | 30 |
| | HLA-C04:01 | 5022 | 95 | 9 | 86 | 2 |
| | HLA-C04:03 | 5022 | 142 | 14 | 128 | 3 |
| Liv30_ILS50523FA1 | HLA-A11:01 | 5291 | 1100 | 696 | 404 | 21 |
| | HLA-A33:03 | 5291 | 1350 | 761 | 589 | 26 |
| | HLA-B15:02 | 5291 | 1564 | 538 | 1026 | 30 |
| | HLA-B58:01 | 5291 | 1015 | 695 | 320 | 19 |
| | HLA-C03:02 | 5291 | 1776 | 681 | 1095 | 34 |
| | HLA-C08:01 | 5291 | 384 | 84 | 300 | 7 |
| Liv31_ILS50523FT2 | HLA-A11:01 | 5673 | 1152 | 708 | 444 | 20 |
| | HLA-A33:03 | 5673 | 1464 | 821 | 643 | 26 |
| | HLA-B15:02 | 5673 | 1613 | 559 | 1054 | 28 |
| | HLA-B58:01 | 5673 | 1084 | 727 | 357 | 19 |
| | HLA-C03:02 | 5673 | 1878 | 741 | 1137 | 33 |
| | HLA-C08:01 | 5673 | 433 | 96 | 337 | 8 |
| Liv32_1176935F | HLA-A02:06 | 5727 | 2042 | 1277 | 765 | 36 |
| | HLA-A11:01 | 5727 | 1171 | 825 | 346 | 20 |
| | HLA-B15:02 | 5727 | 2074 | 611 | 1463 | 36 |
| | HLA-C07:02 | 5727 | 245 | 16 | 229 | 4 |
| | HLA-C08:01 | 5727 | 421 | 78 | 343 | 7 |
| Liv33_1176559F | HLA-A24:02 | 2336 | 460 | 302 | 158 | 20 |
| | HLA-A26:01 | 2336 | 467 | 330 | 137 | 20 |
| | HLA-B38:02 | 2336 | 377 | 42 | 335 | 16 |
| | HLA-B54:01 | 2336 | 411 | 286 | 125 | 18 |
| | HLA-C01:02 | 2336 | 26 | 1 | 25 | 1 |
| | HLA-C07:02 | 2336 | 416 | 55 | 361 | 18 |
| Liv34_1182970F | HLA-A11:01 | 2780 | 844 | 556 | 288 | 30 |
| | HLA-A29:01 | 2780 | 1049 | 581 | 468 | 38 |
| | HLA-B15:02 | 2780 | 1345 | 414 | 931 | 48 |
| | HLA-C04:03 | 2780 | 108 | 22 | 86 | 4 |
| | HLA-C08:01 | 2780 | 221 | 54 | 167 | 8 |
| Liv35_1181680F | HLA-A11:01 | 4422 | 837 | 603 | 234 | 19 |
| | HLA-A33:03 | 4422 | 168 | 51 | 117 | 4 |
| | HLA-B44:03 | 4422 | 11 | 2 | 9 | 0 |
| | HLA-B56:04 | 4422 | 1172 | 456 | 716 | 27 |
| | HLA-C07:06 | 4422 | 35 | 3 | 32 | 1 |
| | HLA-C08:01 | 4422 | 228 | 39 | 189 | 5 |
| Liv36_1182812F | HLA-A11:01 | 4221 | 132 | 26 | 106 | 3 |
| | HLA-A26:01 | 4221 | 88 | 7 | 81 | 2 |
| | HLA-B44:03 | 4221 | 1016 | 448 | 568 | 24 |
| | HLA-B56:04 | 4221 | 77 | 4 | 73 | 2 |
| | HLA-C07:06 | 4221 | 55 | 9 | 46 | 1 |
| | HLA-C08:01 | 4221 | 304 | 56 | 248 | 7 |
| Liv37_1189452F | HLA-A11:01 | 3749 | 748 | 488 | 260 | 20 |
| | HLA-A24:02 | 3749 | 625 | 368 | 257 | 17 |
| | HLA-B15:12 | 3749 | 1282 | 83 | 1199 | 34 |
| | HLA-B15:25 | 3749 | 2093 | 1336 | 757 | 56 |
| | HLA-C03:03 | 3749 | 576 | 204 | 372 | 15 |
| | HLA-C04:03 | 3749 | 75 | 10 | 65 | 2 |
| Liv38_1193117F | HLA-A11:01 | 3383 | 517 | 330 | 187 | 15 |
| | HLA-A24:03 | 3383 | 614 | 387 | 227 | 18 |
| | HLA-B15:02 | 3383 | 1541 | 455 | 1086 | 46 |
| | HLA-C04:03 | 3383 | 113 | 20 | 93 | 3 |
| | HLA-C08:01 | 3383 | 227 | 43 | 184 | 7 |
| Liv39_1192879F | HLA-A11:01 | 1269 | 227 | 164 | 63 | 18 |
| | HLA-B15:02 | 1269 | 392 | 139 | 253 | 31 |
| | HLA-B46:01 | 1269 | 18 | 0 | 18 | 1 |
| | HLA-C01:02 | 1269 | 17 | 0 | 17 | 1 |
| | HLA-C08:01 | 1269 | 72 | 11 | 61 | 6 |
| Liv40_405295F2 | HLA-A02:07 | 2670 | 272 | 46 | 226 | 10 |
| | HLA-A02:65 | 2670 | 85 | 4 | 81 | 3 |
| | HLA-B38:02 | 2670 | 429 | 37 | 392 | 16 |
| | HLA-B46:01 | 2670 | 23 | 0 | 23 | 1 |
| | HLA-C01:12 | 2670 | 505 | 24 | 481 | 19 |
| | HLA-C07:02 | 2670 | 275 | 34 | 241 | 10 |
| Liv41_ILS32961D2 | HLA-A01:01 | 2669 | 407 | 304 | 103 | 15 |
| | HLA-A02:07 | 2669 | 291 | 55 | 236 | 11 |
| | HLA-B46:01 | 2669 | 26 | 0 | 26 | 1 |
| | HLA-B57:01 | 2669 | 436 | 249 | 187 | 16 |
| | HLA-C01:02 | 2669 | 65 | 0 | 65 | 2 |
| | HLA-C06:02 | 2669 | 111 | 36 | 75 | 4 |
| Liv42_ILS33989D2 | HLA-A02:01 | 5871 | 1711 | 1360 | 351 | 29 |
| | HLA-A24:02 | 5871 | 1106 | 625 | 481 | 19 |
| | HLA-B15:02 | 5871 | 2090 | 616 | 1474 | 36 |
| | HLA-B15:12 | 5871 | 1315 | 94 | 1221 | 22 |
| | HLA-C03:03 | 5871 | 1367 | 554 | 813 | 23 |
| | HLA-C08:01 | 5871 | 635 | 128 | 507 | 11 |
| Liv43_ILS22892S1 | HLA-A02:07 | 3964 | 365 | 59 | 306 | 9 |
| | HLA-A33:03 | 3964 | 858 | 526 | 332 | 22 |
| | HLA-B46:01 | 3964 | 56 | 0 | 56 | 1 |
| | HLA-B58:01 | 3964 | 900 | 620 | 280 | 23 |
| | HLA-C01:02 | 3964 | 90 | 1 | 89 | 2 |
| | HLA-C03:02 | 3964 | 1291 | 438 | 853 | 33 |
| Liv44_ILS24966D3-DS2 | HLA-A02:03 | 2995 | 1203 | 992 | 211 | 40 |
| | HLA-A33:03 | 2995 | 498 | 308 | 190 | 17 |
| | HLA-B13:01 | 2995 | 127 | 7 | 120 | 4 |
| | HLA-B58:01 | 2995 | 493 | 329 | 164 | 16 |
| | HLA-C03:02 | 2995 | 820 | 274 | 546 | 27 |
| | HLA-C03:04 | 2995 | 597 | 213 | 384 | 20 |
| Liv45_ILS30194D2 | HLA-A02:03 | 4119 | 1513 | 1306 | 207 | 37 |
| | HLA-A24:02 | 4119 | 900 | 523 | 377 | 22 |
| | HLA-B15:12 | 4119 | 533 | 45 | 488 | 13 |
| | HLA-B40:01 | 4119 | 580 | 416 | 164 | 14 |
| | HLA-C03:03 | 4119 | 613 | 201 | 412 | 15 |
| | HLA-C07:02 | 4119 | 725 | 94 | 631 | 18 |
| Liv46_ILS32365D1 | HLA-A02:07 | 2352 | 175 | 34 | 141 | 7 |
| | HLA-A24:03 | 2352 | 453 | 308 | 145 | 19 |
| | HLA-B15:01 | 2352 | 839 | 383 | 456 | 36 |
| | HLA-B15:12 | 2352 | 518 | 30 | 488 | 22 |
| | HLA-C01:03 | 2352 | 1 | 0 | 1 | 0 |
| | HLA-C03:03 | 2352 | 498 | 202 | 296 | 21 |
| Liv47_ILS32963D1 | HLA-A11:01 | 2169 | 479 | 352 | 127 | 22 |
| | HLA-A31:01 | 2169 | 345 | 153 | 192 | 16 |
| | HLA-B51:01 | 2169 | 433 | 49 | 384 | 20 |
| | HLA-B51:02 | 2169 | 461 | 88 | 373 | 21 |
| | HLA-C14:02 | 2169 | 371 | 174 | 197 | 17 |
| | HLA-C15:02 | 2169 | 208 | 39 | 169 | 10 |
| Liv48_ILS36088FT2 | HLA-A02:03 | 3772 | 1109 | 893 | 216 | 29 |
| | HLA-A11:01 | 3772 | 505 | 371 | 134 | 13 |
| | HLA-B15:21 | 3772 | 286 | 14 | 272 | 8 |
| | HLA-B56:04 | 3772 | 910 | 376 | 534 | 24 |
| | HLA-C01:02 | 3772 | 44 | 0 | 44 | 1 |
| | HLA-C04:03 | 3772 | 86 | 12 | 74 | 2 |

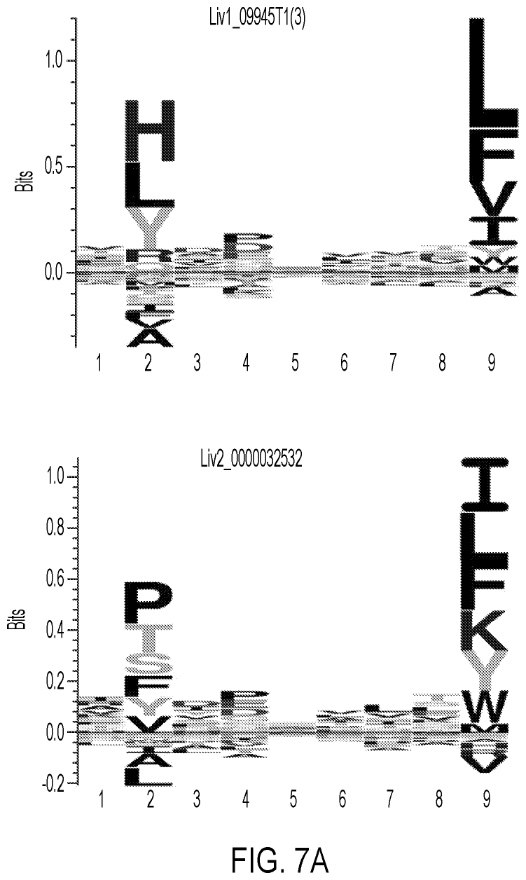

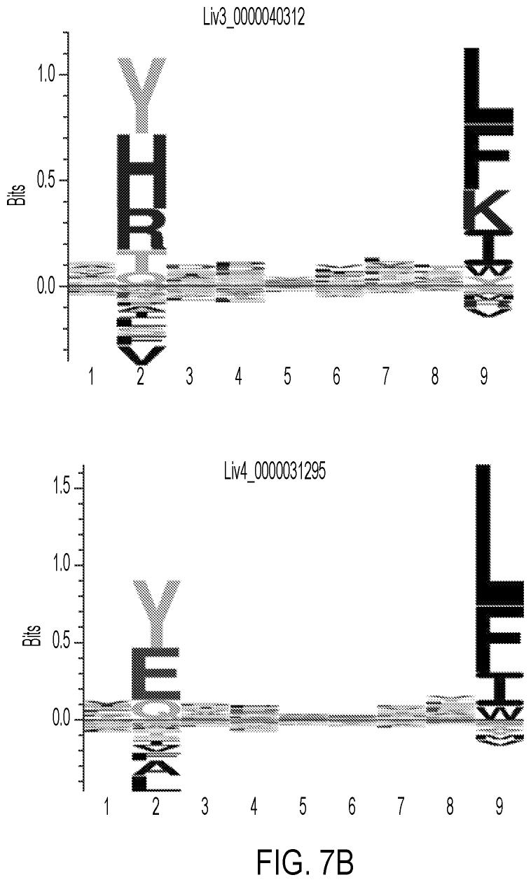

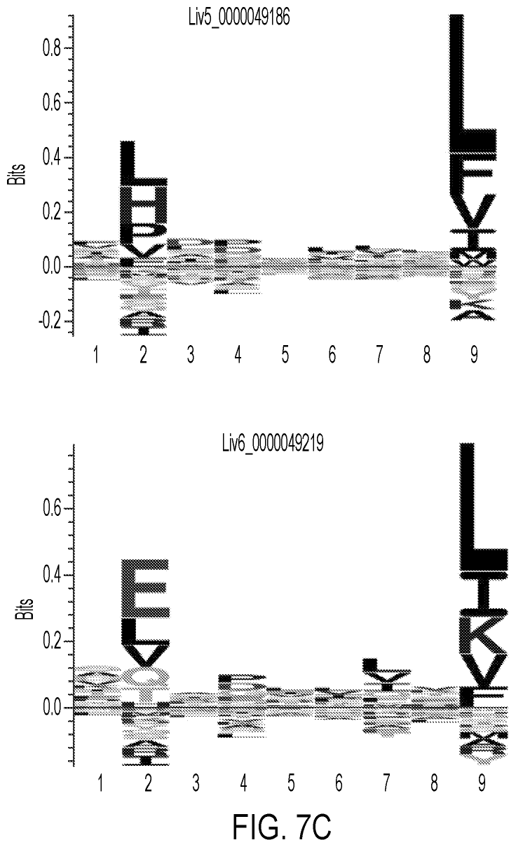

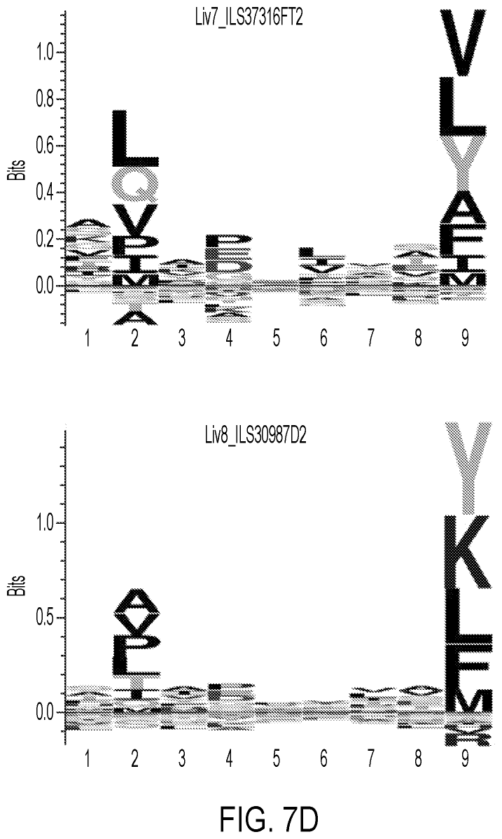

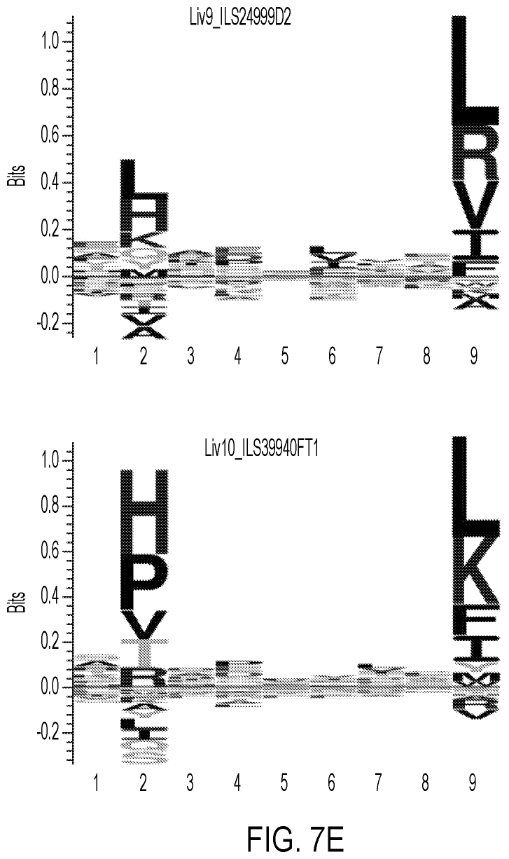

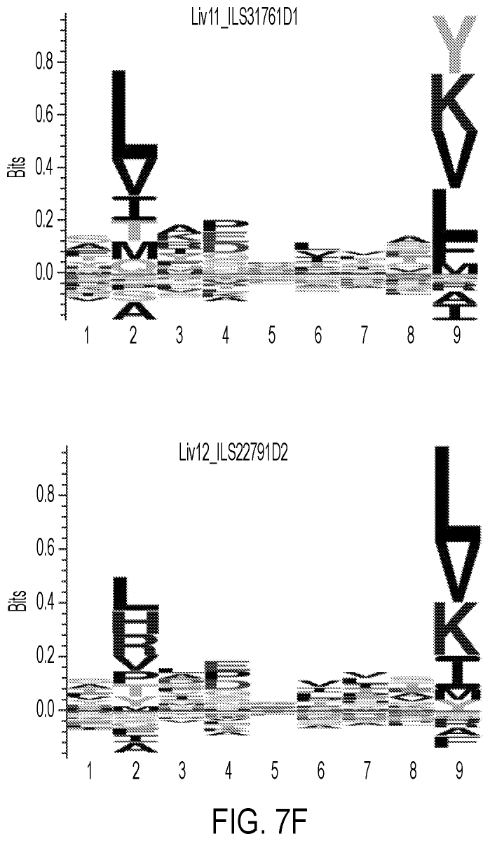

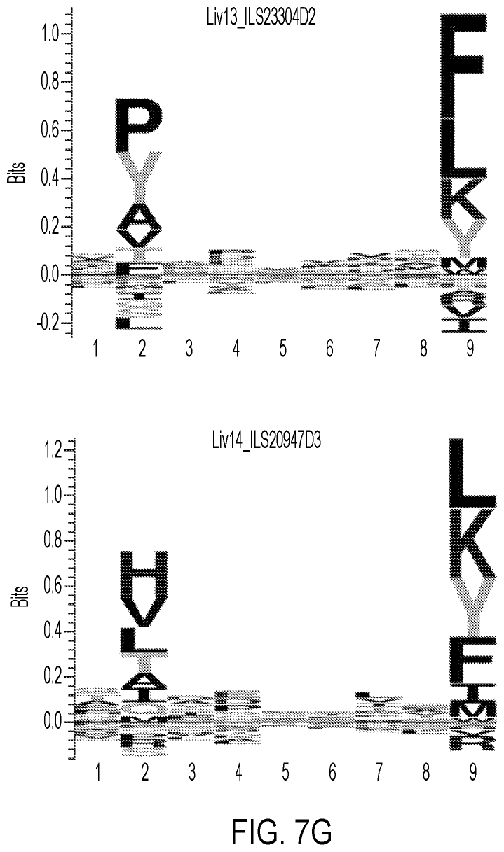

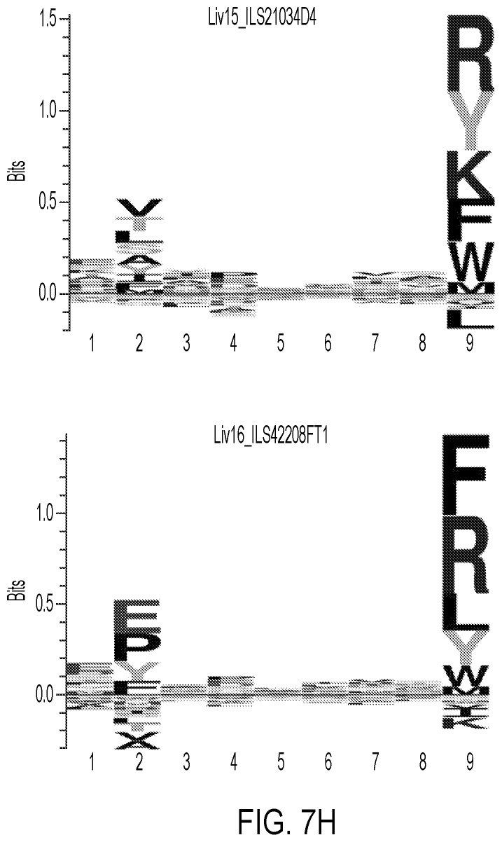

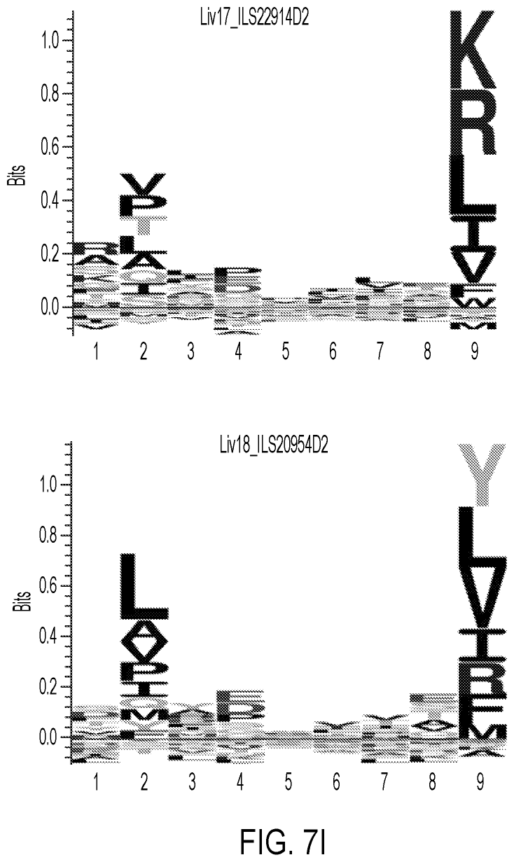

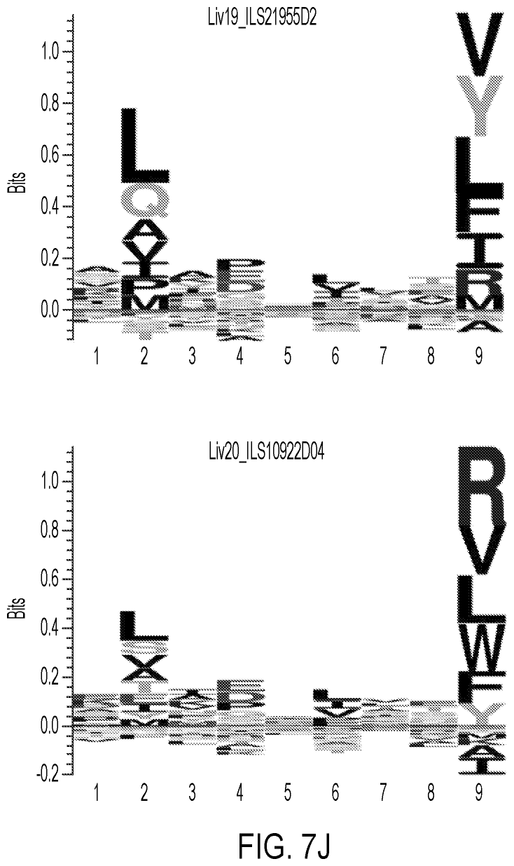

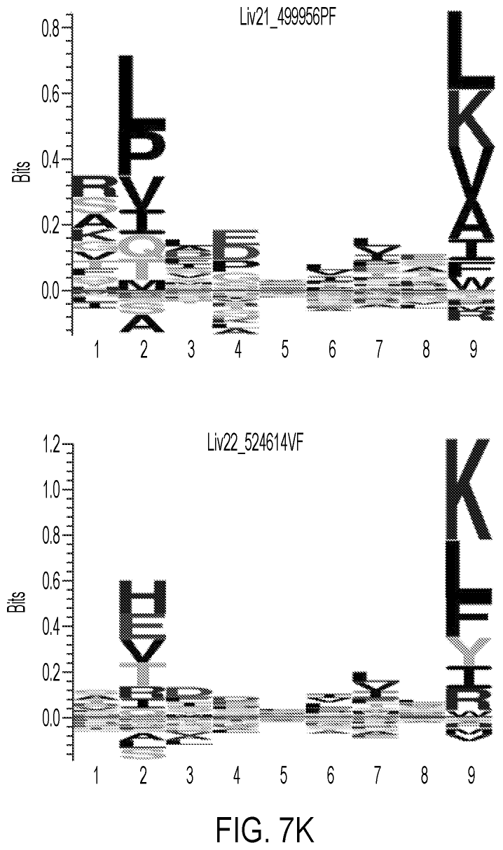

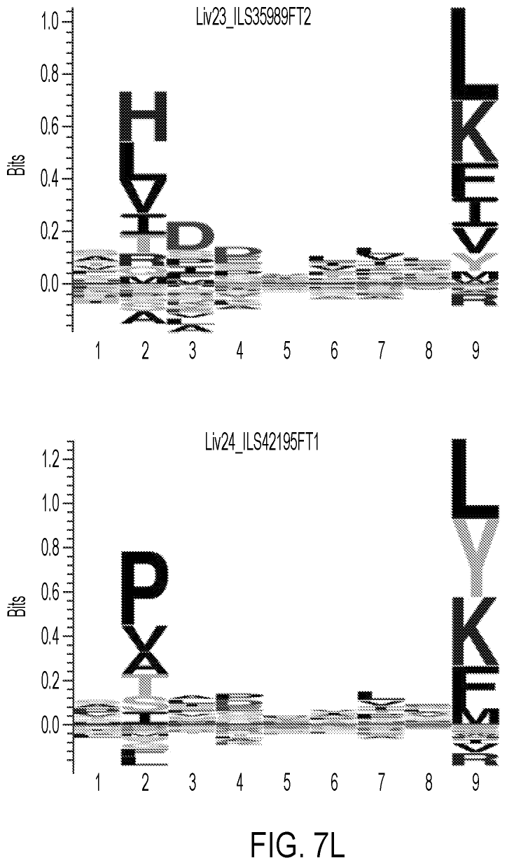

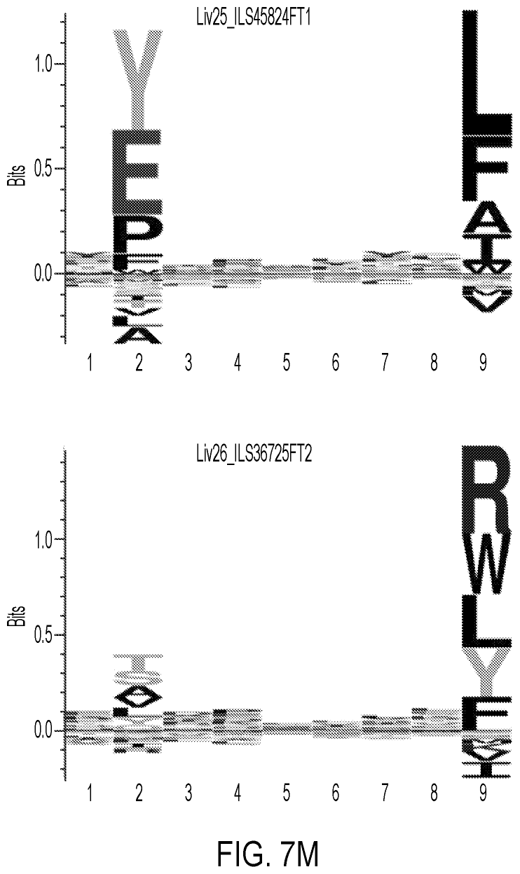

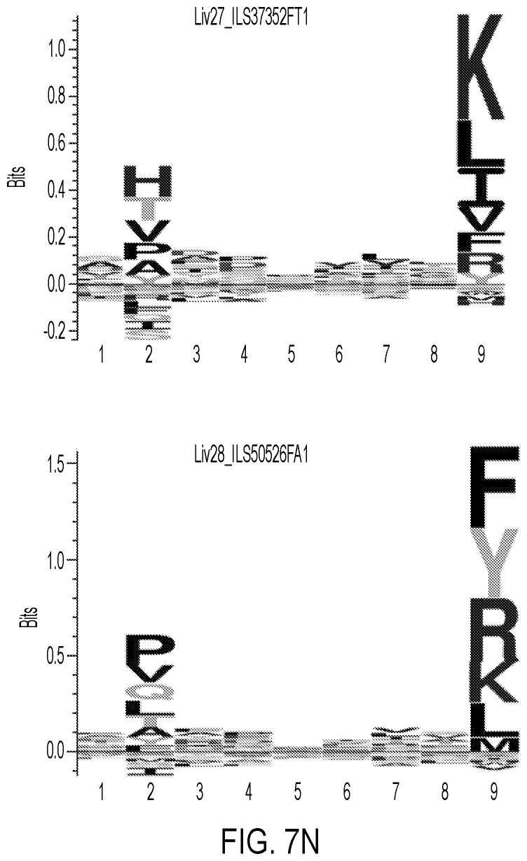

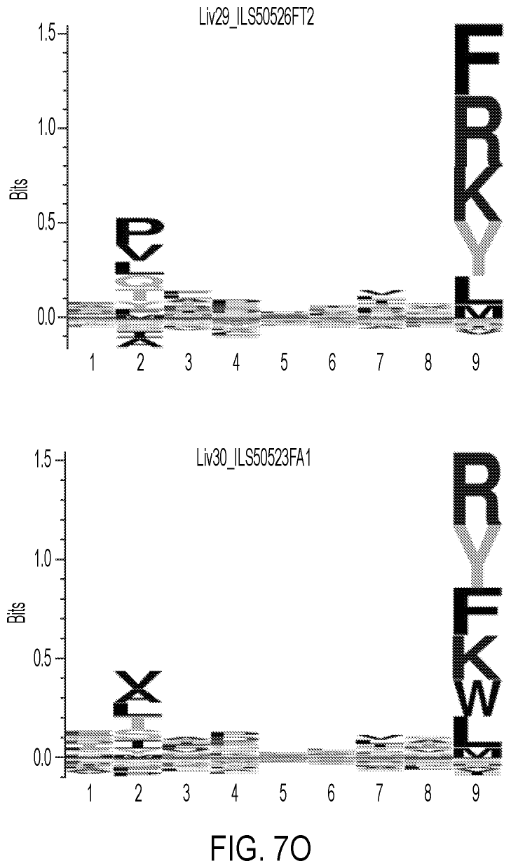

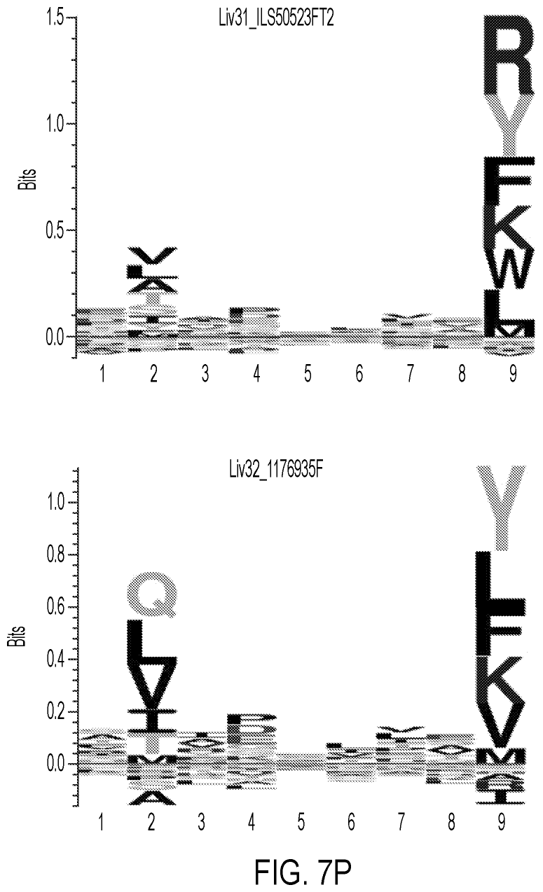

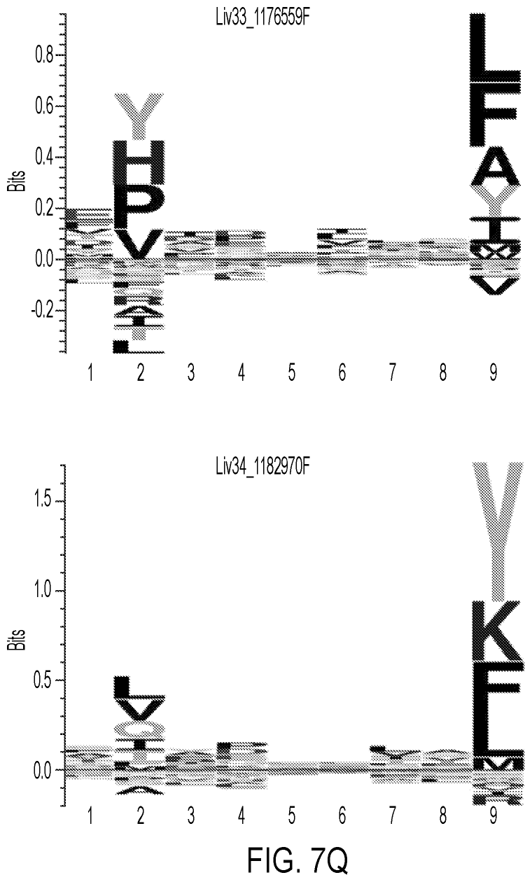

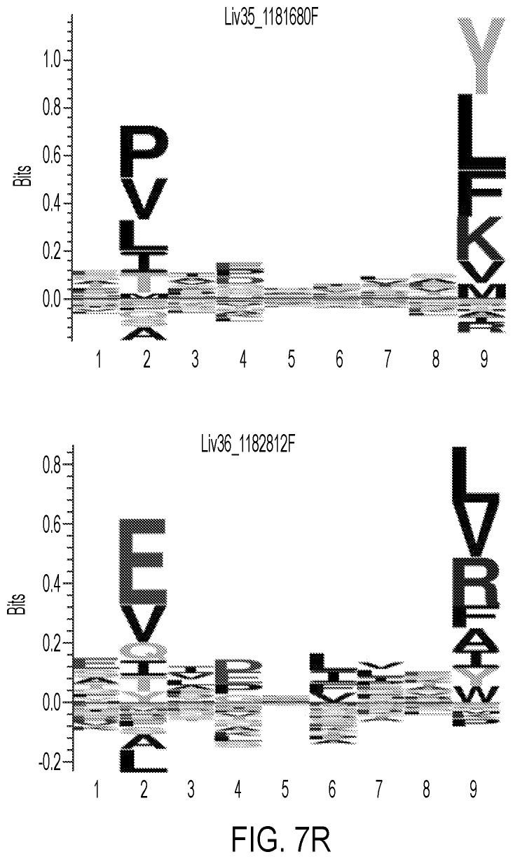

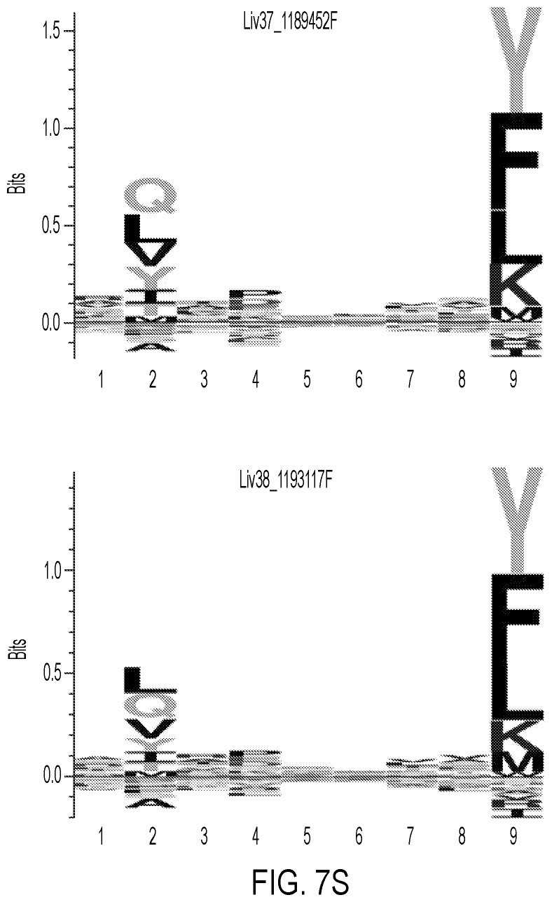

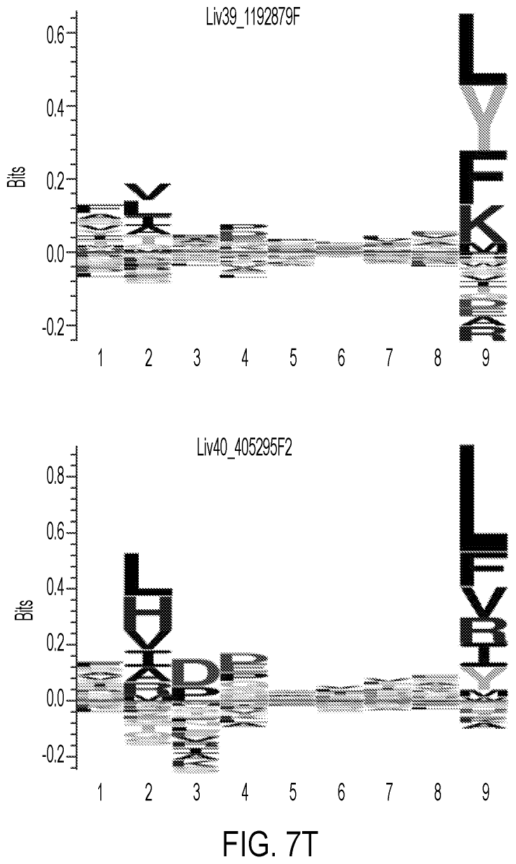

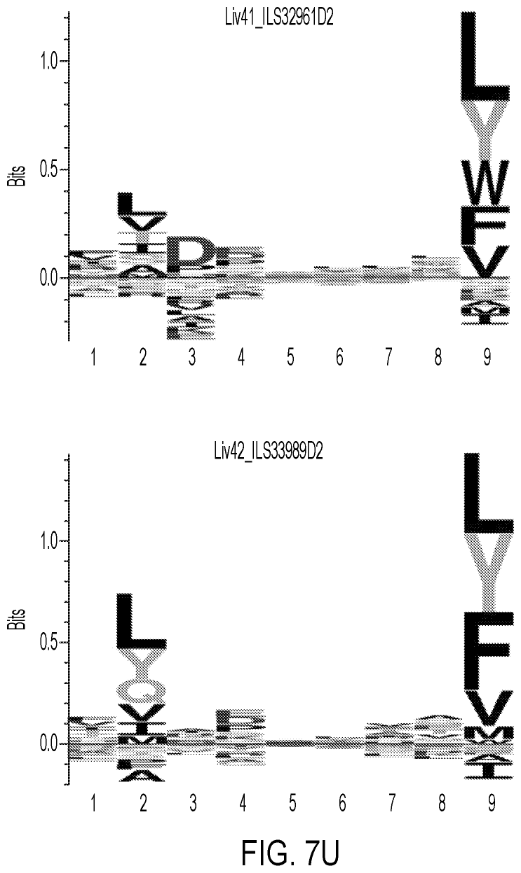

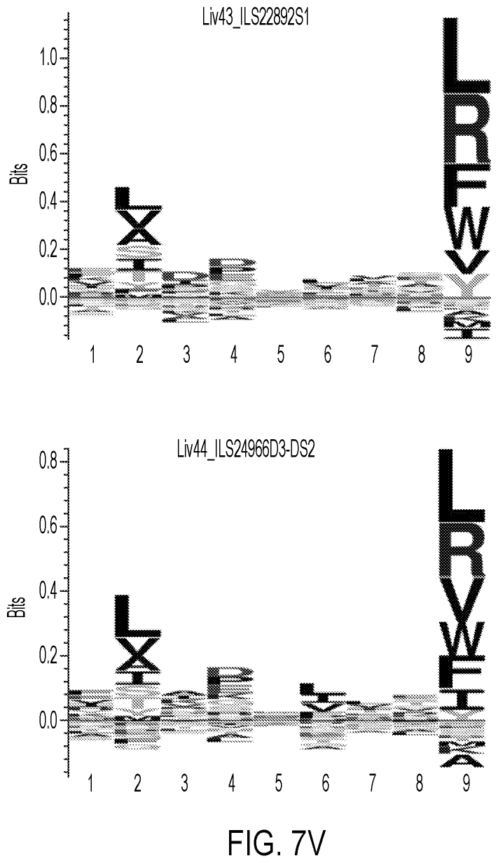

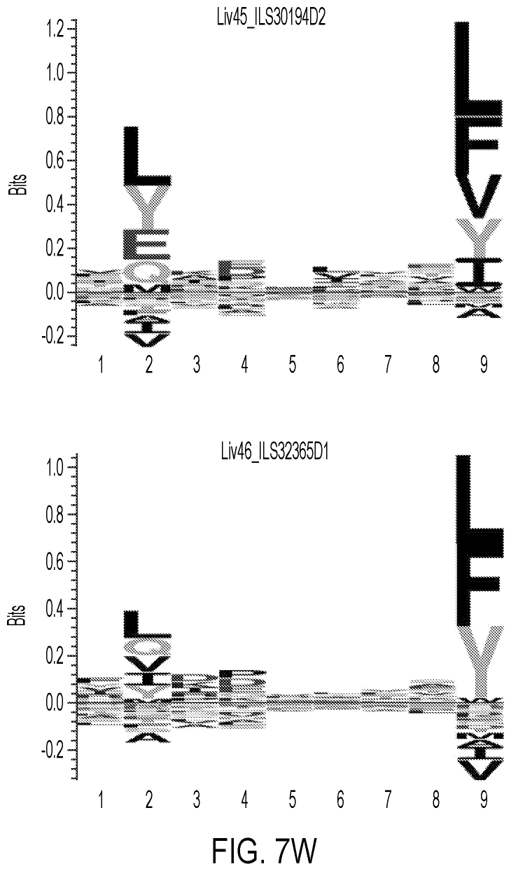

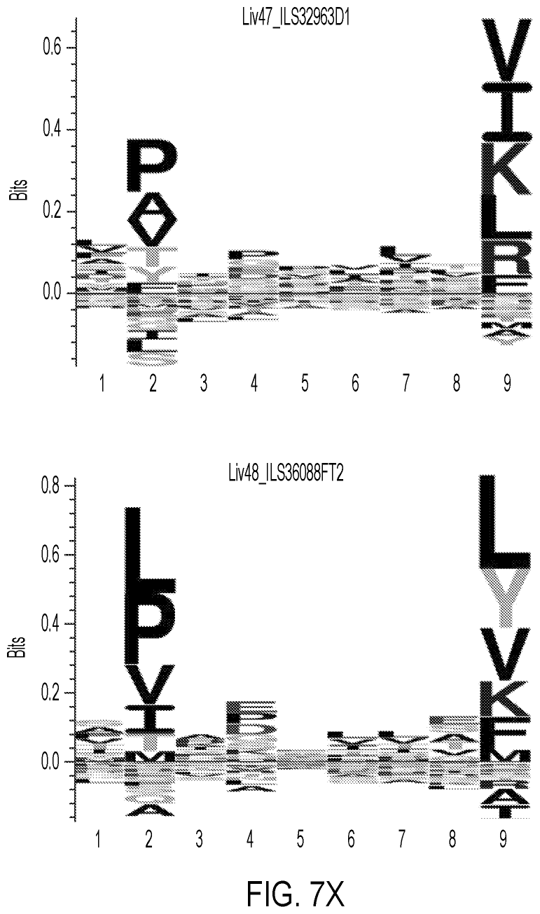

A motif analysis of detected peptides was next performed to identify overrepresented residues at different peptide positions using Seq2Logo (FIGS. 7A-7X). Anchor residues at position 2 and 9, which specify HLA-I binding, were significantly overrepresented in all the tissue samples, with residue identities corresponding to the HLA-I genotype present. For example, hydrophobic anchor residues (i.e. Leu and Val) characteristic of HLA-A02 binders were enriched at positions 2 and 9 of peptides isolated from HLA-A02+ patient samples. Together, the high percentage of predicted HLA binders and the overrepresentation of characteristic anchor residues in isolated peptides provided a high degree of confidence in the HLA-I enrichment and peptide identifications.

### Example 4. Identification of Unique HLA-Associated HBV Peptides Using HBV Genotype-Specific Databases

A major challenge in developing immunotherapeutics against HBV is the genomic variability between HBV strains. Utilizing RNA sequencing, HBV genotypes present in each patient sample were identified (FIG. 1C). In accordance with the reported prevalence of HBV genotypes in Asian populations, HBV genotypes B and C were most identified, having been found in 24 of the 48 samples (12 each). HBV genotypes A and D were detected in only one patient sample each (Table 4). RNA sequencing data for 18 of the samples had low HBV coverage, indicating an absence of HBV virus in these samples, possibly due to viral latency or clearance. No HBV peptides were detected from any of these 18 tissues, further validating the proteogenomic approach described in the present Examples, and highlighting a good correlation between the two complementary methods. In total, HBV peptides were detected in 30 liver samples, with RNA sequencing efforts unable to determine the HBV genotype present in 4 of the samples.

The number of HLA-I-presented HBV peptides likely depends on the degree of HBV infection, proteasomal processing of antigen, and tissue sample quality. Indeed, the number of HBV-specific peptide sequences identified from infected livers varied between samples and generally showed low sequence coverage of HBV proteins (Table 4 and FIG. 1B). In total, 49 unique HBV peptides with lengths of nine to twelve residues were identified using a PEAKS ion cut off score of 20 (Table 8). Table 8 shows HBV peptides and proteins detected by search against patient-specific databases. Peptides were initially filtered by 5% False-Discovery Rate (FDR) followed by −log P of 20 as minimum cut-off score.

TABLE 8
HBV peptides and proteins detected by search against patient-specific
databases.

| | | SEQ | | | | |
|---|---|---|---|---|---|---|
| | HBV Peptides | ID | IonScore | | | HBV |
| Sample ID | (5% FDR) | NO: | (-logP) | Length | m/z | Protein |
| Liv1_09945T1(3) | LTIPQSLDSW | 31 | 43 | 10 | 580.302 | S |
| | TRILTIPQSL | 47 | 33 | 10 | 571.3472 | S |
| | FVGLSPTVWL | 7 | 20 | 10 | 559.8152 | S |
| | MENITSGFLGPL | 36 | 46 | 12 | 639.8233 | S |
| Liv2_0000032532 | — | | | | | |
| Liv3_0000040312 | LTIPQSLDSW | 31 | 33 | 10 | 580.303 | S |
| | TRILTIPQSL | 47 | 22 | 10 | 571.3493 | S |
| | MENITSGFLGPL | 36 | 37 | 12 | 639.823 | S |
| Liv4_0000031295 | — | | | | | |
| Liv5_0000049186 | VGLSPTVWL | 49 | 39 | 9 | 971.5576 | S |
| | FVGLSPTVW | 6 | 38 | 9 | 1005.5411 | S |
| | ITSGFLGPL | 19 | 36 | 9 | 904.514 | S |
| | LTIPQSLDSW | 31 | 48 | 10 | 1159.6012 | S |
| | NITSGFLGPL | 38 | 41 | 10 | 1018.5593 | S |
| | TRILTIPQSL | 47 | 40 | 10 | 571.3502 | S |
| | FVGLSPTVWL | 7 | 33 | 10 | 559.8156 | S |
| | ITSGFLGPLL | 20 | 29 | 10 | 509.3024 | S |
| | LTRILTIPQSL | 33 | 43 | 11 | 627.8931 | S |
| | ENITSGFLGPL | 2 | 32 | 11 | 574.3026 | S |
| | WTSLNFLGGTTV | 50 | 47 | 12 | 648.3353 | S |
| | MENITSGFLGPL | 36 | 32 | 12 | 1278.6306 | S |
| Liv6_0000049219 | TRILTIPQSL | 47 | 29 | 10 | 571.3505 | S |
| Liv7_ILS37316FT2 | — | | | | | |
| Liv8_ILS30987D2 | HLYSHPIIL | 16 | 40 | 9 | 546.8131 | Polymer- |
| | | | | | | ase |
| | LPQEHIVHK | 26 | 34 | 9 | 367.545 | Polymer- |
| | | | | | | ase |
| | LPETTVVRR | 24 | 30 | 9 | 535.8181 | Polymer- |
| | | | | | | ase |
| | TASPISSIF | 44 | 31 | 9 | 922.4841 | S |
| | GTLPQEHIVHK | 13 | 55 | 11 | 629.8475 | Polymer- |
| | | | | | | ase |
| | STLPETTVVRR | 43 | 32 | 11 | 420.2415 | Polymer- |
| | | | | | | ase |
| Liv9_ILS24999D2 | IASGLLGPL | 17 | 25 | 9 | 840.5204 | S |
| | FVGLSPTVWL | 7 | 28 | 10 | 559.8152 | S |
| | NILSPFMPLL | 37 | 23 | 10 | 572.8248 | S |
| | MENIASGLLGPL | 35 | 44 | 12 | 607.8264 | S |
| | IASGLLGPLLVL | 18 | 31 | 12 | 583.3806 | S |
| Liv10_ILS39940FT1 | SAISSTFSK | 41 | 29 | 9 | 464.2422 | S |
| | KPRKGMGTNL | 22 | 35 | 10 | 551.3126 | |
| | GSLPQEHIVQK | 12 | 47 | 11 | 618.3405 | Polymer- |
| | | | | | | ase |
| | STLPETTVVRR | 43 | 27 | 11 | 420.241 | C |
| Liv11_ILS31761D1 | QSPTSNHSL | 40 | 40 | 9 | 970.4584 | S |
| | GLSPTVWLSV | 8 | 31 | 10 | 529.7979 | S |
| | GTLPQEHIVQK | 14 | 50 | 11 | 625.3479 | Polymer- |
| | | | | | | ase |
| Liv12_ILS22791D2 | LPSDFFPSI | 27 | 22 | 9 | 1022.5152 | C |
| | GMLPVCPLL | 10 | 34 | 9 | 942.5134 | S |
| | FLLTRILTI | 3 | 25 | 9 | 545.3545 | S |
| | GMLPVCPLI | 9 | 34 | 9 | 942.5134 | S |
| | TRILTIPQSL | 47 | 32 | 10 | 571.3494 | S |
| | GTLPQEHIVQK | 14 | 54 | 11 | 625.3477 | Polymer- |
| | | | | | | ase |
| | STLPETTVVRR | 43 | 30 | 11 | 629.8578 | Polymer- |
| | | | | | | ase |
| Liv13_ILS23304D2 | — | | | | | |
| Liv14_ILS20947D3 | GTLPQEHIVHK | 13 | 42 | 11 | 420.2349 | Polymer- |
| | | | | | | ase |
| | STLPETTVVRR | 43 | 33 | 11 | 420.2417 | Polymer- |
| | | | | | | ase |
| Liv15_ILS21034D4 | TVSAISSTF | 48 | 32 | 9 | 912.4692 | S |
| | TIPQSLDSW | 46 | 21 | 9 | 523.7634 | S |
| | GSLPQEHIVQK | 12 | 36 | 11 | 618.3413 | Polymer- |
| | | | | | | ase |
| | KILTIPQSLDSW | 21 | 59 | 12 | 700.8957 | S |
| Liv16_ILS42208FT1 | TASAISSTF | 53 | 35 | 9 | 884.4364 | S |
| | IPIPSSWAF | 52 | 38 | 9 | 1017.5414 | S |
| | LPYRPTTGR | 28 | 42 | 9 | 530.7983 | Polymer- |
| | | | | | | ase |
| | FSLTKILTIPQ | 4 | 34 | 11 | 630.8829 | S |
| Liv17_ILS22914D2 | GSLPQEHIVQK | 12 | 44 | 11 | 618.3427 | Polymer- |
| | | | | | | ase |
| Liv18_ILS20954D2 | — | | | | | |
| Liv19_ILS21955D2 | GVWIRTPPAYR | 15 | 34 | 11 | 439.2469 | C |
| Liv20_ILS10922D04 | PQSLDSWLT | 39 | 24 | 9 | 523.7632 | S |
| | LPYRPTTGR | 28 | 24 | 9 | 354.2018 | Polymer- |
| | | | | | | ase |
| | LTIPQSLDSW | 31 | 45 | 10 | 580.3043 | S |
| | NILSPFMPLL | 37 | 40 | 10 | 572.8265 | S |
| | FVGLSPTVWL | 7 | 32 | 10 | 559.8168 | S |
| | FSLTKILTIPQ | 4 | 39 | 11 | 630.8831 | S |
| | LTIPQSLDSWW | 32 | 23 | 11 | 673.3439 | S |
| | KILTIPQSLDSW | 21 | 47 | 12 | 700.8918 | S |
| | MENIASGLLGPL | 35 | 43 | 12 | 607.8276 | S |
| | IASGLLGPLLVL | 18 | 42 | 12 | 583.3824 | S |
| | FSLTKILTIPQS | 5 | 33 | 12 | 674.3987 | S |
| Liv21_499956PF | GSLPQEHIVQK | 12 | 19* | 11 | 412.5624 | Polymer- |
| | | | | | | ase |
| | SAISSTFSK | 41 | 35 | 9 | 464.242 | S |
| Liv22_524614VF | — | | | | | |
| Liv23_ILS35989FT2 | — | | | | | |
| Liv24_ILS42195FT1 | — | | | | | |
| Liv25_ILS45824FT1 | KYTSFPWLL | 23 | 37 | 9 | 577.8159 | Polymer- |
| | | | | | | ase |
| | YPALMPLYA | 51 | 27 | 9 | 519.7706 | Polymer- |
| | | | | | | ase |
| | LYAAVTNFL | 34 | 23 | 9 | 506.2791 | Polymer- |
| | | | | | | ase |
| | MENIASGLLGPL | 35 | 34 | 12 | 607.8266 | S |
| Liv26_ILS36725FT2 | — | | | | | |
| Liv27_ILS37352FT1 | GSLPQEHIVQK | 12 | 41 | 11 | 412.5654 | Polymer- |
| | | | | | | ase |
| | SAISSTFSK | 41 | 35 | 9 | 464.2455 | S |
| Liv28_ILS50526FA1 | STLPETTVVRR | 43 | 33 | 11 | 420.2422 | Polymer- |
| | | | | | | ase |
| | LPFRPTTGR | 25 | 27 | 9 | 348.8699 | Polymer- |
| | | | | | | ase |
| | ASRELVVSY | 1 | 23 | 9 | 512.2773 | C |
| | GTLPQEHIVHK | 13 | 37 | 11 | 420.2355 | Polymer- |
| | | | | | | ase |
| Liv29_ILS50526FT2 | — | | | | | |
| Liv30_ILS50523FA1 | MENIASGLLGPL | 35 | 38 | 12 | 607.8255 | S |
| | LTIPQSLDSW | 31 | 37 | 10 | 580.3024 | S |
| | SAISSTFSK | 41 | 33 | 9 | 464.2425 | S |
| | VGLSPTVWL | 49 | 33 | 9 | 486.2813 | S |
| | FVGLSPTVWL | 7 | 32 | 10 | 559.8156 | S |
| | FVGLSPTVW | 6 | 27 | 9 | 503.273 | S |
| Liv31_ILS50523FT2 | MENIASGLLGPL | 35 | 36 | 12 | 607.8255 | S |
| | SAISSTFSK | 41 | 35 | 9 | 464.2427 | S |
| | LTIPQSLDSW | 31 | 34 | 10 | 580.3034 | S |
| | TCIPIPSSW | 45 | 31 | 9 | 502.2487 | S |
| | FVGLSPTVWL | 7 | 25 | 10 | 559.8158 | S |
| Liv32_1176935F | — | | | | | |
| Liv33_1176559F | — | | | | | |
| Liv34_1182970F | — | | | | | |
| Liv35_1181680F | STISSTFSK | 42 | 31 | 9 | 479.2466 | S |
| | FVGLSPTVWL | 7 | 30 | 10 | 559.8156 | S |
| | GSLPQEHIVQK | 12 | 23 | 11 | 412.5617 | Polymer- |
| | | | | | | ase |
| | MENIASGLLGPL | 35 | 26 | 12 | 607.8254 | S |
| Liv36_1182812F | — | | | | | |
| Liv37_1189452F | FVGLSPTVWL | 7 | 24 | 10 | 559.8156 | S |
| | MENIASGLLGPL | 35 | 23 | 12 | 607.8256 | S |
| Liv38_1193117F | LQDPRVRAL | 29 | 35 | 9 | 534.3187 | S |
| | FVGLSPTVW | 6 | 25 | 9 | 503.2733 | S |
| | HLYSHPIIL | 16 | 36 | 9 | 546.8134 | S |
| | ASRELVVSY | 1 | 30 | 9 | 512.2762 | C |
| | LQDPRVRALY | 30 | 32 | 10 | 410.9025 | S |
| | FVGLSPTVWL | 7 | 31 | 10 | 559.815 | S |
| | STLPETTVVRR | 43 | 38 | 11 | 420.2418 | Polymer- |
| | | | | | | ase |
| | GSLPQEHIIQK | 11 | 35 | 11 | 417.2343 | Polymer- |
| | | | | | | ase |
| | MENIASGLLGPL | 35 | 39 | 12 | 607.826 | S |
| Liv39_1192879F | — | | | | | |
| Liv40_405295F2 | FVGLSPTVWL | 7 | 29 | 10 | 559.8152 | Polymer- |
| | | | | | | ase |
| Liv41_ILS32961D2 | — | | | | | |
| Liv42_ILS33989D2 | — | | | | | |
| Liv43_ILS22892S1 | LTIPQSLDSW | 31 | 30 | 10 | 580.3041 | S |
| | FVGLSPTVWL | 7 | 24 | 10 | 559.8157 | S |
| Liv44_ILS24966D3- | FVGLSPTVW | 6 | 29 | 9 | 503.2723 | S |
| DS2 | LTIPQSLDSW | 31 | 36 | 10 | 580.3026 | S |
| | FVGLSPTVWL | 7 | 31 | 10 | 559.8146 | S |
| Liv45_ILS30194D2 | FLLTRILTI | 3 | 23 | 9 | 545.354 | S |
| | TASPISSIF | 44 | 21 | 9 | 461.7469 | S |
| | TASPLSSIF | 54 | 21 | 9 | 461.7469 | S |
| Liv46_ILS32365D1 | — | | | | | |
| Liv47_ILS32963D1 | GTLPQEHIVHK | 13 | 38 | 11 | 420.2349 | Polymer- |
| | | | | | | ase |
| Liv48_ILS36088FT2 | GTLPQEHIVHK | 13 | 42 | 11 | 420.2341 | Polymer- |
| | | | | | | ase |

* Peptide GSLPQEHIVQK (SEQ ID NO: 12), detected once with -logP of 19 was considered as a real identification (see sample Liv21_499956PF). For all other peptides, a score cut-off of 20 was used.

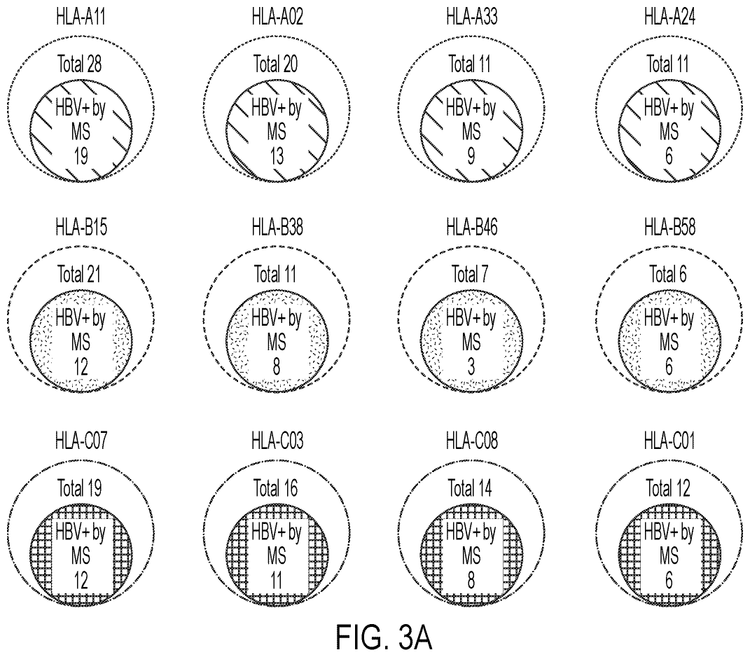

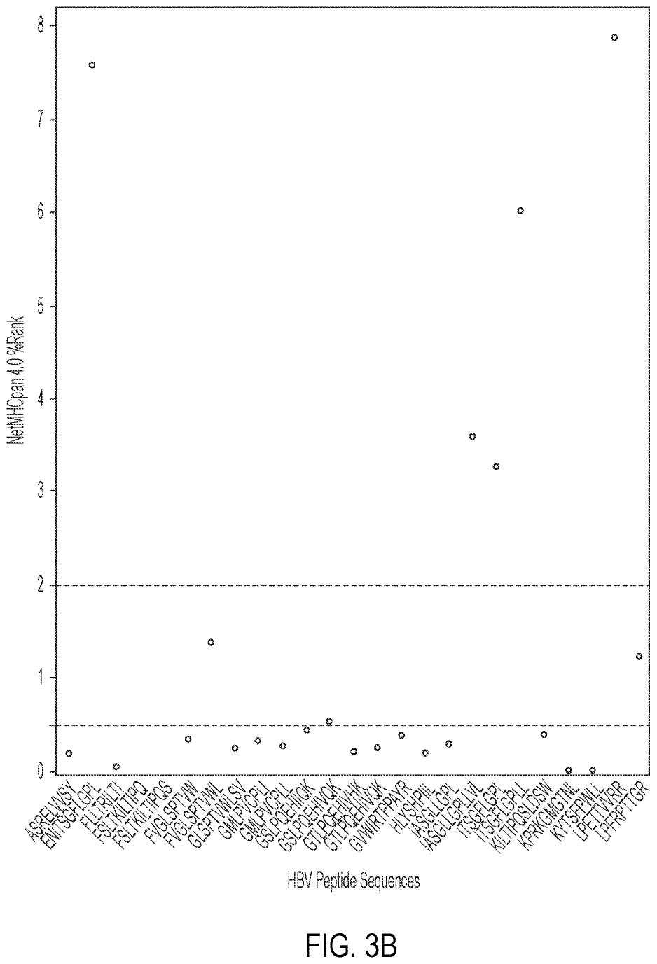

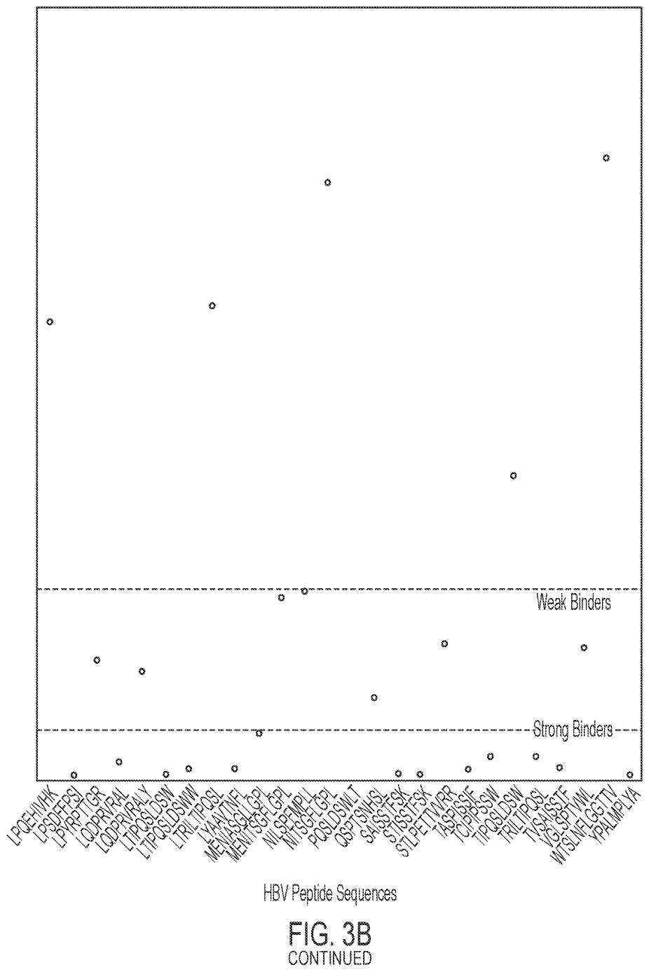

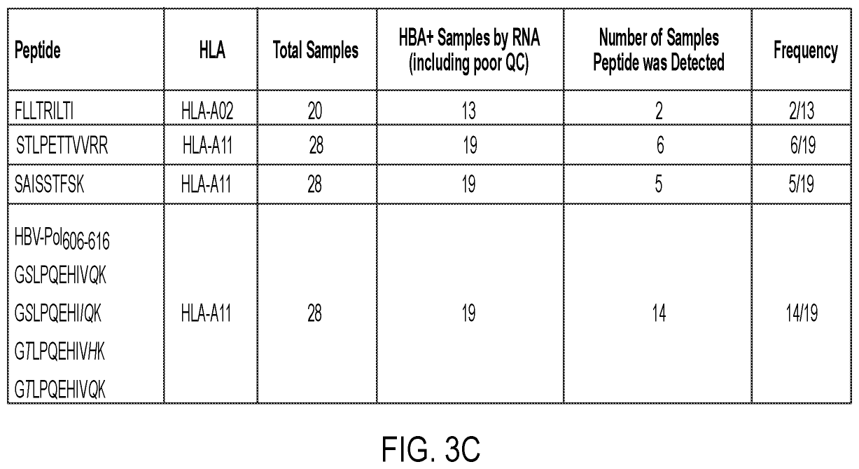

A majority of the viral peptides detected were NetMHC pan 4.0-predicted binders of the HLA-I allele present in the corresponding patient sample (FIG. 3B and Table 9). The top four HLA genotypes from HLA-A and HLA-B with highest number of HBV peptides detected as shown in FIG. 3A. A high percentage of patient samples were TILA-02 and HLA-A11 genotypes due to the Asian population being the source of the samples procured. In particular, FIG. 3B shows HBV peptide sequences identified from immunopeptidomics of patient liver samples, and their predicted binding to patient HLA alleles predicted by Pan NetMHC 4.0. Strong binders were ranked below 0.5 and weak binders between 0.5-2, according to NetMHC prediction. For the HBV peptides with predicted binding to more than one allele, the affinity rank for the strongest binding is shown. Target HBV peptides and the frequencies of detection of the target HBV peptides in HBV+ samples is shown in FIG. 3C. Table 9 displays the complete list of HBV peptides along with their binding affinities to the patient HLA alleles predicted by Pan NetMHC 4.0.

TABLE 9
HBV Peptides detected from different patient liver samples, along with their binding
predictions to the respective patient alleles by NetMHC Pan 4.0.

| | | SEQ | | | | | | | | | | | | |
|---|---|---|---|---|---|---|---|---|---|---|---|---|---|---|
| Sam- | | ID | HLA-A02:01 | | HLA-A24:02 | | HLA-B38:01 | | HLA-B39:01 | | HLA-C12:03 | | | |
| ple | Peptide | NO: | nM | Rank | nM | Rank | nM | Rank | nM | Rank | nM | Rank | | |
| Liv1 | LTIPQSLDS | 31 | 29096 | 39.37 | 12489 | 6.62 | 26885 | 15.16 | 37231 | 48.09 | 4627 | 7.76 | | |
| | W | | | | | | | | | | | | | |
| | TRILTIPQS | 47 | 8196 | 12.71 | 27317 | 18.15 | 5073 | 1.15 | 854 | 0.72 | 4038 | 7.01 | | |
| | L | | | | | | | | | | | | | |
| | FVGLSPTVW | 7 | 419 | 2.55 | 24208 | 14.84 | 21551 | 9.45 | 13454 | 8.21 | 1703 | 3.79 | | |
| | L | | | | | | | | | | | | | |
| | MENITSGFL | 36 | 7407 | 11.90 | 30723 | 22.85 | 11592 | 3.29 | 5582 | 3.30 | 7342 | 11.08 | | |
| | GPL | | | | | | | | | | | | | |
| | | SEQ | | | | | | | | | | | | |
| Sam- | | ID | HLA-A11:01 | | HLA-A24:02 | | HLA-B27:06 | | HLA-B38:02 | | HLA-C07:02 | | | |
| ple | Peptide | NO: | nM | Rank | nM | Rank | nM | Rank | nM | Rank | nM | Rank | | |
| Liv3 | LTIPQSLDS | 31 | 22309 | 19.77 | 12489 | 6.62 | 37207 | 44.09 | 30443 | 21.74 | 22764 | 20.36 | | |
| | W | | | | | | | | | | | | | |
| | TRILTIPQS | 47 | 30356 | 33.71 | 27317 | 18.15 | 86 | 0.21 | 4732 | 1.14 | 1404 | 0.85 | | |
| | L | | | | | | | | | | | | | |
| | MENITSGFL | 36 | 22612 | 20.15 | 30723 | 22.85 | 14203 | 8.34 | 7347 | 1.91 | 26054 | 25.66 | | |
| | GPL | | | | | | | | | | | | | |
| | | SEQ | | | | | | | | | | | | |
| Sam- | | ID | HLA-A02:01 | | HLA-A03:01 | | HLA-B07:02 | | HLA-B38:01 | | HLA-C04:01 | | HLA-C07:02 | |
| ple | Peptide | NO: | nM | Rank | nM | Rank | nM | Rank | nM | Rank | nM | Rank | nM | Rank |
| Liv5 | VGLSPTVWL | 49 | 13145 | 17.54 | 31679 | 39.08 | 20241 | 13.42 | 22288 | 10.15 | 18983 | 3.47 | 7917 | 5.12 |
| | FVGLSPTVW | 6 | 26225 | 34.18 | 33023 | 43.40 | 22636 | 15.99 | 20623 | 8.66 | 22588 | 5.86 | 11386 | 7.70 |
| | ITSGFLGPL | 19 | 1045 | 4.15 | 15697 | 11.92 | 4745 | 3.27 | 22083 | 9.95 | 21864 | 5.30 | 7871 | 5.08 |
| | LTIPQSLDS | 31 | 29096 | 39.37 | 25461 | 24.22 | 31090 | 30.32 | 26885 | 15.16 | 34733 | 28.34 | 22764 | 20.36 |
| | W | | | | | | | | | | | | | |
| | NITSGFLGP | 38 | 3357 | 7.61 | 25073 | 23.53 | 10658 | 6.34 | 28602 | 17.49 | 31234 | 18.32 | 23461 | 21.37 |
| | L | | | | | | | | | | | | | |
| | TRILTIPQS | 47 | 8196 | 12.71 | 26200 | 25.57 | 17618 | 11.00 | 5073 | 1.15 | 27795 | 11.77 | 1404 | 0.85 |
| | L | | | | | | | | | | | | | |
| | FVGLSPTVW | 7 | 419 | 2.55 | 26623 | 26.38 | 13183 | 7.82 | 21551 | 9.45 | 21273 | 4.85 | 10487 | 7.01 |
| | L | | | | | | | | | | | | | |
| | ITSGFLGPL | 20 | 2130 | 6.01 | 17226 | 13.32 | 15639 | 9.50 | 27652 | 16.19 | 26167 | 9.45 | 15068 | 11.00 |
| | L | | | | | | | | | | | | | |
| | LTRILTIPQ | 33 | 9300 | 13.79 | 27204 | 27.54 | 8266 | 5.02 | 26409 | 14.56 | 33625 | 24.76 | 12493 | 8.62 |
| | SL | | | | | | | | | | | | | |
| | ENITSGFLG | 2 | 17098 | 21.73 | 32737 | 42.42 | 26235 | 20.87 | 19238 | 7.58 | 36231 | 33.87 | 29808 | 33.02 |
| | PL | | | | | | | | | | | | | |
| | WTSLNFLGG | 50 | 2546 | 6.60 | 27633 | 28.46 | 26764 | 21.74 | 22903 | 10.74 | 33027 | 22.99 | 27226 | 27.75 |
| | TTV | | | | | | | | | | | | | |
| | MENITSGFL | 36 | 7407 | 11.90 | 26233 | 25.63 | 16353 | 10.02 | 11592 | 3.29 | 29118 | 13.94 | 26054 | 25.66 |
| | GPL | | | | | | | | | | | | | |
| | | SEQ | | | | | | | | | | | | |
| Sam- | | ID | HLA-A11:01 | | HLA-A30:01 | | HLA-B13:02 | | HLA-B40:01 | | HLA-C06:02 | | HLA-C07:02 | |
| ple | Peptide | NO: | nM | Rank | nM | Rank | nM | Rank | nM | Rank | nM | Rank | nM | Rank |
| Liv6 | TRILTIPQS | 47 | 30356 | 33.71 | 6805 | 15.96 | 18494 | 19.19 | 19520 | 8.15 | 950 | 0.31 | 1404 | 0.85 |
| | L | | | | | | | | | | | | | |
| | | SEQ | | | | | | | | | | | | |
| Sam- | | ID | HLA-A11:01 | | HLA-B15:02 | | HLA-B35:01 | | HLA-C03:03 | | HLA-C08:01 | | | |
| ple | Peptide | NO: | nM | Rank | nM | Rank | nM | Rank | nM | Rank | nM | Rank | | |
| Liv8 | HLYSHPIIL | 16 | 13562 | 11.51 | 505 | 0.73 | 4192 | 3.43 | 48 | 0.20 | 655 | 0.22 | | |
| | LPQEHIVHK | 26 | 13008 | 11.10 | 22135 | 21.79 | 6673 | 4.85 | 28522 | 23.30 | 34859 | 26.64 | | |
| | TASPISSIF | 44 | 21191 | 18.41 | 96 | 0.17 | 14 | 0.06 | 30 | 0.13 | 635 | 0.22 | | |
| | GTLPQEHIV | 13 | 36 | 0.22 | 39313 | 71.32 | 40817 | 63.92 | 45064 | 71.10 | 44156 | 60.17 | | |
| | HK | | | | | | | | | | | | | |
| | STLPETTVV | 43 | 337 | 1.40 | 40261 | 75.52 | 39712 | 58.32 | 41281 | 51.97 | 42232 | 49.57 | | |
| | RR | | | | | | | | | | | | | |
| | | SEQ | | | | | | | | | | | | |
| Sam- | | ID | HLA-A02:03 | | HLA-A33:03 | | HLA-B38:02 | | HLA-B48:03 | | HLA-C07:02 | | HLA-C08:01 | |
| ple | Peptide | NO: | nM | Rank | nM | Rank | nM | Rank | nM | Rank | nM | Rank | nM | Rank |
| Liv9 | IASGLLGPL | 17 | 725 | 5.75 | 27945 | 27.03 | 14479 | 5.04 | 9059 | 1.91 | 9235 | 6.09 | 904 | 0.30 |
| | FVGLSPTVW | 7 | 701 | 5.66 | 23360 | 19.68 | 22286 | 10.89 | 22516 | 8.10 | 10487 | 7.01 | 5825 | 1.86 |
| | L | | | | | | | | | | | | | |
| | NILSPFMPL | 37 | 106 | 1.98 | 9554 | 7.55 | 22579 | 11.18 | 20831 | 6.97 | 10718 | 7.19 | 17258 | 6.79 |
| | L | | | | | | | | | | | | | |
| | MENIASGLL | 35 | 5171 | 14.88 | 31427 | 34.72 | 7379 | 1.92 | 5661 | 1.04 | 27403 | 28.08 | 19912 | 8.49 |
| | GPL | | | | | | | | | | | | | |
| | IASGLLGPL | 18 | 1949 | 9.19 | 38935 | 60.51 | 29079 | 19.47 | 24768 | 9.89 | 26596 | 26.62 | 10688 | 3.60 |
| | LVL | | | | | | | | | | | | | |
| | | SEQ | | | | | | | | | | | | |
| Sam- | | ID | HLA-A11:01 | | HLA-A30:01 | | HLA-B07:02 | | HLA-B38:02 | | HLA-C07:02 | | | |
| ple | Peptide | NO: | nM | Rank | nM | Rank | nM | Rank | nM | Rank | nM | Rank | | |
| Liv10 | SAISSTFSK | 41 | 7 | 0.01 | 79 | 0.40 | 19963 | 13.15 | 34760 | 30.90 | 12903 | 8.98 | | |
| | KPRKGMGTN | 22 | 38152 | 60.46 | 6016 | 14.35 | 5 | 0.01 | 31986 | 24.68 | 8555 | 5.57 | | |
| | L | | | | | | | | | | | | | |
| | GSLPQEHIV | 12 | 90 | 0.53 | 1702 | 5.20 | 32480 | 33.90 | 46421 | 89.07 | 37263 | 53.70 | | |
| | QK | | | | | | | | | | | | | |
| | STLPETTVV | 43 | 337 | 1.40 | 12809 | 28.48 | 39562 | 61.99 | 45766 | 83.92 | 38240 | 57.22 | | |
| | RR | | | | | | | | | | | | | |
| | | SEQ | | | | | | | | | | | | |
| Sam- | | ID | HLA-A02:03 | | HLA-A11:01 | | HLA-B15:02 | | HLA-B46:01 | | HLA-C01:02 | | HLA-C08:01 | |
| ple | Peptide | NO: | nM | Rank | nM | Rank | nM | Rank | nM | Rank | nM | Rank | nM | Rank |
| Liv11 | QSPTSNHSL | 40 | 22308 | 38.77 | 33057 | 41.11 | 20942 | 19.95 | 29740 | 8.88 | 5841 | 0.84 | 10334 | 3.46 |
| | GLSPTVWLS | 8 | 11 | 0.25 | 19517 | 16.62 | 23893 | 24.71 | 33293 | 13.35 | 23921 | 9.50 | 24400 | 12.18 |
| | V | | | | | | | | | | | | | |
| | GTLPQEHIV | 14 | 32670 | 59.41 | 42 | 0.26 | 39529 | 72.29 | 44198 | 57.01 | 43988 | 61.86 | 43990 | 59.19 |
| | QK | | | | | | | | | | | | | |
| | | SEQ | | | | | | | | | | | | |
| Sam- | | ID | HLA-A02:01 | | HLA-A11:01 | | HLA-B39:01 | | HLA-B51:01 | | HLA-C07:02 | | HLA-C14:02 | |
| ple | Peptide | NO: | nM | Rank | nM | Rank | nM | Rank | nM | Rank | nM | Rank | nM | Rank |
| Liv12 | LPSDFFPSI | 27 | 10996 | 15.40 | 30264 | 33.49 | 3756 | 2.31 | 47 | 0.01 | 7131 | 4.53 | 1609 | 3.34 |
| | GMLPVCPLL | 10 | 21 | 0.28 | 22277 | 19.73 | 8690 | 5.04 | 29258 | 14.43 | 4521 | 2.77 | 1683 | 3.45 |
| | FLLTRILTI | 3 | 6 | 0.05 | 22713 | 20.28 | 3699 | 2.28 | 6605 | 1.19 | 877 | 0.56 | 484 | 1.32 |
| | GMLPVCPLI | 9 | 24 | 0.32 | 21488 | 18.75 | 18652 | 12.68 | 21117 | 6.78 | 8899 | 5.83 | 4509 | 7.25 |
| | TRILTIPQS | 47 | 8196 | 12.71 | 30356 | 33.71 | 854 | 0.72 | 35187 | 25.03 | 1404 | 0.85 | 2517 | 4.65 |
| | L | | | | | | | | | | | | | |
| | GTLPQEHIV | 14 | 32439 | 46.93 | 42 | 0.26 | 44676 | 85.81 | 45221 | 72.70 | 37571 | 54.73 | 36365 | 67.53 |
| | QK | | | | | | | | | | | | | |
| | STLPETTVV | 43 | 32125 | 46.15 | 337 | 1.40 | 43690 | 79.81 | 43010 | 56.01 | 38240 | 57.22 | 33963 | 60.12 |
| | RR | | | | | | | | | | | | | |
| | | SEQ | | | | | | | | | | | | |
| Sam- | | ID | HLA-A11:01 | | HLA-B15:02 | | HLA-B38:02 | | HLA-C07:02 | | HLA-C08:01 | | | |
| ple | Peptide | NO: | nM | Rank | nM | Rank | nM | Rank | nM | Rank | nM | Rank | | |
| Liv14 | GTLPQEHIV | 13 | 36 | 0.22 | 39313 | 71.32 | 46344 | 88.48 | 36428 | 50.86 | 44156 | 60.17 | | |
| | HK | | | | | | | | | | | | | |
| | STLPETTVV | 43 | 337 | 1.40 | 40261 | 75.52 | 45766 | 83.92 | 38240 | 57.22 | 42232 | 49.57 | | |
| | RR | | | | | | | | | | | | | |
| | | SEQ | | | | | | | | | | | | |
| Sam- | | ID | HLA-A11:01 | | HLA-A33:03 | | HLA-B15:02 | | HLA-B58:01 | | HLA-C03:02 | | HLA-C14:02 | |
| ple | Peptide | NO: | nM | Rank | nM | Rank | nM | Rank | nM | Rank | nM | Rank | nM | Rank |
| Liv15 | TVSAISSTF | 48 | 10164 | 9.11 | 16682 | 12.49 | 52 | 0.08 | 413 | 1.26 | 48 | 0.23 | 300 | 0.86 |
| | TIPQSLDSW | 46 | 32569 | 39.62 | 36537 | 50.72 | 16278 | 14.06 | 1727 | 3.21 | 10500 | 13.00 | 9536 | 13.67 |
| | GSLPQEHIV | 12 | 90 | 0.53 | 23972 | 20.54 | 39687 | 72.99 | 22222 | 30.20 | 39365 | 67.62 | 35337 | 64.26 |
| | QK | | | | | | | | | | | | | |
| | KILTIPQSL | 21 | 31585 | 36.82 | 39396 | 62.61 | 32184 | 44.40 | 76 | 0.39 | 24553 | 31.83 | 28308 | 45.45 |
| | DSW | | | | | | | | | | | | | |
| | | SEQ | | | | | | | | | | | | |
| Sam- | | ID | HLA-A11:01 | | HLA-A31:01 | | HLA-B13:01 | | HLA-B51:02 | | HLA-C03:04 | | HLA-C15:02 | |
| ple | Peptide | NO: | nM | Rank | nM | Rank | nM | Rank | nM | Rank | nM | Rank | nM | Rank |
| Liv17 | GSLPQEHIV | 12 | 90 | 0.53 | 4154 | 6.59 | 37823 | 67.07 | 44925 | 71.54 | 45332 | 72.86 | 31054 | 36.92 |
| | QK | | | | | | | | | | | | | |
| | | SEQ | | | | | | | | | | | | |
| Sam- | | ID | HLA-A02:03 | | HLA-A31:01 | | HLA-B15:25 | | HLA-B51:01 | | HLA-C04:03 | | HLA-C16:02 | |
| ple | Peptide | NO: | nM | Rank | nM | Rank | nM | Rank | nM | Rank | nM | Rank | nM | Rank |
| Liv19 | GVWIRTPPA | 15 | 23817 | 41.25 | 48 | 0.39 | 8547 | 12.93 | 45890 | 78.82 | 42559 | 48.82 | 32049 | 42.54 |
| | YR | | | | | | | | | | | | | |
| | | SEQ | HLA- | | | | | | | | | | | |
| Sam- | | ID | A02:264 | | HLA-A33:03 | | HLA-B18:02 | | HLA-B58:01 | | HLA-C03:02 | | HLA-C07:04 | |
| ple | Peptide | NO: | nM | Rank | nM | Rank | nM | Rank | nM | Rank | nM | Rank | nM | ank |
| Liv20 | PQSLDSWLT | 39 | 33326 | 61.05 | 41590 | 73.10 | 36616 | 64.94 | 36981 | 68.32 | 45560 | 91.42 | 45505 | 79.38 |
| | LPYRPTTGR | 28 | 35296 | 66.26 | 501 | 1.25 | 19820 | 16.75 | 35212 | 62.45 | 12928 | 15.66 | 36839 | 31.97 |
| | LTIPQSLDS | 31 | 25701 | 44.58 | 31548 | 35.02 | 28505 | 34.59 | 5 | 0.02 | 2437 | 4.30 | 39020 | 39.97 |
| | W | | | | | | | | | | | | | |
| | NILSPFMPL | 37 | 106 | 1.98 | 9554 | 7.55 | 22962 | 21.93 | 9724 | 12.20 | 7363 | 9.62 | 22293 | 7.11 |
| | L | | | | | | | | | | | | | |
| | FVGLSPTVW | 7 | 701 | 5.66 | 23360 | 19.68 | 30445 | 40.52 | 2170 | 3.73 | 1528 | 3.11 | 17570 | 4.05 |
| | L | | | | | | | | | | | | | |
| | FSLTKILTI | 4 | 19652 | 34.70 | 22356 | 18.32 | 27951 | 33.09 | 13474 | 16.73 | 16276 | 19.73 | 36775 | 31.77 |
| | PQ | | | | | | | | | | | | | |
| | LTIPQSLDS | 32 | 33949 | 62.69 | 34486 | 43.42 | 30254 | 39.90 | 15 | 0.10 | 12793 | 15.51 | 42872 | 59.78 |
| | WW | | | | | | | | | | | | | |
| | KILTIPQSL | 21 | 32890 | 59.91 | 39396 | 62.61 | 37533 | 69.14 | 76 | 0.39 | 24553 | 31.83 | 44363 | 70.36 |
| | DSW | | | | | | | | | | | | | |
| | MENIASGLL | 35 | 5171 | 14.88 | 31427 | 34.72 | 4023 | 2.27 | 9494 | 11.94 | 3966 | 6.07 | 30976 | 17.77 |
| | GPL | | | | | | | | | | | | | |
| | IASGLLGPL | 18 | 1949 | 9.19 | 38935 | 60.51 | 33863 | 53.20 | 2582 | 4.22 | 3767 | 5.85 | 35205 | 27.15 |
| | LVL | | | | | | | | | | | | | |
| | FSLTKILTI | 5 | 16108 | 29.66 | 34616 | 43.84 | 34239 | 54.70 | 14390 | 17.92 | 21474 | 26.92 | 36573 | 31.13 |
| | PQS | | | | | | | | | | | | | |
| | | SEQ | | | | | | | | | | | | |
| Sam- | | ID | HLA-A02:01 | | HLA-A11:01 | | HLA-B13:01 | | HLA-B55:02 | | HLA-C01:02 | | HLA-C03:04 | |
| ple | Peptide | NO: | nM | Rank | nM | Rank | nM | Rank | nM | Rank | nM | Rank | nM | Rank |
| Liv21 | GSLPQEHIV | 12 | 34081 | 51.32 | 90 | 0.53 | 37823 | 67.07 | 34522 | 43.88 | 44019 | 62.08 | 45332 | 72.86 |
| | QK | | | | | | | | | | | | | |
| | SAISSTFSK | 41 | 25337 | 32.73 | 7 | 0.01 | 17219 | 21.30 | 12770 | 7.70 | 26724 | 12.36 | 9992 | 7.14 |
| | | SEQ | | | | | | | | | | | | |
| Sam- | | ID | HLA-A24:02 | | HLA-B40:01 | | HLA-B54:01 | | HLA-C01:02 | | HLA-C04:03 | | | |
| ple | Peptide | NO: | nM | Rank | nM | Rank | nM | Rank | nM | Rank | nM | Rank | | |
| Liv25 | KYTSFPWLL | 23 | 9 | 0.01 | 15032 | 5.82 | 35585 | 36.54 | 12731 | 2.85 | 14878 | 2.60 | | |
| | YPALMPLYA | 51 | 35747 | 32.79 | 25619 | 13.13 | 9 | 0.01 | 15616 | 4.01 | 17327 | 3.50 | | |
| | LYAAVTNFL | 34 | 38 | 0.08 | 21205 | 9.31 | 22156 | 11.85 | 5393 | 0.74 | 7215 | 0.85 | | |
| | MENIASGLL | 35 | 34566 | 29.99 | 261 | 0.47 | 22934 | 12.63 | 26965 | 12.64 | 31941 | 15.90 | | |
| | GPL | | | | | | | | | | | | | |
| | | SEQ | | | | | | | | | | | | |
| Sam- | | ID | HLA-A11:01 | | HLA-B38:02 | | HLA-B51:01 | | HLA-C07:02 | | HLA-C14:02 | | | |
| ple | Peptide | NO: | nM | Rank | nM | Rank | nM | Rank | nM | Rank | nM | Rank | | |
| Liv27 | GSLPQEHIV | 12 | 90 | 0.53 | 46421 | 89.07 | 45039 | 71.07 | 37263 | 53.70 | 35337 | 64.26 | | |
| | QK | | | | | | | | | | | | | |
| | SAISSTFSK | 41 | 7 | 0.01 | 34760 | 30.90 | 32246 | 18.90 | 12903 | 8.98 | 8177 | 11.89 | | |
| Sam- | | | HLA-A11:01 | | HLA-A33:03 | | HLA-B15:25 | | HLA-B35:05 | | HLA-C04:01 | | HLA-C04:03 | |
| ple | Peptide | | nM | Rank | nM | Rank | nM | Rank | nM | Rank | nM | Rank | nM | Rank |
| Liv28 | STLPETTVV | 43 | 337 | 1.40 | 1624 | 2.61 | 38740 | 76.20 | 36529 | 58.04 | 39234 | 48.40 | 41937 | 45.41 |
| | RR | | | | | | | | | | | | | |
| | LPFRPTTGR | 25 | 16258 | 13.63 | 496 | 1.24 | 31679 | 54.65 | 4933 | 5.11 | 30972 | 17.71 | 38375 | 30.90 |
| | ASRELVVSY | 1 | 861 | 2.34 | 19925 | 15.56 | 29 | 0.19 | 648 | 1.14 | 32216 | 20.75 | 30419 | 13.57 |
| | GTLPQEHIV | 13 | 36 | 0.22 | 17824 | 13.46 | 34125 | 61.61 | 35959 | 55.91 | 37243 | 38.27 | 41939 | 45.42 |
| | HK | | | | | | | | | | | | | |
| | | SEQ | | | | | | | | | | | | |
| Sam- | | ID | HLA-A11:01 | | HLA-A33:03 | | HLA-B15:02 | | HLA-B58:01 | | HLA-C03:02 | | HLA-C08:01 | |
| ple | Peptide | NO: | nM | Rank | nM | Rank | nM | Rank | nM | Rank | nM | Rank | nM | Rank |
| Liv30 | MENIASGLL | 35 | 33145 | 41.38 | 31427 | 34.72 | 9378 | 7.67 | 9494 | 11.94 | 3966 | 6.07 | 19912 | 8.49 |
| | GPL | | | | | | | | | | | | | |
| | LTIPQSLDS | 31 | 22309 | 19.77 | 31548 | 35.02 | 9916 | 8.10 | 5 | 0.02 | 2437 | 4.30 | 26792 | 14.61 |
| | W | | | | | | | | | | | | | |
| | SAISSTFSK | 41 | 7 | 0.01 | 1439 | 2.44 | 13341 | 11.12 | 5162 | 7.06 | 2767 | 4.71 | 15553 | 5.82 |
| | VGLSPTVWL | 49 | 31128 | 35.65 | 32989 | 38.86 | 25246 | 27.23 | 7509 | 9.68 | 2746 | 4.68 | 4241 | 1.38 |
| | FVGLSPTVW | 7 | 26688 | 26.18 | 23360 | 19.68 | 11341 | 9.28 | 2170 | 3.73 | 1528 | 3.11 | 5825 | 1.86 |
| | L | | | | | | | | | | | | | |
| | FVGLSPTVW | 6 | 33595 | 42.78 | 35713 | 47.70 | 5478 | 4.69 | 65 | 0.35 | 252 | 0.91 | 10582 | 3.56 |
| | | SEQ | | | | | | | | | | | | |
| Sam- | | ID | HLA-A11:01 | | HLA-A33:03 | | HLA-B15:02 | | HLA-B58:01 | | HLA-C03:02 | | HLA-C08:01 | |
| ple | Peptide | NO: | nM | Rank | nM | Rank | nM | Rank | nM | Rank | nM | Rank | nM | Rank |
| Liv31 | MENIASGLL | 35 | 33145 | 41.38 | 31427 | 34.72 | 9378 | 7.67 | 9494 | 11.94 | 3966 | 6.07 | 19912 | 8.49 |
| | GPL | | | | | | | | | | | | | |
| | SAISSTFSK | 41 | 7 | 0.01 | 1439 | 2.44 | 13341 | 11.12 | 5162 | 7.06 | 2767 | 4.71 | 15553 | 5.82 |
| | LTIPQSLDS | 31 | 22309 | 19.77 | 31548 | 35.02 | 9916 | 8.10 | 5 | 0.02 | 2437 | 4.30 | 26792 | 14.61 |
| | W | | | | | | | | | | | | | |
| | TCIPIPSSW | 45 | 34762 | 46.71 | 33234 | 39.56 | 14934 | 12.65 | 36 | 0.23 | 2410 | 4.27 | 24737 | 12.50 |
| | FVGLSPTVW | 7 | 26688 | 26.18 | 23360 | 19.68 | 11341 | 9.28 | 2170 | 3.73 | 1528 | 3.11 | 5825 | 1.86 |
| | L | | | | | | | | | | | | | |
| | | SEQ | | | | | | | | | | | | |
| Sam- | | ID | HLA-A11:01 | | HLA-A33:03 | | HLA-B44:03 | | HLA-B56:04 | | HLA-C07:06 | | HLA-C08:01 | |
| ple | Peptide | NO: | nM | Rank | nM | Rank | nM | Rank | nM | Rank | nM | Rank | nM | Rank |
| Liv35 | STISSTFSK | 42 | 4 | 0.01 | 483 | 1.21 | 26748 | 16.10 | 13708 | 9.45 | 12332 | 5.71 | 20056 | 8.59 |
| | FVGLSPTVW | 7 | 26688 | 26.18 | 23360 | 19.68 | 35119 | 34.93 | 7552 | 4.97 | 12882 | 6.07 | 5825 | 1.86 |
| | L | | | | | | | | | | | | | |
| | GSLPQEHIV | 12 | 90 | 0.53 | 23972 | 20.54 | 40680 | 64.47 | 37529 | 56.25 | 37808 | 48.88 | 44345 | 61.48 |
| | QK | | | | | | | | | | | | | |
| | MENIASGLL | 35 | 33145 | 41.38 | 31427 | 34.72 | 3489 | 1.70 | 7503 | 4.94 | 29103 | 25.33 | 19912 | 8.49 |
| | GPL | | | | | | | | | | | | | |
| | | SEQ | | | | | | | | | | | | |
| Sam- | | ID | HLA-A11:01 | | HLA-A24:02 | | HLA-B15:12 | | HLA-B15:25 | | HLA-C03:03 | | HLA-C04:03 | |
| ple | Peptide | NO: | nM | Rank | nM | Rank | nM | Rank | nM | Rank | nM | Rank | nM | Rank |
| Liv37 | FVGLSPTVW | 7 | 26688 | 26.18 | 24208 | 14.84 | 12171 | 10.40 | 5816 | 9.60 | 1407 | 1.84 | 13585 | 2.25 |
| | L | | | | | | | | | | | | | |
| | MENIASGLL | 35 | 33145 | 41.38 | 34566 | 29.99 | 9948 | 8.24 | 8319 | 12.65 | 4692 | 3.96 | 31941 | 15.90 |
| | GPL | | | | | | | | | | | | | |
| | | SEQ | | | | | | | | | | | | |
| Sam- | | ID | HLA-A11:01 | | HLA-A24:03 | | HLA-B15:02 | | HLA-C04:03 | | HLA-C08:01 | | | |
| ple | Peptide | NO: | nM | Rank | nM | Rank | nM | Rank | nM | Rank | nM | Rank | | |
| Liv38 | LQDPRVRAL | 29 | 36481 | 53.38 | 14501 | 13.34 | 3757 | 3.46 | 1728 | 0.16 | 883 | 0.29 | | |
| | FVGLSPTVW | 6 | 33595 | 42.78 | 3684 | 5.13 | 5478 | 4.69 | 16952 | 3.35 | 10582 | 3.56 | | |
| | HLYSHPIIL | 16 | 13562 | 11.51 | 4823 | 6.05 | 505 | 0.73 | 6041 | 0.66 | 655 | 0.22 | | |
| | ASRELVVSY | 1 | 861 | 2.34 | 24190 | 23.21 | 238 | 0.39 | 30419 | 13.57 | 23276 | 11.14 | | |
| | LQDPRVRAL | 30 | 8322 | 7.94 | 14480 | 13.32 | 880 | 1.12 | 9328 | 1.23 | 16356 | 6.26 | | |
| | Y | | | | | | | | | | | | | |
| | FVGLSPTVW | 7 | 26688 | 26.18 | 16665 | 15.20 | 11341 | 9.28 | 13585 | 2.25 | 5825 | 1.86 | | |
| | L | | | | | | | | | | | | | |
| | STLPETTVV | 43 | 337 | 1.40 | 38111 | 52.34 | 40261 | 75.52 | 41937 | 45.41 | 42232 | 49.57 | | |
| | RR | | | | | | | | | | | | | |
| | GSLPQEHII | 11 | 75 | 0.45 | 37767 | 51.13 | 39891 | 73.89 | 42486 | 48.41 | 44086 | 59.74 | | |
| | QK | | | | | | | | | | | | | |
| | MENIASGLL | 35 | 33145 | 41.38 | 29108 | 30.42 | 9378 | 7.67 | 31941 | 15.90 | 19912 | 8.49 | | |
| | GPL | | | | | | | | | | | | | |
| | | SEQ | | | | | | | | | | | | |
| Sam- | | ID | HLA-A02:07 | | HLA-A02:65 | | HLA-B38:02 | | HLA-B46:01 | | HLA-C01:12 | | HLA-C07:02 | |
| ple | Peptide | NO: | nM | Rank | nM | Rank | nM | Rank | nM | Rank | nM | Rank | nM | Rank |
| Liv40 | FVGLSPTVW | 7 | 6495 | 1.39 | 18458 | 11.45 | 22286 | 10.89 | 21346 | 3.49 | 2930 | 1.80 | 10487 | 7.01 |
| | L | | | | | | | | | | | | | |
| | | SEQ | | | | | | | | | | | | |
| Sam- | | ID | HLA-A02:07 | | HLA-A33:03 | | HLA-B46:01 | | HLA-B58:01 | | HLA-C01:02 | | HLA-C03:02 | |
| ple | Peptide | NO: | nM | Rank | nM | Rank | nM | Rank | nM | Rank | nM | Rank | nM | Rank |
| Liv43 | LTIPQSLDS | 31 | 39075 | 31.83 | 31548 | 35.02 | 17545 | 2.26 | 5 | 0.02 | 30953 | 17.92 | 2437 | 4.30 |
| | W | | | | | | | | | | | | | |
| | FVGLSPTVW | 7 | 6495 | 1.39 | 23360 | 19.68 | 21346 | 3.49 | 2170 | 3.73 | 10211 | 1.98 | 1528 | 3.11 |
| | L | | | | | | | | | | | | | |
| | | SEQ | | | | | | | | | | | | |
| Sam- | | ID | HLA-A02:03 | | HLA-A33:03 | | HLA-B13:01 | | HLA-B58:01 | | HLA-C03:02 | | HLA-C03:04 | |
| ple | Peptide | NO: | nM | Rank | nM | Rank | nM | Rank | nM | Rank | nM | Rank | nM | Rank |
| Liv44 | FVGLSPTVW | 6 | 21960 | 38.22 | 35713 | 47.70 | 5718 | 5.96 | 65 | 0.35 | 252 | 0.91 | 2083 | 2.33 |
| | LTIPQSLDS | 31 | 25701 | 44.58 | 31548 | 35.02 | 5805 | 6.07 | 5 | 0.02 | 2437 | 4.30 | 14486 | 9.99 |
| | W | | | | | | | | | | | | | |
| | FVGLSPTVW | 7 | 701 | 5.66 | 23360 | 19.68 | 7703 | 8.33 | 2170 | 3.73 | 1528 | 3.11 | 1407 | 1.84 |
| | L | | | | | | | | | | | | | |
| | | SEQ | | | | | | | | | | | | |
| Sam- | | ID | HLA-A02:03 | | HLA-A24:02 | | HLA-B15:12 | | HLA-B40:01 | | HLA-C03:03 | | HLA-C07:02 | |
| ple | Peptide | NO: | nM | Rank | nM | Rank | nM | Rank | nM | Rank | nM | Rank | nM | Rank |
| Liv45 | FLLTRILTI | 3 | 7 | 0.11 | 4267 | 2.78 | 3955 | 3.20 | 21960 | 9.86 | 434 | 0.91 | 877 | 0.56 |
| | TASPISSIF | 44 | 17213 | 31.22 | 4738 | 2.97 | 706 | 0.66 | 20737 | 8.99 | 30 | 0.13 | 2738 | 1.65 |
| | TASPLSSIF | 54 | 19726 | 34.81 | 6819 | 3.90 | 721 | 0.67 | 22456 | 10.25 | 45 | 0.19 | 3921 | 2.40 |
| | | SEQ | | | | | | | | | | | | |
| Sam- | | ID | HLA-A11:01 | | HLA-A31:01 | | HLA-B51:01 | | HLA-B51:02 | | HLA-C14:02 | | HLA-C15:02 | |
| ple | Peptide | NO: | nM | Rank | nM | Rank | nM | Rank | nM | Rank | nM | Rank | nM | Rank |
| Liv47 | GTLPQEHIV | 13 | 36 | 0.22 | 2460 | 4.93 | 45220 | 72.68 | 45011 | 72.25 | 36303 | 67.33 | 29120 | 33.02 |
| | HK | | | | | | | | | | | | | |
| | | SEQ | | | | | | | | | | | | |
| Sam- | | ID | HLA-A02:03 | | HLA-A11:01 | | HLA-B15:21 | | HLA-B56:04 | | HLA-C01:02 | | HLA-C04:03 | |
| ple | Peptide | NO: | nM | Rank | nM | Rank | nM | Rank | nM | Rank | nM | Rank | nM | Rank |
| Liv48 | GTLPQEHIV | 13 | 31915 | 57.68 | 36 | 0.22 | 44521 | 82.41 | 39367 | 63.79 | 44260 | 63.76 | 41939 | 45.42 |
| | HK | | | | | | | | | | | | | |

The large surface antigen protein was the source of a few strong HLA-A02 predicted binders (NetMHC pan 4.0) that were identified: GLSPTVWLSV (S₃₄₈₋₃₅₇) (SEQ ID NO: 8), GMLPVCPLL (S₂₇₆₋₂₉₄) (SEQ ID NO: 10), FLLTRILTI (S₁₉₄₋₂₀₂) (SEQ ID NO: 3) and GMLPVCPLI (S₂₇₆₋₂₉₄) (SEQ ID NO: 9), as well as a weak binder NILSPFMPLL (S₃₇₀₋₃₇₉) (SEQ ID NO: 37) and all were found at a relatively low frequencies (Table 8). In contrast, some HLA-A11 associated peptides: STLPETTVVRR (C₁₄₁₋₁₅₁) (SEQ ID NO: 43) from capsid protein and SAISSTFSK (SEQ ID NO: 41) from polymerase were found at relatively higher frequencies (30-50%). HLA-A11 restricted HBV-genotype specific polymerase peptide GSLPQEHIVQK (P₆₀₆₋₆₁₆) (SEQ ID NO: 12) from genotype B and GTLPQEHIVHK (P₆₀₆₋₆₁₆) (SEQ ID NO: 13) and GTLPQEHIVQK (P₆₀₆₋₆₁₆) (SEQ ID NO: 14) from genotype C were detected. The former peptide variant was predicted to be a weak HLA-A11 binder, while the latter two peptides were predicted to be strong binders of HLA-A11 by NetMHC pan 4.0 (FIG. 3B). This polymerase peptide and its variants (GSLPQEHIVQK [SEQ ID NO: 12], GTLPQEHIVHK [SEQ ID NO: 13], and GTLPQEHIVQK [SEQ ID NO: 14]) have a combined frequency of over ˜76% (13 out of 17 samples) in HLA-A11 tissue samples.

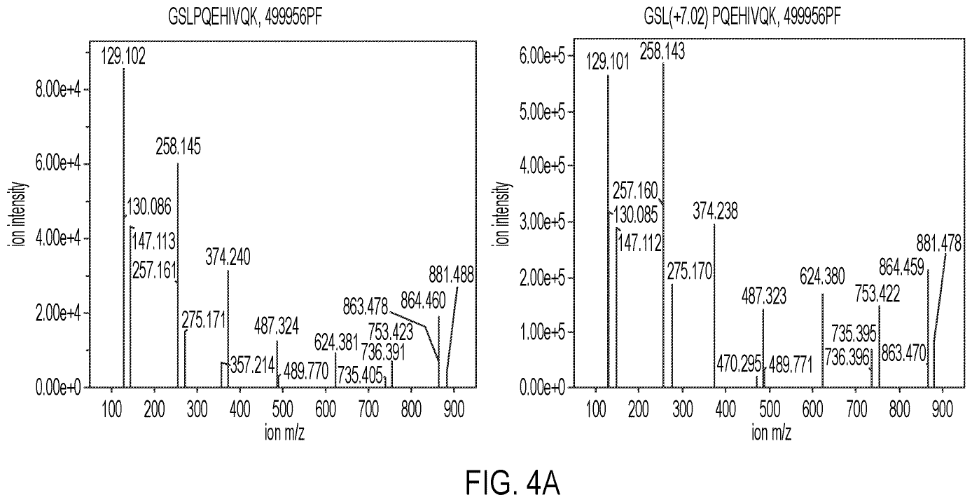

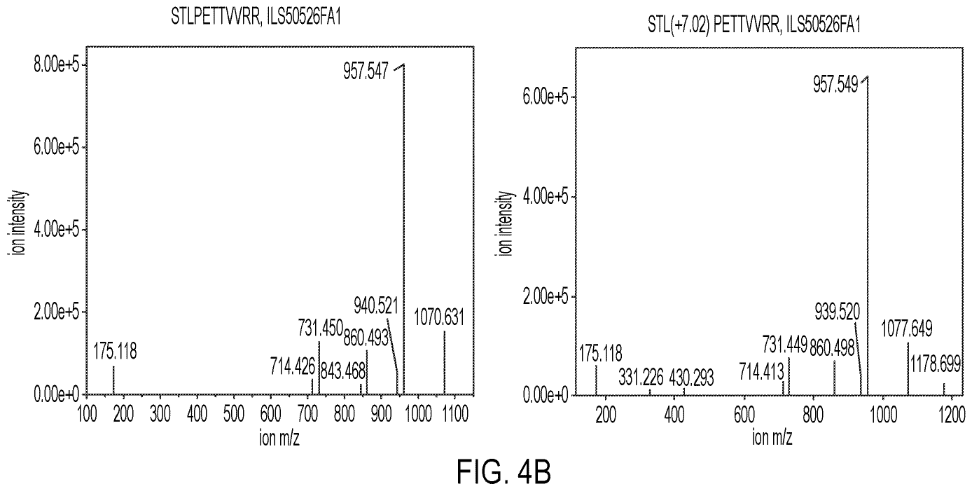

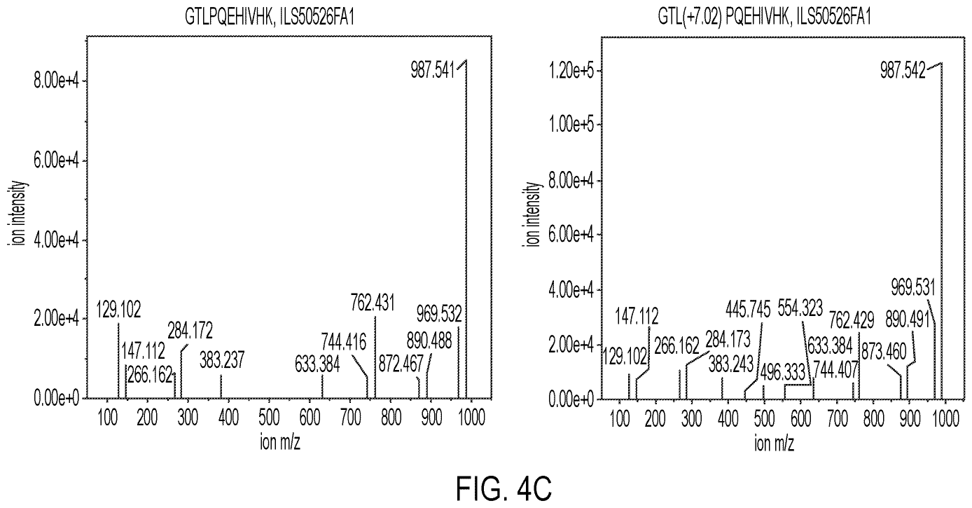

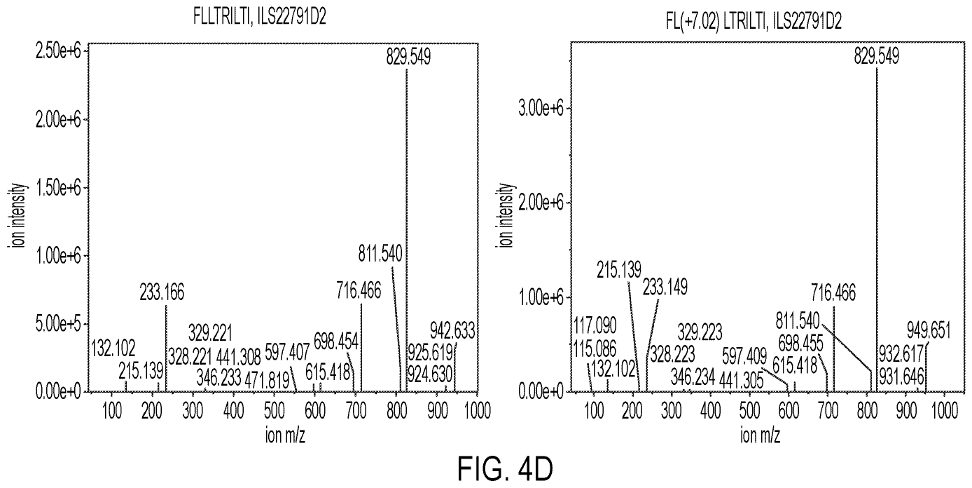

Subsequent analysis using synthetic peptide analogues was performed to validate the peptide identified above. HBV peptides predicted to bind HLA-A02 and HLA-A11 by NetMHC, including FLLTRILTI (S₁₉₄₋₂₀₂) (SEQ ID NO: 3), STLPETTVVRR (C₁₄₁₋₁₅₁) (SEQ ID NO: 43), GSLPQEHIVQK (P₆₀₆₋₆₁₆) (SEQ ID NO: 12) and variants, were validated by retention time alignment and matching the fragmentation patterns of peptides with their synthetic heavy versions (FIG. 4 and Tables 10-13). Table 10 shows MS2 ions from the fragmentation of GSLPQEHIVQK (SEQ ID NO: 12) and its heavy analogue. Table 11 shows MS2 ions from the fragmentation of STLPETTVVRR (SEQ ID NO: 43) and its heavy analogue. Table 12 shows MS2 ions from the fragmentation of GTLPQEHIVHK (SEQ ID NO: 13) and its heavy analogue. Table 13 shows MS2 ions from the fragmentation of FLLTRILTI (SEQ ID NO: 3) and its heavy analogue. Synthetic heavy peptides with sequences analogous to target HBV peptides and a modified heavy leucine (Thermo), 50 fmol, were spiked-in into the HLA-eluted peptides and separated on the nano-LC connected online to the Mass Spectrometer. The MS raw files were searched against the combined database of human Uniprot and HBV with heavy leucine (+7.02) as a variable modification using PEAKSX. The fragmentation pattern (b- and y-ions) of HBV peptides and their heavy analogues along with their co-elution and identification in the same MS1 scan were used for peptide validation. For each peptide, the ion charge was 1, except for peptides marked with an asterisk (*) which had an ion charge of 2. FR, Fraction; Mod, Modification.

TABLE 10
MS2 ions from the fragmentation of GSLPQEHIVQK (SEQ ID NO: 12) and
its heavy analogue.

| | | | | | | | | | | | | | | Ion | |
|---|---|---|---|---|---|---|---|---|---|---|---|---|---|---|---|
| | | | | | | | Sample | | | | Ion | Theo | m/z | Inten- | |
| Peptide | Mass | m/z | RT | z | FR | Scan | ID | Protein | Ion | Pos | m/z | m/z | Error | sity | Mod |
| GSLPQEH | 1234. | 412. | 24.66 | 3 | 64 | F64: | 499956 | Poly- | y1 | 1 | 147. | 147.1 | 0.0002 | 4.33E+ | |
| IVQK | 667 | 5624 | | | | 9446 | PF | merase_ | | | 1126 | 128 | | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y2 | 2 | 275. | 275.1 | 0 | 1.51E+ | |
| IVQK | 667 | 5624 | | | | 9446 | PF | merase_ | | | 1714 | 714 | | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y3 | 3 | 374. | 374.2 | - | 3.16E+ | |
| IVQK | 667 | 5624 | | | | 9446 | PF | merase_ | | | 2401 | 398 | 0.0003 | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y4 | 4 | 487. | 487.3 | - | 1.24E+ | |
| IVQK | 667 | 5624 | | | | 9446 | PF | merase_ | | | 3242 | 238 | 0.0003 | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y5 | 5 | 624. | 624.3 | 0.002 | 8.92E+ | |
| IVQK | 667 | 5624 | | | | 9446 | PF | merase_ | | | 3808 | 828 | | 03 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y6 | 6 | 753. | 753.4 | 0.0022 | 7.72E+ | |
| IVQK | 667 | 5624 | | | | 9446 | PF | merase_ | | | 4232 | 254 | | 03 | |
| (SEQ ID | | | | | | | | 112615 | | | | | | | |
| NO: 12) | | | | | | | | 6_1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y7 | 7 | 881. | 881.4 | - | 4.61E+ | |
| IVQK | 667 | 5624 | | | | 9446 | PF | merase_ | | | 4877 | 839 | 0.0038 | 03 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y1- | 1 | 129. | 129.1 | 0.0002 | 8.64E+ | |
| IVQK | 667 | 5624 | | | | 9446 | PF | merase_ | H₂O | | 102 | 022 | | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y2- | 2 | 257. | 257.1 | 0.0001 | 2.82E+ | |
| IVQK | 667 | 5624 | | | | 9446 | PF | merase_ | H₂O | | 1607 | 608 | | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y6- | 6 | 735. | 735.4 | 0.0094 | 2.55E+ | |
| IVQK | 667 | 5624 | | | | 9446 | PF | merase_ | H₂O | | 4054 | 148 | | 03 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y7- | 7 | 863. | 863.4 | - | 4.51E+ | |
| IVQK | 667 | 5624 | | | | 9446 | PF | merase_ | H₂O | | 4782 | 733 | 0.0049 | 03 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y1- | 1 | 130. | 130.0 | - | 4.60E+ | |
| IVQK | 667 | 5624 | | | | 9446 | PF | merase_ | NH₃ | | 086 | 858 | 0.0002 | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y2- | 2 | 258. | 258.1 | - | 6.04E+ | |
| IVQK | 667 | 5624 | | | | 9446 | PF | merase_ | NH₃ | | 1447 | 444 | 0.0003 | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y3- | 3 | 357. | 357.2 | - | 5.76E+ | |
| IVQK | 667 | 5624 | | | | 9446 | PF | merase_ | NH₃ | | 2136 | 128 | 0.0008 | 03 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y6- | 6 | 736. | 736.3 | 0.0073 | 2.62E+ | |
| IVQK | 667 | 5624 | | | | 9446 | PF | merase_ | NH₃ | | 3911 | 984 | | 03 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y7- | 7 | 864. | 864.4 | - | 1.91E+ | |
| IVQK | 667 | 5624 | | | | 9446 | PF | merase_ | NH₃ | | 4601 | 569 | 0.0032 | 04 | |
| (SEQ ID | | | | | | | | 1126156 | | | | | | | |
| NO: 12) | | | | | | | | _1_1. | | | | | | | |
| | | | | | | | | final. | | | | | | | |
| | | | | | | | | contigs | | | | | | | |
| GSLPQEH | 1234. | 412. | 24.66 | 3 | 64 | F64: | 499956 | Poly- | y8 | 8 | 489. | 489.7 | - | 2.80E+ | |
| IVQK | 667 | 5624 | | | | 9446 | PF | merase_ | /2 | | 7695 | 683 | 0.0012 | 03 | |
| (SEQ ID | | | | | | | | 1126156 | +/ | | | | | | |
| NO: 12) | | | | | | | | _1_1. | | | | | | | |
| | | | | | | | | final. | | | | | | | |
| | | | | | | | | contigs | | | | | | | |
| GSL | 1241. | 414. | 24.69 | 3 | 64 | F64: | 499956 | Poly- | y1 | 1 | 147. | 147.1 | 0.0012 | 2.88E+ | 13C(6) |
| (+7.02) | 6841 | 9017 | | | | 9463 | PF | merase_ | | | 1116 | 128 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y2 | 2 | 275. | 275.1 | 0.0013 | 1.84E+ | 13C(6) |
| (+7.02) | 6841 | 9017 | | | | 9463 | PF | merase_ | | | 17 | 714 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y3 | 3 | 374. | 374.2 | 0.0016 | 2.98E+ | 13C(6) |
| (+7.02) | 6841 | 9017 | | | | 9463 | PF | merase_ | | | 2381 | 398 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y4 | 4 | 487. | 487.3 | 0.0013 | 1.41E+ | 13C(6) |
| (+7.02) | 6841 | 9017 | | | | 9463 | PF | merase_ | | | 3225 | 238 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y5 | 5 | 624. | 624.3 | 0.0032 | 1.71E+ | 13C(6) |
| (+7.02) | 6841 | 9017 | | | | 9463 | PF | merase_ | | | 3795 | 828 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y6 | 6 | 753. | 753.4 | 0.0031 | 1.44E+ | 13C(6) |
| (+7.02) | 6841 | 9017 | | | | | PF | merase_ | | | 4222 | 254 | | 05 | 15N(1) |
| PQEHI | | | | | | 9463 | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64 | 499956 | Poly- | y7 | 7 | 881. | 881.4 | 0.0056 | 8.00E+ | 13C(6) |
| (+7.02) | 6841 | 9017 | | | | 9463 | PF | merase_ | | | 4783 | 839 | | 04 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y1- | 1 | 129. | 129.1 | 0.0012 | 5.66E+ | 13C(6) |
| (+7.02) | 6841 | 9017 | | | | 9463 | PF | merase_ | H₂O | | 1011 | 022 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y2- | 2 | 257. | 257.1 | 0.0013 | 3.31E+ | 13C(6) |
| (+7.02) | 6841 | 9017 | | | | 9463 | PF | merase_ | H2O | | 1595 | 608 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Polym- | y6- | 6 | 735. | 735.4 | 0.0194 | 6.71E+ | 13C(6) |
| (+7.02) | 6841 | 9017 | | | | 9463 | PF | erase_ | H₂O | | 3954 | 148 | | 04 | 15N(1) |
| PQEHI | | | | | | | | 1126156 | | | | | | | Silac |
| VQK | | | | | | | | 1_1_. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y7- | 7 | 863. | 863.4 | 0.0031 | 3.93E+ | 13C(6) |
| (+7.02) | 6841 | 9017 | | | | 9463 | PF | merase_ | H₂O | | 4702 | 733 | | 04 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y1- | 1 | 130. | 130.0 | 0.0007 | 3.18E+ | 13C(6) |
| (+7.02) | 6841 | 9017 | | | | 9463 | PF | merase_ | NH₃ | | 0851 | 858 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y2- | 2 | 258. | 258.1 | 0.001 | 5.85E+ | 13C(6) |
| (+7.02) | 6841 | 9017 | | | | 9463 | PF | merase_ | NH₃ | | 1434 | 444 | | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y4- | 4 | 470. | 470.2 | 0.0021 | 1.97E+ | 13C(6) |
| (+7.02) | 6841 | 9017 | | | | 9463 | PF | merase_ | NH₃ | | 2947 | 968 | | 04 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y6- | 6 | 736. | 736.3 | 0.0026 | 3.07E+ | 13C(6) |
| (+7.02) | 6841 | 9017 | | | | 9463 | PF | merase_ | NH₃ | | 3958 | 984 | | 04 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y7- | 7 | 864. | 864.4 | - | 2.14E+ | 13C(6) |
| (+7.02) | 6841 | 9017 | | | | 9463 | PF | merase_ | NH₃ | | 4594 | 569 | 0.0024 | 05 | 15N(1) |
| PQEHI | | | | | | | | 1126156 | | | | | | | Silac |
| VQK | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |
| GSL | 1241. | 414. | 24.69 | 3 | 64 | F64: | 499956 | Poly- | y8- | | 489. | 489.7 | - | 3.38E+ | 13C(6) |
| (+7.02) | 6841 | 9017 | | | | 9463 | PF | merase_ | /2 | | 7708 | 683 | 0.0024 | 04 | 15N(1) |
| PQEHI | | | | | | | | 1126156 | +/ | | | | | | Silac |
| VQK* | | | | | | | | _1_1. | | | | | | | label, |
| (SEQ ID | | | | | | | | final. | | | | | | | 7.01 |
| NO: | | | | | | | | contigs | | | | | | | 72,3, |
| 108) | | | | | | | | | | | | | | | L |

TABLE 11
MS2 ions from the fragmentation of STLPETTVVRR (SEQ ID NO: 43)
and its heavy analogue.

| | | | | | | | Sam- | | | | | | | Ion | |
|---|---|---|---|---|---|---|---|---|---|---|---|---|---|---|---|
| | | | | | | | ple | | | | Ion | Theo | m/z | inten- | |
| Peptide | Mass | m/z | RT | z | FR | Scan | ID | Protein | Ion | Pos | m/z | m/z | Error | sity | Mod |
| STLPETT | 1257. | 420. | 30.75 | 3 | 34 | F34: | ILS | Poly- | y1 | 1 | 175. | 175.11 | 0.0007 | 7.01E+ | |
| VVRR | 7041 | 2408 | | | | 1255 | 50526 | merase_ | | | 11 | 89 | | 04 | |
| (SEQ ID | | | | | | 4 | FA1 | 1126162 | | | 82 | | | | |
| NO: 43) | | | | | | | | _1_1. | | | | | | | |
| | | | | | | | | final. | | | | | | | |
| | | | | | | | | contigs | | | | | | | |
| STLPETT | 1257. | 420. | 30.75 | 3 | 34 | F34: | ILS | Poly- | y6 | 6 | 731. | 731.45 | 0.0026 | 1.29E+ | |
| VVRR | 7041 | 2408 | | | | 1255 | 50526 | merase_ | | | 44 | 22 | | 05 | |
| (SEQ ID | | | | | | 4 | FA1 | 1126162 | | | 96 | | | | |
| NO: 43) | | | | | | | | _1_1. | | | | | | | |
| | | | | | | | | final. | | | | | | | |
| | | | | | | | | contigs | | | | | | | |
| STLPETT | 1257. | 420. | 30.75 | 3 | 34 | F34: | ILS | Poly- | y7 | 7 | 860. | 860.49 | 0.0016 | 1.06E+ | |
| VVRR | 7041 | 2408 | | | | 1255 | 50526 | merase_ | | | 49 | 48 | | 05 | |
| (SEQ ID | | | | | | 4 | FA1 | 1126162 | | | 32 | | | | |
| NO: 43) | | | | | | | | _1_1. | | | | | | | |
| | | | | | | | | final. | | | | | | | |
| | | | | | | | | contigs | | | | | | | |
| STLPETT | 1257. | 420. | 30.75 | 3 | 34 | F34: | ILS | Poly- | y8 | 8 | 957. | 957.54 | 0.0002 | 8.02E+ | |
| VVRR | 7041 | 2408 | | | | 1255 | 50526 | merase_ | | | 54 | 75 | | 05 | |
| (SEQ ID | | | | | | 4 | FA1 | 1126162 | | | 73 | | | | |
| NO: 43) | | | | | | | | _1_1. | | | | | | | |
| | | | | | | | | final. | | | | | | | |
| | | | | | | | | contigs | | | | | | | |
| STLPETT | 1257. | 420. | 30.75 | 3 | 34 | F34: | ILS | Poly- | y9 | 9 | 107 | 1070.6 | 0.0005 | 1.51E+ | |
| VVRR | 7041 | 2408 | | | | 1255 | 50526 | merase_ | | | 0.6 | 316 | | 05 | |
| (SEQ ID | | | | | | 4 | FA1 | 1126162 | | | 311 | | | | |
| NO: 43) | | | | | | | | _1_1. | | | | | | | |
| | | | | | | | | final. | | | | | | | |
| | | | | | | | | contigs | | | | | | | |
| STLPETT | 1257. | 420. | 30.75 | 3 | 34 | F34: | ILS | Poly- | y6- | 6 | 714. | 714.42 | −0.0003 | 3.78E+ | |
| VVRR | 7041 | 2408 | | | | 1255 | 50526 | merase_ | NH | | 42 | 52 | | 04 | |
| (SEQ ID | | | | | | 4 | FA1 | 1126162 | 3 | | 55 | | | | |
| NO: 43) | | | | | | | | _1_1. | | | | | | | |
| | | | | | | | | final. | | | | | | | |
| | | | | | | | | contigs | | | | | | | |
| STLPETT | 1257. | 420. | 30.75 | 3 | 34 | F34: | ILS | Poly- | y7- | 7 | 843. | 843.46 | −0.0003 | 2.40E+ | |
| VVRR | 7041 | 2408 | | | | 1255 | 50526 | merase_ | NH | | 46 | 78 | | 04 | |
| (SEQ ID | | | | | | 4 | FA1 | 1126162 | 3 | | 81 | | | | |
| NO: 43) | | | | | | | | _1_1. | | | | | | | |
| | | | | | | | | final. | | | | | | | |
| | | | | | | | | contigs | | | | | | | |
| STLPETT | 1257. | 420. | 30.75 | 3 | 34 | F34: | ILS | Poly- | y8- | 8 | 940. | 940.52 | −0.0008 | 5.05E+ | |
| VVRR | 7041 | 2408 | | | | 1255 | 50526 | merase_ | NH | | 52 | 06 | | 04 | |
| (SEQ ID | | | | | | 4 | FA1 | 1126162 | 3 | | 14 | | | | |
| NO: 43) | | | | | | | | _1_1. | | | | | | | |
| | | | | | | | | final. | | | | | | | |
| | | | | | | | | contigs | | | | | | | |
| STL | 1264. | 422. | 30.59 | 3 | 34 | F34: | ILS | Poly- | y1 | 1 | 175. | 175.11 | 0.0007 | 5.86E+ | 13C(6) |
| (+7.02) | 7212 | 5807 | | | | 1246 | 50526 | merase_ | | | 11 | 89 | | 04 | 15N(1) |
| PETTV | | | | | | 4 | FA1 | 1126162 | | | 83 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y2 | 2 | 331. | 331.22 | −0.0057 | 5.93E+ | 13C(6) |
| (+7.02) | 7212 | 5807 | | | | 1246 | 50526 | merase_ | | | 22 | | | 03 | 15N(1) |
| PETTV | | | | | | 4 | FA1 | 1126162 | | | 57 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y3 | 3 | 430. | 430.28 | −0.0045 | 9.83E+ | 13C(6) |
| (+7.02) | 7212 | 5807 | | | | 1246 | 50526 | merase_ | | | 29 | 85 | | 03 | 15N(1) |
| PETTV | | | | | | 4 | FA1 | 1126162 | | | 29 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y6 | 6 | 731. | 731.45 | 0.0032 | 7.53E+ | 13C(6) |
| (+7.02) | 7212 | 5807 | | | | 1246 | 50526 | merase_ | | | 44 | 22 | | 04 | 15N(1) |
| PETTV | | | | | | 4 | FA1 | 1126162 | | | 9 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y7 | 7 | 860. | 860.49 | −0.0031 | 6.70E+ | 13C(6) |
| (+7.02) | 7212 | 5807 | | | | 1246 | 50526 | merase_ | | | 49 | 48 | | 04 | 15N(1) |
| PETTV | | | | | | 4 | FA1 | 1126162 | | | 79 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y8 | 8 | 957. | 957.54 | −0.0011 | 6.39E+ | 13C(6) |
| (+7.02) | 7212 | 5807 | | | | 1246 | 50526 | merase_ | | | 54 | 75 | | 05 | 15N(1) |
| PETTV | | | | | | 4 | FA1 | 1126162 | | | 86 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y9 | 9 | 107 | 1077.6 | −0.0006 | 1.06E+ | 13C(6) |
| (+7.02) | 7212 | 5807 | | | | 1246 | 50526 | merase_ | | | 7.6 | 488 | | 05 | 15N(1) |
| PETTV | | | | | | 4 | FA1 | 1126162 | | | 494 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y10 | 10 | 117 | 1178.6 | −0.0024 | 2.59E+ | 13C(6) |
| (+7.02) | 7212 | 5807 | | | | 1246 | 50526 | merase_ | | | 8.6 | 965 | | 04 | 15N(1) |
| PETTV | | | | | | 4 | FA1 | 1126162 | | | 99 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264 | 422. | 30.59 | 3 | 34 | F34: | ILS | Poly- | y8- | 8 | 939. | 939.53 | 0.0168 | 3.29E+ | 13C(6) |
| (+7.02) | 7212 | 5807 | | | | 1246 | 50526 | merase_ | H2 | | .52 | 7 | | 04 | 15N(1) |
| PETTV | | | | | | 4 | FA1 | 1126162 | O | | 02 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |
| STL | 1264. | 422. | 30.59 | 3 | 34 | F34: | ILS | Poly- | y6- | 6 | 714. | 714.42 | 0.0124 | 2.80E+ | 13C(6) |
| (+7.02) | 7212 | 5807 | | | | 1246 | 50526 | merase_ | NH | | 41 | 52 | | 04 | 15N(1) |
| PETTV | | | | | | 4 | FA1 | 1126162 | 3 | | 28 | | | | Silac |
| VRR | | | | | | | | _1_1. | | | | | | | label, |
| (SEQ ID | | | | | | | | final. | | | | | | | 7.0 |
| NO: 43) | | | | | | | | contigs | | | | | | | 172,3, |
| | | | | | | | | | | | | | | | L |

TABLE 12
MS2 ions from the fragmentation of GTLPQEHIVHK (SEQ ID NO: 13)
and its heavy analogue.

| | | | | | | | Sam- | | | | | | | Ion | |
|---|---|---|---|---|---|---|---|---|---|---|---|---|---|---|---|
| | | | | | | | ple | | | | Ion | Theo | m/z | inten- | |
| Peptide | Mass | m/z | RT | z | FR | Scan | ID | Protein | Ion | Pos | m/z | m/z | Error | sity | Mod |
| GTLPQEH | 1257. | 420. | 20.11 | 3 | 34 | F34:6 | ILS | Poly- | y1 | 1 | 147. | 147.11 | 0.0004 | 8.14E+ | |
| IVHK | 683 | 2345 | | | | 507 | 50526 | merase_ | | | 11 | 28 | | 03 | |
| (SEQ ID | | | | | | | FA1 | 1227150 | | | 24 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y2 | 2 | 284. | 284.17 | −0.0006 | 1.19E+ | |
| IVHK | 683 | 2345 | | | | 507 | 50526 | merase_ | | | 17 | 17 | | 04 | |
| (SEQ ID | | | | | | | FA1 | 1227150 | | | 23 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y3 | 3 | 383. | 383.24 | 0.0031 | 5.94E+ | |
| IVHK | 683 | 2345 | | | | 507 | 50526 | merase_ | | | 23 | 01 | | 03 | |
| (SEQ ID | | | | | | | FA1 | 1227150 | | | 7 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y5 | 5 | 633. | 633.38 | −0.0007 | 5.90E+ | |
| IVHK | 683 | 2345 | | | | 507 | 50526 | merase_ | | | 38 | 31 | | 03 | |
| (SEQ ID | | | | | | | FA1 | 1227150 | | | 38 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y6 | 6 | 762. | 762.42 | −0.0052 | 2.05E+ | |
| IVHK | 683 | 2345 | | | | 507 | 50526 | merase_ | | | 43 | 57 | | 04 | |
| (SEQ ID | | | | | | | FA1 | 1227150 | | | 09 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y7 | 7 | 890. | 890.48 | −0.0039 | 4.86E+ | |
| IVHK | 683 | 2345 | | | | 507 | 50526 | merase_ | | | 48 | 43 | | 03 | |
| (SEQ ID | | | | | | | FA1 | 1227150 | | | 82 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y8 | 8 | 987. | 987.53 | −0.0035 | 8.48E+ | |
| IVHK | 683 | 2345 | | | | 507 | 50526 | merase_ | | | 54 | 7 | | 04 | |
| (SEQ ID | | | | | | | FA1 | 1227150 | | | 05 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y1- | 1 | 129. | 129.10 | 0.0005 | 1.90E+ | |
| IVHK | 683 | 2345 | | | | 507 | 50526 | merase_ | H2 | | 10 | 22 | | 04 | |
| (SEQ ID | | | | | | | FA1 | 1227150 | O | | 18 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y2- | 2 | 266. | 266.16 | −0.0006 | 6.55E+ | |
| IVHK | 683 | 2345 | | | | 507 | 50526 | merase_ | H2 | | 16 | 11 | | 03 | |
| (SEQ ID | | | | | | | FA1 | 1227150 | O | | 17 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y6- | 6 | 744. | 744.41 | −0.0004 | 5.49E+ | |
| IVHK | 683 | 2345 | | | | 507 | 50526 | merase_ | H2 | | 41 | 51 | | 03 | |
| (SEQ ID | | | | | | | FA1 | 1227150 | O | | 55 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y7- | 7 | 872. | 872.47 | 0.0064 | 4.75E+ | |
| IVHK | 683 | 2345 | | | | 507 | 50526 | merase_ | H2 | | 46 | 37 | | 03 | |
| (SEQ ID | | | | | | | FA1 | 1227150 | O | | 73 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTLPQEH | 1257. | 420. | 20.11 | 3 | 34 | F34:6 | ILS | Poly- | y8- | 8 | 969. | 969.52 | −0.0052 | 1.83E+ | |
| IVHK | 683 | 2345 | | | | 507 | 50526 | merase_ | H2 | | 53 | 64 | | 04 | |
| (SEQ ID | | | | | | | FA1 | 1227150 | O | | 17 | | | | |
| NO: 13) | | | | | | | | _1_1. | | | | | | | |
| | | | | | | | | final. | | | | | | | |
| | | | | | | | | contigs | | | | | | | |
| | | | | | | | | _recon- | | | | | | | |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y1 | 1 | 147. | 147.11 | 0.0006 | 7.87E+ | 13C(6) |
| (+7.02) | 7001 | 5737 | | | | 600 | 50526 | merase_ | | | 11 | 28 | | 03 | 15N(1) |
| PQEHI | | | | | | | FA1 | 1227150 | | | 22 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y2 | 2 | 284. | 284.17 | −0.0008 | 1.17E+ | 13C(6) |
| (+7.02) | 7001 | 5737 | | | | 600 | 50526 | merase_ | | | 17 | 17 | | 04 | 15N(1) |
| PQEHI | | | | | | | FA1 | 1227150 | | | 25 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y3 | 3 | 383. | 383.24 | −0.0024 | 8.46E+ | 13C(6) |
| (+7.02) | 7001 | 5737 | | | | 600 | 50526 | merase_ | | | 24 | 01 | | 03 | 15N(1) |
| PQEHI | | | | | | | FA1 | 1227150 | | | 25 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y4 | 4 | 496. | 496.32 | −0.0088 | 5.00E+ | 13C(6) |
| (+7.02) | 7001 | 5737 | | | | 600 | 50526 | merase_ | | | 33 | 42 | | 03 | 15N(1) |
| PQEHI | | | | | | | FA1 | 1227150 | | | 3 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y5 | 5 | 633. | 633.38 | −0.001 | 7.42E+ | 13C(6) |
| (+7.02) | 7001 | 5737 | | | | 600 | 50526 | merase_ | | | 38 | 31 | | 03 | 15N(1) |
| PQEHI | | | | | | | FA1 | 1227150 | | | 41 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y6 | 6 | 762. | 762.42 | −0.0037 | 2.49E+ | 13C(6) |
| (+7.02) | 7001 | 5737 | | | | 600 | 50526 | merase_ | | | 42 | 57 | | 04 | 15N(1) |
| PQEHI | | | | | | | FA1 | 1227150 | | | 93 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y7 | 7 | 890. | 890.48 | −0.0068 | 1.16E+ | 13C(6) |
| (+7.02) | 7001 | 5737 | | | | 600 | 50526 | merase_ | | | 49 | 43 | | 04 | 15N(1) |
| PQEHI | | | | | | | FA1 | 1227150 | | | 11 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y8 | 8 | 987. | 987.53 | −0.0049 | 1.23E+ | 13C(6) |
| (+7.02) | 7001 | 5737 | | | | 600 | 50526 | merase_ | | | 54 | 7 | | 05 | 15N(1) |
| PQEHI | | | | | | | FA1 | 1227150 | | | 19 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y1- | 1 | 129. | 129.10 | 0.0004 | 9.25E+ | 13C(6) |
| (+7.02) | 7001 | 5737 | | | | 600 | 50526 | merase_ | H2 | | 10 | 22 | | 03 | 15N(1) |
| PQEHI | | | | | | | FA1 | 1227150 | O | | 18 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y2- | 2 | 266. | 266.16 | −0.0013 | 1.07E+ | 13C(6) |
| (+7.02) | 7001 | 5737 | | | | 600 | 50526 | merase_ | H2 | | 16 | 11 | | 04 | 15N(1) |
| PQEHI | | | | | | | FA1 | 1227150 | O | | 24 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y6- | 6 | 744. | 744.41 | 0.0085 | 5.41E+ | 13C(6) |
| (+7.02) | 7001 | 5737 | | | | 600 | 50526 | merase_ | H2 | | 40 | 51 | | 03 | 15N(1) |
| PQEHI | | | | | | | FA1 | 1227150 | O | | 66 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y8- | 8 | 969. | 969.52 | −0.0043 | 2.75E+ | 13C(6) |
| (+7.02) | 7001 | 5737 | | | | 600 | 50526 | merase_ | H2 | | 53 | 64 | | 04 | 15N(1) |
| PQEHI | | | | | | | FA1 | 1227150 | O | | 07 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y7- | 7 | 873. | 873.45 | −0.0024 | 9.06E+ | 13C(6) |
| (+7.02) | 7001 | 5737 | | | | 600 | 50526 | merase_ | NH | | 45 | 73 | | 03 | 15N(1) |
| PQEHI | | | | | | | FA1 | 1227150 | 3 | | 97 | | | | Silac |
| VHK | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y7- | 7 | 445. | 445.74 | −0.0033 | 3.82E+ | 13C(6) |
| (+7.02) | 7001 | 5737 | | | | 600 | 50526 | merase_ | /2 | | 74 | 21 | | 03 | 15N(1) |
| PQEHI | | | | | | | FA1 | 1227150 | +/ | | 54 | | | | Silac |
| VHK* | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |
| GTL | 1264. | 422. | 20.29 | 3 | 34 | F34:6 | ILS | Poly- | y9- | 9 | 554. | 554.31 | −0.0039 | 4.95E+ | 13C(6) |
| (+7.02) | 7001 | 5737 | | | | 600 | 50526 | merase_ | /2 | | 32 | 91 | | 03 | 15N(1) |
| PQEHI | | | | | | | FA1 | 1227150 | +/ | | 3 | | | | Silac |
| VHK* | | | | | | | | _1_1. | | | | | | | label, |
| (SEQ ID | | | | | | | | final. | | | | | | | 7.0 |
| NO: 13) | | | | | | | | contigs | | | | | | | 172,3, |
| | | | | | | | | _recon- | | | | | | | L |
| | | | | | | | | sti- | | | | | | | |
| | | | | | | | | tuted_ | | | | | | | |
| | | | | | | | | genome | | | | | | | |

TABLE 13
MS2 ions from the fragmentation of FLLTRILTI (SEQ ID NO: 3)
and its heavy analogue.

| | | | | | | | Sam- | | | | | | | Ion | |
|---|---|---|---|---|---|---|---|---|---|---|---|---|---|---|---|
| | | | | | | | ple | | | | Ion | Theo | m/z | Inten- | |
| Peptide | Mass | m/z | RT | z | FR | Scan | ID | Protein | Ion | Pos | m/z | m/z | Error | sity | Mod |
| FLLTRIL | 1088. | 545. | 99.4 | 2 | 6 | F6:5 | ILS | S_837_bp | y1 | 1 | 132. | 132.10 | 0.0002 | 7.65E+ | |
| TI | 6958 | 3545 | | | | 2577 | 22791 | _1126159 | | | 1017 | 19 | | 04 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y2 | 2 | 233. | 233.14 | −0.0166 | 6.30E+ | |
| TI | 6958 | 3545 | | | | 2577 | 22791 | _1126159 | | | 1662 | 96 | | 05 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y3 | 3 | 346. | 346.23 | 0.001 | 7.25E+ | |
| TI | 6958 | 3545 | | | | 2577 | 22791 | _1126159 | | | 2327 | 36 | | 03 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y5 | 5 | 615. | 615.41 | 0.0005 | 6.66E+ | |
| TI | 6958 | 3545 | | | | 2577 | 22791 | _1126159 | | | 4183 | 88 | | 04 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y6 | 6 | 716. | 716.46 | 0.0002 | 6.51E+ | |
| TI | 6958 | 3545 | | | | 2577 | 22791 | _1126159 | | | 4663 | 65 | | 05 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y7 | 7 | 829. | 829.55 | 0.0012 | 2.37E+ | |
| TI | 6958 | 3545 | | | | 2577 | 22791 | _1126159 | | | 5494 | 05 | | 06 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y8 | 8 | 942. | 942.63 | 0.002 | 3.12E+ | |
| TI | 6958 | 3545 | | | | 2577 | 22791 | _1126159 | | | 6326 | 46 | | 05 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y2- | 2 | 215. | 215.13 | 0.0003 | 5.79E+ | |
| TI | 6958 | 3545 | | | | 2577 | 22791 | _1126159 | H2O | | 1387 | 9 | | 04 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y3- | 3 | 328. | 328.22 | 0.0021 | 1.29E+ | |
| TI | 6958 | 3545 | | | | 2577 | 22791 | _1126159 | H2O | | 221 | 31 | | 04 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y4- | 4 | 441. | 441.30 | −0.0009 | 3.07E+ | |
| TI | 6958 | 3545 | | | | 2577 | 22791 | _1126159 | H2O | | 308 | 71 | | 03 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y5- | 5 | 597. | 597.40 | 0.0009 | 5.93E+ | |
| TI | 6958 | 3545 | | | | 2577 | 22791 | _1126159 | H2O | | 4073 | 83 | | 04 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y6- | 6 | 698. | 698.45 | 0.0017 | 1.24E+ | |
| TI | 6958 | 3545 | | | | 2577 | 22791 | _1126159 | H2O | | 4542 | 59 | | 05 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y7- | 7 | 811. | 811.54 | −0.0001 | 1.89E+ | |
| TI | 6958 | 3545 | | | | 2577 | 22791 | _1126159 | H2O | | 54 | | | 05 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y8- | 8 | 924. | 924.62 | −0.0054 | 3.11E+ | |
| TI | 6958 | 3545 | | | | 2577 | 22791 | _1126159 | H2O | | 6295 | 4 | | 04 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y3- | 3 | 329. | 329.20 | −0.0146 | 5.69E+ | |
| TI | 6958 | 3545 | | | | 2577 | 22791 | _1126159 | NH3 | | 2213 | 67 | | 03 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y8- | 8 | 925. | 925.60 | −0.0118 | 6.56E+ | |
| TI | 6958 | 3545 | | | | 2577 | 22791 | _1126159 | NH3 | | 6194 | 76 | | 03 | |
| (SEQ ID | | | | | | | D2 | _1_1. | | | | | | | |
| NO: 3) | | | | | | | | final. | | | | | | | |
| | | | | | | | | contigs | | | | | | | |
| FLLTRIL | 1088. | 545. | 99.4 | 2 | 6 | F6:5 | ILS | S_837_bp | y8 | 8 | 471. | 471.81 | −0.0014 | 2.07E+ | |
| TI | 6958 | 3545 | | | | 2577 | 22791 | _1126159 | /2 | | 8187 | 73 | | 03 | |
| (SEQ ID | | | | | | | D2 | _1_1. | +/ | | | | | | |
| NO: 3) | | | | | | | | final. | | | | | | | |
| | | | | | | | | contigs | | | | | | | |
| FL | 1095. | 548. | 99.5 | 2 | 6 | F6:5 | ILS | S_837_bp | y1 | 1 | 132. | 132.10 | 0.0002 | 1.24E+ | 13C |
| (+7.02) | 7129 | 863 | 1 | | | 2641 | 22791 | _1126159 | | | 1017 | 19 | | 05 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y2 | 2 | 233. | 233.14 | 0.0006 | 3.47E+ | 13C |
| (+7.02) | 7129 | 863 | 1 | | | 2641 | 22791 | _1126159 | | | 149 | 96 | | 05 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y3 | 3 | 346. | 346.23 | 0.0001 | 8.63E+ | 13C |
| (+7.02) | 7129 | 863 | 1 | | | 2641 | 22791 | _1126159 | | | 2335 | 36 | | 03 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y5 | 5 | 615. | 615.41 | 0.0013 | 1.07E+ | 13C |
| (+7.02) | 7129 | 863 | 1 | | | 2641 | 22791 | _1126159 | | | 4175 | 88 | | 05 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y6 | 6 | 716. | 716.46 | 0.0007 | 9.08E+ | 13C |
| (+7.02) | 7129 | 863 | 1 | | | 2641 | 22791 | _1126159 | | | 4658 | 65 | | 05 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y7 | 7 | 829. | 829.55 | 0.0014 | 3.43E+ | 13C |
| (+7.02) | 7129 | 863 | 1 | | | 2641 | 22791 | _1126159 | | | 5491 | 0 5 | | 06 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y8 | 8 | 949. | 949.65 | 0.0009 | 4.73E+ | 13C |
| (+7.02) | 7129 | 863 | 1 | | | 2641 | 22791 | _1126159 | | | 6509 | 17 | | 05 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y2- | 2 | 215. | 215.13 | 0.0003 | 7.52E+ | 13C |
| (+7.02) | 7129 | 863 | 1 | | | 2641 | 22791 | _1126159 | H2O | | 1387 | 9 | | 04 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y3- | 3 | 328. | 328.22 | 0.0002 | 1.50E+ | 13C |
| (+7.02) | 7129 | 863 | 1 | | | 2641 | 22791 | _1126159 | H2O | | 2229 | 31 | | 04 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y4- | 4 | 441. | 441.30 | 0.0022 | 6.47E+ | 13C |
| (+7.02) | 7129 | 863 | 1 | | | 2641 | 22791 | _1126159 | H2O | | 3049 | 71 | | 03 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y5- | 5 | 597. | 597.40 | −0.0003 | 8.40E+ | 13C |
| (+7.02) | 7129 | 863 | 1 | | | 2641 | 22791 | _1126159 | H2O | | 4086 | 83 | | 04 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y6- | 6 | 698. | 698.45 | 0.0009 | 1.92E+ | 13C |
| (+7.02) | 7129 | 863 | 1 | | | 2641 | 22791 | _1126159 | H2O | | 4551 | 59 | | 05 | (6) |
| LTRILTI | | | | | | | D2 | _1_1. | | | | | | | 15N |
| (SEQ ID | | | | | | | | .final | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y7- | 7 | 811. | 811.54 | 0.0002 | 2.23E+ | 13C |
| (+7.02) | 7129 | 863 | 1 | | | 2641 | 22791 | _1126159 | H2O | | 5397 | | | 05 | (6) |
| LTRILTI | | | | | | | D2 | _1_1. | | | | | | | 15N |
| (SEQ ID | | | | | | | | .final | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y8- | 8 | 931. | 931.64 | −0.0049 | 4.35E+ | 13C |
| (+7.02) | 7129 | 863 | 1 | | | 2641 | 22791 | _1126159 | H2O | | 6461 | 12 | | 04 | (6) |
| LTRILTI | | | | | | | D2 | _1_1. | | | | | | | 15N |
| (SEQ ID | | | | | | | | .final | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y1- | 1 | 115. | 115.07 | −0.0115 | 1.89E+ | 13C |
| (+7.02) | 7129 | 863 | 1 | | | 2641 | 22791 | _1126159 | NH3 | | 0864 | 49 | | 03 | (6) |
| LTRILTI | | | | | | | D2 | _1_1. | | | | | | | 15N |
| (SEQ ID | | | | | | | | .final | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y3- | 3 | 329. | 329.20 | −0.0167 | 1.62E+ | 13C |
| (+7.02) | 7129 | 863 | 1 | | | 2641 | 22791 | _1126159 | NH3 | | 2234 | 67 | | 03 | (6) |
| LTRILTI | | | | | | | D2 | _1_1. | | | | | | | 15N |
| (SEQ ID | | | | | | | | .final | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y8- | 8 | 932. | 932.62 | 0.0083 | 1.01E+ | 13C |
| (+7.02) | 7129 | 863 | 1 | | | 2641 | 22791 | _1126159 | NH3 | | 6165 | 48 | | 04 | (6) |
| LTRILTI | | | | | | | D2 | _1_1. | | | | | | | 15N |
| (SEQ ID | | | | | | | | final. | | | | | | | (1) |
| NO: 3) | | | | | | | | contigs | | | | | | | Si- |
| | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |
| FL | 1095. | 548. | 99.5 | 2 | 6 | F6:5 | ILS | S_837_bp | y2 | 2 | 117. | 117.07 | −0.015 | 1.01E+ | 13C |
| (+7.02) | 7129 | 863 | 1 | | | 2641 | 22791 | _1126159 | /2 | | 0898 | 48 | | 03 | (6) |
| LTRIL | | | | | | | D2 | _1_1. | +/ | | | | | | 15N |
| TI* | | | | | | | | final. | | | | | | | (1) |
| (SEQ ID | | | | | | | | contigs | | | | | | | Si- |
| NO: 3) | | | | | | | | | | | | | | | lac |
| | | | | | | | | | | | | | | | la- |
| | | | | | | | | | | | | | | | bel, |
| | | | | | | | | | | | | | | | 7.0 |
| | | | | | | | | | | | | | | | 172, |
| | | | | | | | | | | | | | | | 2, L |

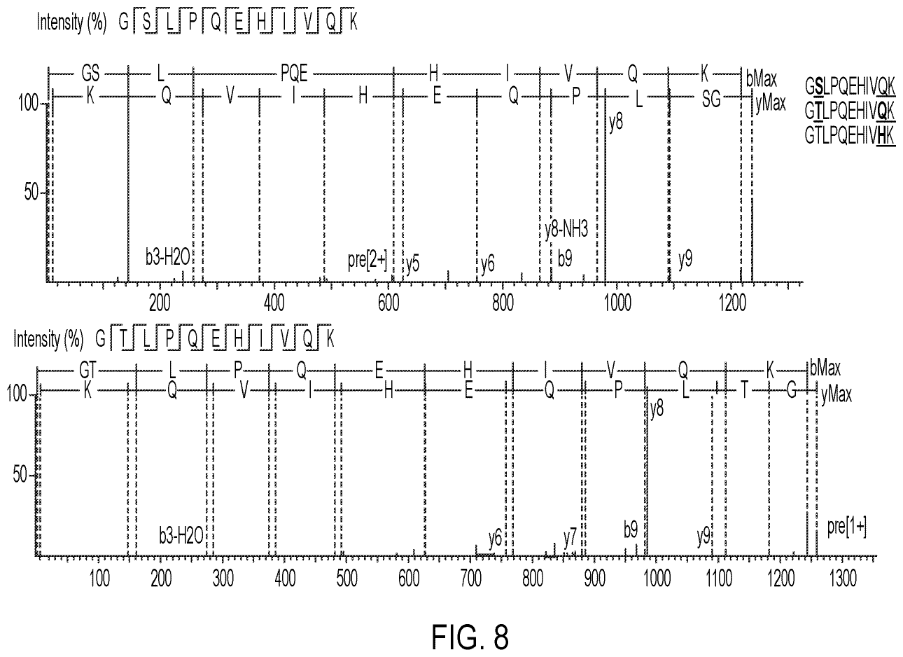

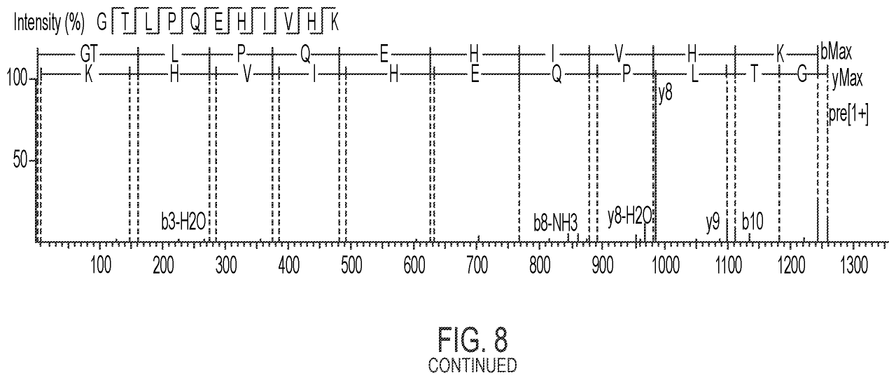

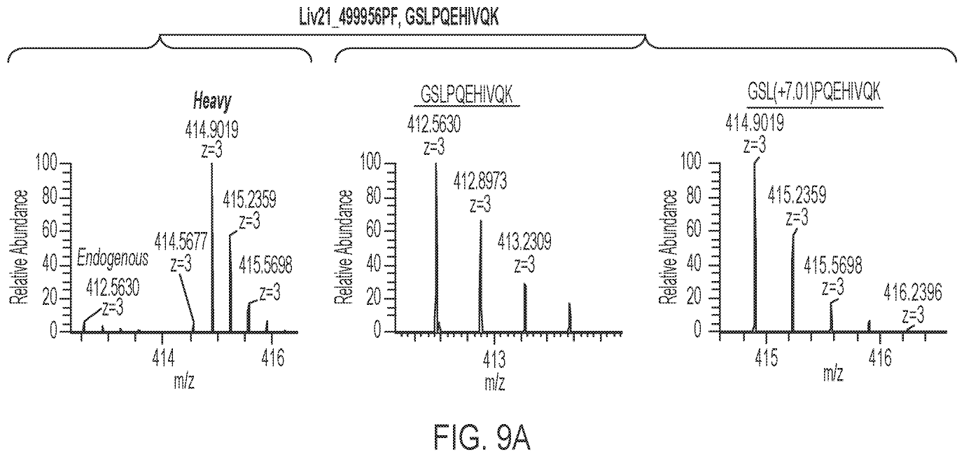

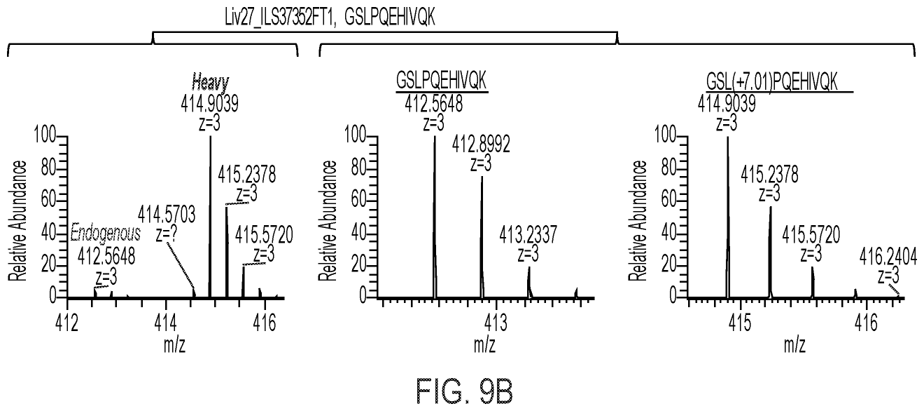

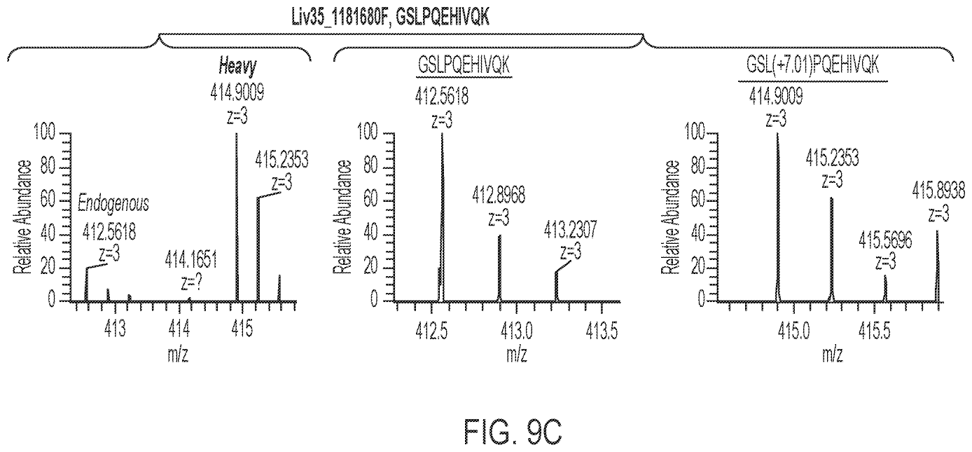

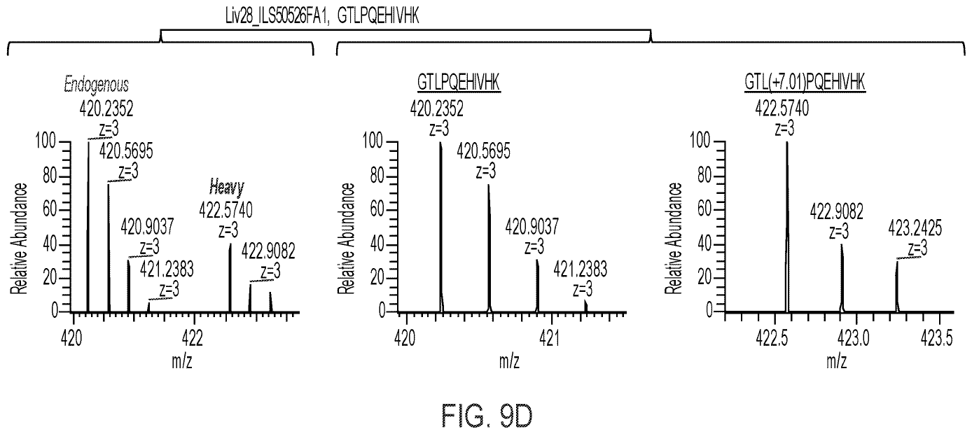

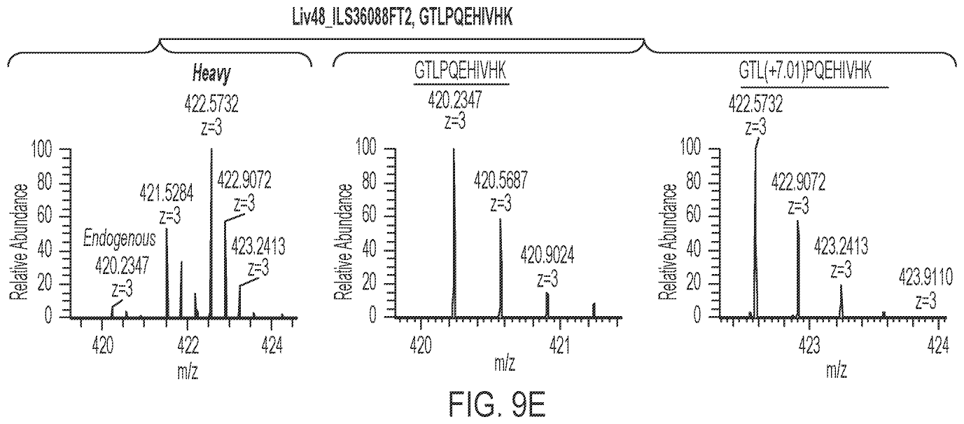

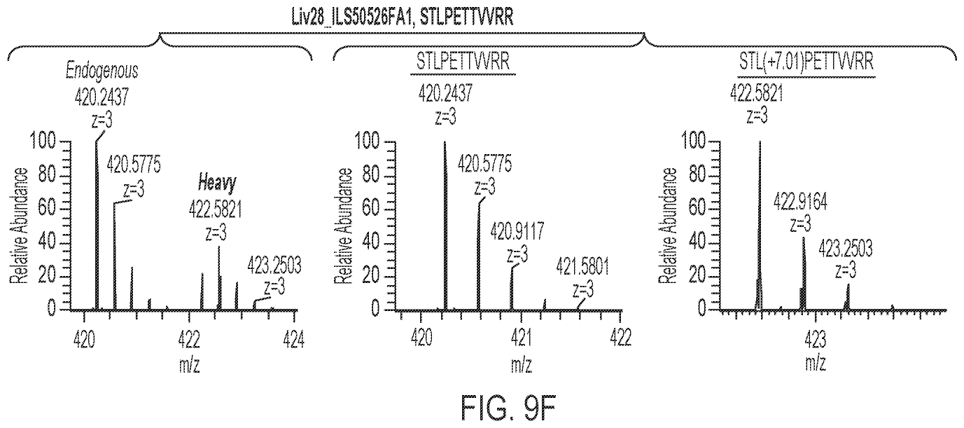

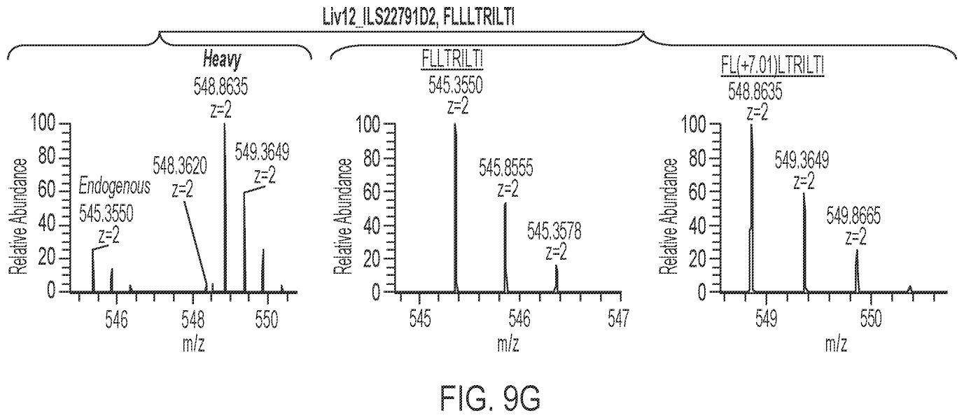

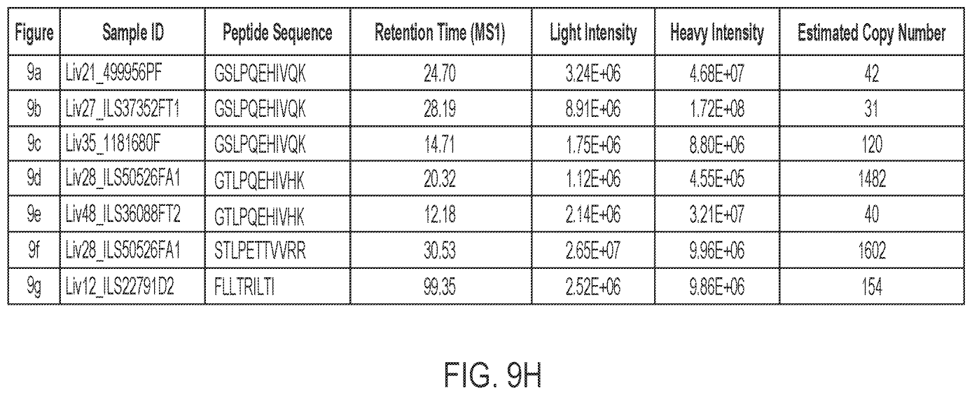

The synthetic peptides used for this analysis differed from the HBV peptides by a heavy Leucine (¹³C(6)¹⁵N(1), +7.0172) and were mixed (50 fmol) with HLA-eluted peptides prior to LC-MS/MS analysis. All peptides tested eluted at the same retention time and in the same MS1 scan as their heavy analogues in the LC-MS/MS run and also exhibited a similar fragmentation profile (FIGS. 9A-9H) and also exhibited a similar fragmentation profile. The extracted ion chromatogram was also utilized to estimate the expression levels or the copy number of some of the target peptides, including the polymerase peptide variants (copy number of ˜100-500/cell), from the intensity of corresponding heavy peptide analogue parent ions. Even at relatively low surface presentation, the three-polymerase peptide P₆₀₆₋₆₁₆ variants could be differentiated based on the fragment ions (FIG. 8).

The present disclosure describes the identification of unique HBV peptides. 34 unique peptides were detected from the envelope protein out of 49 total HLA-I restricted HBV specific peptides from patient liver samples. The detection of STLPETTVVRR (C₁₄₁₋₁₅₁) (SEQ ID NO: 43) from HBV capsid correlates with previous studies. By leveraging patient-specific databases generated by RNAseq analysis, peptide GSLPQEHIVQK (P₆₀₆₋₆₁₆) (SEQ ID NO: 12) and its variants from HBV polymerase, as the most frequent HBV epitope in the patient population analyzed, were identified. This peptide was a predicted HLA-A11 binder and appeared in more than 80% of the HLA-A11+ patient population described herein.

The reconstruction of the HBV genomes present in each sample of the present disclosure provided insight into regions of the HBV genome which can be integrated into hepatocytes during HCC development. The region of the genome encompassing the small surface antigen protein, X protein, and the latter half of the polymerase protein was frequently detected. The region of the genome encompassing the capsid protein and the first half of the polymerase protein were either not detected or only partially detected.

The combined use of the RNAseq data and LC/MS-MS data reported herein demonstrate an approach to finding and selecting appropriate peptide targets for T cell-based therapeutics. As a non-limiting example, the ideal peptide target can be highly abundant on the surface of cells, found commonly across patients, conserved across multiple HBV genotypes, and fall in a region of the HBV genome that is commonly integrated in HCC. Using the methods reported herein, peptides may be identified that fit all or certain aspects of the above-described criteria. Peptide GSLPQEHIVQK (P₆₀₆₋₆₁₆) (SEQ ID NO: 12) was frequently found in the HLA-A11 patient samples and falls in an HBV genome region that is commonly integrated. T cell-based therapeutics may be designed, e.g., around the regions of peptide GSLPQEHIVQK (P₆₀₆₋₆₁₆) (SEQ ID NO: 12) that are conserved. Use of the approach described herein to examine more patient samples representing different HLA alleles can lead to identification of peptides that fit all or certain aspects of the above-listed criteria.

Below are the methods used in the Examples described above.

HBV-Positive HCC Tissue Procurement. All patient tissues were purchased from BioIVT in fresh frozen form. The patient details including age, sex and diagnostic tests are provided in Table 4 and Tables 5A-5B. Tissues were cryopreserved until sample preparation.

Tissue Lysis, and HLA Affinity Enrichment. The tissues were pulverized using SPEX SamplePrep Freezer/Mill Dual-Chamber Cryogenix Grinder in liquid nitrogen, and lysed in ice-cold lysis buffer (1% NP-40, 150 mM NaCl, 50 mM Tris-HCl pH 8.0 and 10 mM EDTA pH 8.0) supplemented with HALT protease and phosphatase inhibitors on ice using sonication. The volume of lysis buffer was determined by the tissue weight (example 5 ml lysis buffer for 0.5 g tissue).

Anti-HLA Class I (W6/32) was conjugated to NHS-sepharose beads by overnight incubation in coupling buffer (0.2 M NaHCO₃+0.5 M NaCl pH 8.3) in 4° C. The reaction was quenched with 0.1 M Tris-HCl pH 8.5, and the beads were washed with the same Tris-HCl solution and 0.1 M acetate buffer.

The pre-cleared tissue lysate was passed through a column packed with 1 ml of HLA Class-I beads bed under gravity. The column was subsequently washed with Seppro Dilution Buffer and 20 mM Tris-HCl pH 8, and HLA-peptide complex was eluted with 0.1M glycine pH 2.7.

Sample Preparation for Mass Spectrometry. The glycine eluate was loaded onto the C18 Sep-Pak, followed by selective elution of peptides by 30% ACN/0.1% TFA. The peptides were further cleaned up, and analyzed by nano-LC-MS/MS.

Liquid Chromatography with tandem mass spectrometry (LC-MS/MS). HLA peptides as described above were loaded onto a nanoViper Acclaim PepMap100 C18 trap column (75 μm i.d.×2 cm, 3 μm, 100 Å) and were separated using a nanoViper Acclaim PepMap RSLC C18 column (75 μm i.d.×25 cm, 2 μm, 100 Å) heated to 40° C. and retrofitted with a New Objective SilicaTip (7 cm) with a distal conductive coating at the inlet end of the emitter. The gradient was delivered by an EASY-nLC 1200 HPLC system at 300 nL/min. The following 120-minute elution gradient with mobile phase A (Water/0.1% formic acid) and B (80% Acetonitrile/0.1% formic acid) was used: 3% B at 3 min, linear to 35% B at 100 min, and linear to 45% B at 123 min. The peptides eluted from the column were ionized via Flex ion source at 1.9 kV and analyzed by the Thermo Fusion Lumos Tribrid mass spectrometer using Xcalibur 4.1.31.9. The data acquisition was performed in data-dependent mode, where survey scans were carried out in the high field Orbitrap analyzer (range of m/z 300-1500 at a resolution of 60,000) with the automatic gain control target of 4.0E5 and maximal ion fill time of 100 ms. The MS/MS analyses were performed by 1.2 m/z precursor ion isolation with the quadrupole, applying normalized HCD (higher-energy collisional dissociation) collision energy of 32%, and analysis of fragment ions in the Orbitrap at a resolution of 15,000. The dynamic exclusion window was set to 6 seconds, monoisotopic precursor selection (MIPS) to peptide, maximum injection time to 100 ms, and charge states unknown. +1-+4 charge states were included and the advanced peak determination was toggled on.

For FAIMS-enabled experiments, the settings were identical except the FAIMS device was placed between the nanoelectrospray source and the mass spectrometer. FAIMS separations were performed with the following settings: inner and outer electrode temperature was set to 100° C. (except where noted), FAIMS carrier gas flow of 5.0 L/min, asymmetric waveform with DV −5000 V, entrance plate voltage 250 V, and CV settling time of 25 ms. The FAIMS carrier gas was N₂, and the ion separation gap is 1.5 mm. The noted CVs were applied to the FAIMS electrodes. For external stepping or single CV experiments, the selected CV was applied to all scans throughout the analysis. For internal CV stepping experiments, each of the selected CVs was applied to sequential survey scans and MS/MS cycles (1 s); the MS/MS CV was always paired with the appropriate CV from the corresponding survey scan.

HLA Genotyping for Patient Samples. For sample preparation and sequencing, DNA sample quantity was determined by fluorescence and quality was assessed by running 25 ng of sample on a 1% pre-cast agarose gel. Samples with high molecular weight gDNA with a majority of the DNA fragments greater than 20 kb and a concentration of no less than 10 ng/ul passed the quality assessment. The DNA samples were normalized to 10 ng/ul and 50 ng was used to amplify the full-length HLA amplicons. The targets were amplified in three pools of variant tolerant primers optimized for similar binding temperatures and PCR conditions with LA Taq DNA Polymerase. The resulting amplicon pools were combined equimolarly as determined by automated capillary electrophoresis and fluorescence. DNA libraries were prepared for Illumina-based sequencing with a custom NEB kit. The amplicons were enzymatically fragmented to a mean insert size of 250 bp and universal adapters were ligated onto the DNA fragments. Unique 10 base pair barcode sequences were added to the DNA fragments during PCR with NEBNext Ultra II Q5 Master Mix to facilitate highly multiplexed sequencing. The samples were pooled and sequenced using 150 base pair paired-end sequencing on an Illumina Nextseq 500.

For data analysis, upon completion of sequencing, raw data from each Illumina Nextseq run was gathered in local buffer storage and uploaded to a local high-performance computing platform for automated analysis. The FASTQ-formatted reads were converted from the BCL files and assigned to samples identified by specific barcodes using the bcl2fastq conversion software (Illumina Inc., San Diego, CA). All the reads in sample-specific FASTQ files were subjected to HLA typing analysis using an updated version of PHLAT program with the reference sequences consisting of GRCh38 genomic sequences and HLA type reference sequences in the IPD-IMGT/HLA database v3.30.0.

RNA sequencing. Total RNA was extracted from human liver tissue using MagMAX kit. Strand-specific RNA-seq libraries were prepared from 1 μg RNA using KAPA stranded mRNA-Seq Kit (KAPA Biosystems). Twelve-cycle PCR was performed to amplify libraries. The amplified libraries were size-selected at 400-600 bp using PippinHT. Sequencing was performed on Illumina HiSeq®2500 (Illumina) by multiplexed paired-read run with 2×100 cycles.

Mapping of Patient Liver RNA Sequences to HBV Reference Genome. Bulk RNA-seq reads were aligned to reference genome ayw (NC_003977.2) using minimap2 (v2.17). Alignments were then sorted by coordinates and quality controlled by samtools flagstats (v1.9) and bedtools genomeCoverageBed (v2.17.0) inspection. Duplicates were then marked and removed by the Picard toolkit (v2.18.2).

Workflow to Reconstruct HBV Genomes from Patient Liver RNAseq Data. Paired-end Illumina RNA reads from each sample were de novo assembled into large contigs using megahit (options: --min-count 3 --k-min 27 --k-max 127 --prune-level 2) and mapped using BLAST to HBV reference genome, ayw (NC_003977.2) to select HBV specific sequences. BLAST parameters for sequence comparisons included outfmt ‘7 std sgi stitle’; minimum E value=0.001; cost to open a gap=5; cost to extend a gap=2; length of best perfect match=11; reward for a nucleotide match=2; reward for a nucleotide mis-match=−3. Contigs without BLAST matches were discarded as well as BLAST results with E values greater than 0.001, percentage identity below 79% or alignment length of less than 50 nucleotides. Overlapping contigs were merged using custom scripts and final sequence that covered the entire or partial length of the reference sequence with the highest identity was selected. The final sequence was mapped against the reference genome to extract and translate all coding sequences. Non-redundant protein sequences were then added to customized databases for peptide identification by mass-spectrometry.

Workflow to identify integration sites from Patient Liver RNAseq Data. Contigs matching the HBV reference genome (ayw) were extracted and mapped against human reference genome (GRCh38) using BLAST with parameters described above. Contigs matching both HBV and human (hybrid contigs) were collected for annotation. The HBV and human integration sites of the hybrid contigs were annotated against the genome features in HBV ayw GenBank file and human GRCh38 GTF file.

Identification of HBV Peptides from HLA-I Immunopeptidomics of Patient Liver Samples. All mass spectrometry raw files were searched against a consolidated database of human UniProtKB (Homo sapiens) and HBV protein sequences, obtained from RNAseq of patient liver samples as described above, with PEAKS DB search engine. PEAKSX+ (PEAKS Studio 10.5, Bioinformatics Solutions Inc.) was used for de-novo-assisted database search with precursor mass tolerance of 8 ppm, and fragment ion tolerance of 0.02 Da. Enzyme selectivity was set to none, and methionine oxidation as the only variable modification with two maximum allowed modifications per peptide. The search was performed with a 5% false discovery rate (FDR) at peptide level, and peptides were further filtered based on −log P score of 20 (corresponding to 1% FDR).

### Example 5. Identification of HBV Antigens or Epitopes

The present Example relates to nucleic acid assay methods and compositions for identifying HBV antigens or epitopes.

RNA or DNA is sequenced from a cancer patient's liver tumor and/or healthy tissue to identify sequences which may contain HBV-associated insertions in genes expressed in the tumor cell.

Tumor material is assessed for HBV-associated insertions by sequencing such as by RNA sequencing. Sequencing data are used to investigate HBV-associated insertions expressed in genes. Peptide stretches comprising any of the identified HBV-associated insertions are created in silico and are filtered through the application of prediction algorithms or used to identify MHC-associated epitopes via mass spectrometry data. Where appropriate, suitable MHC-binding HBV epitope sets are used to identify physiologically relevant HBV epitope-specific T cell responses via functional assays and/or MHC multimer-based screens within both or either of CD8+ and/or CD4+ T cell populations.

HBV epitopes are determined by sequencing the genome and/or exome of tumor tissue and/or healthy tissue from a liver cancer patient using next generation sequencing (NGS) approaches. Genes selected based on the presence of HBV-associated insertions and ability to act as an antigen are sequenced using NGS technology. NGS applies to, without limitation, genome sequencing, genome resequencing, epigenome characterization, DNA-protein interactions (ChIP-sequencing), and transcriptome profiling (RNA-Seq). Similar to DNA-based assays using, e.g., NGS or massively parallel sequencing (MPS) approaches, RNA from tumors is analyzed by conversion to cDNA and generation of a library suitable for sequencing.

Assays are employed to identify HBV epitopes in biological samples. Suitable assays used to identify HBV epitopes in biological samples include, but are not limited to, proteomics, NGS, solution hybridization, array hybridization nucleic amplification, polymerase chain reaction (PCR), RT-PCR, quantitative PCR, branched DNA (bDNA) assay, rolling circle amplification (RCA), in situ hybridization, Northern hybridization, hybridization protection assay (HPA), single molecule hybridization detection, Invader assay, and/or Oligo Ligation Assay (OLA), hybridization, and array analysis.

The sequencing data derived from determining the presence of HBV epitopes in a cancer patient is analyzed to predict personal HBV peptides that can bind to HLA molecules of the individual. The data are analyzed using a computer. The sequence data are analyzed, in particular, for the presence of HBV antigens.

HBV antigens are assessed by their affinity to MHC molecules.

Neural network-based learning approaches with validated binding and non-binding peptides are used in prediction algorithms for the major HLA-A and -B alleles. Algorithms are used for predicting missense mutations that create strong binding peptides to a cancer patient's cognate MHC molecules. A set of peptides representative of optimal HBV epitopes for each patient is identified and prioritized. HBV epitope prediction algorithms are used to predict binding of candidate peptides to MHC class I molecules or MHC class II molecules.

In some cases, a peptide binding tool can be one of the following: Antibody Epitope Prediction, ANTIGENIC, BepiPred, CTLPred, DiscoTope, EPIPREDICT, Epitope Cluster Analysis, Epitope Conservancy Analysis, EUiPro, HLA Peptide Binding Predictions, HLABinding, MAPPP, MHCBench, MHC-I processing predictions, Mosaic Vaccine Tool Suite, NetChop, NetCTL, NetMHC, NetMHCII, NetMHCpan, nHLAPred-I, OptiTope, PAProC, POPI, PREDEP, Prediction of Antigenic Determinants, ProPred, ProPred-1, RankPep, SMM, SVMHC, TAPPred, VaxiJen, or combinations thereof. Additional exemplary programs are used such BIMAS or SYFPEITHI, Rankpep.

In some cases, the Immune Epitope Database and Analysis Resource (IEDB) (Vita R, et al. Nucleic Acids Res. 2015; 43 (Database issue): D405-D412) is used to identify a suitable tumor HBV antigen. Such algorithms predict peptide binding to different MHC class I variants based on artificial neural networks (ANN), providing predicted IC50 as an output. NetMHC (Lundegaard C, et al. Nucleic Acids Res. 2008; 36 (Web Server issue): W509-W512.) is used. Programs such as SMMPMBEC (Kim Y, et al. BMC Bio informatics. 2009; 10:394) and/or SMM (Peters B, et al. BMC Bio informatics. 2005; 6: 132) are used. These programs use position-weight matrices to describe statistical preferences from peptide-MHC I binding data. This approach suppresses noise caused, for example, by a limited number of data points present in the training set and/or experimental error.

In some instances, single nucleotide polymorphisms SNPs are removed from candidate HBV antigens or HBV epitopes. SNPs contain a range of molecular variation: (1) SNPs, (2) multinucleotide polymorphisms (MNPs), (3) short deletion and insertion polymorphisms (indels/DIPs), (4) micro satellite markers or short tandem repeats (STRs), (5) heterozygous sequences, and (6) named variants.

Proteomic-based methods for identifying tumor specific HBV antigens such as direct protein sequencing are used. Protein sequencing of enzymatic digests using multidimensional MS techniques including tandem mass spectrometry (MS/MS) is used to identify HBV antigens. High-throughput methods for de novo sequencing of unknown proteins is used, for example, to analyze the proteome of a cancer patient's tumor to identify expressed HBV antigens. In some instances, meta-shotgun protein sequencing is used to identify expressed HBV antigens.

Tumor specific HBV antigens are identified using MHC multimers to identify HBV antigen-specific T-cell responses. For example, high-throughput analysis of HBV antigen-specific T-cell responses in cancer patient samples may be performed using MHC tetramer-based screening approaches. Such tetramer-based screening approaches are used for the identification of tumor specific HBV antigens, or as a secondary screening protocol to assess HBV antigens to which a patient may have already been exposed, which may support the selection of candidate HBV antigens. Where appropriate, filters are applied to eliminate (1) epitopes with lower binding affinity than the corresponding wild-type sequences and/or (2) epitopes predicted to be poorly processed by the immunoproteasome. Candidate mutated peptides are synthesized and screened to identify T cell HBV antigens.

Pulsing antigen presenting cells (APCs) with relatively long synthetic peptides that encompass minimal T cell epitopes is used to identify HBV epitopes. Nonsynonymous mutated epitopes are identified in tumors by evaluating the response of CD4+ tumor infiltrating lymphocytes (TIL) to autologous B cells that are pulsed with peptides encompassing individual mutations. Use of this approach results in the identification of mutated cell epitopes. A peptide screening assay is performed based on the combination of two peptide libraries: (1) overlapping long-peptides (2) peptides according to MHC-binding prediction. Screening leads to identification of mutated HBV-reactive T cells isolated from liver tumor.

A tandem minigene screening approach is used to identify HBV epitopes. A tandem minigene construct comprised, for example, without limitation 6 to 24 minigenes that encoded polypeptides comprising a mutated amino acid residue flanked on the N- and/or C-terminus by, e.g., 12 amino acids. Tandem minigene constructs are synthesized and used to transfect autologous APCs and/or cell lines co-expressing autologous HLA molecules. Using this approach, HBV epitopes are identified in cancer patients, e.g., hepatocellular carcinoma (HCC) patients.

HBV epitopes are identified using an approach combining whole-exome/transcriptome sequencing analysis, MHC binding prediction, and mass spectrometric technique to detect peptides eluted from HLA molecules. Predicted high-binding peptides are confirmed by mass spectrometry.

### Example 6. Determination of MHC Binding Capacity

Candidate peptides according to the present disclosure are tested for their MHC binding capacity (affinity). The individual peptide-MHC (pMHC) complexes are produced by UV-ligand exchange. A UV-sensitive peptide is cleaved upon UV-irradiation, and exchanged with the peptide of interest. Peptide candidates that effectively bind and stabilize the peptide-receptive MHC molecules prevent dissociation of the MHC complexes. To determine the yield of the exchange reaction, an ELISA is performed based on the detection of the light chain (β2m) of stabilized MHC complexes. Briefly, 96-well plates are coated with streptavidin, washed, and blocked. Refolded HLA-A monomers serve as standards, covering a pre-determined concentration range. Peptide-MHC monomers of the UV-exchange reaction are diluted in blocking buffer. Samples are incubated, washed, incubated with HRP conjugated anti-β2m, washed again and detected with a chromogenic substrate solution that is stopped per the manufacturer's protocol. Absorption is measured, for example, at 450 nm. Candidate peptides that show a high exchange yield are preferred for generation and production of antibodies or fragments thereof, and/or T cell receptors or fragments thereof. Candidate peptides demonstrate avidity to the MHC molecules and prevent dissociation of the MHC complexes.

### Example 7. Preparation of Peptide-MHC (pMHC) Complexes

This example relates to a method for the preparation of soluble recombinant HLA loaded with an HBV-derived peptide.

Class I HLA molecules (HLA-heavy chain and HLA light-chain (β2m)) are expressed separately in E. coli as inclusion bodies using suitable expression vectors. HLA-heavy chain additionally comprises a C-terminal biotinylation tag which replaces, for example, the transmembrane and/or cytoplasmic domains. E. coli cells are lysed and inclusion bodies processed to approximately 80% purity.

Inclusion bodies of β2m and heavy chain are denatured separately in denaturation buffer. Refolding buffer is prepared. Synthetic peptides are dissolved to a final concentration and added to the refold buffer. Then β2m followed by heavy chain are added. Refolding is performed to completion.

The refold mixture is then dialyzed. The protein solution is subsequently filtered through a filter and loaded onto an exchange column (pre-equilibrated). Protein is eluted such as by way of a linear salt gradient using additional purifier. HLA-peptide complex is eluted, and peak fractions are collected. A cocktail of protease inhibitors is added and the fractions are chilled on ice.

Biotin-tagged pHLA molecules are buffer exchanged into a buffer using a fast desalting column equilibrated in the same buffer. Upon elution, the protein-containing fractions are chilled on ice and protease inhibitor cocktail is added. Biotinylation reagents are then added. The mixture is then allowed to incubate.

The biotinylated pHLA molecules are further purified, for example, by gel filtration chromatography using purifier with a column pre-equilibrated with filtered PBS. The biotinylated pHLA mixture is concentrated to a final volume, loaded onto the column and developed. Biotinylated pHLA molecules elute, e.g., as a single peak. Fractions containing protein are pooled, chilled on ice, and protease inhibitor cocktail is added. Protein concentration is determined and aliquots of biotinylated pHLA molecules are stored frozen.

Such peptide-MHC (pMHC) complexes are used in soluble form or immobilized through their C-terminal biotin moiety on to a solid support, to be used for the detection of T cells and T cell receptors which bind the peptide-MHC complex. For example, such complexes are used in panning phage libraries, performing ELISA assays and/or preparing sensor chips for measurements of affinity and binding kinetics.

### Example 8. Identification of T Cell Receptors (TCRs) that Bind to pMHC Complexes

Antigen binding T cell receptors (TCRs) are obtained using peptides disclosed herein to pan a TCR phage library. The library is constructed using α- and β-chain sequences obtained from a natural repertoire. The random combination of these α- and β-chain sequences occurs during library creation, thereby producing a non-natural repertoire of α/β chain combinations.

TCRs obtained from the library are assessed by enzyme-linked immunoassay (ELISA) to confirm specific antigen recognition. ELISA plates are coated with streptavidin and incubated with the biotinylated peptide-HLA complex. TCR-bearing phage clones are added to each well and detection is carried out using an HRP antibody conjugate. Bound antibody is detected using a peroxidase Substrate System. An absence of binding to alternative peptide-HLA complexes indicated that the TCR is not highly cross reactive.

Further confirmation that TCRs can bind a peptide-HLA complex of the disclosure is obtained by surface plasmon resonance (SPR) using isolated TCRs. In this case α- and β-chain sequences are expressed in E. coli as soluble TCRs. Binding of the soluble TCRs to the complexes is analyzed by surface plasmon resonance. Biotinylated peptide-HLA monomers are prepared and immobilized on a streptavidin-coupled sensor chip. To measure affinity, serial dilutions of the soluble TCRs are flowed over the immobilized peptide-HLAs and the response values at equilibrium are determined for each concentration. Data are analyzed, for example, by plotting the specific equilibrium binding against protein concentration followed by a least squares fit to the Langmuir binding equation, assuming a 1:1 interaction.

TCRs that specifically recognize peptide-HLA complexes of the disclosure are obtained from the library. Data generated according to the above-described experiments confirm that antigen specific TCRs can be isolated.

### Example 9. Characterization of Binding to MHC and Stability of pMHC Complex

T2 cell-based peptide binding assay. T2 cells which do not express the transporter associated with antigen processing (TAP), and as such do not assemble stable MHC class I on the cell surface, are pulsed with different concentrations of peptides (controls or HBV peptide of interest [POI]), washed, detected with fluorescently-tagged antibody recognizing MHC class I (e.g., A2 allele), and run through a FACS Scan analyzer. The difference between the MFI (mean fluorescence intensity) corresponding to a given concentration of POI and the negative control (non-MHC binder) is a function of the number of stabilized pMHC complexes displayed on the cell surface. Therefore, at limiting concentrations of the peptide, it is largely a measurement of Kₒₙ, and at saturation levels of the peptide it is a measurement of both Kₒₙ and K_{off}. The binding is quantified by two related factors: relative affinity (1/RA) and half maximal binding (the peptide concentration responsible for 50% of the signal corresponding to saturation). Relative affinity, RA, is binding normalized to a reference (e.g., a wild-type peptide in instances where a mutant POI is being tested), e.g., the ratio between half maximal binding of control relative to POI. The higher the 1/RA index and the lower the half maximal binding, the higher the Kₒₙ of the interaction between the POI and the MHC.

Characterization of binding and stability by ELISA. Avidin-coated microtiter plates containing class I monomer loaded with a placeholder peptide are used to evaluate peptide binding, affinity, and off-rate. The monomer-coated plates are supplied as part of a kit, e.g., the iTopia Epitope Discovery System Kit. Assay buffers, anti-MHC-FITC mAb and β2-microglobulin and control peptides are also supplied with the kits.

Binding assay: POIs are first evaluated for their ability to bind each MHC molecule by binding assay. This assay measures the ability of individual peptides to bind HLA molecules under optimal standardized binding conditions. Monomer-coated plates are first stripped, releasing the placeholder peptide and leaving only the MHC heavy chain bound to the plate. Test peptides are then introduced under optimal folding conditions, along with the anti-MHC-FITC mAb. Plates were incubated. The anti-MHC-FITC mAb binds preferentially to a refolded MHC complex. Thus, the fluorescence intensity resultant from each peptide is related to the peptide's capacity to complex with MHC molecule. Each peptide's binding is evaluated relative to a positive control peptide, and the results are expressed, for example, as percent (%) binding.

Affinity assay: For the affinity assay, after the initial stripping of the placeholder peptide, increasing concentrations of POI are added to a series of wells and incubated under the conditions described previously. Plates are read on the fluorometer. Dose response curves are generated. The amount of peptide required to achieve 50% of the maximum is recorded as ED50 value.

Off-Rate assay: Plates are washed after incubation under conditions to remove excess peptide. The plates are then incubated on allele-specific monomer plates. The plates are measured at multiple time points (e.g., 0, 0.5, 1, 1.5, 2, 4, 6 and 8 hrs) for relative fluorescence intensity. The time required for 50% of the peptide to dissociate from the MHC monomer is defined as the T½ value (hrs).

iScore calculation: an iScore is a multi-parameter calculation provided within the iTopia software. Its value is calculated based on the binding, affinity, and stability data.

### Example 10. Determination of Responses Against Tumor Cells

A suitable number of groups of mice are immunized with a plasmid expressing HBV peptides disclosed herein by direct inoculation. By way of a non-limiting example, mice are inoculated into lymph nodes, e.g., inguinal lymph nodes, with plasmids at an appropriate concentration at day 0, and at subsequent days over the time course of the experiment, e.g., at days 3, 14, and 17. In certain cases, this is followed by one or more additional peptide boost(s) on following days, e.g., days 28 and 31, using a negative control peptide and POI. Splenocytes are stimulated ex vivo with POI and tested against Chromium-51 (⁵¹Cr)⁻-labeled tumor cells at various E:T ratios.

### Example 11. In Vivo Assessment of Enhanced Immunity Against HBV Peptides

A suitable number of groups of mice are immunized with a plasmid expressing HBV peptides disclosed herein by direct inoculation. By way of a non-limiting example, mice are inoculated into lymph nodes, e.g., inguinal lymph nodes, with plasmids at an appropriate concentration at day 0, and at subsequent days over the time course of the experiment, e.g., at days 3, 14, and 17. In certain cases, this is followed by one or more additional peptide boost(s) on following days, e.g., days 28 and 31, using a negative control peptide and POI.

To evaluate the in vivo response against HBV peptides, splenocytes were isolated from littermate control mice and incubated with one or more appropriate concentrations of POI for a pre-determined period of time. These cells were then stained with CFSEhi fluorescence and intravenously co-injected into immunized mice with an equal number of control splenocytes stained with CFSElo fluorescence. After a pre-determined period of time, the specific elimination of target cells was measured by removing spleen and peripheral blood mononuclear cells (PBMCs) from challenged animals and measuring CFSE fluorescence by flow cytometry. The relative depletion of the populations corresponding to peptide loaded splenocytes was calculated relative to the control (unloaded) population and expressed as percent (%) specific lysis.

### Example 12. Testing of POI's Increased Immunogenicity and Ability to Overcome Tolerization

POIs are used in in vitro for immunization of blood to generate cytotoxic T lymphocytes (CTLs).

PBMCs from normal donors are purified from buffy coats by centrifugation in standard sterile medium designed for isolating lymphocytes. Cultures are carried out using autologous plasma (AP). For in vitro generation of peptide-specific CTL, autologous dendritic cells (DCs) are used as antigen presenting cells (APCs). DCs are generated and CTLs are induced with DCs and peptides from PBMCs. Monocyte-enriched cell fractions are cultured to induce maturation. Specific numbers of CD8+-enriched T lymphocytes and peptide-pulsed DCs are co-cultured. Cultures are restimulated on various days with autologous irradiated peptide-pulsed DCs. Immunogenicity is assayed using in vitro cytotoxicity and cytokine production assays.

### Example 13. CD8+ T Cell Responses Against Peptides

Based on the predicted or experimentally verified HLA-binding peptides, whether T cells can be generated to recognize the tumor-specific peptides is determined. Peptides with appropriate binding scores are synthesized. To generate T cells of desired specificity, T cells are stimulated with peptide-pulsed (individual peptide or peptide pool) autologous APCs such as dendritic cells and/or CD40L-expanded autologous B cells on a predetermined schedule, for example, in the presence of IL-2 and IL-7. After a number of rounds of stimulation, the expanded CD8+ cells are tested on ELISpot for evidence of reactivity against the peptide based on IFNγ secretion.

### Example 14. Cytokine Production Assay

For cytokine (e.g., IL-2 and IFNγ) production assays, T cells are harvested after contact with the peptide-pulsed APC, centrifuged, and both cell pellets and supernatants are collected. Cytokine production is measured by ELISA from the supernatant.

### Example 15. In Vivo Activation of Viral Peptide-Specific T Cells as a Method to Limit Viral Production

For in vivo studies, the ability of the viral peptide in the context of MHC I molecules (e.g., HLA-A2, HLA-A24, HLA-A11) to enhance a CD8+ T cell response as a method to limit viral production following AAV-HBV challenge is tested in transgenic mice that express human HLA molecule(s):

(i) MHC class I molecule(s) displaying the HBV viral peptide of interest (POI) or (ii) a control MHC class I molecule displaying the OVA₂₅₇₋₂₆₄ peptide (OVA) are generated.

A cohort of transgenic mice that express human HLA molecule(s) is initially injected with the MHC I molecule/peptide or MHC I molecule/OVA complexes and the level of effector and memory CD8+ T cells is monitored at different time points post-injection (from spleen and/or blood). Mock injected mice will serve as a negative control group in this experiment. A second cohort of mice are then injected with the MHC I molecule/POI complex and subsequently challenged using AAV-HBV, at post-injection time points that will be determined according to the measurements of effector and memory CD8+ T cells levels from the first experiment. Control groups are constituted with (i) mice not injected with the MHC I molecule/POI complex but challenged with AAV-HBV, (ii) mice injected with MHC I molecule/OVA complexes, and (iii) mice injected with MHC I molecule/POI complex but not challenged with AAV-HBV. The viral burden is analyzed at different time points post-injection to determine MHC I molecule/POI limitation of viral infection from CD8+ T cells activation.

### Example 16. In Vivo Activation of Antigen-Specific T Cells by the Viral Peptide as a Method to Decrease Viral Production

For in vivo studies, the ability of the HBV viral peptide of interest (POI) in the context of MHC I molecules (e.g., HLA-A2, HLA-A24, HLA-A11) to enhance a T cell response against HBV virus following AAV-HBV challenge is tested in transgenic mice that express human HLA molecule(s). More particularly, the in vivo activation of peptide-specific CD8+ T cells after (i) POI immunization, (ii) acute challenge with AAV-HBV, and/or (iii) chronic challenge with AAV-HBV, is compared.

(i) MHC class I molecule(s) displaying the HBV viral POI or (ii) a control MHC class I molecule displaying the OVA₂₅₇₋₂₆₂ peptide (OVA) are generated.

Transgenic mice that express human HLA molecule(s) are first challenged with AAV-HBV. The MHC class I molecule/peptide or MHC class I molecule/OVA complexes produced for this example are then injected 1 day and 7 days after infection with the acute challenge, and 1 day, 7 days, and 14 days after the chronic challenge. The viral burden is analyzed at different time points post-peptide injection by blood collection and post-mortem organs collection. At those time points, the levels of effector and memory CD8+ T cells are measured and compared to the control conditions to determine the capacity of MHC class I molecule/peptide to decrease viral production in response to an AAV-HBV challenge.

### Example 17. Enhancement of a Peptide-Specific CD8+ T Cell Immunity in a Peptide-Presenting Tumor Model

For in vivo studies, the ability of the HBV peptide of interest in the context of MHC I molecules to enhance a CD8+ T cell response against peptide-presenting tumors is tested in a general purpose strain mice, e.g., C57Bl/6 mice, grafted with a peptide-expressing tumor cell line.

The peptide-expressing tumor cell line is first grown in vitro, then injected (e.g., subcutaneously) into the mice. After tumor cell injection, when tumors are of palpable size, the HBV peptide, for example, in a specific groove of the MHC I or control peptide (e.g., ovalbumin [OVA] peptide) in an alternate groove of the MHC I molecule, are injected intratumorally. Tumor cell volume is measured. Tumor, blood, and/or spleen is collected, and homogenized in single cell suspensions, when applicable. The level of CD8+ T cells in the homogenized samples is determined at all suitable time points.

Below are additional example methods that may be used in accordance with the disclosure.

Splenocytes and antigen presenting cell primary cultures. Spleens of adult general purpose strain mice, e.g., C57Bl/6 mice, are excised and placed in cold buffer. Tissues are then homogenized to break apart the spleens. Dissociated cells are centrifuged. After centrifugation, the cell pellet is resuspended in a suitable volume of lysis buffer designed to remove red blood cells, e.g., ACK (Ammonium-Chloride-Potassium) lysis buffer, and incubated in the buffer. After incubation, the cell suspension is added to buffer, and centrifuged. The cell pellet is then resuspended in buffer and the cell solution is filtered. The filtered cell suspension is centrifuged again, and the subsequent pellet is resuspended in a suitable volume of buffer. Total spleen cells are counted, and global antigen-presenting cells (APC) are sorted using anti-MHC class II Microbeads and appropriate sorting technology. After elution, the MHC class II-positive cell fraction is collected and resuspended at a given concentration in APC cell medium.

Pulsing of APCs with peptides. APC are incubated with HBV or control peptides. Peptide-pulsed APC are harvested and washed prior to addition to any of various suitable T cell lines (e.g., J.RT3-T3.5 derived cells), after infection with the viral particles. The T cells are co-cultured with the pulsed APC and subsequently used in proliferation, luciferase and/or cytokines production assays.

T cell activation post transduction. Alternatively, to co-culture with peptides-pulsed APCs, suitable T cell lines (e.g., J.RT3-T3.5-derived cell lines) are activated after transduction with either phytohemagglutinin (PHA), phorbol 12-myristate 13-acetate (PMA), or a combination of PHA and PMA.

Another alternative method to activate T cells after transduction is to use soluble or immobilized antibodies such as, but not limited to anti-CD3 and/or anti-CD28 monoclonal antibodies. Soluble antibodies are added at an appropriate concentration. In some experiments, plates pre-coated with anti-CD3 antibodies, for example, are used in combination with soluble anti-CD28 antibody.

Another alternative method to activate T cell lines is activating beads coupled with anti-CD3/anti-CD28 antibodies. After transduction, infected cells are counted, and anti-CD3/anti-CD28 beads are added to the culture medium at a given ratio.

Cells staining and FACS analysis. Fluorescence-activated cell sorting (FACS) is performed after the transduction. Transduced cells are counted and seeded into a cell culture plate. Cells are spun, washed, and then spun again any number of times. Cells are incubated with a dye for determining viability of cells, e.g., Live/Dead™ Fixable Near-IR stain, washed, and incubated with Fc block in buffer, e.g., FACS stain buffer. After being washed any number of times again with FACS stain buffer, cells are subsequently incubated with antibodies such as, but not limited to, fluorescent-labeled antibodies targeting TCR and control. Cells are washed with FACS stain buffer, fixed, washed again, and resuspended in FACS stain buffer. Samples are run for analysis with a FACS analyzer.

Proliferation and cytokine production assays. To measure T cell proliferation, 3H-thymidine is added to assay cultures following contact with the peptide-pulsed APC. Following incubation, cultures are harvested onto filter bottom microplates. MicroScint 20 scintillation fluid or the like is added to each well, and plates are counted on a Scintillation Counter.

For transcription factor activity analysis and cytokines production assay, cells are harvested after contact with the peptide-pulsed APC, centrifuged, and cell pellets and supernatants are collected.

Cell pellets are processed for RNA extraction for transcriptomics analysis via qPCR. Cytokine production is measured by ELISA from the supernatant. Cell pellets are processed for luciferase detection assay to measure programmed cell death, e.g., AP1 activity.

Splenocytes isolation and CD8+ T cell culture. Spleens of adult general purpose strain mice, e.g., C57Bl/6 mice, are excised and placed in cold buffer. Tissues are then homogenized to break apart the spleens. Dissociated cells are centrifuged. After centrifugation, the cell pellet is resuspended in a suitable volume of lysis buffer designed to remove red blood cells, e.g., ACK (Ammonium-Chloride-Potassium) lysis buffer, and incubated in the buffer. After incubation, the cell suspension is added to buffer, and centrifuged. The cell pellet is then resuspended in buffer and the cell solution is filtered. The filtered cell suspension is centrifuged again and the subsequent pellet is resuspended in a suitable volume of buffer. CD8+ T cells are then isolated such as, for example, by using a CD8a+ T cell Isolation Kit.

Peptide immunization of mice. For immunization, a suitable amount of HBV peptide or control peptide is diluted in buffer and emulsified at a given ratio with an adjuvant such as, but not limited to, complete Freund's adjuvant (CFA) or incomplete Freund's adjuvant (IFA) using, for example, a double syringes system, to reach a final desired volume of peptide/adjuvant emulsion. A measured volumed (e.g., 200 μl) of the peptide/adjuvant emulsion is then injected by a given route of administration, for example, subcutaneously, into mice (e.g., C57Bl/6 mice) under appropriate anesthesia in appropriate location(s).

### Quantification of Cytotoxicity Activity by Flow Cytometry.

Preparation of target cells: For preparation of target cells (e.g., autologous B cells), the target cells are isolated from splenocytes of OT-1 or P14 mice by using a Mouse B cell Kit (or the like, according to the manufacturer's instruction), and are subsequently stimulated for a predetermined duration in the presence of a set concentration of IFN-γ. Targets cells are then counted, divided into tubes, and washed in buffer. A proportion of the target cells are stained with a high concentration (e.g., 0.2 M) of CFSE (CFSEHigh) and a separate proportion with a low concentration (e.g., 0.02 M) of CFSE (CFSELow) in buffer. After the incubation, cells are pelleted and resuspended in an appropriate media to quench the labeling reaction. Target cells stained with the low concentration of CFSE are pulsed by adding the HBV peptides or control OVA peptides at a suitable final concentration under appropriate culture conditions. Both target cells stained with different concentration of CFSE are then washed, resuspended at a measured concentration in media and mixed with an appropriate ratio, such as a 1:1 ratio (CFSEHigh:CFSELow).

Effector cells: Total CD8+ T cells containing the effector cells are enriched from splenocytes of P14 and OT-1 mice using a Negative Selection Human CD8 T cell isolation Kit. CD8+ T cells are counted, resuspended in complete T cell medium, and serially diluted volume:volume in a given volume of complete T cell medium. From each dilution, a given volume of cells are seeded in duplicate to multi-well cell culture plate. The mixed target cells are added to each dilution of effector cells. To measure basal apoptosis, a number of wells are seeded with target cells alone. Cell mixtures are incubated under standard cell culture conditions.

Flow cytometry staining and acquisition: Cells are transferred to multi-well cell culture plate, washed in FACS staining buffer and stained with, for example, iTag Tetramer/APC-H-2Kb OVA9@(MBL), iTag Tetramer/APC-H-2Db HBV® Alexa Fluor® and Live/Dead® Fixable Near-IR stain (ThermoFisher) under appropriate conditions. Cells are then washed in FACS stain buffer before staining with, for example, Fluorescently-labeled monoclonal antibody that specifically binds to CD8 alpha such as, but not limited to, BV421 αCD8a. Cells are washed with FACS stain buffer and resuspended fixative. Acquisition is performed and all cells are acquired. Post-acquisition data analysis performed.

Preparation of vectors encoding peptides. A therapeutic DNA or RNA vaccine comprising polynucleotides or vectors encoding polynucleotides to be used is prepared by GMP manufacturing of the plasmid vaccine according to regulatory authorities' guidelines. The vaccine is appropriately formulated, for example, by dissolving in a saline solution, at a suitable concentration. The vaccine may be administered either intradermal or intramuscular with or without following electroporation or alternatively with a jet injector.

### Example 18. Flow Cytometry Binding of a Pan HLA-A Antibody Against K562/HLA-A11 Cells Pulsed with HBV POL Peptides

The present Example was designed to assess loading of HBV POL₆₀₆₋₆₁₆ peptides on HLA-A11 in K562 cells engineered to express human HLA-A11 (K562/HLA-A11 cells). To assess loading of HBV POL₆₀₆₋₆₁₆ peptides on HLA-A11, K562 cells engineered to express human HLA-A11 (K562/HLA-A11 cells) were pulsed by resuspending the cells in AIM V medium (Gibco. Catalog #31035-025) at a density of 1×10⁶ cells/ml followed by the addition of 10 μg/ml human β2-microglobulin (hB2M; EMID Millipore Catalog #475828) and either 100 μg/ml or 33 μg/ml of the indicated peptide (Table 14).

TABLE 14
List of Peptides Tested

| | Sequence of Peptide |
|---|---|
| Name of Peptide | amino acid) |
| HBV POL₆₀₆₋₆₁₆ V1 | GSLPQEHIVQK |
| | (SEQ ID NO: 12) |
| HBV POL₆₀₆₋₆₁₆ V2 | GTLPQEHIVHK |
| | (SEQ ID NO: 13) |
| HBV POL₆₀₆₋₆₁₆ V3 | GTLPQEHIVQK |
| | (SEQ ID NO: 14) |
| HBV POL₆₀₆₋₆₁₆ V4 | GSLPQEHIIQK |
| | (SEQ ID NO: 11) |
| HBV POL₆₀₆₋₆₁₆ V5 | GTLPQDHIVQK |
| | (SEQ ID NO: 110) |
| HBV POL₆₀₆₋₆₁₆ V6 | GSLPQDHIIQK |
| | (SEQ ID NO: 111) |
| HBV POL₆₀₆₋₆₁₆ V7 | GTLPQEHIVLK |
| | (SEQ ID NO: 112) |

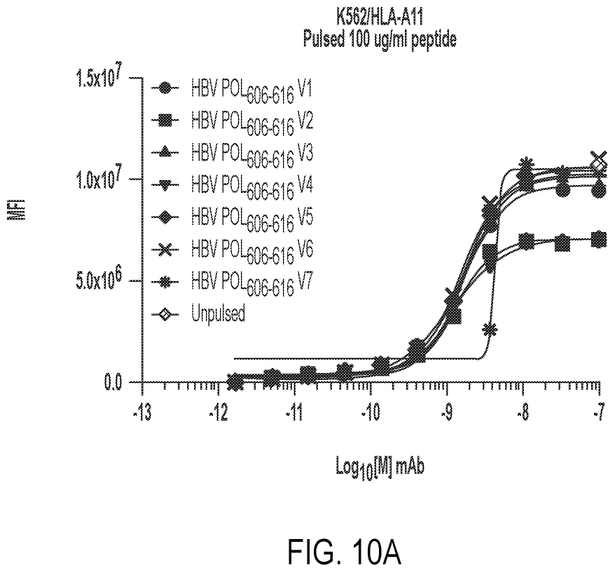

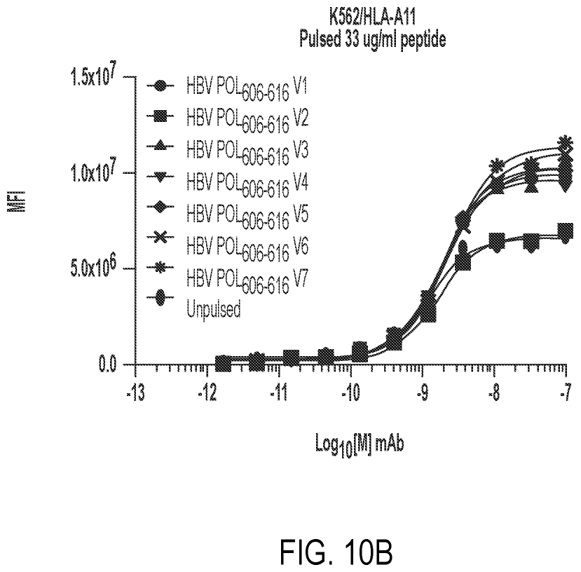

K562/HLA-A11 cells were then incubated overnight at 37° C. Peptide-pulsed cells were then harvested and plated in staining buffer (PBS, without Calcium and Magnesium (Corning, Reference #21-031-CV)+2% FBS (Seradigm, Lot #238B15)) at a density of 200,000 cells per well in a 96-well V-Bottom plate. Cells were incubated with three-fold serial dilutions (5.1 pM-100 nM) with a pan HLA-A antibody (Novus, Catalog #DDX0250P-100) for 30 minutes at 4° C., washed once in staining buffer, and incubated with an Alexa-647 conjugated secondary antibody (Jackson ImmunoResearch, Catalog #115-606-071) at 5 g/ml for 30 minutes at 4° C. Cells were again washed once in staining buffer and stained with viability dye (CellTrace™ Violet Cell Proliferation Kit, Thermo-Fisher Cat #C34557) for 30 minutes at 4° C. Lastly, cells were washed once in staining buffer and fixed using a 50% solution of BD Cytofix (BD, Catalog #554655). Samples were run and analyzed on an IntelliCyt iQue flow cytometer to calculate mean fluorescence intensity (MFI) on live cells. MFI values were plotted in GraphPad Prism. The secondary antibody alone condition (i.e., no primary antibody) for each dose-response curve was also included in the analysis as a continuation of the three-fold serial dilution and was represented as the lowest dose. MFI binding curves are shown in FIGS. 10A-10B. The signal-to-noise (S/N) was calculated by dividing the maximum MFI on the pulsed cells by the maximum MFI on un-pulsed K562/HLA-A11 cells (Table 15). Peptides were determined to load if the S/N from pulsed cells/un-pulsed cells was greater than 1.1. All peptides except HBV POL₆₀₆₋₆₁₆ V2 (GTLPQEHIVHK (SEQ ID NO: 13)) had a S/N>1.1 indicating that the peptides loaded on HLA-A11.

TABLE 15
Cell Binding S/N of a Pan-HLA-A Antibody to K562
cells Pulsed with HBV POL₆₀₆₋₆₁₆ Peptides

| | S/N | S/N |
|---|---|---|
| | Peptide | Peptide |
| | Concentration | Concentration |
| Peptide | (100 μg/ml) | (33 μg/ml) |
| HBV POL₆₀₆₋₆₁₆ V1 | 1.4 | 1.6 |
| HBV POL₆₀₆₋₆₁₆ V2 | 1.1 | 1.0 |
| HBV POL₆₀₆₋₆₁₆ V3 | 1.5 | 1.6 |
| HBV POL₆₀₆₋₆₁₆ V4 | 1.5 | 1.5 |
| HBV POL₆₀₆₋₆₁₆ Pol V5 | 1.5 | 1.7 |
| HBV POL₆₀₆₋₆₁₆ Pol V6 | 1.6 | 1.6 |
| HBV POL₆₀₆₋₆₁₆ Pol V7 | 1.6 | 1.7 |
| Un-pulsed | 1.0 | 1.0 |

The claimed subject matter is not to be limited in scope by the specific embodiments described herein. Indeed, various modifications of the claimed subject matter in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An isolated peptide comprising an amino acid sequence that is at least 90% identical to the amino acid sequence of any one of SEQ ID NOs: 1-54 and 110-112, or a pharmaceutically acceptable salt thereof, or a fragment or derivative thereof, wherein the isolated peptide is 8-12 amino acids in length.

2. The isolated peptide of claim 1, wherein the isolated peptide comprises an amino acid sequence of any one of SEQ ID NOs: 1-54 and 110-112.

3. The isolated peptide of claim 1 or 2, wherein the isolated peptide consists essentially of an amino acid sequence of any one of SEQ ID NOs: 1-54 and 110-112.

4. The isolated peptide of any one of claims 1-3, wherein the isolated peptide consists of an amino acid sequence of any one of SEQ ID NOs: 1-54 and 110-112.

5. An isolated peptide comprising two or more amino acid sequences selected from any one of SEQ ID NO: 1-54 and 110-112, or a pharmaceutically acceptable salt thereof, or a fragment or derivative thereof.

6. An isolated peptide, wherein the isolated peptide consists of an amino acid sequence GX₁LPQX₂HIX₃X₄K (SEQ ID NO: 107), wherein X₁ is S or T, X₂ is E or D, X₃ is V or I, and X₄ is Q, H or L, or a pharmaceutically acceptable salt thereof, or a fragment or derivative thereof.

7. The isolated peptide of any one of claims 1-6, wherein the isolated peptide comprises one or more reverse peptide bonds, one or more non-peptide bonds, one or more D-isomers of amino acids, one or more chemical modifications, or any combination thereof.

8. The isolated peptide of any one of claims 1-7, wherein the isolated peptide is produced by expression in a heterologous host cell.

9. The isolated peptide of any one of claims 1-7, wherein the isolated peptide is produced synthetically.

10. The isolated peptide of any one of claims 1-9, wherein the isolated peptide, or pharmaceutically acceptable salt thereof, or fragment or derivative thereof induces a hepatitis B virus (HBV)-specific immune response in a subject when presented in a complex with a major histocompatibility complex (MHC) molecule on the surface of an antigen presenting cell (APC).

11. A fusion protein comprising one or more isolated peptides of any one of claims 1-10 fused to one or more heterologous molecules.

12. The fusion protein of claim 11, wherein the one or more heterologous molecules enhance a peptide-specific immune response in a subject.

13. The fusion protein of claim 11, wherein the one or more heterologous molecules mediate peptide delivery to a specific site within a subject.

14. The fusion protein of any one of claims 11-13, wherein the one or more heterologous molecules are a MHC molecule, or a fragment or derivative thereof.

15. A conjugate comprising one or more isolated peptides of any one of claims 1-10 conjugated to one or more heterologous molecules.

16. The conjugate of claim 15, wherein the one or more heterologous molecules enhance a peptide-specific immune response in a subject.

17. The conjugate of claim 15, wherein the one or more heterologous molecules mediate peptide delivery to a specific site within a subject.

18. The conjugate of any one of claims 15-17, wherein the one or more heterologous molecules are an MHC molecule, or a fragment or derivative thereof.

19. The conjugate of any one of claims 15-17, wherein the one or more peptides are conjugated to a particle.

20. An oligomeric complex comprising two or more isolated peptides of any one of claims 1-10.

21. A non-covalent complex comprising the isolated peptide of any one of claims 1-10 and an MHC molecule, or a fragment or derivative thereof.

22. The non-covalent complex of claim 21, wherein the MHC molecule, or the fragment thereof, is a class I MHC molecule.

23. The non-covalent complex of claim 22, wherein the class I MHC molecule is a class I human leukocyte antigen (HLA) molecule.

24. The non-covalent complex of claim 21, wherein the MHC molecule, or the fragment thereof, is a class II MHC molecule.

25. The non-covalent complex of claim 24, wherein the class II MHC molecule is a class II HLA molecule.

26. A fusion protein comprising the isolated peptide of any one of claims 1-10 and an MHC molecule, or a fragment or derivative thereof.

27. The fusion protein of claim 26, wherein the MHC molecule, or the fragment thereof, is a class I MHC molecule.

28. The fusion protein of claim 27, wherein the class I MHC molecule is a class I human leukocyte antigen (HLA) molecule.

29. The fusion protein of claim 28, wherein the MHC molecule, or the fragment thereof, is a class II MHC molecule.

30. The fusion protein of claim 29, wherein the class II MHC molecule is a class II HLA molecule.

31. A conjugate comprising the isolated peptide of any one of claims 1-10 and a MHC molecule, or a fragment or derivative thereof.

32. The conjugate of claim 31, wherein the MHC molecule, or the fragment thereof, is a class I MHC molecule.

33. The conjugate of claim 32, wherein the class I MHC molecule is a class I human leukocyte antigen (HLA) molecule.

34. The conjugate of claim 31, wherein the MHC molecule, or the fragment thereof, is a class II MHC molecule.

35. The conjugate of claim 34, wherein the class II MHC molecule is a class II HLA molecule.

36. A pharmaceutical composition comprising (i) one or more isolated peptides of any one of claims 1-10, one or more fusion proteins of any one of claims 11-14 and 26-30, one or more conjugates of any one of claims 15-19 and 31-35, one or more oligomeric complexes of claim 20, or one or more non-covalent complexes of any one of claims 21-25, or any combination thereof, and (ii) a pharmaceutically acceptable carrier or excipient.

37. The pharmaceutical composition of claim 36, further comprising an adjuvant.

38. An isolated molecule that binds the isolated peptide of any one of claims 1-10, the fusion protein of any one of claims 11-14 and 26-30, the conjugate of any one of claims 15-19 and 31-35, the oligomeric complex of claim 20, or the non-covalent complex of any one of claims 21-25.

39. The isolated molecule of claim 38, wherein the molecule is an antibody or an antigen-binding fragment thereof.

40. The isolated molecule of claim 39, wherein the antibody is a bispecific antibody.

41. The isolated molecule of claim 38, wherein the molecule is an alternative scaffold.

42. The isolated molecule of claim 38, wherein the molecule is a chimeric antigen receptor (CAR).

43. The isolated molecule of claim 38, wherein the molecule is a T cell receptor (TCR).

44. An isolated cell comprising the CAR of claim 42.

45. The isolated cell of claim 44, wherein the isolated cell is an immune cell.

46. The isolated cell of claim 45, wherein the immune cell is a T cell, an NK cell, or a macrophage.

47. An isolated cell comprising the TCR of claim 43.

48. The isolated cell of claim 47, wherein the isolated cell is an immune cell.

49. The isolated cell of claim 48, wherein the immune cell is a T cell, an NK cell, or a macrophage.

50. A pharmaceutical composition comprising (i) the isolated molecule of any one of claims 38-43, or the isolated cell of any one of claims 44-49; and (ii) a pharmaceutically acceptable carrier or excipient.

51. An isolated polynucleotide comprising a nucleotide sequence encoding one or more isolated peptides of any one of claims 1-10 or the fusion protein of any one of claims 11-14 and 26-30.

52. The isolated polynucleotide of claim 51, wherein the nucleotide sequence is operably linked to a promoter.

53. The isolated polynucleotide of claim 51 or claim 52, wherein the isolated polynucleotide comprises DNA.

54. The isolated polynucleotide of claim 51 or claim 52, wherein the isolated polynucleotide comprises RNA.

55. The isolated polynucleotide of claim 54, wherein the RNA is mRNA.

56. The isolated polynucleotide of claim 54, wherein the RNA is self-replicating RNA.

57. A vector comprising the isolated polynucleotide of any one of claims 51-56.

58. The vector of claim 57, wherein the vector is an expression vector.

59. The vector of claim 57 or claim 58, wherein the vector is a viral vector.

60. A host cell comprising the isolated polynucleotide of any one of claims 51-56 or the vector of any one of claims 57-59.

61. The host cell of claim 60, wherein the host cell is a prokaryotic cell.

62. The host cell of claim 60, wherein the host cell is a eukaryotic cell.

63. The host cell of claim 62, wherein the host cell is an APC.

64. A pharmaceutical composition comprising (i) the isolated polynucleotide of any one of claims 51-56, or the vector of any one of claims 57-59; and (ii) a pharmaceutically acceptable carrier or excipient.

65. The pharmaceutical composition of claim 64, wherein the pharmaceutically acceptable carrier is a lipid nanoparticle carrier.

66. A method of inducing an immune response against a hepatitis B viral (HBV) infection in a subject in need thereof, the method comprising administering to the subject a therapeutically effective amount of:
a) one or more isolated peptides of any one of claims 1-10;
b) the fusion protein of any one of claims 11-14 and 26-30;
c) the conjugate of any one of claims 15-19 and 31-35;
d) the oligomeric complex of claim 20;
e) the non-covalent complex of any one of claims 21-25;
f) the pharmaceutical composition of any one of claims 36, 37, 50, 64, and 65;
g) the molecule of any one of claims 38-43;
h) the isolated cell of any one of claims 44-49 and 60-61;
i) the isolated polynucleotide of any one of claims 51-56; or
j) the vector of any one of claims 57-59.

67. A method of inducing an immune response against a hepatitis B viral (HBV) infection in a subject in need thereof, the method comprising administering to the subject a therapeutically effective amount of one or more isolated peptides of any one of claims 1-10.

68. A method of inducing an immune response against an HBV infection in a subject in need thereof, comprising administering to the subject an activated T cell that is produced by contacting a T cell with an APC that presents the isolated peptide of any one of claims 1-10 in complex with an MHC molecule.

69. A method of treating an HBV-induced disease or disorder in a subject in need thereof, the method comprising administering to the subject an effective amount of:
a) one or more isolated peptides of any one of claims 1-10;
b) the fusion protein of any one of claims 11-14 and 26-30;
c) the conjugate of any one of claims 15-19 and 31-35;
d) the oligomeric complex of claim 20;
e) the non-covalent complex of any one of claims 21-25;
f) the pharmaceutical composition of any one of claims 36, 37, 50, 64, and 65;
g) the molecule of any one of claims 38-43;
h) the isolated cell of any one of claims 44-49 and 60-63;
i) the isolated polynucleotide of any one of claims 51-56; or
j) the vector of any one of claims 57-59.

70. A method of preventing or reducing the likelihood of an HBV-induced disease or disorder in a subject in need thereof, the method comprising administering to the subject an effective amount of:
a) one or more isolated peptides of any one of claims 1-10;
b) the fusion protein of any one of claims 11-14 and 26-30;
c) the conjugate of any one of claims 15-19 and 31-35;
d) the oligomeric complex of claim 20;
e) the non-covalent complex of any one of claims 21-25;
f) the pharmaceutical composition of any one of claims 36, 37, 50, 64, and 65;
g) the molecule of any one of claims 38-43;
h) the isolated cell of any one of claims 44-49 and 60-63;
i) the isolated polynucleotide of any one of claims 51-56; or
j) the vector of any one of claims 57-59.

71. A method of treating an HBV-induced disease or disorder in a subject in need thereof, the method comprising administering to the subject an effective amount of one or more isolated peptides of any one of claims 1-10.

72. A method of preventing or reducing the likelihood of an HBV-induced disease or disorder in a subject in need thereof, the method comprising administering to the subject an effective amount of one or more isolated peptides of any one of claims 1-10.

73. The method of any one of claims 70-72, wherein the HBV-induced disease or disorder is a liver inflammation, liver fibrosis, liver cirrhosis, or liver cancer.

74. The method of claim 73, wherein the liver cancer is hepatocellular carcinoma (HCC).

75. A kit comprising:
(i) a) one or more isolated peptides of any one of claims 1-10;
b) the fusion protein of any one of claims 11-14 and 26-30;
c) the conjugate of any one of claims 15-19 and 31-35;
d) the oligomeric complex of claim 20;
e) the non-covalent complex of any one of claims 21-25;
f) the pharmaceutical composition of any one of claims 36, 37, 50, 64, and 65;
g) the molecule of any one of claims 38-43;
h) the isolated cell of any one of claims 44-49 and 60-63;
i) the isolated polynucleotide of any one of claims 51-56; or
j) the vector of any one of claims 57-59; and
(ii) packaging and/or instructions for use for the same.

76. A method for identifying an immunogenic virus-derived peptide, the method comprising:
a) obtaining a plurality of RNA contig sequences derived from an infected subject infected with a virus, wherein the plurality of RNA contig sequences comprises a plurality of virus-derived RNA contig sequences and a plurality of infected-subject endogenous RNA contig sequences;
b) identifying the plurality of virus-derived RNA contig sequences from within the plurality of RNA contig sequences;
c) assembling a viral RNA sequence based on the plurality of virus-derived RNA contig sequences;
d) identifying a protein sequence based on the viral RNA sequence; and
e) identifying the immunogenic virus-derived peptide based at least in part on the identified protein sequence.

77. The method of claim 76, wherein the plurality of RNA contig sequences are derived from one infected subject.

78. The method of claim 76, wherein the infected subject is a human.

79. The method of claim 76, wherein the plurality of virus-derived RNA contig sequences are derived from the virus infecting the infected subject.

80. The method of claim 76, wherein the plurality of infected-subject endogenous RNA contig sequences are derived from RNA endogenous to the infected subject.

81. The method of claim 76, wherein the identifying the plurality of virus-derived RNA contig sequences from within the plurality of RNA contig sequences comprises:
comparing at least a portion of contig sequences of the plurality of RNA contig sequences to a reference viral sequence; and
identifying the plurality of virus-derived RNA contig sequences such that each contig sequence of the plurality of virus-derived RNA contig sequences comprises at least a portion that corresponds to the reference viral sequence.

82. The method of claim 81, wherein each contig sequence of the plurality of virus-derived RNA contig is distinct from the plurality of infected-subject endogenous RNA contig sequences.

83. The method of claim 81, wherein each contig sequence of the plurality of virus-derived RNA contig sequences lacks infected-subject endogenous RNA contig sequences.

84. The method of claim 81, wherein the reference viral sequence comprises a reference genome.

85. The method of claim 84, wherein the reference genome comprises a hepatitis B virus genome.

86. The method of claim 76, wherein the assembling the viral RNA sequence based on the plurality of virus-derived RNA contig sequences comprises:
overlapping common sequence portions at ends of at least a portion of the plurality of virus-derived RNA contig sequences such that the at least a portion of the plurality of virus-derived RNA contig sequences overlap linearly to assemble the viral RNA sequence.

87. The method of claim 76, wherein the identifying a protein sequence based on the viral RNA sequence such that the identified protein sequence includes a translation of the viral RNA sequence comprises:
identifying the protein sequence without requiring a comparison to a database of viral proteins.

88. The method of claim 76, wherein the identifying the protein sequence based on the viral RNA sequence such that the identified protein sequence includes a translation of the viral RNA sequence further comprises:
identifying a plurality of protein sequences each based on the viral RNA sequence such that each of the plurality of protein sequences respectively include a translation of the viral RNA sequence, and identifying the protein sequence as a frequently occurring protein sequence within the plurality of protein sequences.

89. The method of claim 76, wherein the protein sequence identified based on the viral RNA sequence is associated with a single infected subject.

90. The method of claim 89, wherein the identifying the immunogenic virus-derived peptide based at least in part on the protein sequence comprises:
identifying an MHC molecule associated with the single infected subject;
identifying one or more peptides based at least in part on the protein sequence such that the one or more peptides each form a respective MHC-peptide complex with the MHC molecule; and
identifying the immunogenic virus-derived peptide based on the one or more peptides.

91. A non-transitory computer-readable medium configured to communicate with one or more processor(s) of a computational device, the non-transitory computer-readable medium including instructions thereon, that when executed by the processor(s), cause the computational device to:
a) receive, as an input, a plurality of RNA contig sequences derived from an infected subject infected with a virus such that the plurality of RNA contig sequences comprise a plurality of virus-derived RNA contig sequences and a plurality of infected-subject endogenous RNA contig sequences, and wherein the infected subject is infected with a virus;
b) identify the plurality of virus-derived RNA contig sequences from within the plurality of RNA contig sequences;
c) assemble a viral RNA sequence based on the plurality of virus-derived RNA contig sequences;
d) identify a protein sequence based on the viral RNA sequence;
e) identify an immunogenic virus-derived peptide based at least in part on the protein sequence; and
f) provide, as an output, the immunogenic virus-derived peptide.

92. The non-transitory computer-readable medium of claim 91, wherein the plurality of RNA contig sequences are derived from only one infected subject.

93. The non-transitory computer-readable medium of claim 91, wherein the infected subject comprises a human.

94. The non-transitory computer-readable medium of claim 91, wherein the plurality of virus-derived RNA contig sequences are derived from the virus infecting the infected subject.

95. The non-transitory computer-readable medium of claim 91, wherein the plurality of infected-subject endogenous RNA contig sequences are derived from RNA endogenous to the infected subject.

96. The non-transitory computer-readable medium of claim 91, wherein the instructions which cause the computational device to identify the plurality of virus-derived RNA contig sequences from within the plurality of RNA contig sequences further comprise instructions, that when executed by the processor(s), cause the computational device to:
compare at least a portion of contig sequences of the plurality of RNA contig sequences to a reference viral sequence; and
identify the plurality of virus-derived RNA contig sequences such that each contig sequence of the plurality of virus-derived RNA contig sequences comprises at least a portion that corresponds to the reference viral sequence.

97. The non-transitory computer-readable medium of claim 96, wherein each contig sequence of the plurality of virus-derived RNA contig is distinct from the plurality of infected-subject endogenous RNA contig sequences.

98. The non-transitory computer-readable medium of claim 96, wherein each contig sequence of the plurality of virus-derived RNA contig sequences lacks portions of infected-subject endogenous RNA contig sequences.

99. The non-transitory computer-readable medium of claim 96, wherein the reference viral sequences comprises a reference genome.

100. The non-transitory computer-readable medium of claim 99, wherein the reference genome comprises a hepatitis B virus genome.

101. The non-transitory computer-readable medium of claim 91, wherein the instructions which cause the computational device to assemble the viral RNA sequence based on the plurality of virus-derived RNA contig sequences further comprise instructions, that when executed by the processor(s), cause the computational device to:
overlap common sequence portions at ends of at least a portion of the plurality of virus-derived RNA contig sequences such that the at least a portion of the plurality of virus-derived RNA contig sequences overlap linearly to assemble the viral RNA sequence.

102. The non-transitory computer-readable medium of claim 91, wherein the instructions which cause the computational device to identify a protein sequence based on the viral RNA sequence such that the protein sequence includes a translation of the viral RNA sequence further comprise instructions, that when executed by the processor, cause the computational device to:
identify the protein sequence without requiring a comparison to a database of viral proteins.

103. The non-transitory computer-readable medium of claim 91, wherein the protein sequence identified based on the viral RNA sequence is a novel protein.

104. The non-transitory computer-readable medium of claim 91, wherein the protein sequence identified based on the viral RNA sequence is associated with a single infected subject.

105. The non-transitory computer-readable medium of claim 104, wherein the instructions which cause the computational device to identify a protein sequence based on the viral RNA sequence such that the protein sequence includes a translation of the viral RNA sequence further comprise instructions, that when executed by the processor(s), cause the computational device to:
identify a plurality of protein sequences each based on the viral RNA sequence such that each of the plurality of protein sequences respectively include a translation of the viral RNA sequence; and
identify the protein sequence as a frequently occurring protein sequence within the plurality of protein sequences.

106. The non-transitory computer-readable medium of claim 104, wherein the instructions which cause the computational device to identify the immunogenic virus-derived peptide based at least in part on the protein sequence further comprise instructions, that when executed by the processor(s), cause the computational device to:
identify a major histocompatibility complex (MHC) molecule associated with the single infected subject;
identify one or more peptides based at least in part on the protein sequence such that the one or more peptides are each capable of forming a respective MHC-peptide complex with the MHC molecule; and
identify the immunogenic virus-derived peptide based on the one or more peptides.

107. The non-transitory computer-readable medium of claim 91, wherein the instructions, when executed by the processor(s), further cause the computational device to:
store the protein sequence to a database such that the protein sequence is associated with the infected subject within the database.

108. A method for identifying an integration site of a viral gene within a subject gene, the method comprising:
a) obtaining a plurality of RNA contig sequences derived from an infected subject infected with a virus such that the plurality of RNA contig sequences comprise a plurality of virus-derived RNA contig sequences, a plurality of infected-subject endogenous RNA contig sequences, and a plurality of hybrid RNA contig sequences comprising viral and infected-subject endogenous portions;
b) identifying the plurality of hybrid RNA contig sequences from within the plurality of RNA contig sequences;
c) comparing, for at least a portion of the plurality of hybrid RNA contig sequences, infected-subject endogenous portions to a subject reference genome; and
d) identifying, based at least in part on the comparison of infected-subject endogenous portions to the subject reference genome, an integration site comprising the subject gene.

109. The method of claim 108, wherein the plurality of RNA contig sequences are derived from one infected subject.

110. The method of claim 108, wherein the infected subject is a human.

111. The method of claim 108, wherein the plurality of virus-derived RNA contig sequences are derived from the virus infecting the infected subject.

112. The method of claim 111, wherein the virus is a hepatitis B virus.

113. The method of claim 108, wherein the plurality of infected-subject endogenous RNA contig sequences are derived from RNA endogenous to the infected subject.

114. The method of claim 108, wherein the subject reference genome comprises the human genome.

115. A non-transitory computer-readable medium configured to communicate with one or more processor(s) of a computational device, the non-transitory computer-readable medium including instructions thereon, that when executed by the processor(s), cause the computational device to:
a) receive, as an input, a plurality of RNA contig sequences derived from an infected subject infected with a virus such that the plurality of RNA contig sequences comprise a plurality of virus-derived RNA contig sequences, a plurality of infected-subject endogenous RNA contig sequences, and plurality of hybrid RNA contig sequences comprising viral and infected-subject endogenous portions;
b) identify the plurality of hybrid RNA contig sequences from within the plurality of RNA contig sequences;
c) compare, for at least a portion of the plurality of hybrid RNA contig sequences, infected-subject endogenous portions to a subject reference genome;
d) identify, based at least in part on the comparison of infected-subject endogenous portions to the subject reference genome, an integration site comprising a subject gene; and
e) provide, as an output, the integration site.

116. The non-transitory computer-readable medium of claim 115, wherein the plurality of RNA contig sequences are derived from only one infected subject.

117. The non-transitory computer-readable medium of claim 115, wherein the infected subject comprises a human.

118. The non-transitory computer-readable medium of claim 115, wherein the plurality of virus-derived RNA contig sequences are derived from a virus infecting the infected subject.

119. The non-transitory computer-readable medium of claim 118, wherein the virus comprises a hepatitis B virus.

120. The non-transitory computer-readable medium of claim 115, wherein the plurality of infected-subject endogenous RNA contig sequences are derived from RNA endogenous to the infected subject.

121. The non-transitory computer-readable medium of claim 115, wherein the subject reference genome comprises the human genome.
